(19) United States
(12) Patent Application Publication
(10) Pub. No.: US 2025/0105357 A1
(43) Pub. Date: Mar. 27, 2025

(54) ELECTROLYTE SOLVENTS AND METHODS FOR LITHIUM METAL AND LITHIUM ION BATTERIES

(71) Applicant: The Board of Trustees of the Leland Stanford Junior University, Stanford, CA (US)

(72) Inventors: Yi CUI, Stanford, CA (US); Zhenan BAO, Stanford, CA (US); Yuelang CHEN, Stanford, CA (US); Zhiao YU, Stanford, CA (US); Yangju LIN, Stanford, CA (US)

(73) Assignee: The Board of Trustees of the Leland Stanford Junior University, Stanford, CA (US)

(21) Appl. No.: 18/702,761

(22) PCT Filed: Oct. 21, 2022
(86) PCT No.: PCT/US2022/047472

§ 371 Date: Apr. 18, 2024

Related U.S. Application Data
(60) Provisional application No. 63/270,506, filed on Oct. 21, 2021.
(60) Provisional application No. 63/283,828, filed on Nov. 29, 2021.

(51) Int. Cl. H01M 10/0569; C07C 43/12; C07C 69/96; H01M 10/052; H01M 10/0525; H01M 10/0568

(52) U.S. Cl. CPC H01M 10/0569; C07C 43/126; C07C 69/96; H01M 10/052; H01M 10/0525; H01M 10/0568; H01M 2300/0034; H01M 2300/0037

(57) ABSTRACT

A molecular design principle utilizes a steric hindrance effect to tune the solvation structures of Li+ ions. By substituting the methoxy groups on DME with larger-sized ethoxy groups, the resulting 1,2-diethoxyethane (DEE) has weaker solvation ability and consequently more anion-rich inner solvation shells, both of which enhance interfacial stability at cathode and anode. According to certain additional aspects, the present embodiments relate to a family of fluorinated-1,2-diethyoxyethane (fluorinated-DEE) molecules that are readily synthesized in large scales to use as the electrolyte solvents. Selected positions on 1,2-diethyoxyethane (DEE, distinct from the diethyl ether are functionalized with various numbers of fluorine atoms through iterative tuning, to reach a balance between CE, oxidative stability, and ionic conduction. Paired with 1.2 M lithium bis(fluorosulfonyl)imide (LiFSI), these fluorinated-DEE-based, single-salt single-solvent electrolytes are thoroughly characterized. In addition, a family of fluorinated ethyl methyl carbonates are designed and synthesized. Different numbers of F atoms are finely tuned to yield monofluoroethyl methyl carbonate (F1EMC), difluoroethyl methyl carbonate (F2EMC) and trifluoroethyl methyl carbonate (F3EMC). The cycling behavior of several types of lithium-ion pouch cells were systematically investigated to understand the impact of fluorination degree.

### CROSS-REFERENCE TO RELATED APPLICATIONS

The present application claims priority to U.S. Provisional Patent Application No. 63/270,506 filed Oct. 21, 2021, and to U.S. Provisional Patent Application No. 63/283,828 filed Nov. 29, 2021, the contents of all such applications being incorporated herein by reference in their entirety.

### STATEMENT OF GOVERNMENT SPONSORED RESEARCH

This invention was made with Government support under contract DE-AC02-76SF000515 awarded by the Department of Energy. The Government has certain rights in the invention.

### TECHNICAL FIELD

The present embodiments relate generally to batteries, and more particularly to molecular design strategies to achieve favorable ion solvation structures for stable operation of lithium metal and lithium ion batteries, and to a family of fluorinated-1,2-diethyoxyethane (fluorinated-DEE) molecules, to a family of fluorinated carbonates, to a family of ethylene glycol ethers, and to a family of acetals that are readily synthesized in large scales to use as the electrolyte solvents.

### BACKGROUND

Current electrolyte formulations used in commercial lithium ion batteries are incompatible with lithium metal anode due to low coulombic efficiency and lithium dendrite formation during battery cycling. Ether-based electrolytes are promising alternatives. However, the coulombic efficiency remains unsatisfactory for commercial battery operations. The design of commercially viable ether and carbonate molecules that are simultaneously compatible with Li metal anodes (or graphite, graphite-silicon composite, and silicon anodes) and high-voltage cathodes is lacking. Although PCT application No. US20/048423 filed Aug. 28, 2020 (S19-364) dramatically advanced the state of the art in this technology, certain opportunities for technological improvement remain to address the above and other challenges.

### SUMMARY

In accordance with first general aspects, the present embodiments include at least two design strategies for ether molecules as electrolytes in lithium metal and lithium ion batteries. (1) Functional groups with various levels of steric hindrance can be leveraged to tune the solvation ability of ether solvents. (2) The arrangement of oxygen atoms can be modified to tune the solvation ability of ether solvents. Several nonfluorinated ether solvents designed based on the strategies above are paired with one or more lithium salts or additives to create electrolytes. Such electrolytes enable high lithium coulombic efficiency, dendrite prevention, good ionic conductivity, and good tolerance to battery operational voltage.

In accordance with second general aspects, the present embodiments relate to a family of fluorinated-1,2-diethyoxyethane (fluorinated-DEE) molecules that are readily synthesized in large scales to use as the electrolyte solvents. Selected positions on 1,2-diethyoxyethane (DEE, distinct from the diethyl ether previously reported) are functionalized with various numbers of fluorine atoms through iterative tuning, to reach a balance between CE, oxidative stability, and ionic conduction (FIG. 1a). Paired with 1.2 M lithium bis(fluorosulfonyl)imide (LiFSI), these fluorinated-DEE-based, single-salt single-solvent electrolytes are thoroughly characterized. Their Li⁺-solvent binding energies and geometries (from density functional theory [DFT] calculations), solvation environments (from solvation free energy measurements, ⁷Li-nuclear magnetic resonance [NMR], molecular dynamics [MD] simulations and diffusion-ordered spectroscopy [DOSY]), and results in batteries (measured ion conductivities and cell overpotentials) are found to be tightly correlated with each other. The above studies lead to an unexpected finding: partially-fluorinated, locally-polar —CHF₂ group results in higher ionic conduction than fully-fluorinated —CF₃ while still maintaining excellent electrode stability. Specifically, the best-performing F4DEE and F5DEE solvents both contain —CHF₂ group(s). In addition to high ionic conductivity and low, stable overpotential, they achieve ˜99.9% average CE for Li metal anode as well as fast activation, i.e., the CEs of the Li∥copper (Cu) half cells reach >99.3% from the second cycle. Aluminum (Al) corrosion was also significantly suppressed due to the oxidative stability that originated from suitable amount of fluorination. These features enabled ˜270 cycles in thin-Li (50-μm-thick) high-loading-NMC811 (LiNi_{0.8}Mn_{0.1}Co_{0.1}O₂, ˜4.9 mAh cm⁻²) full batteries and >140 cycles in fast-cycling anode-free Cu∥microparticle-LFP (LiFePO₄, ˜2.1 mAh cm⁻²) pouch cells, both of which stand among the state-of-the-art performances. It is worth noting that anode-free cells based on microparticle-LFP are rarely studied due to its low conductivity and limited-excess Li inventory compared to NMC (lithium nickel manganese cobalt oxide) cells. The long-cycling, high-rate Cu∥microparticle-LFP pouch cells demonstrated in this work thus fill the gap and allow for opportunities for low-cost Li metal batteries. The rational design process behind the electrolyte family presented in our work and our comprehensive investigation of its properties can be used to further develop the electrolytes towards practical Li metal batteries and fast cycling anode-free cells. In addition, a family of fluorinated ethyl methyl carbonates are designed and synthesized. Different numbers of F atoms are finely tuned to yield monofluoroethyl methyl carbonate (F1EMC), difluoroethyl methyl carbonate (F2EMC) and trifluoroethyl methyl carbonate (F3EMC). The cycling behavior of several types of lithium-ion pouch cells, including graphite (Gr)/single-crystalline LiNi_{0.8}Mn_{0.1}Co_{0.1}O₂ (SC-NMC811), Gr-SiOₓ/LiNi_{0.6}Mn_{0.2}Co_{0.2}O₂ (NMC622), high-voltage Gr/LiNi_{0.5}Mn_{1.5}O₄ (LNMO), Gr/layered Li-rich Mn-based oxide (LLMO) and fast-charging Gr/NMC622, were systematically investigated to understand the impact of fluorination degree. Compared to the commercially available F3EMC, the partially-fluorinated F1EMC and F2EMC in some cases showed improved cycling stability, which can be attributed to their locally-polar —CH₂F and —CHF₂ groups and thus fast ion conduction than —CF₃. This work suggests that highly or fully fluorinated solvents are not necessarily desirable; instead, fluorination degree needs to be rationally and finely tuned for optimized lithium-ion cell performance.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIG. 1 illustrates a hypothesized molecular design that utilizes steric hindrance effect from the end substituents to tune the solvation properties of solvent molecules according to embodiments.

FIGS. 2a-2h illustrates example solvation structures of the electrolytes according to embodiments.

FIGS. 3a-3e are graphs illustrating electrochemical stability of 1 M and 4 M LiFSI/DME and LiFSI/DEE electrolytes according to embodiments.

FIGS. 4a-4p are SEM images and graphs illustrating electrode morphologies and compositions in various electrolytes according to embodiments.

FIGS. 5a & 5b are graphs illustrating Li|NMC811 full cell performance under stringent conditions of high-loading NMC811 according to embodiments.

FIG. 6 illustrates molecular structures of 1,2-diethoxyethane (DEE) and diethyl ether.

FIGS. 7a-7c are graphs illustrating aspects of electrolytes according to embodiments.

FIG. 8 is a graph illustrating Raman spectra of 1 M and 4 M LiFSI/DME and DEE according to embodiments.

FIGS. 9a-9d illustrate geometry and energy of Li⁺-DME and Li⁺-DEE according to embodiments.

FIGS. 10a & 10b are graphs illustrating distributions of possible inner solvation shell compositions of 4 M LiFSI/DEE and 4 M LiFSI/DME according to embodiments.

FIGS. 11a-11d are graphs illustrating distributions of various Li+ coordination environments according to embodiments.

FIG. 12 illustrates Oxidation stability of various electrolytes on Pt electrode according to embodiments.

FIG. 13 illustrates long-term cycling of Li|Cu half cells in various electrolytes at 1 mAh cm⁻² capacity according to embodiments.

FIG. 14 illustrates cycling of Li|Cu half cells in 4 M electrolytes at 5 mAh cm⁻² capacity according to embodiments.

FIG. 15 illustrates Ionic conductivities of 1 M and 4 M LiFSI/DME and LiFSI/DEE according to embodiments.

FIGS. 16a-16d are SEM images of Li metal deposition on bare Cu in various electrolytes according to embodiments.

FIGS. 17a-17e are graphs illustrating surface XPS spectra of cycled Li electrodes in various electrolytes according to embodiments.

FIGS. 18a-18d are SEM images of Al electrodes after being held at 5.5 V (vs. Li⁺/Li) in various electrolytes according to embodiments.

FIGS. 19a & 19b are graphs illustrating Leakage currents during Al corrosion in various electrolytes at 5.5 V according to embodiments.

FIG. 20 illustrates SEM images of Al electrodes after being held at 4.4 V (vs. Li⁺/Li) in various electrolytes according to embodiments.

FIGS. 21a-21c are graphs illustrating leakage currents during Al corrosion in various electrolytes at 4.4 V (vs. Li⁺/Li) according to embodiments.

FIGS. 22a-22f are graphs illustrating XPS depth profiles of Al electrodes after being held at 5.5 V (vs. Li⁺/Li) according to embodiments.

FIGS. 23a-23f are graphs illustrating XPS depth profiles of Al electrodes after being held at 5.5 V (vs. Li⁺/Li) according to embodiments.

FIGS. 24a-24d are Voltage profiles of Li|NMC811 full cells using each electrolyte according to embodiments.

FIGS. 25a-25d provides a Summary of electrolytes and their properties investigated in embodiments.

FIG. 26 illustrates example Functional groups with various levels of steric hindrance that can be leveraged to tune the solvation ability of ether solvents according to embodiments.

FIG. 27 illustrates aspects of DEE, DnPE, DnBE that show improved CE compared to DME according to embodiments.

FIG. 28 illustrates aspects of DEE, DnPE, DnBE that show improved oxidative stability compared to DME.

FIG. 29 illustrates an example arrangement of oxygen atoms that can be modified to tune the solvation ability of ether solvents according to embodiments.

FIG. 30 illustrates aspects of 1M LiFSI/DMM and DEM that show very quick activation to reach >99% CE according to embodiments.

FIG. 31 illustrates aspects of 1M LiFSI/DMM and DEM that show improved oxidative stability compared to DME according to embodiments.

FIG. 32 illustrates aspects of 4M LiFSI/DMM and DEM that achieve quicker activation than DME according to embodiments.

FIG. 33 illustrates aspects of 4M LiFSI/DMM and DEM that show similar or slightly better oxidative stability compared to DME according to embodiments.

FIG. 34 illustrates a summary of aspects of embodiments.

FIGS. 35a-35d illustrate Solvent coordination geometry as an effective design strategy for LMB electrolytes according to embodiments.

FIGS. 36a & 36b illustrate aspects of Static solvation structures of 0.9 M and 3 M LiFSI in acetals (DMM and DEM) and ethylene glycol ethers (DME and DEE) according to embodiments.

FIGS. 37a-37f are graphs illustrating Electrochemical stability of 0.9 M and 3 M LiFSI in DMM and DEM according to embodiments.

FIG. 38 provides SEM images of the initial Li deposition morphology in 3 M LiFSI in DMM, DEM and DEE according to embodiments.

FIGS. 39a-39c illustrate aspects of ion transport analysis according to embodiments.

FIGS. 40a-40g are graphs illustrating LFP-based full cells cycled with 3 M LiFSI/DMM and 3 M LiFSI/DEM according to embodiments.

FIG. 41 is a graph illustrating ¹J_{CH} coupling constants of anomeric —CH₂— of DMM and DEM with various concentrations of LiFSI according to embodiments.

FIG. 42 are graphs illustrating Repeated Li∥Cu CE measurement by a modified Aurbach method at room temperature according to embodiments.

FIG. 43 is a graph illustrating Li∥Cu CE of 3 M LiFSI in DMM, DEM and DEE measured by the modified Aurbach method at 0° C. according to embodiments.

FIG. 44 is a graph illustrating Temperature-dependent ionic conductivities of 3 M LiFSI in DMM, DEM and DEE according to embodiments.

FIG. 45 is a graph illustrating Li∥Cu CE of 3 M LiFSI in DEE measured by the modified Aurbach method at −20° C. according to embodiments.

FIGS. 46a-46d are graphs illustrating Oxidative stability of the electrolytes measured by LSV using Al (a-b) and Pt (c-d) as the working electrode according to embodiments.

FIG. 47 provides SEM images of the initial Li deposition morphology in 3 M LiFSI in DMM according to embodiments.

FIG. 48 provides SEM images of the initial Li deposition morphology in 3 M LiFSI in DEM according to embodiments.

FIG. 49 provides SEM images of the initial Li deposition morphology in 3 M LiFSI in DEE according to embodiments.

FIGS. 50a-50d are graphs illustrating Concentration-dependent ionic conductivities of LiFSI in DME (a), DEE (b), DMM (c) and DEM (d) according to embodiments.

FIGS. 51a-51d are graphs illustrating Concentration-dependent molar conductivities of LiFSI according to embodiments.

FIGS. 52a & 52b are graphs illustrating Self-diffusion coefficients of solvents, Li⁺ and FSI⁻ in 0.9 M and 3 M electrolytes according to embodiments.

FIG. 53 is a graph illustrating Viscosity of 0.9 M and 3 M electrolytes according to embodiments.

FIG. 54 is a graph illustrating Inverse Haven ratios (1/HR) of 0.9 M and 3 M electrolytes according to embodiments.

FIG. 55 is a graph illustrating Ionic conductivities of 0.9 M and 3 M electrolytes according to embodiments.

FIG. 56 is a Zoomed-in view of FIG. 39b showing overpotential at different stages of Li∥Li cycling.

FIGS. 57a-57f are graphs illustrating Impedance of Li∥Li cells over cycling with 3 M LiFSI according to embodiments.

FIG. 58 is a Zoomed-in view of FIG. 39c showing overpotential under high current densities.

FIGS. 59a-59g provides corresponding CE values of cells in FIG. 40.

FIGS. 60a-60c provides Direct comparison of FDEE electrolytes with DMM and DEM electrolytes in Cu∥micro-LFP pouch cells according to embodiments.

FIGS. 61a-61d are graphs illustrating Anode-free Cu∥micro-LFP pouch cells (nominally ˜210 mAh, ˜2.1 mAh cm⁻², 2.5 to 3.65 V, 0.5 mL electrolyte, 1C=200 mA) cycled at various rates according to embodiments.

FIGS. 62a-62f are Voltage curves of anode-free Cu∥micro-LFP pouch cells cycled at various charge and discharge rates according to embodiments.

FIGS. 63a-63e are Voltage curves of anode-free Cu∥micro-LFP pouch cells cycled at various charge and discharge rates according to embodiments.

FIGS. 64a-64c are Voltage curves of thin-Li∥micro-LFP coin cells cycled at various charge and discharge current densities according to embodiments.

FIGS. 65a-65c are Voltage curves of thin-Li∥micro-LFP coin cells cycled at various charge and discharge current densities according to embodiments.

FIGS. 66a & 66b illustrate Ionic conductivities of evaluated electrolytes with (a) and without (b) separators according to embodiments.

FIGS. 67a-67f are graphs illustrating electrochemical stability of 4 M LiFSI/EtPrE, 4 M LiFSI/DnPE, and 3 M LiFSI/DnBE electrolytes according to embodiments.

FIGS. 68a-68i are graphs illustrating Discharge capacities of Li∥NMC811 full cells according to embodiments.

FIGS. 69a-69f illustrate example step-by-step design principles of the fluorinated-DEE solvent family of embodiments.

FIGS. 70a-70d illustrate example Ionic conductivity and cycling overpotential of FDMB and fluorinated-DEE electrolytes of embodiments.

FIGS. 71a-71m illustrate aspects of an example Theoretical and experimental study on the Li⁺ solvation structures and the structure-property correlations of embodiments.

FIGS. 72a-72f illustrate example Li metal efficiency and high-voltage stability of embodiments.

FIGS. 73a-73k illustrate example Full-cell performance of FDMB and fluorinated-DEE based electrolytes of embodiments.

FIGS. 74a-74q illustrate example Li metal morphological behavior and SEI components in fluorinated-DEE based electrolytes of embodiments.

FIGS. 75a-75c illustrate an example Summary and overall evaluation of fluorinated-DEE electrolytes of embodiments.

FIGS. 76a & 76b illustrate example chemical structures according to embodiments.

FIGS. 77a-77c illustrate Boiling points of synthesized fluorinated-DEEs and Viscosities of 1.2 M LiFSI in fluorinated-DEEs according to embodiments.

FIGS. 78a & 78b illustrate Ionic conductivities of developed electrolytes and control electrolytes according to embodiments.

FIGS. 79a-79f are EIS plots of Li∥Li symmetric cells with cycling according to embodiments.

FIG. 80 provides Voltage profiles of Li∥Cu half cell using 1 M LiFSI/FDMB at different cycle numbers according to embodiments.

FIGS. 81a & 81b are Voltage profiles of Li∥Cu half cell using 1.2 M LiFSI/DEE at different cycle numbers according to embodiments.

FIGS. 82a & 82b are Voltage profiles of Li∥Cu half cell using 1.2 M LiFSI/F3DEE at different cycle numbers according to embodiments.

FIGS. 83a & 83b are Voltage profiles of Li∥Cu half cell using 1.2 M LiFSI/F6DEE at different cycle numbers according to embodiments.

FIGS. 84a & 84b are Voltage profiles of Li∥Cu half cell using 1.2 M LiFSI/F4DEE at different cycle numbers according to embodiments.

FIGS. 85a & 85b are Voltage profiles of Li∥Cu half cell using 1.2 M LiFSI/F5DEE at different cycle numbers according to embodiments.

FIGS. 86a-86f illustrate Electrostatic potential (ESP) of different solvent molecules according to embodiments.

FIGS. 87a-87e illustrate 19F-NMR (376 MHz) spectra of pure fluorinated-DEEs and 1.2 M LiFSI in fluorinated-DEES according to embodiments.

FIGS. 88a-88c illustrate MD simulation results of 1 M LiFSI/FDMB according to embodiments.

FIGS. 89a-89c illustrate MD simulation results of 1.2 M LiFSI/DEE according to embodiments.

FIGS. 90a-90c illustrate MD simulation results of 1.2 M LiFSI/F3DEE according to embodiments.

FIGS. 91a-91c illustrate MD simulation results of 1.2 M LiFSI/F6DEE according to embodiments.

FIGS. 92a-92c illustrate MD simulation results of 1.2 M LiFSI/F4DEE according to embodiments.

FIGS. 93a-93d illustrate MD simulation results of 1.2 M LiFSI/F5DEE according to embodiments.

FIGS. 94a-94e illustrate Fitting results of internal reference DOSY NMR according to embodiments.

FIGS. 95a & 95b provide 7Li NMR (194 MHz) results of 1 M LiFSI/FDMB (extracted from ref.1) and 1.2 M LiFSI in fluorinated-DEEs according to embodiments.

FIG. 96 provides Solvation energy (ΔGsolvation) measurements of fluorinated-DEE electrolytes according to embodiments.

FIGS. 97a-97c provide FTIR results of 1.2 M LiFSI in fluorinated-DEEs according to embodiments.

FIGS. 98a-98f are graphs illustrating Long cycling of conventional (thin spring) Li∥Cu half cells at 0.5 mA cm−2 and 1 mAh cm−2, using fluorinated-DEE electrolytes according to embodiments.

FIGS. 99a-99f illustrate aspects of Li∥Cu half cells according to embodiments.

FIGS. 100a-100d illustrate aspects of cycling CE of Li∥Cu half cells at high currents and high capacities according to embodiments.

FIGS. 101a-101f illustrate aspects of Li∥Cu half cells with fluorinated-DEE based electrolytes according to embodiments.

FIGS. 102a & 102b illustrate aspects of LSV of Li∥Al coin cells using fluorinated-DEE electrolytes according to embodiments.

FIGS. 103a-103f illustrate Potatiostatic polarization of Li∥Al coin cells using fluorinated-DEE electrolytes according to embodiments.

FIGS. 104a-1041 illustrate HOMO and LUMO levels of different fluorinated-DEE molecules according to embodiments.

FIGS. 105a-105e illustrate Cycling performance of thin Li∥4.9 mAh cm−2 NMC811 coin cells using fluorinated-DEE electrolytes according to embodiments.

FIGS. 106a-106f illustrate Charge/discharge curves of 50 m Li∥˜4.9 mAh cm−2 NMC811 coin cells using fluorinated-DEE electrolytes according to embodiments.

FIGS. 107a-107f illustrate Voltage polarization of Li∥NMC811 or microparticle-LFP coin cells according to embodiments.

FIGS. 108a-108c illustrate EIS plots (a) and fitting results (b,c) of Cu NMC532 pouch cells after 40 cycles at 0.2 C charge 0.3 C discharge according to embodiments.

FIGS. 109a-109c illustrate Battery structure (a) and cycling performance (b,c) of 25 m Li∥3.8 mAh cm−2 NMC811 industrial free-standing pouch cells using fluorinated-DEE electrolytes according to embodiments.

FIGS. 110a-110h illustrate Cycling performance of 20 m Li∥˜2.2 mAh cm−2 NMC811 coin cells using fluorinated-DEE electrolytes according to embodiments.

FIGS. 111a-111f are Charge/discharge curves of 20 m Li∥˜2.2 mAh cm−2 NMC811 coin cells using fluorinated-DEE electrolytes according to embodiments.

FIGS. 112a-112f are Charge/discharge curves of 750 m Li∥˜2 mAh cm−2 microparticle-LFP coin cells using fluorinated-DEE electrolytes according to embodiments.

FIG. 113 provides Rate capability tests fluorinated-DEE electrolytes using m Li∥˜2 mAh cm−2 microparticle-LFP coin cells according to embodiments.

FIGS. 114a-114f illustrate Cycling performance of Cu∥˜2.1 mAh cm−2 microparticle-LFP anode-free pouch cells using fluorinated-DEE electrolytes according to embodiments.

FIGS. 115a-115c provide images of the Cu∥microparticle-LFP pouch cells using 1.2 M LiFSI/F4DEE and 1.2 M LiFSI/F5DEE according to embodiments.

FIGS. 116a-116e are SEM and optical images of the Cu side in Cu∥microparticle-LFP pouch cells according to embodiments.

FIGS. 117a-117d are SEM and optical images of the Cu side in Cu∥microparticle-LFP pouch cells according to embodiments.

FIGS. 118a-118e are SEM images of the Cu side in Cu∥NMC532 pouch cells cycled at 0.2 C charge 0.3 C discharge according to embodiments.

FIGS. 119a-119e are XPS O1s depth profiles of cycled Li metal electrodes using fluorinated-DEE electrolytes according to embodiments.

FIGS. 120a-120e are XPS S2p depth profiles of cycled Li metal electrodes using fluorinated-DEE electrolytes according to embodiments.

FIGS. 121a-121e are XPS C1s depth profiles of cycled Li metal electrodes using fluorinated-DEE electrolytes according to embodiments.

FIGS. 122a-122e are Cryo-TEM images of Li metal deposits using fluorinated-DEE electrolytes according to embodiments.

FIGS. 123a-123d illustrate Different elemental ratios obtained from cryo-EDS of Li metal deposits using fluorinated-DEE electrolytes.

FIGS. 124a-124e are Cryo-EDS plots of Li metal deposits using fluorinated-DEE electrolytes.

FIGS. 125a-125f illustrate Atomic ratio by XPS with different depths of NMC811 cathodes after 30 cycles according to embodiments.

FIGS. 126a-126f are Cross-sectional SEM images of NMC811 cathodes after 30 cycles according to embodiments.

FIG. 127 illustrates a Synthetic scheme of fluorinated-DEEs studied in embodiments.

FIG. 128 illustrates 1H-NMR of 2-(2,2-difluoroethoxy)ethanol (400 MHz, CDCl3, δ/ppm) according to embodiments.

FIG. 129 illustrates 13C-NMR of 2-(2,2-difluoroethoxy)ethanol (100 MHz, CDCl3, δ/ppm) according to embodiments.

FIG. 130 illustrates 19F-NMR of 2-(2,2-difluoroethoxy)ethanol (376 MHz, CDCl3, δ/ppm) according to embodiments.

FIG. 131 illustrates 1H-NMR of F3DEE (400 MHz, CDCl3, δ/ppm) according to embodiments.

FIG. 132 illustrates 13C-NMR of F3DEE (100 MHz, CDCl3, δ/ppm) according to embodiments.

FIG. 133 illustrates 19F-NMR of F3DEE (376 MHz, CDCl3, δ/ppm) according to embodiments.

FIG. 134 illustrates 1H-NMR of F6DEE (400 MHz, CDCl3, δ/ppm) according to embodiments.

FIG. 135 illustrates 13C-NMR of F6DEE (100 MHz, CDCl3, δ/ppm) according to embodiments.

FIG. 136 illustrates 19F-NMR of F6DEE (376 MHz, CDCl3, δ/ppm) according to embodiments.

FIG. 137 illustrates 1H-NMR of F4DEE (400 MHz, CDCl3, δ/ppm) according to embodiments.

FIG. 138 illustrates 13C-NMR of F4DEE (100 MHz, CDCl3, δ/ppm) according to embodiments.

FIG. 139 illustrates 19F-NMR of F4DEE (376 MHz, CDCl3, δ/ppm) according to embodiments.

FIG. 140 illustrates 1H-NMR of F5DEE (400 MHz, CDCl3, δ/ppm) according to embodiments.

FIG. 141 illustrates 13C-NMR of F5DEE (100 MHz, CDCl3, δ/ppm) according to embodiments.

FIG. 142 illustrates 19F-NMR of F5DEE (376 MHz, CDCl3, δ/ppm) according to embodiments.

FIGS. 143a & 143b illustrate Molecular structures of fluorinated-EMCs according to embodiments.

FIGS. 144a & 144b illustrates Synthetic procedures of F1EMC (a) and F2EMC (b) according to embodiments.

FIGS. 145a-145h illustrate ESP distribution of fluorinated-EMCs and coordination structures and binding energies of Li⁺-fluorinated-EMCs according to embodiments.

FIGS. 146a-146d illustrate ⁷Li- and ¹⁹F-NMR of fluorinated-EMCs and 1 M LiPF₆ in fluorinated-EMCs according to embodiments.

FIGS. 147a-147d illustrate Ionic conductivity of the electrolytes measured in coin cells according to embodiments.

FIGS. 148a-148f illustrate cycling behavior of Gr/SC-NMC811 pouch cells using different electrolytes according to embodiments.

FIGS. 149a-149f illustrate Cycling behavior of Gr-SiOₓ/NMC622 pouch cells using different electrolytes according to embodiments.

FIGS. 150a-150e are SEM and EDS images of Gr-SiOₓ anodes after ˜350 cycles using different electrolytes according to embodiments.

FIGS. 151a & 151b illustrate elemental composition results of Gr-SiOₓ anodes after ˜350 cycles using different electrolytes according to embodiments.

FIGS. 152a-152i illustrate Cycling behavior of Gr/LNMO pouch cells using different electrolytes at 1 C charge/discharge according to embodiments.

FIGS. 153a-153j illustrate elemental composition results of Gr anodes by XPS according to embodiments.

FIGS. 154a-154f illustrate Cycling behavior of Gr/LLMO pouch cells using different electrolytes according to embodiments.

FIGS. 155a-155h illustrate Fast-charging cycling behavior of Gr/NMC622 pouch cells using different electrolytes according to embodiments.

FIG. 156 illustrates 1H-NMR of F1EMC (400 MHz, CDCl3, δ/ppm) according to embodiments.

FIG. 157 illustrates 13C-NMR of F1EMC (100 MHz, CDCl3, δ/ppm) according to embodiments.

FIG. 158 illustrates 19F-NMR of F1EMC (376 MHz, CDCl3, δ/ppm) according to embodiments.

FIG. 159 illustrates 1H-NMR of F2EMC (400 MHz, CDCl3, δ/ppm) according to embodiments.

FIG. 160 illustrates 13C-NMR of F2EMC (100 MHz, CDCl3, δ/ppm) according to embodiments.

FIG. 161 illustrates 19F-NMR of F2EMC (376 MHz, CDCl3, δ/ppm) according to embodiments.

FIGS. 162a-162(c) illustrate Oxidative stability test using CV according to embodiments.

FIGS. 163a & 163b illustrate F is (a) and P 2p (b) XPS depth profiling spectra of Gr-SiOₓ anodes according to embodiments.

FIGS. 164a & 164b illustrate F is (a) and Mn 2p (b) XPS depth profiling spectra of Gr anodes according to embodiments.

FIGS. 165a-165c illustrate F is (a), Ni 2p (b), and Mn 2p (c) XPS depth profiling spectra of LNMO cathodes according to embodiments.

### DETAILED DESCRIPTION

The present embodiments will now be described in detail with reference to the drawings, which are provided as illustrative examples of the embodiments so as to enable those skilled in the art to practice the embodiments and alternatives apparent to those skilled in the art. Notably, the figures and examples below are not meant to limit the scope of the present embodiments to a single embodiment, but other embodiments are possible by way of interchange of some or all of the described or illustrated elements. Moreover, where certain elements of the present embodiments can be partially or fully implemented using known components, only those portions of such known components that are necessary for an understanding of the present embodiments will be described, and detailed descriptions of other portions of such known components will be omitted so as not to obscure the present embodiments. Embodiments described as being implemented in software should not be limited thereto, but can include embodiments implemented in hardware, or combinations of software and hardware, and vice-versa, as will be apparent to those skilled in the art, unless otherwise specified herein. In the present specification, an embodiment showing a singular component should not be considered limiting; rather, the present disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present embodiments encompass present and future known equivalents to the known components referred to herein by way of illustration.

### I. Steric Effect-Tuned Ion Solvation Enabling Stable Cycling of High-Voltage Lithium Metal Battery

### I. A. 1. Introduction

Lithium (Li) metal has the highest theoretical specific capacity (3860 mAh g⁻¹), the lowest standard reduction potential (−3.04 V vs. standard hydrogen electrode) and nearly the lowest solid density (0.534 g cm−³), making it an ideal material for battery anode. (Liu, J.; Bao, Z.; Cui, Y.; Dufek, E. J.; Goodenough, J. B.; Khalifah, P.; Li, Q.; Liaw, B. Y.; Liu, P.; Manthiram, A.; et al. Pathways for Practical High-Energy Long-Cycling Lithium Metal Batteries. Nat. Energy 2019, 4 (3), 180-186. https://doi.org/10.1038/s41560-019-0338x; Albertus, P.; Babinec, S.; Litzelman, S.; Newman, A. Status and Challenges in Enabling the Lithium Metal Electrode for High-Energy and Low-Cost Rechargeable Batteries. Nat. Energy 2018, 3 (1), 16-21. https://doi.org/10.1038/s41560-017-0047-2; Cao, Y.; Li, M.; Lu, J.; Liu, J.; Amine, K. Bridging the Academic and Industrial Metrics for Next-Generation Practical Batteries. Nat. Nanotechnol. 2019, 14 (3), 200-207. https://doi.org/10.1038/s41565-019-0371-8). However, the success of lithium metal batteries (LMBs) has been limited partly due to the highly reactive nature of Li. The major challenge associated with Li metal anode is the low Coulombic efficiency (CE) resulted from side reactions that cause continuous loss of active Li reservoir and consumption of electrolyte. Suitable electrolytes should form a protective solid electrolyte interphase (SEI) to inhibit further reactions between Li and electrolytes. (Peled, E.; Menkin, S. Review-SEI: Past, Present and Future. J. Electrochem. Soc. 2017, 164 (7), A1703-A1719. https://doi.org/10.1149/2.1441707jes; Tikekar, M. D.; Choudhury, S.; Tu, Z.; Archer, L. A. Design Principles for Electrolytes and Interfaces for Stable Lithium-Metal Batteries. Nat. Energy 2016, 1 (9), 1-7. https://doi.org/10.1038/nenergy.2016.114.) However, due to the large volume change of Li anode during cycling, SEI breaks down and the exposed fresh Li continues to react with electrolyte. (Aurbach, D. Review of Selected Electrode-Solution Interactions Which Determine the Performance of Li and Li Ion Batteries. J. Power Sources 2000, 89 (2), 206-218. https://doi.org/10.1016/SO378-7753(00)00431-6; Cohen, Y. S.; Cohen, Y.; Aurbach, D. Micromorphological Studies of Lithium Electrodes in Alkyl Carbonate Solutions Using in Situ Atomic Force Microscopy. J. Phys. Chem. B 2000, 104 (51), 12282-12291. https://doi.org/10.1021/jp002526b.) In addition, inhomogeneity in the SEI aggravates dendritic plating of Li. (Lin, D.; Liu, Y.; Cui, Y. Reviving the Lithium Metal Anode for High-Energy Batteries. Nat. Nanotechnol. 2017, 12 (3), 194-206. https://doi.org/10.1038/nnano.2017.16.) These high-aspect-ratio Li dendrites can easily result in the formation of ‘dead Li’ during long-term cycling. (Fang, C.; Li, J.; Zhang, M.; Zhang, Y.; Yang, F.; Lee, J. Z.; Lee, M. H.; Alvarado, J.; Schroeder, M. A.; Yang, Y.; et al. Quantifying Inactive Lithium in Lithium Metal Batteries. Nature 2019, 572 (7770), 511-515. https://doi.org/10.1038/s41586-019-1481-z.) Moreover, the byproducts of these processes lead to accumulation of thick SEI and ‘dead Li’, which increases cell overpotential and contributes to cell failure.

Electrolytes consisted of lithium hexafluorophosphate (LiPF₆) and carbonate solvents are used almost exclusively in traditional lithium ion batteries (LIBs). (Xu, K. Nonaqueous Liquid Electrolytes for Lithium-Based Rechargeable Batteries. Chem. Rev. 2004, 104 (10), 4303-4417. https://doi.org/10.1021/cr030203g.) In addition to their ability to form stable SEI on graphite anode, the high voltage stability and compatibility with aluminum (Al) current collector constitute another major contribution to their success in LIBs. Id. However, the same electrolytes are incompatible with Li anode due to uncontrollable dendrite growth and low CEs.

Ether-based electrolytes provide higher CE and dendrite suppression on Li anode. However, the pursuit for high-voltage cathodes, such as lithium nickel manganese cobalt oxides (NMC), presents additional challenges. It was thought that ether-based electrolytes were incompatible with high voltage (>4 V vs. Li⁺/Li) cathodes due to poor stability against oxidation. (Li, M.; Wang, C.; Chen, Z.; Xu, K.; Lu, J. New Concepts in Electrolytes. Chem. Rev. 2020, 120 (14), 6783-6819. https://doi.org/10.1021/acs.chemrev.9b00531.) The surprising discovery of high oxidation stability (4.5 V vs. Li⁺/Li) of equimolar LiTFSI-triglyme or LiTFSI-tetraglyme opened up opportunities for new electrolyte designs for high-voltage LMBs. (Pappenfus, T. M.; Henderson, W. A.; Owens, B. B.; Mann, K. R.; Smyrl, W. H. Complexes of Lithium Imide Salts with Tetraglyme and Their Polyelectrolyte Composite Materials. J. Electrochem. Soc. 2004, 151 (2), A209. https://doi.org/10.1149/1.1635384; Yoshida, K.; Nakamura, M.; Kazue, Y.; Tachikawa, N.; Tsuzuki, S.; Seki, S.; Dokko, K.; Watanabe, M. Oxidative-Stability Enhancement and Charge Transport Mechanism in Glyme-Lithium Salt Equimolar Complexes. J. Am. Chem. Soc. 2011, 133 (33), 13121-13129. https://doi.org/10.1021/ja203983r.) More recently, various ether-based low concentration electrolytes (Yu, Z.; Wang, H.; Kong, X.; Huang, W.; Tsao, Y.; Mackanic, D. G.; Wang, K.; Wang, X.; Huang, W.; Choudhury, S.; et al. Molecular Design for Electrolyte Solvents Enabling Energy-Dense and Long-Cycling Lithium Metal Batteries. Nat. Energy 2020, 5 (7), 526-533. https://doi.org/10.1038/s41560-020-0634-5; Holoubek, J.; Liu, H.; Wu, Z.; Yin, Y.; Xing, X.; Cai, G.; Yu, S.; Zhou, H.; Pascal, T. A.; Chen, Z.; et al. Tailoring Electrolyte Solvation for Li Metal Batteries Cycled at Ultra-Low Temperature. Nat. Energy 2021, 6, 303-313. https://doi.org/10.1038/s41560-021-00783-z; Amanchukwu, C. V.; Yu, Z.; Kong, X.; Qin, J.; Cui, Y.; Bao, Z. A New Class of Ionically Conducting Fluorinated Ether Electrolytes with High Electrochemical Stability. J. Am. Chem. Soc. 2020, 142 (16), 7393-7403. https://doi.org/10.1021/jacs.9b11056), high concentration electrolytes (HCEs) (Qian, J.; Henderson, W. A.; Xu, W.; Bhattacharya, P.; Engelhard, M.; Borodin, O.; Zhang, J. G. High Rate and Stable Cycling of Lithium Metal Anode. Nat. Commun. 2015, 6, 6362. https://doi.org/10.1038/ncomms7362; Jiao, S.; Ren, X.; Cao, R.; Engelhard, M. H.; Liu, Y.; Hu, D.; Mei, D.; Zheng, J.; Zhao, W.; Li, Q.; et al. Stable Cycling of High-Voltage Lithium Metal Batteries in Ether Electrolytes. Nat. Energy 2018, 3 (9), 739-746. https://doi.org/10.1038/s41560-018-0199-8; Ren, X.; Zou, L.; Jiao, S.; Mei, D.; Engelhard, M. H.; Li, Q.; Lee, H.; Niu, C.; Adams, B. D.; Wang, C.; et al. High-Concentration Ether Electrolytes for Stable High-Voltage Lithium Metal Batteries. ACS Energy Lett. 2019, 4 (4), 896-902. https://doi.org/10.1021/acsenergvlett 9100381; Chen, J.; Fan, X.; Li, Q.; Yang, H.; Khoshi, M. R.; Xu, Y.; Hwang, S.; Chen, L.; Ji, X.; Yang, C.; et al. Electrolyte Design for LiF-Rich Solid-Electrolyte Interfaces to Enable High-Performance Microsized Alloy Anodes for Batteries. Nat. Energy 2020, 5 (5), 386-397. https://doi.org/10.1038/s41560-020-0601-1; Chen, J.; Li, Q.; Pollard, T. P.; Fan, X.; Borodin, O.; Wang, C. Electrolyte Design for Li Metal-Free Li Batteries. Mater. Today 2020, 39 (October), 118-126. https://doi.org/10.1016/j.mattod.2020.04.004), and localized high concentration electrolytes (LHCEs) (Lee, M. S.; Roev, V.; Jung, C.; Kim, J. R.; Han, S.; Kang, H. R.; Im, D.; Kim, I. S. An Aggregate Cluster-Dispersed Electrolyte Guides the Uniform Nucleation and Growth of Lithium at Lithium Metal Anodes. ChemistrySelect 2018, 3 (41), 11527-11534. https://doi.org/10.1002/slct.201800757; Huang, F.; Ma, G.; Wen, Z.; Jin, J.; Xu, S.; Zhang, J. Enhancing Metallic Lithium Battery Performance by Tuning the Electrolyte Solution Structure. J. Mater. Chem. A 2018, 6 (4), 1612-1620. https://doi.org/10.1039/c7ta08274f; Ren, X.; Zou, L.; Cao, X.; Engelhard, M. H.; Liu, W.; Burton, S. D.; Lee, H.; Niu, C.; Matthews, B. E.; Zhu, Z.; et al. Enabling High-Voltage Lithium-Metal Batteries under Practical Conditions. Joule 2019, 3 (7), 1662-1676. https://doi.org/10.1016/j.joule 2019 05 006; Cao, X.; Ren, X.; Zou, L.; Engelhard, M. H.; Huang, W.; Wang, H.; Matthews, B. E.; Lee, H.; Niu, C.; Arey, B. W.; et al. Monolithic Solid-Electrolyte Interphases Formed in Fluorinated Orthoformate-Based Electrolytes Minimize Li Depletion and Pulverization. Nat. Energy 2019, 4 (9), 796-805. https://doi.org/10.1038/s41560-019-0464-5; Liu, H.; Holoubek, J.; Zhou, H.; Chen, A.; Chang, N.; Wu, Z.; Yu, S.; Yan, Q.; Xing, X.; Li, Y.; et al. Ultrahigh Coulombic Efficiency Electrolyte Enables Li∥SPAN Batteries with Superior Cycling Performance. Mater. Today 2021, 42 (xx), 17-28. https://doi.org/10.1016/j.mattod.2020.09 035; Cao, X.; Jia, H.; Xu, W.; Zhang, J.-G. Review—Localized High-Concentration Electrolytes for Lithium Batteries. J. Electrochem. Soc. 2021, 168 (1), 010522. https://doi.org/10.1149/1945-7111/abd60e) were developed. The combination of lithium bis(fluorosulfonyl)imide (LiFSI) and 1,2-dimethoxyethane (DME) was one of the most common electrolytes due to commercial availability, high salt solubility, good Li CE, dendrite suppression and high ionic conductivity.

Through a series of systematic studies, Zhang and Xu et al. demonstrated that DME is one of the best solvents to date for LHCEs to stabilize both Li anode and Ni-rich NMC cathodes. (Ren, X.; Gao, P.; Zou, L.; Jiao, S.; Cao, X.; Zhang, X.; Jia, H.; Engelhard, M. H.; Matthews, B. E.; Wu, H.; et al. Role of Inner Solvation Sheath within Salt-Solvent Complexes in Tailoring Electrode/Electrolyte Interphases for Lithium Metal Batteries. Proc. Natl. Acad. Sci. U.S.A 2020, 117 (46), 28603-28613. https://doi.org/10.1073/pnas.2010852117) The instability of DME under high voltage has been well known. Several strategies were proposed to solve this issue. For example, high-concentration dual-salt designs, such as LiFSI-lithium bis(trifluoromethanesulfonyl)imide (LiTFSI) in DME (Alvarado, J.; Schroeder, M. A.; Pollard, T. P.; Wang, X.; Lee, J. Z.; Zhang, M.; Wynn, T.; Ding, M.; Borodin, O.; Meng, Y. S.; et al. Bisalt Ether Electrolytes: A Pathway towards Lithium Metal Batteries with Ni-Rich Cathodes. Energy Environ. Sci. 2019, 12 (2), 780-794. https://doi.org/10.1039/c8ee02601g) and LiTFSI-lithium difluoro(oxalato)borate (LiDFOB) in DME, improved electrolyte stability at NMC-type cathodes due to improved passivation from the interplay between anions. A high concentrations of 1:1 (by mol) LiFSI-DME was also reported to improve the stability of electrolyte at NMC333 and NMC811 cathodes. However, these strategies still failed to address the intrinsic instability of DME. A promising alternative path is to carefully design new ether molecules to enhance high-voltage stability while maintaining or even boosting Li metal performance. For example, our groups previously reported a 2,2,3,3-tetrafluoro-1,4-dimethoxybutane (FDMB) ether solvent, which showed much improved high-voltage stability. (Wang, H.; Huang, W.; Yu, Z.; Huang, W.; Xu, R.; Zhang, Z.; Bao, Z.; Cui, Y. Efficient Lithium Metal Cycling over a Wide Range of Pressures from an Anion-Derived Solid-Electrolyte Interphase Framework. 2021, 35, 58. https://doi.org/10.1021/acsenergylett.0c02533) The single-salt single-solvent electrolyte of 1 M LiFSI/FDMB enabled high-voltage long-cycling LMBs. Beyond ether fluorination, a methodology of designing ether molecules that are simultaneously compatible with Li metal anodes and high-voltage cathodes is still lacking. Particularly, the non-fluorinated ether solvents deserve more attention due to their cost-effectiveness and eco-friendliness. (Flamme, B.; Rodriguez Garcia, G.; Weil, M.; Haddad, M.; Phansavath, P.; Ratovelomanana-Vidal, V.; Chagnes, A. Guidelines to Design Organic Electrolytes for Lithium-Ion Batteries: Environmental Impact, Physicochemical and Electrochemical Properties. Green Chem. 2017, 19 (8), 1828-1849. https://doi.org/10.1039/c7gc00252a) Therefore, there is an urgent demand for new molecular design principles.

Herein, we present a new molecular design principle where steric hindrance is leveraged to tune the solvation ability of ether solvents. Based on experimental and computational studies, we discovered that by simply substituting the methoxy groups on DME with slightly larger-sized ethoxy groups, the solvation property of the resulting 1,2-diethoxyethane (DEE) (FIG. 6) is drastically different from DME. Despite the compatibility of DEE with Li anodes was recently reported (Pham, T. D.; Lee, K. Simultaneous Stabilization of the Solid/Cathode Electrolyte Interface in Lithium Metal Batteries by a New Weakly Solvating Electrolyte. Small 2021, 2100133, 1-12. https://doi.org/10.1002/smll.202100133), we developed, in our work, a fundamental understanding on the molecular and interfacial origins of high voltage stability and high Li CEs of LiFSI/DEE electrolytes. In addition, full-cell performance was evaluated under stringent conditions of high-loading NMC811 (ca. 4.8 mAh cm⁻²), thin Li (50 μm thick), relatively high charge/discharge current densities (0.8/1.3 mA cm⁻²) and high cut-off voltage (4.4 V), where 4 M LiFSI/DEE sustained 182 cycles while 4 M LiFSI/DME only achieved 94 cycles until 80% capacity retention. Overall, we demonstrate that DEE is a promising replacement for DME in high-voltage LMBs. More importantly, this work illustrates that the steric hindrance effect offers a new handle to tune the solvation properties of electrolyte solvents.

FIG. 1 illustrates the hypothesized molecular design utilizes steric hindrance effect from the end substituents to tune the solvation properties of solvent molecules.

### I. A. 2. Results and Discussion

### I. A. 2. A. Steric Effect on Li⁺ Solvation

The common electrolyte 1 M LiFSI/DME possesses good ion solvation and high ionic conductivity. However, the insufficient Li CE, poor oxidation stability and Al corrosion problem render 1 M LiFSI/DME incompatible with high-voltage full cells such as LiINMC811. Based on the recent understanding on the roles of FSI⁻ in solvation shells and anion-derived interfaces, we propose the following molecular designs: 1) ethylene glycol middle segment should be preserved for desirable chelation with Li⁺ and consequently sufficient solubility of Li⁺ salt for high ionic conductivity; 2) by replacing the terminal methoxy groups with more sterically hindered functional groups, we hypothesize the increased steric hindrance could control and weaken the solvation ability of the two oxygen atoms, and thereby promoting the presence of FSI⁻ in the inner solvation shell; 3) such reduced solvation ability could remedy Al corrosion by allowing the build-up of a qualitied passivation layer (von Aspern, N.; Röschenthaler, G. V.; Winter, M.; Cekic-Laskovic, I. Fluorine and Lithium: Ideal Partners for High-Performance Rechargeable Battery Electrolytes. Angew. Chemie—Int. Ed. 2019, 58 (45), 15978-16000. https://doi.org/10.1002/anie.201901381; Xue, W.; Huang, M.; Li, Y.; Zhu, Y. G.; Gao, R.; Xiao, X.; Zhang, W.; Li, S.; Xu, G.; Yu, Y.; et al. Ultra-High-Voltage Ni-Rich Layered Cathodes in Practical Li Metal Batteries Enabled by a Sulfonamide-Based Electrolyte. Nat. Energy 2021, 6, 495-505. https://doi.org/10.1038/s41560-021-00792-y). Based on the designs above, we hypothesize that DEE is a potentially weaklier solvating solvent than DME, and therefore could induce more favorable interfacial properties and long-term cycling stability (FIG. 1). To verify our design principles and hypothesis above, DME and DEE are compared at LiFSI concentrations of 1 M and 4 M, which are representative of low and high concentration electrolytes respectively.

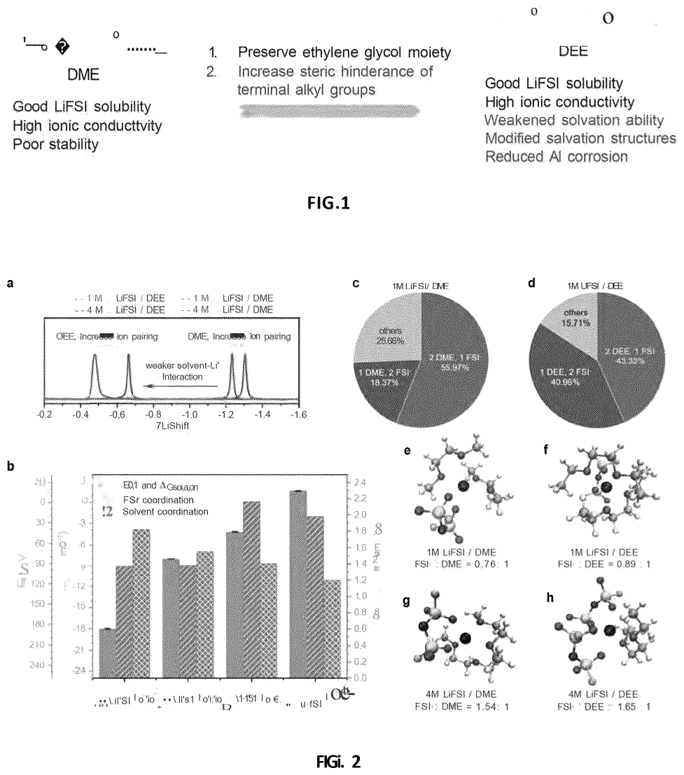

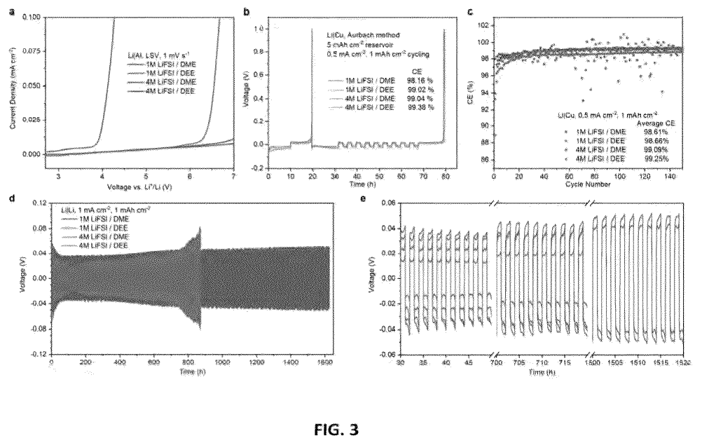

FIGS. 2a-2h illustrate example solvation structures of the electrolytes: (a) 7Li NMR of each electrolyte. All samples were characterized neat. The chemical shifts are referenced to 1 M LiCl in D₂O at 0 ppm. Peak intensities are normalized for clarity. (b) Left Y-axes: open circuit voltages (E_{cell}) and corresponding solvation energies (ΔGₛₒₗᵥₐₜᵢₒₙ) of the electrolytes (blue); right Y-axis: number of FSI⁻ (red slashes) and solvents (red crisscross) in the inner solvation shell. E_{cell} and ΔGₛₒₗᵥₐₜᵢₒₙ values are in reference to 1 M LiFSI in DEC. ΔGₛₒₗᵥₐₜᵢₒₙ was explained in detail in Kim, S. C.; Kong, X.; Vilá, R. A.; Huang, W.; Chen, Y.; Boyle, D. T.; Yu, Z.; Wang, H.; Bao, Z.; Qin, J.; et al. Potentiometric Measurement to Probe Solvation Energy and Its Correlation to Lithium Battery Cyclability. J. Am. Chem. Soc. 2021, 143 (27), 10301-10308. https://doi.org/10.1021/jacs.1c03868. Coordination numbers were calculated using MD simulation. (c-d) The distributions of possible inner solvation shell compositions of 1 M LiFSI/DME and DEE from MD simulation. For each electrolyte, two of the most probable compositions are shown and the rest are grouped as “others”. (e-h) Structures of the most probably inner solvation shells and average FSI⁻ to solvent ratios of the four electrolytes from MD simulation.

We first performed nuclear magnetic resonance (NMR) measurements on DME and DEE electrolytes to study their solvation ability. 7Li NMR is sensitive to the coordinating species in the solvation shell. An upfield (more negative) shift indicates increased electron density around Li⁺ due to either stronger solvent binding or stronger anion binding. (Amanchukwu, C. V.; Kong, X.; Qin, J.; Cui, Y.; Bao, Z. Nonpolar Alkanes Modify Lithium-Ion Solvation for Improved Lithium Deposition and Stripping. Adv. Energy Mater. 2019, 9 (41), 1-11. https://doi.org/10.1002/aenm.2019-02116) Upon increasing LiFSI concentration from 1 M to 4 M, the 7Li peak shifts upfield for both DME and DEE samples (FIG. 2a), which is indicative of increased ion pairing at a higher concentration. (Zhang, J. G.; Xu, W.; Xiao, J.; Cao, X.; Liu, J. Lithium Metal Anodes with Nonaqueous Electrolytes. Chem. Rev. 2020, 120 (24), 13312-13348. https://doi.org/10.1021/acs.chemrev.0c00275; Lukatskaya, M. R.; Feldblyum, J. I.; Mackanic, D. G.; Lissel, F.; Michels, D. L.; Cui, Y.; Bao, Z. Concentrated Mixed Cation Acetate “Water-in-Salt” Solutions as Green and Low-Cost High Voltage Electrolytes for Aqueous Batteries. Energy Environ. Sci. 2018, 11 (10), 2876-2883. https://doi.org/10.1039/c8ee00833g) Regardless of LiFSI concentration, DME-based electrolytes are more upfield shifted than DEE-based electrolytes (FIG. 2a). The stronger solvation ability of DME than DEE results in stronger solvent-Li+ interaction, which leads to more electron density around Li+ and upfield shift in NMR for DME samples. 19F NMR of FSI− shows opposite trends in DME and DEE as LiFSI concentration increases (FIG. 7a) likely due to the complex interactions of FSI− with other components in the solvation shell. 1H NMR of protons adjacent to ether oxygens on DEE and DME all shift upfield with increasing LiFSI concentration (FIGS. 7b-c), which corresponds to increased fractions of solvent molecules participating in the solvation shell.

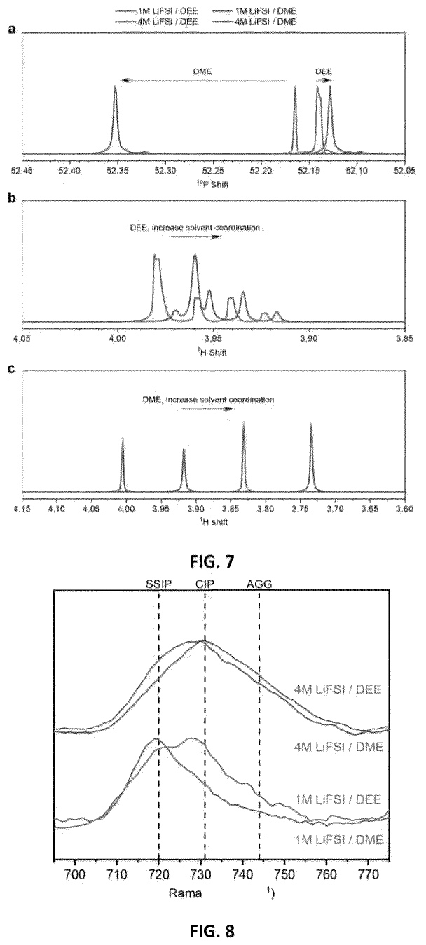

Raman spectroscopy was carried out to investigate the coordination environment of FSI−. The convoluted peaks at around 710 to 760 cm⁻¹ correspond to FSI− vibrational modes. The wavenumbers increase in the order of solvent-separated ion pairs (SSIP), contact ion pairs (CIP), and ion aggregates (AGG) (FIG. 8). (Yamada, Y.; Yaegashi, M.; Abe, T.; Yamada, A. A Superconcentrated Ether Electrolyte for Fast-Charging Li-Ion Batteries. Chem. Commun. 2013, 49, 11194-11196. https://doi.org/10.1039/c3cc46665e; Cao, X.; Zou, L.; Matthews, B. E.; Zhang, L.; He, X.; Ren, X.; Engelhard, M. H.; Burton, S. D.; El-Khoury, P. Z.; Lim, H. S.; et al. Optimization of Fluorinated Orthoformate Based Electrolytes for Practical High-Voltage Lithium Metal Batteries. Energy Storage Mater. 2021, 34, 76-84. https://doi.org/10.1016/j.ensm.2020.08 035; Jiang, Z.; Zeng, Z.; Liang, X.; Yang, L.; Hu, W.; Zhang, C.; Han, Z.; Feng, J.; Xie, J. Fluorobenzene, A Low-Density, Economical, and Bifunctional Hydrocarbon Cosolvent for Practical Lithium Metal Batteries. Adv. Funct. Mater. 2021, 31, 2005991. https://doi.org/10.1002/adfm.202005991) Therefore, the position of peak maximum and the relative intensity of shoulder peaks provide qualitative information on the relative amount of SSIP, CIP and AGG. The peak intensity of CIP and AGG relative to SSIP is higher for 1M LiFSI/DEE than for 1 M LiFSI/DME (FIG. 8), indicating stronger Li+-FSI− interactions and weaker solvation ability of DEE than DME. Compared to their 1 M counterparts, both 4 M LiFSI/DME and DEE show more CIP and AGG relative to SSIP (FIG. 8). In addition, 4 M LiFSI/DEE exhibits a larger AGG shoulder peak and a smaller SSIP shoulder peak than 4 M LiFSI/DME (FIG. 8), which further demonstrates the weaker solvation ability of DEE than DME.

To confirm the difference in solvation ability of DME and DEE, solvation energy (ΔGsolvation) of each electrolyte was measured. The open-circuit potential (Ecell) of a cell with symmetric Li electrodes and asymmetric electrolytes is related to ΔGsolvation. In essence, a more negative Ecell corresponds to a more positive ΔGsolvation, which suggests the sample electrolyte is weaklier solvating to Li⁺ than the reference electrolyte (1 M LiFSI in diethyl carbonate (DEC)). Ecell becomes less positive or more negative in the order of 1 M LiFSI/DME>1 M LiFSI/DEE>4 M LiFSI/DME>4 M LiFSI/DEE, and ΔGsolvation follows the opposite trend (FIG. 2b, blue columns). Based on Ecell and ΔGsolvation, 4 M electrolytes are more weakly solvating than 1 M electrolytes for both DME and DEE. In addition, at the same concentration, DEE is weaklier solvating than DME. Both observations are consistent with 7Li NMR and Raman results.

Molecular dynamics (MD) simulations were carried out to provide more detailed information on solvation structures. Various Li⁺ solvation shells and their probabilities in each electrolyte are listed in Table S1. The average numbers of FSI⁻ and solvent (DME or DEE) coordinating to Li⁺ in the inner solvation shell for each electrolyte are shown in FIG. 2b (red columns). The average number of solvent molecules in the inner solvation shell decreases in the order of 1 M LiFSI/DME>1 M LiFSI/DEE>4 M LiFSI/DME>4 M LiFSI/DEE. This decrease in solvent fraction with increasing salt concentration is consistent with previous knowledge on HCEs. (Perez Beltran, S.; Cao, X.; Zhang, J. G.; Balbuena, P. B. Localized High Concentration Electrolytes for High Voltage Lithium-Metal Batteries: Correlation between the Electrolyte Composition and Its Reductive/Oxidative Stability. Chem. Mater. 2020, 32 (14), 5973-5984. https://doi.org/10.1021/acs.chemmater.0c00987) At both 1 M and 4 M concentrations, there are fewer DEE than DME molecules in the solvation shell, which again indicates the weaker solvation ability of DEE than DME as shown in 7Li NMR, Raman spectra, and ΔGsolvation measurements. The trend in FSI− coordination numbers is less straightforward. At 1 M concentration, both DME and DEE electrolytes have similar numbers of FSI− in the inner solvation shell, whereas at 4 M concentration, the inner solvation shell of DME electrolyte has more FSI− than that of DEE. More detailed analyses below are carried out to explain these results.

At 1 M concentration, there is a large excess of solvent and Li+ should be well solvated. Therefore, we hypothesize that the composition of solvation shell at 1 M should reflect the relative coordination ability of solvent and anion. The two most probable solvation structures are 2 solvent molecules, 1 FSI− (Structure 1) and 1 solvent molecule, 2 FSI− (Structure 2) for both 1 M LiFSI/DME (FIG. 2c) and 1 M LiFSI/DEE (FIG. 2d). There is a large preference for Structure 1 (55.97%) over Structure 2 (18.37%) in 1 M LiFSI/DME, whereas the difference is minimal (43.33% and 40.96%) in 1 M LiFSI/DEE. Therefore, there is a stronger tendency for DME to coordinate with Li+ than DEE in the inner solvation shell, indicating the weaker solvation ability of DEE than DME. An alternative explanation is that the dielectric constant of DME is slightly higher than that of DEE, which results in more ion pairing in DEE. However, given that the average number of FSI− in the inner solvation shell is similar for 1 M LiFSI/DME and 1 M LiFSI/DEE (FIG. 2b), this explanation cannot be the main reason for the different distributions of solvation structures.

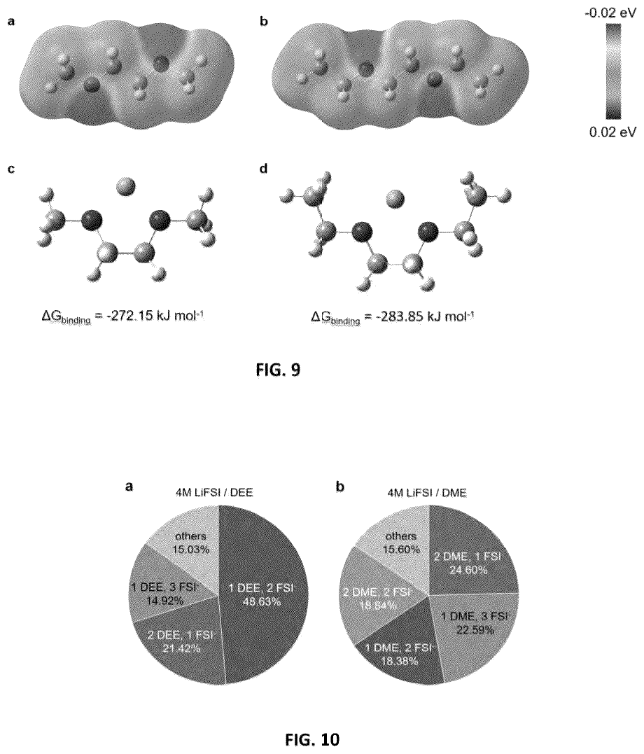

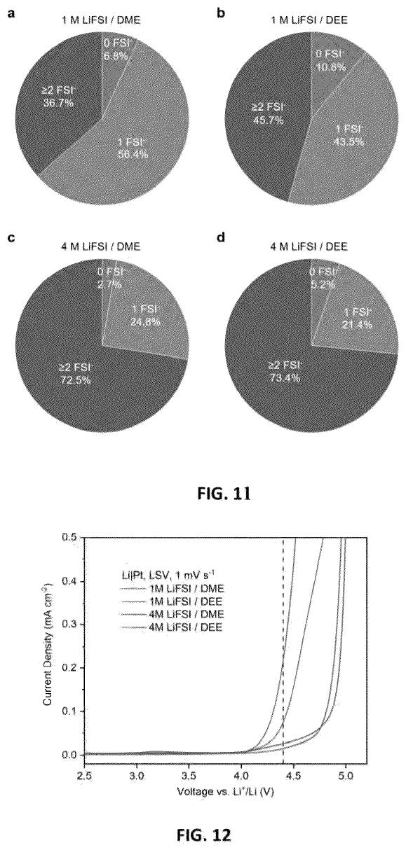

There may be two possible reasons for the apparent weaker solvation ability of DEE than DME—weaker Lewis basicity of DEE oxygens and stronger steric hindrance of ethoxy groups on DEE. To deconvolute the two, density-functional theory (DFT) calculations were carried out. DFT shows similar electrostatic potential (ESP) distribution on DME and DEE (FIGS. 9a-b). We then calculated the binding energy (ΔGbinding) between 1 solvent molecule and 1 Li⁺, which captures the intrinsic coordination ability of DME and DEE without steric effect from multiple molecules within the inner solvation shell. ΔGbinding is slightly more negative for DEE than DME by about 10 kJ mol⁻¹ (FIGS. 9c-d), which indicates an intrinsically stronger chelation ability of DEE than DME. This leads us to further conclude that the apparent weaker solvation ability of DEE compared to DME in solution does not come from the difference in Lewis basicity of the oxygen atoms in DEE vs. DME, but rather originates from the increased steric hindrance of ethoxy groups compared to methoxy groups. Indeed, comparing the most probable solvation structures of 1 M LiFSI/DME (FIG. 2e) and 1 M LiFSI/DEE (FIG. 2f), the latter must accommodate 4 extra carbons and 8 extra hydrogens, which results in additional energy penalty for DEE coordination. As a result, DEE has a lower average coordination number to Li+ due to its bulkier size compared to DME. At 4 M concentration, the steric effect of DEE results in a strong preference (48.63%) for 1 DEE and 2 FSI− coordination (FIGS. 2h and 10a), whereas the smaller DME prefers 2 DME, 1 FSI− coordination (24.60%) and even a four-molecule coordination of 1 DME, 3 FSI− (22.59%) (FIGS. 2g and 10b). Overall, in the order of 1 M LiFSI/DME<1 M LiFSI/DEE<4 M LiFSI/DME<4 M LiFSI/DEE, the anion-to-solvent ratios increase (FIGS. 2e-h), and the probability of multiple FSI− within Li+ solvation shell increases (FIGS. 11a-d), both of which are consistent with the weaker solvation at higher concentration as well as the weaker solvation in DEE than in DME.

Based on the evidence above, we conclude that the steric hindrance effect can indeed tune the solvation ability of ether solvents, which in turn modifies solvation structures of Li⁺. Such changes in solvation structures are expected to influence electrochemical properties of electrolytes and ultimately cycling performance of LMBs

FIG. 3 illustrates electrochemical stability of 1 M and 4 M LiFSI/DME and LiFSI/DEE electrolytes: (a) Oxidation stability on Al current collector; each cell was scanned from V_{oc} to 7 V (vs. Li⁺/Li); the data of 1 M LiFSI/DME is reproduced with permission. Copyright 2020 Nature Publishing Group; (b) Modified Aurbach measurement (Adams, B. D.; Zheng, J.; Ren, X.; Xu, W.; Zhang, J. G. Accurate Determination of Coulombic Efficiency for Lithium Metal Anodes and Lithium Metal Batteries. Adv. Energy Mater. 2018, 8 (7), 1-11. https://doi.org/10.1002/aenm.201702097) of Li CEs; (c) Li CEs during the first 150 cycles; the average stabilized CEs are calculated from 50 to 150 cycles; (d) long-term cycling of Li|Li half cells; and (e) zoomed-in voltage curves during different stages of Li|Li half-cell cycling.

I. A. 2. b. Electrochemical Stability

Based on the weaker solvation ability of DEE relative to DME, we expect improved electrochemical stability in LiFSI/DEE compared to LiFSI/DME. The oxidation stability of each electrolyte was tested by linear sweep voltammetry (LSV) (FIG. 3a). Al was selected as the working electrode to mimic the realistic environment in full cells where Al is typically used as the cathode current collector. The leakage current from 1 M LiFSI/DME increases sharply below 4 V (vs. Li+/Li) due to severe Al corrosion. This onset voltage is lower than the value measured using inert Pt electrode (FIG. 12), which does not reflect the real battery conditions. In stark contrast to DME, 1 M LiFSI/DEE does not show significant increase in leakage current on Al electrode until around 6 V (vs. Li+/Li). Upon increasing LiFSI concentration to 4 M, the leakage current on Al electrode dramatically decreases for both DME and DEE, which is consistent with previous reports on DME electrolytes. Both electrolytes appear to be stable with Al electrode up to 7 V (vs. Li+/Li) with DEE having a slightly lower leakage current than DME. In addition, LSV using non-reactive Pt electrode shows decreasing leakage current at 4.4 V (vs. Li+/Li) in the order of 1 M LiFSI/DME>1 M LiFSI/DEE>4 M LiFSI/DME>4 M LiFSI/DEE (FIG. 12). These results suggest DEE is more suitable than DME for high voltage LMBs.

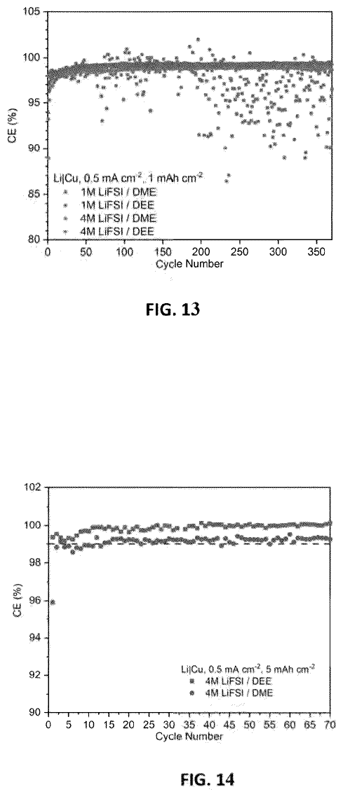

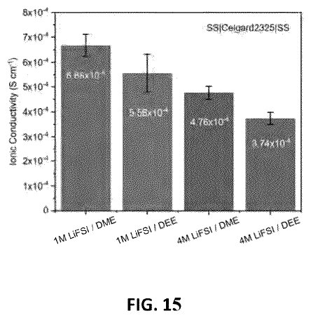

We then investigated the compatibility of DEE with Li anode. The Li CEs were determined by a modified Aurbach method (FIG. 3b). Id. With 1 M LiFSI, CE of DEE reaches 99.02% and outperforms that of DME at 98.16%. Such high CE even at a normal salt concentration indicates superior compatibility of DEE with Li. At higher LiFSI concentration of 4 M, both DME and DEE show improved CEs (99.04% and 99.38% respectively) compared to their 1 M counterparts, which agrees with previous findings on HCEs. (Qian, J.; Adams, B. D.; Zheng, J.; Xu, W.; Henderson, W. A.; Wang, J.; Bowden, M. E.; Xu, S.; Hu, J.; Zhang, J. G. Anode-Free Rechargeable Lithium Metal Batteries. Adv. Funct. Mater. 2016, 26 (39), 7094-7102. https://doi.org/10.1002/adfm.201602353; Zeng, Z.; Murugesan, V.; Han, K. S.; Jiang, X.; Cao, Y.; Xiao, L.; Ai, X.; Yang, H.; Zhang, J. G.; Sushko, M. L.; et al. Non-Flammable Electrolytes with High Salt-to-Solvent Ratios for Li-Ion and Li-Metal Batteries. Nat. Energy 2018, 3 (8), 674-681. https://doi.org/10.1038/s41560-018-0196-y; Maeyoshi, Y.; Ding, D.; Kubota, M.; Ueda, H.; Abe, K.; Kanamura, K.; Abe, H. Long-Term Stable Lithium Metal Anode in Highly Concentrated Sulfolane-Based Electrolytes with Ultrafine Porous Polyimide Separator. ACS Appl. Mater. Interfaces 2019, 11 (29), 25833-25843. https://doi.org/10.1021/acsami.9b05257) The higher CE of DEE compared to DME at 4 M further showcases the improved stability of DEE at Li anode. In addition, the results from long-term cycling of Li|Cu half cells corroborate with those from Aurbach method. For clarity, only the first 150 cycles are shown in FIG. 3c and the complete cycling data is available in FIG. 13. The cycling is unstable using 1 M LiFSI/DME and CE fluctuates significantly. In contrast, 1 M LiFSI/DEE shows stable cycling with an average CE of 98.66%. At a higher concentration of 4 M LiFSI, the average CEs further improve for both DME and DEE (99.09% and 99.25% respectively). The CE values obtained from Aurbach method and long-term cycling are slightly different likely due to the properties of substrates-deposited Li in Aurbach method vs. Cu in long-term cycling. Li|Cu cells with 4 M electrolytes were also cycled at 5 mAh cm⁻² capacity (FIG. 14) to match a realistic full cell areal capacity. The CE in 4 M LiFSI/DEE is higher than 4 M LiFSI/DME although both quickly reach >99%. Furthermore, Li|Li symmetric cells were used to investigate the long-term stability and overpotential of DEE and DME (FIGS. 3d-e). DME and DEE with 1 M LiFSI exhibit stable cycling for about 750 hours before a sharp increase in overpotential followed by cell shorting. At 4 M concentration, both DME and DEE show stable cycling for over 1600 hours with only a minor increase in overpotential before testing was terminated without cell failure. The magnitudes of overpotentials during the initial period of Li|Li cycling follow the trend of electrolyte ionic conductivities (FIG. 15). All four electrolytes show reasonably low overpotentials due to good ionic conductivities. Agreeing with our design principle, the preserved ethylene glycol moiety on DEE enables good ionic conductivity similar to DME.

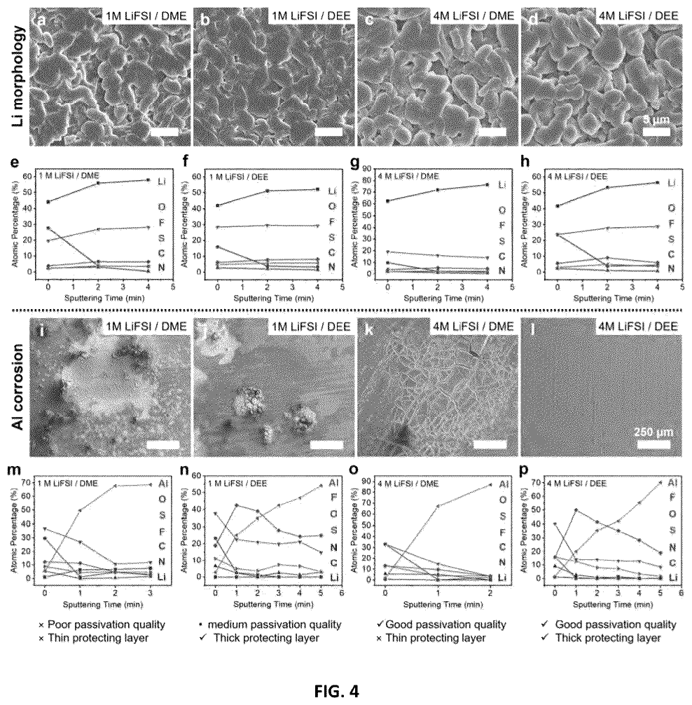

FIG. 4 illustrates Electrode morphologies and compositions in various electrolytes: (a-d) SEM images of Li metal deposition on Cu (1st cycle plating, 0.5 mA cm⁻², 1 mAh cm⁻²) (scale bar=5 μm); (e-h) XPS depth profiles of Li electrodes cycled in various electrolytes (Li|Li cells, 10 cycles at 1 mA cm⁻², 1 mAh cm⁻²); (i-l) SEM images of Al current collectors after being held at 5.5 V (vs. Li⁺/Li) in various electrolytes for about 20 hours (scale bar=250 μm); (m-p) XPS depth profiles of Al electrodes after being held at 5.5 V (vs. Li⁺/Li) in various electrolytes for about 23 hours.

I. A. 2. c. Electrode-Electrolyte Interfaces

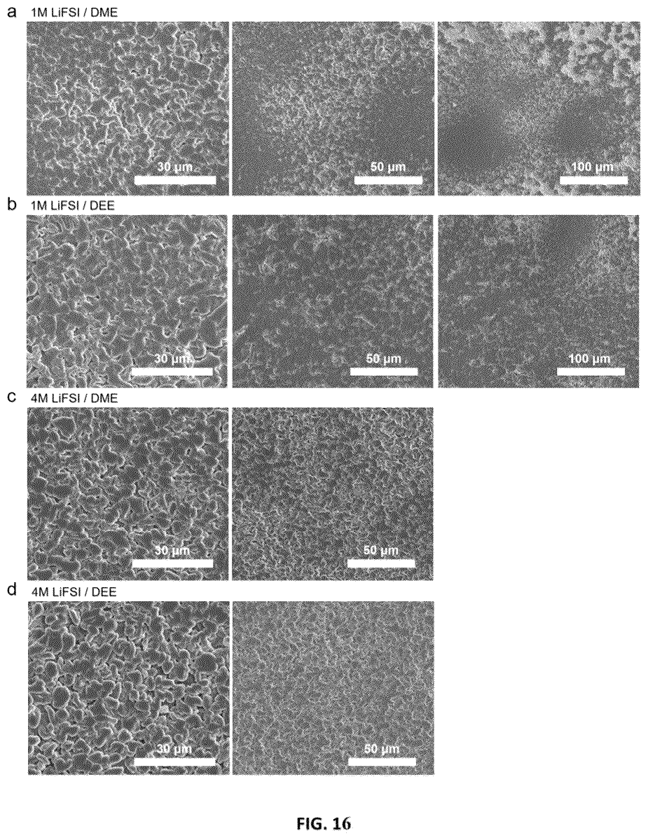

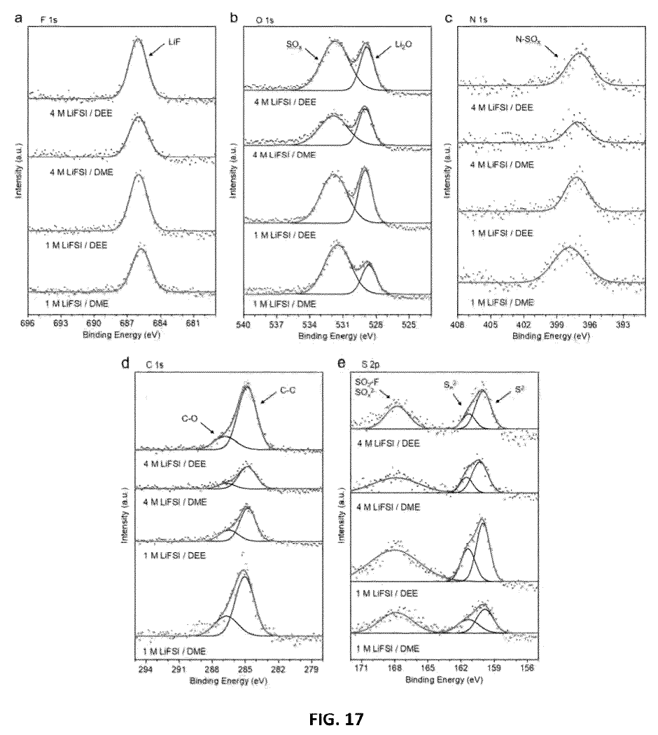

The origin of the improved electrochemical stability of LiFSI/DEE was further investigated at electrode-electrolyte interfaces. The Li deposition morphology on bare Cu is shown in FIGS. 4a-d and FIG. 16. In all four electrolytes, Li is deposited as chunky particles that are several microns in size, which is consistent with previous reports on DME-based electrolytes. The similar morphology is not surprising considering all four electrolytes show Li CE above 98% (FIGS. 3b-c). From X-Ray Photoelectron Spectroscopy (XPS), the SEI compositions (FIGS. 17a-e) were similar for the four electrolytes due to similar decomposition pathways for ether electrolytes. The overall SEI from 1 M LiFSI/DEE has more F, O and S and less C compared to that from 1 M LiFSI/DME (FIGS. 4e-f), indicating more anion decomposition in DEE-based electrolyte. In addition, 1 M LiFSI/DEE results in more complete reduction of FSI− as evidenced by the increased intensities from oxide and sulfide (FIGS. 17b,e). Comparing the inner SEI (after 2- and 4-minute sputtering) from 4 M LiFSI/DME and 4 M LiFSI/DEE, the latter has higher percentages of F, O and S derived from FSI⁻ (FIGS. 4g-h). Interestingly, the outer SEI (without sputtering) from 4 M LiFSI/DEE is rich in organic components (FIG. 4h), which could improve flexibility of SEI and further contribute to better stability. 8 Overall, at both 1 M and 4 M LiFSI concentrations, these is a stronger tendency to form anion-derived SEI in DEE than in DME, which leads to improved CEs (FIGS. 3b-c).

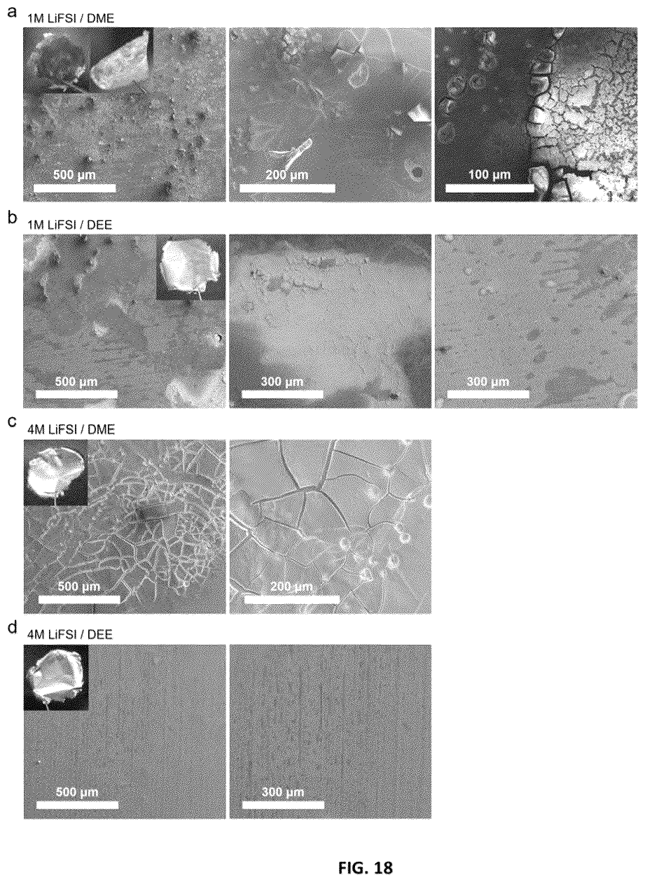

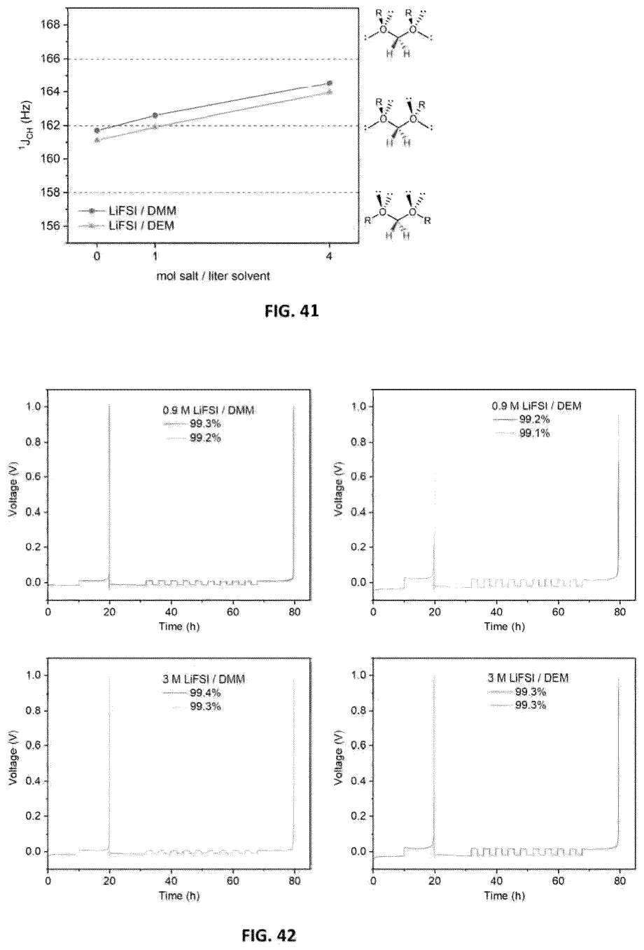

The oxidation stability of LiFSI/DME and LiFSI/DEE was further studied. One major issue of imide salts (eg. LiFSI and LiTFSI) is Al corrosion at high voltage due to the inability to form AlF₃ and LiF passivation layer on Al surface. (Ma, T.; Xu, G. L.; Li, Y.; Wang, L.; He, X.; Zheng, J.; Liu, J.; Engelhard, M. H.; Zapol, P.; Curtiss, L. A.; et al. Revisiting the Corrosion of the Aluminum Current Collector in Lithium-Ion Batteries. J. Phys. Chem. Lett. 2017, 8 (5), 1072-1077. https://doi.org/10.1021/acs.jpclett.6b02933; Abouimrane, A.; Ding, J.; Davidson, I. J. Liquid Electrolyte Based on Lithium Bis-Fluorosulfonyl Imide Salt: Aluminum Corrosion Studies and Lithium Ion Battery Investigations. J. Power Sources 2009, 189 (1), 693-696. https://doi.org/10.1016/j.jpowsour.2008.08.077; McOwen, D. W.; Seo, D. M.; Borodin, O.; Vatamanu, J.; Boyle, P. D.; Henderson, W. A. Concentrated Electrolytes: Decrypting Electrolyte Properties and Reassessing Al Corrosion Mechanisms. Energy Environ. Sci. 2014, 7 (1), 416-426. https://doi.org/10.1039/c3ee42351d; Matsumoto, K.; Inoue, K.; Nakahara, K.; Yuge, R.; Noguchi, T.; Utsugi, K. Suppression of Aluminum Corrosion by Using High Concentration LiTFSI Electrolyte. J. Power Sources 2013, 231, 234-238. https://doi.org/10.1016/j.jpowsour.2012.12.028) Indeed, after holding Al electrode at 5.5 V (vs. Li+/Li) in 1 M LiFSI/DME for about 20 hours, severe Al corrosion occurs as evidenced by extensively pitting, roughening and cracking across the entire surface (FIGS. 4i and 18a). In addition, the discoloration of Al and electrolyte (FIG. 18a insert) further indicates the occurrence of side reactions between Al and 1 M LiFSI/DME under high voltage. In contrast, the corrosion of Al electrode is less severe in 1 M LiFSI/DEE after holding at 5.5 V (vs. Li+/Li), where some areas remain clear (FIGS. 4j and 18b). However, the discoloration of electrolyte is still observed (FIG. 18b insert). Upon increasing salt concentration, Al electrode in 4 M LiFSI/DME still shows extensive formation of cracks and pits (FIGS. 4k and 18c), although the surface appears smoother than the case of 1 M LiFSI/DME. In stark contrast with other electrolytes, 4 M LiFSI/DEE shows no corrosion on Al electrode (FIGS. 41 and 18d).

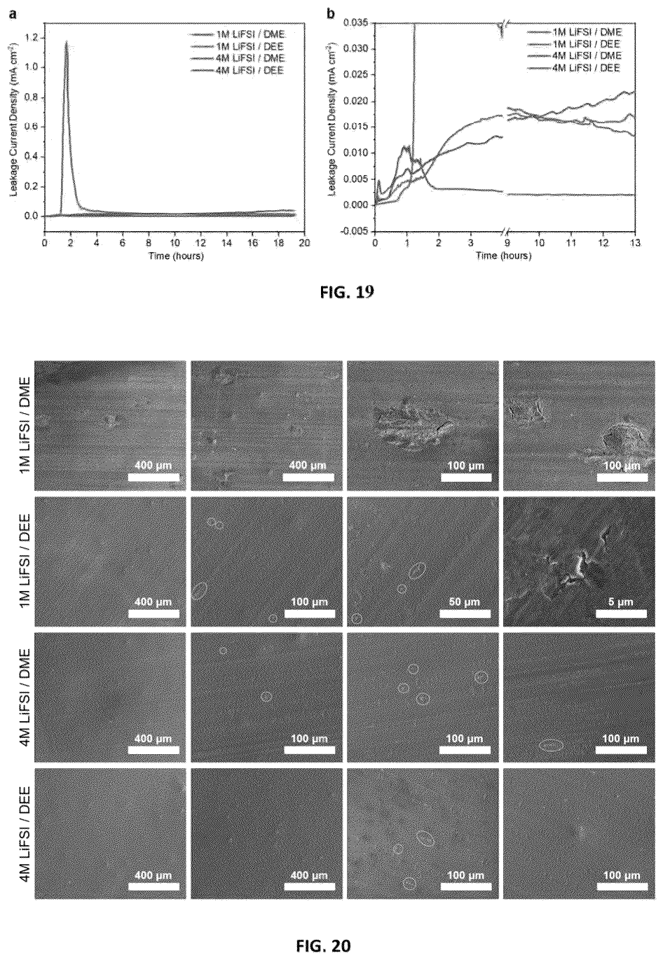

The results from SEM images can be corroborated by the leakage current during voltage hold. Consistent with LSV results (FIG. 3a), the leakage current is significantly higher for 1 M LiFSI/DME (FIG. 19a), resulting in severe Al corrosion. During the 1st hour, as voltage slowly increases, the leakage current follows the order of 4M LiFSI/DEE>4M LiFSI/DME>1M LiFSI/DEE>1M LiFSI/DME (FIG. 19b). The leakage current from this stage likely originates from Al3+ dissolution and complexation with FSI⁻ to form Al(FSI)x. (Yamada, Y.; Chiang, C. H.; Sodeyama, K.; Wang, J.; Tateyama, Y.; Yamada, A. Corrosion Prevention Mechanism of Aluminum Metal in Superconcentrated Electrolytes. ChemElectroChem 2015, 2 (11), 1687-1694. https://doi.org/10.1002/celc.201500235) This trend in leakage current quickly reverses after 1 hour due to passivation by Al(FSI)x. Electrolytes with weaker solvation ability is unable to dissolve a large amount of Al(FSI)x complexes, thereby achieving the best passivation. Id. At the later stage of voltage holding, the leakage current remains very low for 4 M LiFSI/DEE, which results in no corrosion as seen in SEM images. Finally, it is worth noting that both 1 M LiFSI/DME and 1 M LiFSI/DEE only remain stable below 4.5 V (vs. Li+/Li) on Pt electrode (FIG. 12). Therefore, the improved stability of Al electrode in DEE is due to surface passivation rather than the intrinsic oxidation stability of DEE.

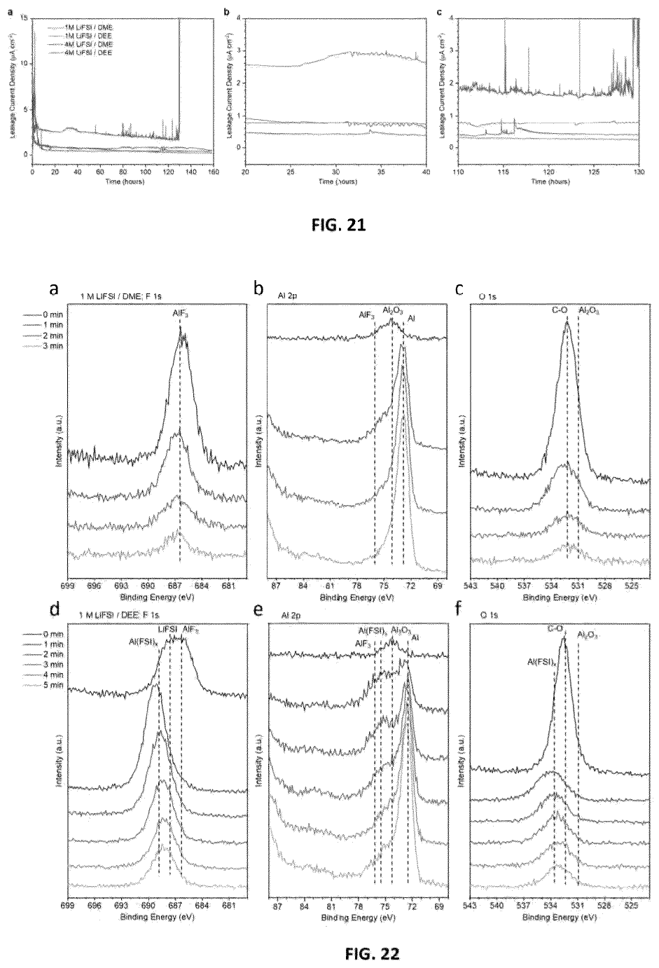

The Al corrosion experiment was also carried out at 4.4 V (vs. Li+/Li) to match the upper cutoff voltage of Ni-rich NMC cathodes. Obvious cracking and pitting of Al are observed using 1 M LiFSI/DME, whereas only small pits are observed with 1 M LiFSI/DEE (FIG. 20). For both 4 M LiFSI/DME and DEE, the pitting is even more subtle (FIG. 20). The lower leakage current from DEE electrolytes compared to DME electrolytes with the same LiFSI concentrations during 4.4 V holding indicates the former is more stable against Al corrosion (FIG. 21).

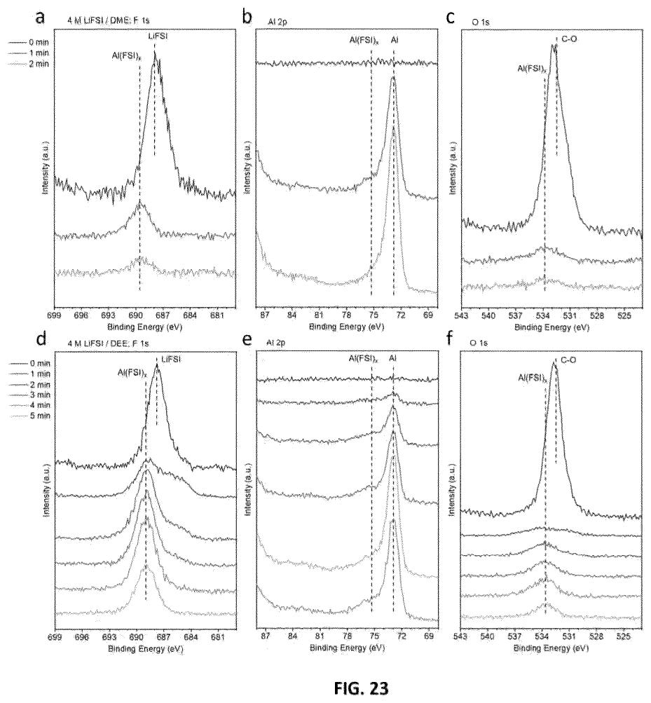

The surface layer on Al electrode was characterized by XPS to further study the passivation behavior of each electrolyte. The same corrosion protocol as above was carried out at 5.5 V. Based on XPS depth profiles, 1 M LiFSI/DME results in very thin surface layer on Al as is evident from the quick increase of Al and diminishing of other elements within 2 minutes of sputtering (FIG. 4m). The surface is rich in O, C, F and S (FIG. 4m). The XPS spectra reveal AlF₃, Al₂O₃ and organic ether species as the major components of the surface layer (FIGS. 22a-c) (Tateishi, K.; Waki, A.; Ogino, H.; Ohishi, T.; Murakami, M. Formation of Al₂O₃ Film and AlF₃ Containing Al₂O₃ Film by an Anodic Polarization of Aluminum in Ionic Liquids. Electrochemistry 2012, 80 (8), 556-560; Theivaprakasam, S.; Girard, G.; Howlett, P.; Forsyth, M.; Mitra, S.; MacFarlane, D. Passivation Behaviour of Aluminium Current Collector in Ionic Liquid Alkyl Carbonate (Hybrid) Electrolytes. NPJ Mater. Degrad. 2018, 2, 13. https://doi.org/10.1038/s41529-018-0033-6), which indicates decomposition of both solvent and anion. Although AlF₃ was reported to effectively passivate Al electrode in LiPF₆-carbonate electrolytes, it was found incapable of passivation in LiTFSI-carbonate electrolyte. In addition, the solvent-derived organic and Al₂O₃ components likely further contribute to the poor passivation. In contrast, the passivation layer from 1 M LiFSI/DEE is much thicker and richer in F compared to 1 M LiFSI/DME (FIG. 4n). The top surface composition appears similar to that from 1 M LiFSI/DME but with the addition of LiFSI (FIGS. 22d-f). The higher surface F content indicates increased anion decomposition (FIG. 4n). Underneath the surface, the composition from 1 M LiFSI/DEE differs dramatically from 1 M LiFSI/DME as a new set of peaks appear at higher binding energy (FIGS. 22d-f), which were assigned to a thick layer rich in Al(FSI)x. Compared to DME, the solvation ability of DEE is weaker, which enables the accumulation of Al(FSI)x and additional unidentified fluorinated species. This layer more effectively passivates Al surface compared to the case in 1 M LiFSI/DME. However, the presence of Al₂O₃ and AlF₃ on the surface (FIGS. 22d-f), which results from electrolyte decomposition, indicates the quality of the passivation layer is suboptimal. Similar to 1 M LiFSI/DME, 4 M LiFSI/DME also has a thin protecting layer (FIG. 4o). However, the top surface is free of Al (FIG. 23b), which is a strong indication of good passivation. The layer underneath the surface is quite thin but rich in Al(FSI)x (FIGS. 23a-c). The strong solvation ability of DME likely prevents the build-up of a thick passivation layer even at 4 M LiFSI concentration. The most stable electrolyte, 4 M LiFSI/DEE, forms a thick passivation layer that is very rich in F (FIG. 4p). The top surface is free of Al and the signal of Al only becomes significant after 2 minutes of sputtering (FIG. 23e), which indicates very good passivation. The layer underneath is thick and abundant in Al(FSI)x and additional unidentified fluorinated species (FIGS. 23d-f), likely as a result of poor solvation ability of DEE at a high salt concentration. It is worth pointing out that in addition to Al(FSI)x, additional unidentified fluorinated species (based on atomic percentages) are also important for the passivation of Al electrode. Additional work is required to accurately identify these species.

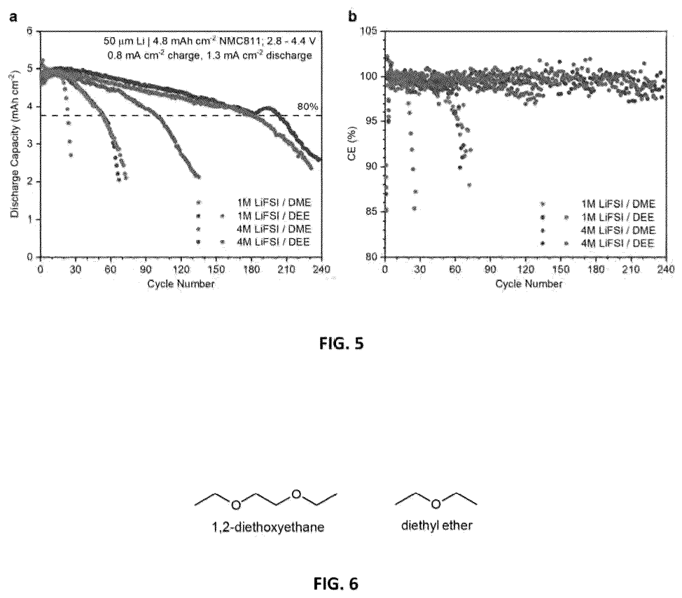

FIG. 5 illustrates LiINMC811 full cell performance under stringent conditions of high-loading NMC811 (ca. 4.8 mAh cm⁻²), thin Li (50 μm thick, N/P=2), and relatively lean electrolyte condition (E/C=8 mL Ah⁻¹). All cells were cycled between 2.8 and 4.4 V. Two formation cycles at 0.4 mA cm⁻² charge and discharge were followed by long-term cycling at 0.8 mA cm⁻² charge and 1.3 mA cm⁻² discharge. For long-term cycling, a constant-voltage hold at 4.4 V was implemented until current drops to 0.2 mA cm⁻². FIG. 5(a) illustrates discharge capacity and FIG. 5(b) CE. Repeated cells using DEE electrolytes are shown.

I. A. 2. d. Full-Cell Performance

Finally, to experimentally verify the enhanced stability of DEE compared to DME, the full-cell performance of these electrolytes was tested under stringent conditions. To demonstrate the high-voltage stability of DEE, the state-of-the-art NMC811 cathode was selected due to its high reactivity and high specific capacity as a consequence of high Ni content. (Manthiram, A. A Reflection on Lithium-Ion Battery Cathode Chemistry. Nat. Commun. 2020, 11 (1), 1-9. https://doi.org/10.1038/s41467-020-15355-0) A high cut-off voltage at 4.4 V and relatively large charge and discharge current of 0.8 mA cm⁻² and 1.3 mA cm⁻² respectively were used. A high cathode loading of ca. 4.8 mAh cm⁻² helped mimic the condition in realistic high-energy-density batteries where “deep” cycling of Li anode is required. 1 The thickness of Li (50 μm thick, N/P=2) and the volume of electrolyte (E/C=8 mL Ah⁻¹) were limited to reflect a realistic cycling condition.

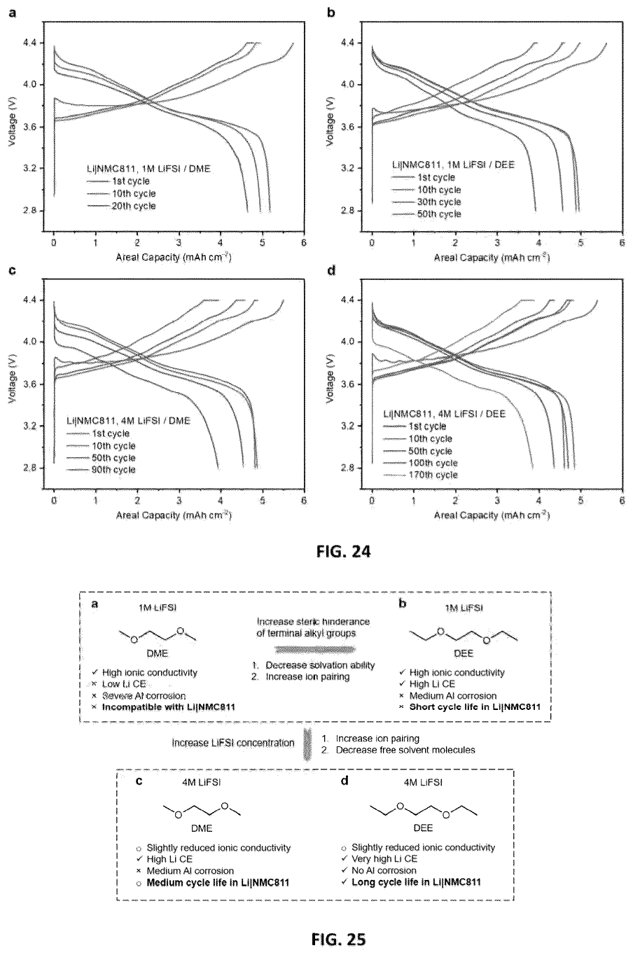

The areal discharge capacities during long-term cycling are compared in FIG. 5a and the corresponding CEs are shown in FIG. 5b. The voltage profiles are shown in FIG. 24. The capacity of the full cell using 1 M LiFSI/DME sharply decreases after 20 cycles due to quick consumption of Li reservoir and/or electrolyte1, which results from poor electrolyte stability at both Li anode and high-voltage NMC811 cathode. In contrast, 1 M LiFSI/DEE sustains 50 cycles before reaching 80% capacity and no sharp capacity decay is observed. This improvement of DEE over DME is consistent with the higher Li CE and oxidation stability as discussed above. Upon increasing the LiFSI concentration to 4 M, both DME and DEE electrolytes achieve longer cycle lives (94 cycles and 182 cycles respectively until 80% capacity retention) due to improved Li CE and high voltage stability, which is consistent with previous reports on HCEs. Interestingly, even at such a high salt concentration, the advantage of DEE over DME remains evident. The Li anode performance of HCEs is often attributed to the increased contribution of anion and the decreased contribution of solvent to the SEI (hence the term “anion-derived SEI”). Similarly, the limited amount of non-coordinating solvent molecules in HCEs reduces side reactions between cathode and electrolyte. Our results build upon previous knowledge and further demonstrate that despite the importance of FSI− anion in HCEs, solvent properties can still significantly impact the performance of LMBs.

### I. A. 3. Conclusion

Despite the common choice of LiFSI/DME in recent designs of advanced electrolytes, the instability of DME at both Li anodes and high-voltage cathodes makes it suboptimal for long-term cycling. Beyond fluorination, molecular design strategies that enable stable operation of high-voltage LMBs in ether electrolytes are still lacking. In this work, we report a molecular design principle that leverages the steric hindrance effect to tune the solvation ability of ether molecules. Guided by both experimental and computational studies, we identified DEE as a weaklier solvating molecule compared to DME. When paired with 1 M and 4 M LiFSI, DEE exhibits higher Li CEs and voltage stability than the DME counterparts due to improved interfacial properties. Under stringent full-cell cycling conditions of ca. 4.8 mAh cm⁻² NMC811 and 50 μm thin Li between 2.8 and 4.4 V, 4 M LiFSI/DEE sustained 182 cycles while 4 M LiFSI/DME only cycled for 94 cycles until 80% capacity retention. A summary of the relevant electrolyte properties is shown in FIG. 25. Our work advances from the current emphasis on anion-derived properties to further demonstrate the significance of solvent design. Overall, we demonstrate that DEE is a more suitable solvent than DME for high-voltage LMBs. More importantly, this new design strategy utilizing the steric effect of solvent molecules opens up new opportunities for future molecular design of electrolyte solvents.

### I. A. 4. Experimental Section

### Materials

DEE (98%) and DME (anhydrous, 99.5%, inhibitor-free) were purchased from Sigma-Aldrich. DEE (99%, ACROS) was also purchased from Fisher Scientific. Sodium hydride (60%, dispersion in Paraffin liquid) was purchased from TCI. LiFSI was purchased from Arkema. Celgard 2325 separator (25 μm thick, polypropylene/polyethylene/polypropylene) was purchased from Celgard. Cu current collector (25 μm thick) was purchased from Alfa Aesar. Thin lithium foil (50 μm) was purchased from Uniglobe Kisco Inc. Lithium chips (600 μm), 2032-type battery casings, stainless steel spacers, springs and Al-clad coin cell cases were purchased from MTI. NMC811 cathode sheets (ca. 4.8 mAh cm⁻², 20.47 mg cm⁻² active materials) were purchased from Targray.

### Solvent Purification

DEE was purified by vacuum distillation for three times. A small amount of sodium hydride was added before the second and third distillation to remove water. The pure product was stored in an Ar-filled glovebox. DME is of high purity and was not distilled. Fresh Li foil was added to both solvents inside the glovebox to further remove trace amount of water.

### Electrolyte Preparation

Electrolytes were prepared by dissolving 1 M or 4 M of LiFSI in DME or DEE. The molarities were calculated based on the moles of salt and the volumes of solvents. The electrolytes were filtered through 1 μm PTFE syringe filters before use.

### Electrochemical Measurements

2032-type coin cells were used for all electrochemical measurements under ambient conditions. Battery fabrication was carried out in an Ar-filled glovebox. One piece of Celgard 2325 was used as separator. Thick Li foil with fresh surface of 7/16” in diameter and 40 μL of electrolyte were used unless otherwise specified. Oxidation stability of electrolytes was measured by linear sweep voltammetry on Li|Al and Li|Pt cells using Biologic VSP300. The voltage swept from open-circuit voltage to 7 V vs. Li+/Li at a rate of 1 mV s⁻¹. The leakage current density was calculated based on an electrode area of 2.11 cm⁻². Symmetric cells with two stainless steel electrodes and electrolyte-soaked separator were assembled to measure bulk impedance using Biologic VSP. Li|Cu, Li|Li and Li|NMC cells were tested on Land or Arbin battery testing stations. CEs were measured by a modified Aurbach method on Li|Cu cells. The Cu surface was conditioned by plating 5 mAh cm⁻² of Li and stripping to 1 V at 0.5 mA cm⁻². Then, a Li reservoir of 5 mAh cm⁻² was plated onto Cu, followed by 10 cycles of Li plating and striping at 1 mAh cm⁻² and 0.5 mA cm⁻². Finally, all Li on Cu was stripped to 1 V at 0.5 mA cm⁻². For the long-term cycling of Li|Cu cells, the Cu surface was conditioned by holding at 0.01 V for 5 hours, and then cycling between 0 and 1 V at 0.2 mA cm⁻² for 10 cycles. During cycling, 1 mAh cm⁻² of Li was plated onto Cu and was then stripped to 1 V at 0.5 mA cm⁻². In addition, 5 mAh cm⁻² capacity was also used for Li|Cu cycling. Li|Li symmetric cells were cycled at 1 mA cm⁻² for 1 mAh cm⁻². LiINMC811 full cells were fabricated using 50 μm thin Li (ca. 10 mAh cm⁻²), very high-loading NMC811 cathode (ca. 4.8 mAh cm⁻²) and relatively lean electrolyte amount (40 μL). Al-clad cathode cases were used. Al foil was placed inside the cathode cases to avoid defects in the Al cladding. Full cells were cycled between 2.8 and 4.4 V. Two formation cycles were performed at 0.4 mA cm⁻² charge and discharge current. For long-term cycling, cells were charged at 0.8 mA cm⁻², held at 4.4 V until current <0.2 mA cm⁻², and discharged at 1.3 mA cm⁻².

### Materials Characterization

The surface morphologies of Al and Li were imaged by SEM on FEI Magellan 400 XHR Scanning Electron Microscope. Li|Al coin cells were fabricated as described above using 80 μL of electrolyte. Al corrosion was carried out using Biologic VSP system. First, LSV was performed from open-circuit voltage to 5.5 V or 4.4 V vs. Li+/Li at a rate of 1 mV s⁻¹. Then, the voltage was held at 5.5 V for about 20 hours or at 4.4 V for about 160 hours. The cells were disassembled, and the Al foil was rinsed with the corresponding DME or DEE solvent. Li deposition morphology on Cu was studied by depositing 1 mAh cm⁻² of Li at 0.5 mA cm⁻² in Li|Cu cells. The Cu substrates were conditioned by holding at 0.01 V for 5 hours, and then cycling between 0 and 1 V at 0.2 mA cm⁻² for 10 cycles before Li deposition. After Li deposition, the cells were disassembled, and the Cu electrodes were rinsed with the corresponding DME or DEE solvent.

The surface compositions of Al and Li were characterized by PHI VersaProbe 3 XPS with monochromatized Al(Ka) Source (1486 eV) and focused ion gun. An air-tight vessel was used to transfer samples without exposure to air. Al corrosion was carried out as described above at 5.5 V for about 23 hours. The obtained Al electrodes were only briefly rinsed to avoid significant dissolution of surface layer. Li|Li cells were cycled at 1 mA cm⁻², 1 mAh cm⁻² for 10 cycles. The Li electrode that was stripped in the final step was rinsed by the corresponding solvent and characterized.

7Li NMR was performed on Varian Inova 500 MHz NMR. 19F and 1H NMR were performed on Varian 400 MHz NMR. The temperature was set at 25° C. Each electrolyte was injected into a capillary tube, which was sealed by a PTFE cap and was inserted into an NMR tube containing an external standard solution. The samples were locked and shimmed using the external standard. The chemical shifts were referenced to the standard solutions: 1 M LiCl in D₂O for 7Li (0 ppm), 0.1 M 4-fluoronitrobenzene in CDCl3 for 19F (−102 ppm) and 1H (7.24 ppm).

Raman spectra were collected on Horiba XploRA+ Confocal Raman with 532 nm excitation laser. The electrolytes were sealed in quartz cuvettes.

Solvation energy measurement was recently developed by our groups. The home-made apparatus is composed of a T-shaped glass flange assembled in between a H-cell. The apparatus is composed of three chambers, each containing a different electrolyte (test electrolyte, reference electrolyte, salt bridge electrolyte), and two porous junctions that separate the three chambers. Four layers of 25 μm PE/PP/PE separators (Celgard 2325) were used as porous junctions. Two pieces of fresh lithium foil were used as electrodes. Each electrode was connected to a potentiometer (Biologic VMP3) to measure Ecell Voltage was recorded after stabilization, which typically takes up to three minutes.

### Theoretical Calculations

The molecular geometries and coordination energies were optimized and calculated by DFT using Gaussian 09 package at the B3LYP/6-311G+(d, p) level. ESPs were generated using SCF density matrix.

Molecular dynamics simulations were carried out using Gromacs 2018 (Abraham, M. J.; Murtola, T.; Schulz, R.; Pill, S.; Smith, J. C.; Hess, B.; Lindah, E. Gromacs: High Performance Molecular Simulations through Multi-Level Parallelism from Laptops to Supercomputers. SoftwareX 2015, 1-2, 19-25. https://doi.org/10.1016/j.softx.2015.06.001), with electrolyte molar ratios taken from those used in the experimental work. Molecular forces were calculated using the Optimized Potentials for Liquid Simulations all atom (OPLS-AA) force field. (Jorgensen, W. L.; Maxwell, D. S.; Tirado-Rives, J. Development and Testing of the OPLS All-Atom Force Field on Conformational Energetics and Properties of Organic Liquids. J. Am. Chem. Soc. 1996, 118 (45), 11225-11236. https://doi.org/10.1021/ja9621760) Topology files and bonded and Lennard-Jones parameters were generated using the LigParGen server. (Dodda, L. S.; De Vaca, I. C.; Tirado-Rives, J.; Jorgensen, W. L. LigParGen Web Server: An Automatic OPLS-AA Parameter Generator for Organic Ligands. Nucleic Acids Res. 2017, 45 (W1), W331-W336. https://doi.org/10.1093/nar/gkx312) Atomic partial charges were calculated by fitting the molecular ESP at atomic centers in Gaussian16 using the Meller-Plesset second-order perturbation method with a cc-pVTZ basis set. (Gaussian 16, Revision B.01, M. J. Frisch, D. J. Fox et Al, Gaussian, Inc., Wallingford CT, 2016). Due to the use of a non-polarizable force field, partial charges for charged ions were scaled by 0.8 to account for electronic screening, which has been shown to improve predictions of interionic interactions. (Humphrey, W.; Dalke, A.; Schulten, K. VMD: Visual Molecular Dynamics. J. Mol. Graph. 1996, 14, 33-38; Leontyev, I.; Stuchebrukhov, A. Accounting for Electronic Polarization in Non-Polarizable Force Fields. Phys. Chem. Chem. Phys. 2011, 13 (7), 2613-2626. https://doi.org/10.1039/c0cp01971b) The simulation procedure consisted of an energy minimization using the steepest descent method followed by a 8 ns equilibration step using a Berendsen barostat and a 40 ns production run using a Parrinello-Rahman barostat, both at a reference pressure of 1 bar with timesteps of 2 fs. A Nose-Hoover thermostat was used throughout with a reference temperature of 300 K. The particle mesh Ewald method was used to calculate electrostatic interactions, with a real space cutoff of 1.2 nm and a Fourier spacing of 0.12 nm. The Verlet cutoff scheme was used to generate pairlists. A cutoff of 1.2 nm was used for non-bonded Lennard-Jones interactions. Periodic boundary conditions were applied in all directions. Bonds with hydrogen atoms were constrained. Convergence of the system energy, temperature, and box size were checked to verify equilibration. The final 30 ns of the production run were used for the analysis. Density profiles and RDFs were generated using Gromacs, while visualizations were generated with VMD. (Self, J.; Fong, K. D.; Persson, K. A. Transport in Superconcentrated LiPF6 and LiBF4/Propylene Carbonate Electrolytes. ACS Energy Lett. 2019, 4 (12), 2843-2849. https://doi.org/10.1021/acsenergylett.9b02118) Solvation shell statistics were calculated using the MDAnalysis Python package (Allouche, A. Software News and Updates Gabedit—A Graphical User Interface for Computational Chemistry Softwares. J. Comput. Chem. 2012, 32, 174-182. https://doi.org/10.1002/jcc) by histogramming the observed first solvation shells for lithium ions during the production simulation, using a method similar to previous work.14 The cutoff distance for each species in the first solvation shell was calculated from the first minimum occurring in the RDF (referenced to lithium ions) after the initial peak.

Commercial Applications of the present embodiments include:

1. These electrolytes could be implemented in lithium metal batteries of various cathode chemistries and cell form factors. The use of these electrolytes is fully compatible with current manufacturing processes.

2. The synthesis of 1,2-diethoxyethane and its isomers could be adopted for large scale manufacturing.

Advantages and improvements over existing methods, devices or materials of the present embodiments include:

1. Higher lithium coulombic efficiency compared to electrolytes using other nonfluorinated solvents (eg. 1,2-dimethoxyethane)

2. Better tolerance towards high voltage compared to electrolytes using other nonfluorinated solvents (eg. 1,2-dimethoxyethane)

3. Lower cost, environmental and health impact compared to fluorinated solvents (eg. 1,1,2,2-tetrafluoroethyl-2,2,3,3-tetrafluoropropylether)

### I. A. 5 Supplemental Information

FIG. 6 illustrates Molecular structures of 1,2-diethoxyethane (DEE) and diethyl ether. It is worth noting that the DEE reported here is not to be confused with diethyl ether (commonly known as “ether”), which was recently developed for low-temperature LMBs (Holoubek, J.; Liu, H.; Wu, Z.; Yin, Y.; Xing, X.; Cai, G.; Yu, S.; Zhou, H.; Pascal, T. A.; Chen, Z.; et al. Tailoring Electrolyte Solvation for Li Metal Batteries Cycled at Ultra-Low Temperature. Nat. Energy 2021, 6, 303-313. https://doi.org/10.1038/s41560-021-00783-z; Liu, H.; Holoubek, J.; Zhou, H.; Chen, A.; Chang, N.; Wu, Z.; Yu, S.; Yan, Q.; Xing, X.; Li, Y.; et al. Ultrahigh Coulombic Efficiency Electrolyte Enables Li∥SPAN Batteries with Superior Cycling Performance. Mater. Today 2021, 42 (xx), 17-28. https://doi.org/10.1016/j.mattod.2020.09.035) and which shares the same acronym.

FIG. 7 illustrates (a) 19F NMR of FSI− in each electrolyte; (b) 1H NMR of protons adjacent to ether oxygens on DEE; (c) 1H NMR of protons adjacent to ether oxygens on DME. The chemical shifts are referenced to 0.1 M 4-fluoronitrobenzene in CDCl3 at −102 ppm for 19F and 7.24 ppm for 1H. Peak intensities are normalized for clarity. All samples were characterized neat with an external reference.

FIG. 8 illustrates Raman spectra of 1 M and 4 M LiFSI/DME and DEE. The peak intensities are normalized. The Raman bands arise from FSI⁻ vibrations. The wavenumbers are dependent on the coordination environment of FSI−: solvent-separated ion pairs (SSIP), contact ion pairs (CIP), ion aggregates (AGG).

TABLE S1
Inner solvation shell compositions around Li+ and their
corresponding probabilities in each electrolyte calculated from MD simulations.

| 1M LiFSI/DME | | | 1M LiFSI/DEE | | | 4M LiFSI/DME | | | 4M LiFSI/DEE | | |
|---|---|---|---|---|---|---|---|---|---|---|---|
| FSI- | DME | Probability | FSI- | DEE | Probability | FSI- | DME | Probability | FSI- | DEE | Probability |
| 0 | 3 | 6.825 | 0 | 2 | 10.542 | 0 | 3 | 2.706 | 0 | 2 | 5.177 |
| 1 | 2 | 55.968 | 0 | 3 | 0.283 | 1 | 2 | 24.601 | 1 | 2 | 21.421 |
| 1 | 3 | 0.459 | 1 | 2 | 43.329 | 1 | 3 | 0.189 | 2 | 1 | 48.631 |
| 2 | 1 | 18.373 | 1 | 3 | 0.165 | 2 | 1 | 18.376 | 2 | 2 | 1.619 |
| 2 | 2 | 11.319 | 2 | 1 | 40.96 | 2 | 2 | 18.84 | 3 | 0 | 1.783 |
| 3 | 0 | 0.204 | 2 | 2 | 1.148 | 3 | 0 | 2.19 | 3 | 1 | 14.918 |
| 3 | 1 | 6.057 | 3 | 1 | 3.52 | 3 | 1 | 22.585 | 4 | 0 | 6.036 |
| 3 | 2 | 0.121 | | | | 3 | 2 | 0.561 | 4 | 1 | 0.109 |
| 4 | 0 | 0.111 | | | | 4 | 0 | 5.115 | 5 | 0 | 0.255 |
| 4 | 1 | 0.545 | | | | 4 | 1 | 2.845 | | | |
| | | | | | | 5 | 0 | 1.943 | | | |

FIG. 9 illustrates DFT calculations. ESP of (a) DME and (b) DEE; optimized binding geometry and energy of (c) Li+-DME and (d) Li+-DEE.

FIG. 10 illustrates The distributions of possible inner solvation shell compositions of (a) 4 M LiFSI/DEE and (b) 4 M LiFSI/DME from MD simulation. For each electrolyte, the major compositions are shown and the rest are grouped as “others”.

FIG. 11 illustrates The distributions of various Li+ coordination environments for (a) 1 M LiFSI/DME, (b) 1 M LiFSI/DEE, (c) 4 M LiFSI/DME, and (d) 4 M LiFSI/DEE. The Li+ coordination environments are categorized based on the number of FSI− in the inner solvation shells of Li+.

Note: The percentages of 0 FSI− are higher in DEE than in DME at both 1 M and 4 M LiFSI concentrations. These results might be mistaken as evidence for stronger binding ability of DEE in bulk solution (which contradicts with other experimental and computational results). However, this is not the case upon closer inspection of solvation structures (Table S1). In 1 M and 4 M LiFSI/DME, the FSI−-free solvation shell is consisted of 3 DME, whereas in 1 M and 4 M LiFSI/DEE, it is made of 2 DEE. This further supports the stronger steric effect of DEE than DME—it is difficult to include three DEE in the Li⁺ inner solvation shell while three DME that are smaller in size could be accommodated.

FIG. 12 illustrates Oxidation stability of various electrolytes on Pt electrode. Each cell was scanned from Voc to 7 V (vs. Li+/Li). The reference line indicates 4.4 V.

FIG. 13 illustrates Long-term cycling of Li|Cu half cells in various electrolytes at 1 mAh cm−2 capacity.

FIG. 14 illustrates Cycling of Li|Cu half cells in 4 M electrolytes at 5 mAh cm−2 capacity.

FIG. 15 illustrates Ionic conductivities of 1 M and 4 M LiFSI/DME and LiFSI/DEE. Impedance measurements on SS|Celgard2325|SS coin cells were used to calculate the ionic conductivities. These values reflect the ionic conductivities in actual full cells. Three replicates were made for each electrolyte.

FIG. 16 illustrates Additional SEM images of Li metal deposition on bare Cu in various electrolytes (1st cycle plating, 0.5 mA cm−2, 1 mAh cm−2).

FIG. 17 illustrates Surface XPS spectra of cycled Li electrodes in various electrolytes (Li|Li cells, 10 cycles at 1 mA cm−2, 1 mAh cm−2). (a) F 1s, (b) O 1s, (c) N 1s, (d) C is, (e) S 2p.

Note: The absolute intensities of O, F and S are higher while that of Li is slightly lower from 4 M LiFSI/DEE compared to 4 M LiFSI/DME. As a result, the Li percentage appears much higher in 4 M LiFSI/DME. In addition, the relative sensitivity factor for Li is XPS is large while the peak intensities are low. This could further introduce error in atomic percentages.

FIG. 18 provides Additional SEM images of Al electrodes after being held at 5.5 V (vs. Li⁺/Li) in various electrolytes for ca. 20 hours. (a) 1 M LiFSI/DME. Several representative surface morphologies, including pits, roughened surface and cracks, are shown. The inserts are optical images of discolored Al (left) and electrolyte (right) after corrosion. (b) 1 M LiFSI/DEE. Areas of severe corrosion and clear surfaces are shown. The insert is an optical image of Al with discolored electrolyte after corrosion. (c) 4 M LiFSI/DME. Cracks and pits are the dominant morphologies. The insert is an optical image of Al after corrosion. (d) 4 M LiFSI/DEE. No corrosion is observed. The surface features are native to the Al sheet used. The insert is an optical image of Al after corrosion experiment.

FIG. 19 illustrates Leakage currents during Al corrosion in various electrolytes at 5.5 V (vs. Li+/Li) for about 20 hours. (a) Leakage current over time; (b) zoomed-in view.

FIG. 20 provides SEM images of Al electrodes after being held at 4.4 V (vs. Li+/Li) in various electrolytes for ca. 160 hours (except for 1 M LiFSI/DME, which became unstable after ca. 130 hours). The white circles indicate small pits with clear sharp edges as a result of Al corrosion.

FIG. 21 provides Leakage currents during Al corrosion in various electrolytes at 4.4 V (vs. Li+/Li). (a) Leakage current over time; zoom-in view at (b) early and (c) late stages of the experiment. The stabilized leakage currents in DEE electrolytes are lower than those in DME electrolytes at the same LiFSI concentrations.

FIG. 22 illustrates XPS depth profiles of Al electrodes after being held at 5.5 V (vs. Li+/Li) for about 23 hours in (a-c) 1 M LiFSI/DME and (d-f) 1 M LiFSI/DEE. (a,d) F Is, (b,e) Al 2p, and (c,f) O is spectra. The Ar+ sputtering rate is consistent for all samples (5 kV, 3 A, 1×1 mm).

FIG. 23 illustrates XPS depth profiles of Al electrodes after being held at 5.5 V (vs. Li+/Li) for about 23 hours in (a-c) 4 M LiFSI/DME and (d-f) 4 M LiFSI/DEE. (a,d) F Is, (b,e) Al 2p, and (c,f) O is spectra. The Ar+ sputtering rate is consistent for all samples (5 kV, 3 A, 1×1 mm).

FIG. 24 illustrates voltage profiles of LiINMC811 full cells using each electrolyte. The 1st cycle was carried out at 0.4 mA cm−2 charge and discharge. All other cycles were performed at 0.8 mA cm−2 charge and 1.3 mA cm−2 discharge with a constant voltage hold at 4.4 V until current drops to 0.2 mA cm−2.

FIG. 25 provides a Summary of electrolytes and their properties investigated in this work: (a) 1M LiFSI/DME; (b) 1M LiFSI/DEE; (c) 4M LiFSI/DME; (d) 4M LiFSI/DEE.

### I. B Design of Non-Fluorinated Ether Solvents for Lithium Metal Battery Electrolytes

Two design strategies for non-fluorinated ether solvents as electrolytes in lithium metal batteries. Functional groups with various levels of steric hindrance can be leveraged to tune the solvation ability of ether solvents. The arrangement of oxygen atoms can be modified to tune the solvation ability of ether solvents.

The figures demonstrate how the resulting electrolytes enable high lithium coulombic efficiency and good high-voltage tolerance:

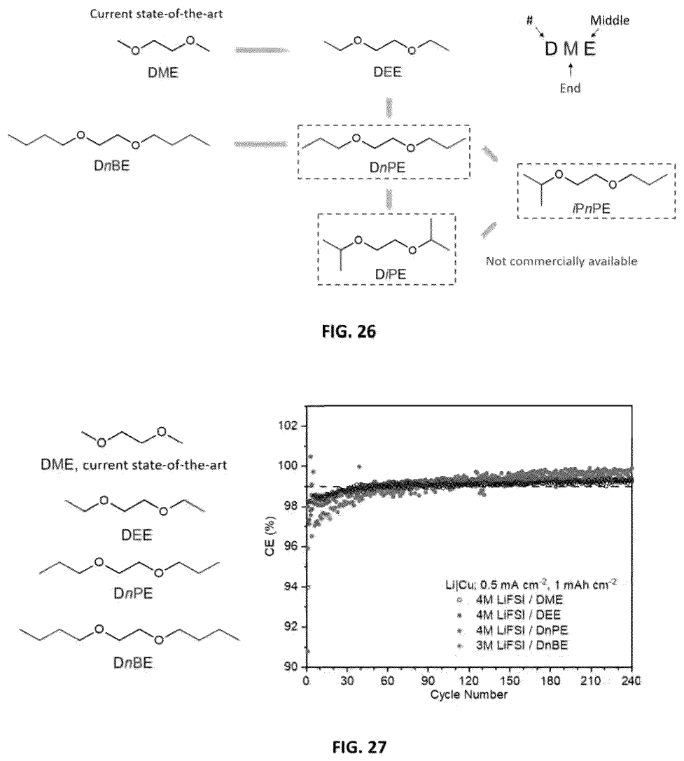

FIG. 26 illustrates Functional groups with various levels of steric hindrance can be leveraged to tune the solvation ability of ether solvents.

FIG. 27 illustrates how DEE, DnPE, DnBE show improved CE compared to DME

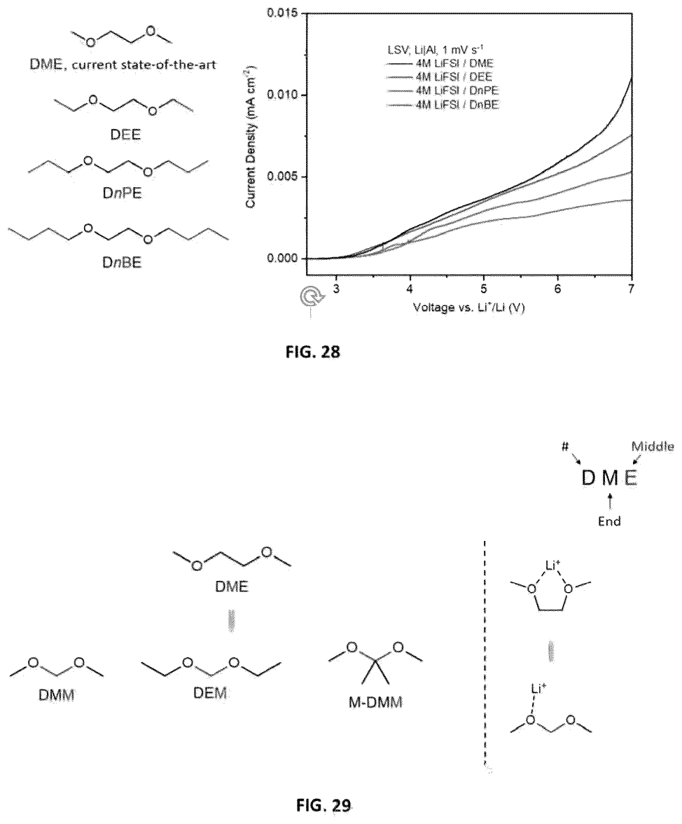

FIG. 28 illustrates how DEE, DnPE, DnBE show improved oxidative stability compared to DME.

FIG. 29 illustrates The arrangement of oxygen atoms can be modified to tune the solvation ability of ether solvents.

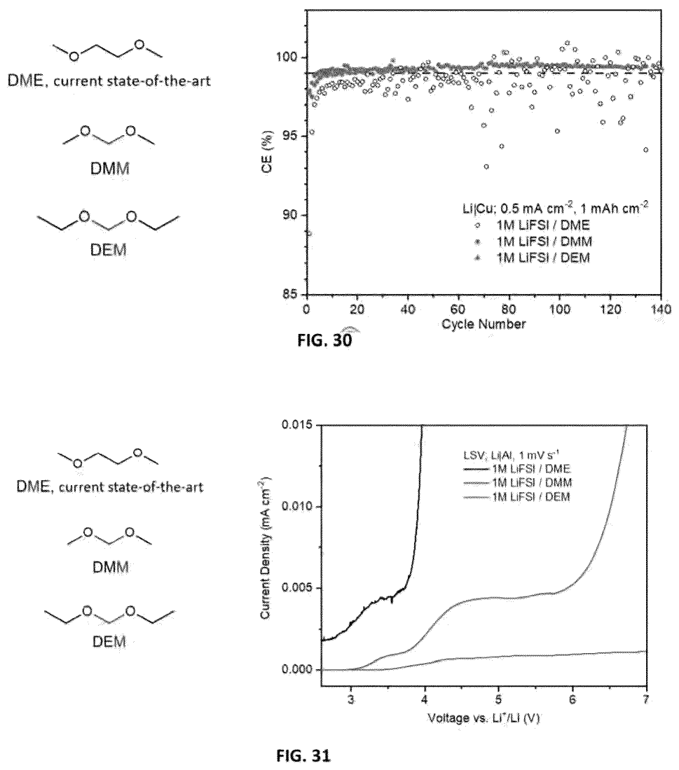

FIG. 30 illustrates how 1M LiFSI/DMM and DEM show very quick activation to reach >99% CE.

FIG. 31 illustrates how 1M LiFSI/DMM and DEM show improved oxidative stability compared to DME.

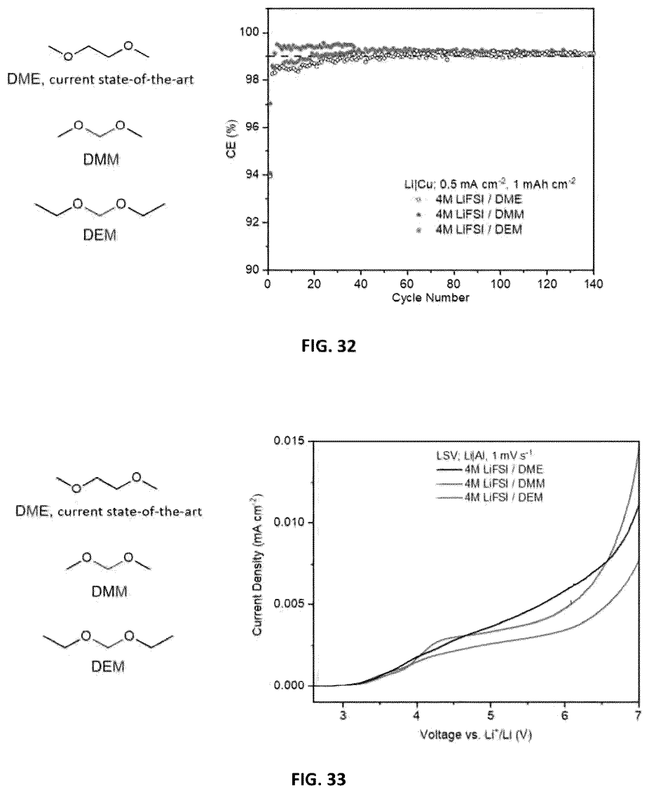

FIG. 32 illustrates how 4M LiFSI/DMM and DEM achieve quicker activation than DME.

FIG. 33 illustrates how 4M LiFSI/DMM and DEM show similar or slightly better oxidative stability compared to DME

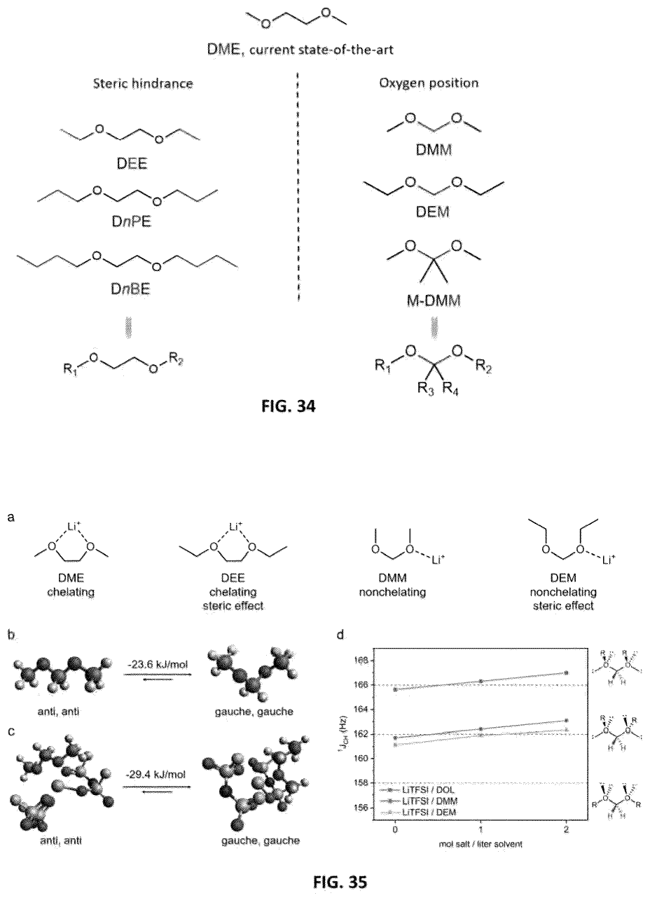

FIG. 34 presents a summary of the above and other aspects.

### I. C. Acetal-Based Electrolyte Simultaneously Enables Lithium Metal Stability and Fast Ion Transport

### I. C. 1. Introduction

Lithium-metal (Li) anode has attracted enormous research interest due to its low redox potential and high specific capacity. However, its high reactivity poses significant challenge to battery stability. (Lin, D.; Liu, Y.; Cui, Y. Reviving the Lithium Metal Anode for High-Energy Batteries. Nat. Nanotechnol. 2017, 12 (3), 194-206. https://doi.org/10.1038/nnano.2017.16.) The commercial carbonate electrolytes are incompatible with Li metal due to the poor quality of solid electrolyte interface (SEI). During charge and discharge, the large volume change of Li metal leads to SEI damage. The resulting inhomogeneity on electrode surface leads to the undesirable growth of high-aspect-ratio Li. In addition, the repeated damage and repair of SEI results in low CE and quick consumption of electrolyte and Li reservoir.

Electrolyte design is arguably the most effective strategy to overcome the issue of SEI instability. (Wang, H.; Yu, Z.; Kong, X.; Kim, S. C.; Boyle, D. T.; Qin, J.; Bao, Z.; Cui, Y. Liquid Electrolyte: The Nexus of Practical Lithium Metal Batteries. Joule 2022, 6 (3), 588-616. https://doi.org/10.1016/j.joule.2021.12 018.) In recent years, numerous advanced electrolytes have reached Li metal Coulombic efficiency (CE) of >99% with bulky Li deposition morphology. (Hobold, G. M.; Lopez, J.; Guo, R.; Minafra, N.; Banerjee, A.; Shirley Meng, Y.; Shao-Horn, Y.; Gallant, B. M. Moving beyond 99.9% Coulombic Efficiency for Lithium Anodes in Liquid Electrolytes. Nat. Energy 2021, 6 (10), 951-960. https://doi.org/10.1038/s41560-021-00910-w.) Some of the most effective designs include standard concentration electrolytes, (Yu, Z.; Wang, H.; Kong, X.; Huang, W.; Tsao, Y.; Mackanic, D. G.; Wang, K.; Wang, X.; Huang, W.; Choudhury, S.; et al. Molecular Design for Electrolyte Solvents Enabling Energy-Dense and Long-Cycling Lithium Metal Batteries. Nat. Energy 2020, 5 (7), 526-533. https://doi.org/10.1038/s41 560-020-0634-5; Yu, Z.; Rudnicki, P. E.; Zhang, Z.; Huang, Z.; Celik, H.; Oyakhire, S. T.; Chen, Y.; Kong, X.; Kim, S. C.; Xiao, X.; et al. Rational Solvent Molecule Tuning for High-Performance Lithium Metal Battery Electrolytes. Nat. Energy 2022, 7 (1), 94-106. https://doi.org/10.1038/s4 1560-021-00962-y; Holoubek, J.; Liu, H.; Wu, Z.; Yin, Y.; Xing, X.; Cai, G.; Yu, S.; Zhou, H.; Pascal, T. A.; Chen, Z.; et al. Tailoring Electrolyte Solvation for Li Metal Batteries Cycled at Ultra-Low Temperature. Nat. Energy 2021, 6, 303-313. https://doi.org/10.1038/s41560-021-00783-z; Xue, W.; Huang, M.; Li, Y.; Zhu, Y. G.; Gao, R.; Xiao, X.; Zhang, W.; Li, S.; Xu, G.; Yu, Y.; et al. Ultra-High-Voltage Ni-Rich Layered Cathodes in Practical Li Metal Batteries Enabled by a Sulfonamide-Based Electrolyte. Nat. Energy 2021, 6, 495-505. https:/doi.org/10.1038/s41560-021-00792-y; Fan, X.; Chen, L.; Borodin, O.; Ji, X.; Chen, J.; Hou, S.; Deng, T.; Zheng, J.; Yang, C.; Liou, S. C.; et al. Non-Flammable Electrolyte Enables Li-Metal Batteries with Aggressive Cathode Chemistries. Nat. Nanotechnol. 2018, 13 (8). https://doi.org/10.1038/s41565-018-0183-2) high concentration electrolytes (HCEs), (Chen, Y.; Yu, Z.; Rudnicki, P.; Gong, H.; Huang, Z.; Kim, S. C.; Lai, J.-C.; Kong, X.; Qin, J.; Cui, Y.; et al. Steric Effect Tuned Ion Solvation Enabling Stable Cycling of High-Voltage Lithium Metal Battery. J. Am. Chem. Soc. 2021, 143 (44), 18703-18713. https:/doi.org/10.1021/jacs.1c09006; Qian, J.; Henderson, W. A.; Xu, W.; Bhattacharya, P.; Engelhard, M.; Borodin, O.; Zhang, J. G. High Rate and Stable Cycling of Lithium Metal Anode. Nat. Commun. 2015, 6, 6362. https://doi.org/10.1038/ncomms7362; Xue, W.; Shi, Z.; Huang, M.; Feng, S.; Wang, C.; Wang, F.; Lopez, J.; Qiao, B.; Xu, G.; Zhang, W.; et al. FSI-Inspired Solvent and “Full Fluorosulfonyl” Electrolyte for 4 v Class Lithium-Metal Batteries. Energy Environ. Sci. 2020, 13 (1), 212-220. https://doi.org/10.1039/c9ee02538c; Suo, L.; Xue, W.; Gobet, M.; Greenbaum, S. G.; Wang, C.; Chen, Y.; Yang, W.; Li, Y.; Li, J. Fluorine-Donating Electrolytes Enable Highly Reversible 5-V-Class Li Metal Batteries. Proc. Natl. Acad. Sci. U.S.A 2018, 115 (6), 1156-1161. https://doi.org/10.1073/pnas.1712895115; Fan, X.; Chen, L.; Ji, X.; Deng, T.; Hou, S.; Chen, J.; Zheng, J.; Wang, F.; Jiang, J.; Xu, K.; et al. Highly Fluorinated Interphases Enable High-Voltage Li-Metal Batteries. Chem 2018, 4 (1), 174-185. https://doi.org/10.1016/j.chempr.2017.10 017; Zheng, J.; Fan, X.; Ji, G.; Wang, H.; Hou, S.; DeMella, K. C.; Raghavan, S. R.; Wang, J.; Xu, K.; Wang, C. Manipulating Electrolyte and Solid Electrolyte Interphase to Enable Safe and Efficient Li—S Batteries. Nano Energy 2018, 50 (April), 431-440. https://doi.org/10.1016/j.nanoen.2018.05.065) localized high concentration electrolytes (LHCEs), (Lee, M. S.; Roev, V.; Jung, C.; Kim, J. R.; Han, S.; Kang, H. R.; Im, D.; Kim, I. S. An Aggregate Cluster-Dispersed Electrolyte Guides the Uniform Nucleation and Growth of Lithium at Lithium Metal Anodes. ChemistrySelect 2018, 3 (41), 11527-11534. https://doi.org/10.1002/slet.201800757; Huang, F.; Ma, G.; Wen, Z.; Jin, J.; Xu, S.; Zhang, J. Enhancing Metallic Lithium Battery Performance by Tuning the Electrolyte Solution Structure. J. Mater. Chem. A 2018, 6 (4), 1612-1620. https://doi.org/10.1039/c7ta08274f; Ren, X.; Zou, L.; Cao, X.; Engelhard, M. H.; Liu, W.; Burton, S. D.; Lee, H.; Niu, C.; Matthews, B. E.; Zhu, Z.; et al. Enabling High-Voltage Lithium-Metal Batteries under Practical Conditions. Joule 2019, 3 (7), 1662-1676. https://doi.org/10.1016/j.joule.2019.05.006; Cao, X.; Ren, X.; Zou, L.; Engelhard, M. H.; Huang, W.; Wang, H.; Matthews, B. E.; Lee, H.; Niu, C.; Arey, B. W.; et al. Monolithic Solid-Electrolyte Interphases Formed in Fluorinated Orthoformate-Based Electrolytes Minimize Li Depletion and Pulverization. Nat. Energy 2019, 4 (9), 796-805. https://doi.org/10.1038/s41560-019-0464-5; Liu, H.; Holoubek, J.; Zhou, H.; Chen, A.; Chang, N.; Wu, Z.; Yu, S.; Yan, Q.; Xing, X.; Li, Y.; et al. Ultrahigh Coulombic Efficiency Electrolyte Enables Li∥SPAN Batteries with Superior Cycling Performance. Mater. Today 2021, 42 (xx), 17-28. https://doi.org/10.1016/j.mattod.2020.09.035; Chen, S.; Zheng, J.; Mei, D.; Han, K. S.; Engelhard, M. H.; Zhao, W.; Xu, W.; Liu, J.; Zhang, J. G. High-Voltage Lithium-Metal Batteries Enabled by Localized High-Concentration Electrolytes. Adv. Mater. 2018, 30 (21). https://doi.org/10.1002/adma.201706102; Yoo, D.; Yang, S.; Kim, K. J.; Choi, J. W. Fluorinated Aromatic Diluent for High-Performance Lithium Metal Batteries. Angew. Chemie 2020, 132 (35), 14979-14986. https://doi.org/10.1002/ange.202003663; Niu, C.; Liu, D.; Lochala, J. A.; Anderson, C. S.; Cao, X.; Gross, M. E.; Xu, W.; Zhang, J. G.; Whittingham, M. S.; Xiao, J.; et al. Balancing Interfacial Reactions to Achieve Long Cycle Life in High-Energy Lithium Metal Batteries. Nat. Energy 2021, 6 (7), 723-732. https://doi.org/10.1038/s41560-021-00852-3) electrolytes with additives (Li, W.; Yao, H.; Yan, K.; Zheng, G.; Liang, Z.; Chiang, Y. M.; Cui, Y. The Synergetic Effect of Lithium Polysulfide and Lithium Nitrate to Prevent Lithium Dendrite Growth. Nat. Commun. 2015, 6 (May). https://doi.org/10.1038/ncomms8436; Zhang, H.; Zeng, Z.; He, R.; Wu, Y.; Hu, W.; Lei, S.; Liu, M.; Cheng, S.; Xie, J. 1,3,5-Trifluorobenzene and Fluorobenzene Co-Assisted Electrolyte with Thermodynamic and Interfacial Stabilities for High-Voltage Lithium Metal Battery. Energy Storage Mater. 2022, 48 (November 2021), 393-402. https://doi.org/10.1016/j.ensm.2022.03.034; Eldesoky, A.; Louli, A. J.; Benson, A.; Dahn, J. R. Cycling Performance of NMC811 Anode-Free Pouch Cells with 65 Different Electrolyte Formulations. J. Electrochem. Soc. 2021, 168 (12), 120508. https://doi.org/10.1149/1945-7111/ac39e3; Zhang, W.; Lu, Y.; Wan, L.; Zhou, P.; Xia, Y.; Yan, S.; Chen, X.; Zhou, H.; Dong, H.; Liu, K. Engineering a Passivating Electric Double Layer for High Performance Lithium Metal Batteries. Nat. Commun. 2022, 13 (1). https://doi.org/10.1038/s41467-022-29761-z), multi-salt electrolytes (Miao, R.; Yang, J.; Feng, X.; Jia, H.; Wang, J.; Nuli, Y. Novel Dual-Salts Electrolyte Solution for Dendrite-Free Lithium-Metal Based Rechargeable Batteries with High Cycle Reversibility. J. Power Sources 2014, 271, 291-297. https://doi.org/10.1016/j.jpowsour.2014.08.011; Qiu, F.; Li, X.; Deng, H.; Wang, D.; Mu, X.; He, P.; Zhou, H. A Concentrated Ternary-Salts Electrolyte for High Reversible Li Metal Battery with Slight Excess Li. Adv. Energy Mater. 2019, 9 (6). https://doi.org %10 1002/aenm.201803372; Weber, R.; Genovese, M.; Louli, A. J.; Hames, S.; Martin, C.; Hill, I. G.; Dahn, J. R. Long Cycle Life and Dendrite-Free Lithium Morphology in Anode-Free Lithium Pouch Cells Enabled by a Dual-Salt Liquid Electrolyte. Nat. Energy 2019, 4 (8), 683-689. https://doi.org/10.1038/s41560-019-0428-9; Louli, A. J.; Eldesoky, A.; Weber, R.; Genovese, M.; Coon, M.; deGooyer, J.; Deng, Z.; White, R. T.; Lee, J.; Rodgers, T.; et al. Diagnosing and Correcting Anode-Free Cell Failure via Electrolyte and Morphological Analysis. Nat. Energy 2020, 5 (9), 693-702. https://doi.org/10.1038/s41560-020-0668-8), and suspension electrolytes (Kim, M. S.; Zhang, Z.; Rudnicki, P. E.; Yu, Z.; Wang, J.; Wang, H.; Oyakhire, S. T.; Chen, Y.; Kim, S. C.; Zhang, W.; et al. Suspension Electrolyte with Modified Li+ Solvation Environment for Lithium Metal Batteries. Nat. Mater. 2022. https://doi.org/10.1038/s41563-021-01172-3). Among them, the combination of LiFSI with rationally designed solvent(s) is one of the most successful strategies. (Niu, C.; Liu, D.; Lochala, J. A.; Anderson, C. S.; Cao, X.; Gross, M. E.; Xu, W.; Zhang, J.; Whittingham, M. S.; Xiao, J.; et al. Balancing Interfacial Reactions to Achieve Long Cycle Life in High-Energy Lithium Metal Batteries. Nat. Energy 2021. https://doi.org/10.1038/s41560-021-00852-3) By carefully controlling the solvation structure of Li⁺, the reactivity of electrolyte can be designed to form FSI−-derived inorganic-rich SEI, which swells less in the electrolyte to remain mechanically robust and chemically passivating (Zhang, Z.; Li, Y.; Xu, R.; Zhou, W.; Li, Y.; Oyakhire, S. T.; Wu, Y.; Xu, J.; Wang, H.; Yu, Z.; et al. Capturing the Swelling of Solid-Electrolyte Interphase in Lithium Metal Batteries. Science (80-.). 2022, 375 (6576), 66-70. https://doi.org/10.1126/science.abi8703). Following this design, our groups developed several solvents that enable quick and effective passivation of Li anode, where the initial CE reaches >99% within less than 5 cycles and stable CE reaches 99.9% after 100 cycles.

Despite the number of solvent molecules reported for fine-tuning the reactivity of FSI⁻ anion, the variety of molecular design principles is very limited. Solvent fluorination, which tunes the Lewis basicity of solvents, and thereby their solvation ability, has been the most prominent method. However, it is of great interest to develop additional molecular design principles. We recently reported steric hindrance effect as another effective design strategy. (Chen, Y.; Yu, Z.; Rudnicki, P.; Gong, H.; Huang, Z.; Kim, S. C.; Lai, J. C.; Kong, X.; Qin, J.; Cui, Y.; et al. Steric Effect Tuned Ion Solvation Enabling Stable Cycling of High-Voltage Lithium Metal Battery. J. Am. Chem. Soc. 2021, 143 (44), 18703-18713. https://doi.org/10.1021/jacs.1c09006.) However, given the vast tunability of organic molecules, the molecular design space remains largely unexplored.

Herein, we demonstrate solvent coordination geometry for effective tuning of Li⁺ solvation structure and electrolyte reactivity. The special non-linear geometry of simple acetals leads to single-oxygen coordination with Li⁺ instead of chelation. As a result, dimethoxymethane (DMM) and diethoxymethane (DEM) are more weakly solvating than the ethylene glycol ether counterparts. When paired with LiFSI, both DMM and DEM showed high CE >99%. In particular, the DMM electrolytes enabled fast activation of Li∥Cu cells to reach 99% CE within 3 to 5 cycles. In addition to Li CE, ion transport is crucial for the practical application of lithium metal batteries (LMBs). Interestingly, despite being more weakly solvating, 3 M LiFSI/DMM showed slightly lower overpotential than 3 M LiFSI/DEE in Li∥Li cells due to similar ionic conductivity and higher limiting current fraction. The fast activation of CE, high average CE, fast ion transport, and low overpotential make 3 M LiFSI/DMM a promising candidate for anode-free LMBs with high-rate capability.

### I. C. 2. Results

I. C. 2. a. Molecular Structures and Design Principle

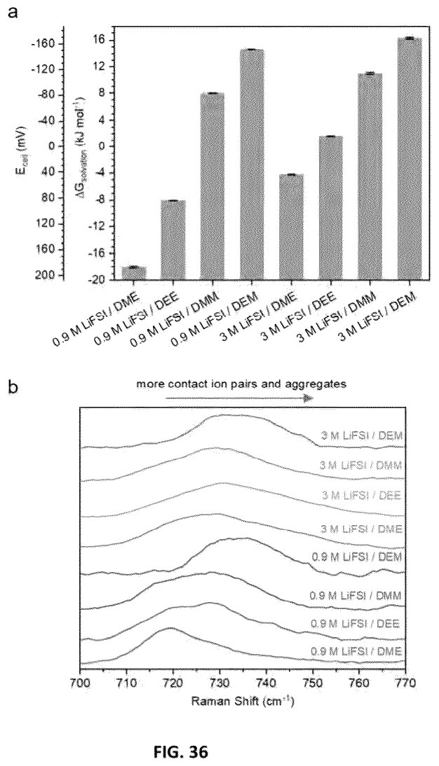

FIG. 35 illustrates Solvent coordination geometry as an effective design strategy for LMB electrolytes: (a) The most stable coordination geometries of solvent molecules with Li⁺. Ethylene glycol ethers are bidentate ligands that form a stable five-membered ring with Li⁺, whereas acetals are monodentate ligands. (b-c) DFT calculated ground state energy difference between [anti, anti] and [gauche, gauche] conformations of DMM as a single molecule (b) and in a typical solvation shell of 2 FSI⁻ and 1 DMM around Li⁺ (c). (d) ¹J_{CH} coupling constants of anomeric —CH₂— of DOL, DMM and DEM with various concentrations of LiTFSI. The corresponding molecular geometries for different ranges of ¹J_{CH} are shown on the right. (Anderson, J. E.; Heki, K.; Hirota, M.; Jorgensen, F. S. Setting the Anomeric Effect against Steric Effects in Simple Acyclic Acetals. Non-Anomeric Non-Classical Conformations. An N.M.R. and Molecular Mechanics Investigation. J. Chem. Soc. Chem. Commun. 1987, No. 8, 554-555. https://doi.org/10.1039/C39870000554.)

Various ethylene glycol ethers, such 1,2-dimethoxyethane (DME) and 1,2-diethoxyethane (DEE), are among the most popular solvents for Li anode due to their cathodic stability. Despite relatively low permittivity (Flamme, B.; Rodriguez Garcia, G.; Weil, M.; Haddad, M.; Phansavath, P.; Ratovelomanana-Vidal, V.; Chagnes, A. Guidelines to Design Organic Electrolytes for Lithium-Ion Batteries: Environmental Impact, Physicochemical and Electrochemical Properties. Green Chem. 2017, 19 (8), 1828-1849. https://doi.org/10.1039/c7gc00252a), these ethers exhibit good Li salt solubility due to chelation effect—the bidentate ligands can form a stable five-membered ring with Li⁺ (FIG. 35a). However, the strong solvent coordination with Li⁺ renders less ion pairs and aggregates, which is unfavorable for the formation of anion-derived SEI. Previously, we utilized fluorine substitution and steric effect to weaken the coordination ability of ethylene glycol ethers. Herein, we report a third molecular design strategy, where Li⁺-solvent interaction is significantly weakened by controlling the coordination geometry of solvent molecules.

We hypothesize that acetals (FIG. 35a) have weaker coordination ability than ethylene glycol ethers based on the following rationales. First, chelation effect is diminished by shortening the distance between two oxygens. Due to the formation of a highly unstable four-membered ring, the chelation of acetals with Li⁺ is unlikely. Second, acetals with small substituents favor non-linear geometry due to hyperconjugation. (Abe, A.; Inomata, K.; Tanisawa, E.; Ando, I. Conformation and Conformational Energies of Dimethoxymethane and 1,1-Dimethoxyethane. J. Mol. Struct. 1990, 238, 315-323; Carey, F. A.; Sundberg, R. J. Advanced Organic Chemistry, 5th ed.; Springer: New York, 2007). As a result, the electron density on each oxygen is pointing in the opposite directions. This further creates a barrier for chelation since significant changes in dihedral angles are required. Overall, we expect acetals to be monodentate ligands (FIG. 35a). In this study, two acetals, dimethoxymethane (DMM) and diethoxymethane (DEM), are selected as their structures are analogous to DME and DEE with the only difference being the coordination geometry (FIG. 35a).

Previous work confirmed the [gauche, gauche] conformation of DMM and DEM. However, it is unclear whether Li⁺ coordination could alter their molecular conformation. Therefore, we used density functional theory (DFT) calculation to determine the optimal coordination geometry. For pure DMM, [gauche, gauche] is more stable than [anti, anti] by 23.6 kJ/mol (FIG. 35b) as expected. We then constructed a solvation complex consisted of 1 Li⁺, 2 FSI⁻ and 1 DMM, which is common for weakly solvating electrolytes and which will be confirmed in the later section. Similar to pure DMM, the solvation complex with [gauche, gauche] DMM is more stable than that with [anti, anti] DMM by 29.4 kJ/mol. The optimized structures show that [anti, anti] DMM is bidentate whereas [gauche, gauche] DMM is monodentate. Consistent with our rationales above, the free energy gain of chelation is minimal compared to the energy penalty of breaking hyperconjugation in DMM. Overall, the DFT calculations indicate that DMM remains [gauche, gauche] when coordinated with Li⁺. We expect the same behavior in DEM since the steric hindrance is similar for ethyl or methyl group with anomeric hydrogens—the non-linear geometry should be significantly more stable in DEM as well.

The DFT results above are cross validated by 1D NMR experiments. The carbon-proton one-bond coupling constant at anomeric position (¹J_{CH}) is dependent on conformation. (Tvaroska, I.; Taravel, F. R. Carbon-Proton Coupling Constants in the Conformational Analysis of Sugar Molecules. In Advances in Carbohydrate Chemistry and Biochemistry; 1995; Vol. 51, pp 15-61. https://doi.org/10.1016/S0065-2318(08)60191-2) For acetals, ¹J_{CH} <158 Hz corresponds to [anti, anti] conformation, ¹J_{CH} ˜162 Hz corresponds to [gauche, gauche] with R groups on the opposite sides, and ¹J_{CH} >166 Hz corresponds to [gauche, gauche (alternative)] with R groups on the same side (FIG. 35d). A cyclic acetal, 1,3-dioxolane (DOL), was used as a control since it cannot adopt [anti, anti] conformation. The ¹J_{CH} values of DOL, DMM and DEM were measured with various LiTFSI concentrations. Here, LiTFSI was used instead of LiFSI because the latter readily initiates polymerization of DOL. Both pure DMM and DEM have ¹J_{CH} close to 162 Hz corresponding to [gauche, gauche], whereas pure DOL shows ¹J_{CH} right below 166 Hz due to a puckered conformation. (Lemieux, R. U.; Stevens, J. D.; Fraser, R. R. Observations on the Karplus Curve in Relation To the Conformation of the 1,3-Dioxolane Ring. Can. J. Chem. 1962, 40 (10), 1955-1959. https://doi.org/10.1139/v62-300.) As LiTFSI concentration increases, all three acetals show slightly increased ¹J_{CH}, which is attributed to the slight change in dihedral angles upon Li⁺ coordination. DMM and DEM samples follow the same trend as DOL samples, which indicates DMM and DEM do not adopt [anti, anti] conformation when coordinated with Li⁺. All ¹J_{CH} values of DMM and DEM samples are around 162 Hz, corresponding to [gauche, gauche] conformation with and without LiTFSI. The same experiment was carried out using LiFSI in DMM and DEM (FIG. 41), where the same trend is observed.

Based on DFT calculation and NMR experiment above, we conclude that both DMM and DEM remain [gauche, gauche] when coordinated with Li⁺. This molecular geometry prevents DMM and DEM from chelating with Li⁺ due to the distance between two oxygens as well as the orientation of lone pair electron density on each oxygen (FIG. 35a). Therefore, we predict that the solvating ability of DMM and DEM are weaker than that of DME and DEE.

I. C. 2. b. Static Solvation Structures

To verify our prediction above, the static solvation structures of LiFSI in various solvents were investigated. For each solvent, 1 and 4 moles of LiFSI per liter of solvent were prepared, corresponding to standard (˜0.9 M) and high (˜3 M) concentration electrolytes respectively. The density, molarity and molality for each electrolyte are listed in Supplementary Table S1.

FIG. 36 illustrates Static solvation structures of 0.9 M and 3 M LiFSI in acetals (DMM and DEM) and ethylene glycol ethers (DME and DEE): (a) Open circuit voltages (E_{cell}) and their corresponding solvation energies (ΔGₛₒₗᵥₐₜᵢₒₙ) of the electrolytes. The measurement was explained in detail in Kim, S. C.; Kong, X.; Vilá, R. A.; Huang, W.; Chen, Y.; Boyle, D. T.; Yu, Z.; Wang, H.; Bao, Z.; Qin, J.; et al. Potentiometric Measurement to Probe Solvation Energy and Its Correlation to Lithium Battery Cyclability. J. Am. Chem. Soc. 2021, 143 (27), 10301-10308. https://doi.org/10.1021/jacs.1c03868. The reference electrolyte is 1 M LiFSI in DEC (ΔGₛₒₗᵥₐₜᵢₒₙ=0). The data of ethylene glycol ethers were reproduced from above. (b) Raman spectra of the electrolytes. The convoluted peaks between 700 and 760 cm⁻¹ correspond to FSI⁻ in various solvation environments: solvent-separated ion pairs (SSIP), contact ion pairs (CIP) and ion aggregates (AGG) from low to high wavenumber.

We first probed the solvation environments of Li⁺ by solvation energy measurement. The open-circuit potential of a concentration cell with symmetric Li metal electrodes and asymmetric electrolytes is related to the difference in free energy of Li⁺ solvation in each electrolyte. In a more weakly solvating electrolyte, entropy (AS) is less positive since ion aggregation leads to less randomness for Li⁺, and enthalpy (AH) is less negative due to weaker solvent-Li⁺ interactions (keep in mind that LiFSI dissolution here is exothermic at constant pressure). Therefore, the overall free energy (AG) is less negative or more positive in a more weakly solvating electrolyte relative to the reference. ΔGₛₒₗᵥₐₜᵢₒₙ increases in the order of DME<DEE<DMM<DEM at both 0.9 M and 3 M, corresponding to increasingly weak solvation of Li⁺. Notable, despite being fluorine-free, DMM and DEM electrolytes show a similar range of ΔGₛₒₗᵥₐₜᵢₒₙ as some fluorinated DEE electrolytes, which demonstrates the strong impact of solvent coordination geometry on solvation ability. As concentration increases from 0.9 M to 3 M, the change in ΔGₛₒₗᵥₐₜᵢₒₙ is smaller for DMM and DEM compared to DME and DEE due to the weak solvating ability of acetals even at low concentrations.

The degree of ion interactions in each electrolyte was characterized by Raman spectroscopy. The convoluted peaks between 700 and 760 cm⁻¹ correspond to FSI⁻ in various solvation environments. A shift to higher wavenumber indicates a larger proportion of contact ion pairs and aggregates relative to solvent-separated ion pairs. (Yamada, Y.; Yaegashi, M.; Abe, T.; Yamada, A. A Superconcentrated Ether Electrolyte for Fast-Charging Li-Ion Batteries. Chem. Commun. 2013, 49, 11194-11196. https://doi.org/10.1039/c3cc46665e; Cao, X.; Zou, L.; Matthews, B. E.; Zhang, L.; He, X.; Ren, X.; Engelhard, M. H.; Burton, S. D.; El-Khoury, P. Z.; Lim, H. S.; et al. Optimization of Fluorinated Orthoformate Based Electrolytes for Practical High-Voltage Lithium Metal Batteries. Energy Storage Mater. 2021, 34, 76-84. https://doi.org/10.1016/j.ensm.2020.08.035; Jiang, Z.; Zeng, Z.; Liang, X.; Yang, L.; Hu, W.; Zhang, C.; Han, Z.; Feng, J.; Xie, J. Fluorobenzene, A Low-Density, Economical, and Bifunctional Hydrocarbon Cosolvent for Practical Lithium Metal Batteries. Adv. Funct. Mater. 2021, 31, 2005991. https://doi.org/10.1002/adfm.202005991.) At both 0.9 M and 3 M, the wavenumber increases in the order of DME<DEE≈DMM<DEM, indicating increasing proportion of FSI⁻ in contact ion pairs and aggregates. The general trend is similar to that of ΔGₛₒₗᵥₐₜᵢₒₙ except for DEE and DMM electrolytes having similar Raman shifts. This discrepancy is likely due to the difference in anion-solvent interactions (Popov, I.; Sacci, R. L.; Sanders, N. C.; Matsumoto, R. A.; Thompson, M. W.; Osti, N. C.; Kobayashi, T.; Tyagi, M.; Mamontov, E.; Pruski, M.; et al. Critical Role of Anion-Solvent Interactions for Dynamics of Solvent-in-Salt Solutions. J. Phys. Chem. C 2020, 124 (16), 8457-8466. https://doi.org/10.1021/acs.jpcc.9b10807)—ΔGₛₒₗᵥₐₜᵢₒₙ measurement probes Li⁺ solvation environment whereas Raman spectroscopy probes FSI⁻ solvation environment.

I. C. 2. c. Electrochemical Stability

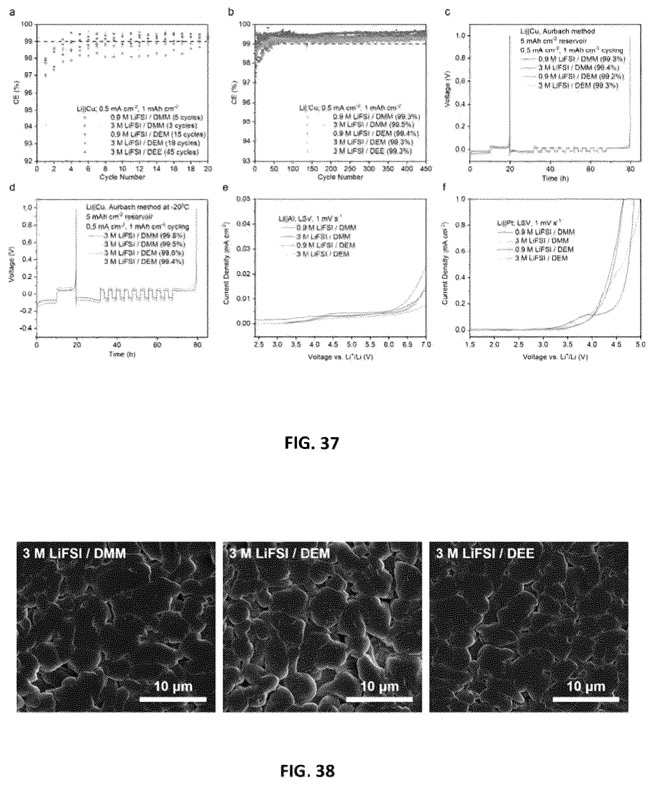

FIG. 37 illustrates Electrochemical stability of 0.9 M and 3 M LiFSI in DMM and DEM: (a) Initial CE of Li∥Cu cells. The number of cycles to reach 99% is indicated for each electrolyte. (b) Long-term cycling of Li∥Cu cells. The stabilized average CE were calculated after the 50^{th} cycle. Abnormal cycles due to instrument failure were omitted in the calculation. The champion cell was used for calculation when there are replicates. The data of 3 M LiFSI/DEE were reproduced from above. (c-d) Li∥Cu CE measured by the modified Aurbach method (Adams, B. D.; Zheng, J.; Ren, X.; Xu, W.; Zhang, J. G. Accurate Determination of Coulombic Efficiency for Lithium Metal Anodes and Lithium Metal Batteries. Adv. Energy Mater. 2018, 8 (7), 1-11. https://doi.org/10.1002/aenm.201702097) at room temperature (c) and −20° C. (d). (e-f) Oxidative stability of the electrolytes measured by LSV using Al (e) and Pt (f) as the working electrode.

The benefits of weakly solvating electrolytes for stabilizing electrode-electrolyte interfaces have been well documented, which motivated us to further investigate the electrochemical stability of acetal electrolytes. Li∥Cu half-cell CE of the acetal electrolytes were benchmarked against the previous state-of-the-art organofluorine-free 3 M LiFSI in DEE. In the initial 20 cycles, 0.9 M and 3 M LiFSI in DMM and DEM significantly outperformed 3 M LiFSI in DEE (FIG. 37a). Remarkably, 0.9 M and 3 M LiFSI in DMM reached >99% CE within 5 and 3 cycles respectively. To the best of our knowledge, they are the first organofluorine-free electrolytes to achieve fast CE activation (>99% within 5 cycles at 0.5 mA cm⁻² and 1 mAh mA cm⁻²), a property that was only observed in a few organofluorine electrolytes. This is highly desirable for anode-free LMBs. The stabilized average CE calculated after the 50 cycle were above 99% for all five electrolytes tested, among which 3 M LiFSI/DMM was the highest at 99.5% (FIG. 37b). The cycle life of Li∥Cu half cells was less than 200 cycles in the 0.9 M acetal electrolytes, which was significantly shorter than that in the 3 M electrolytes (FIG. 37b). This instability was likely due to unfavorable morphology after prolonged cycling instead of side reactions since CE were similar at both concentrations.

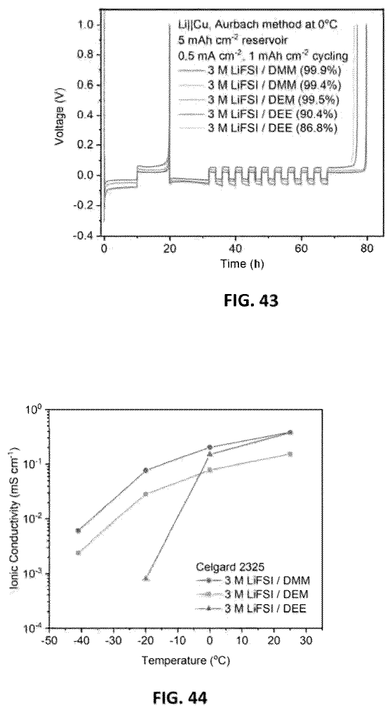

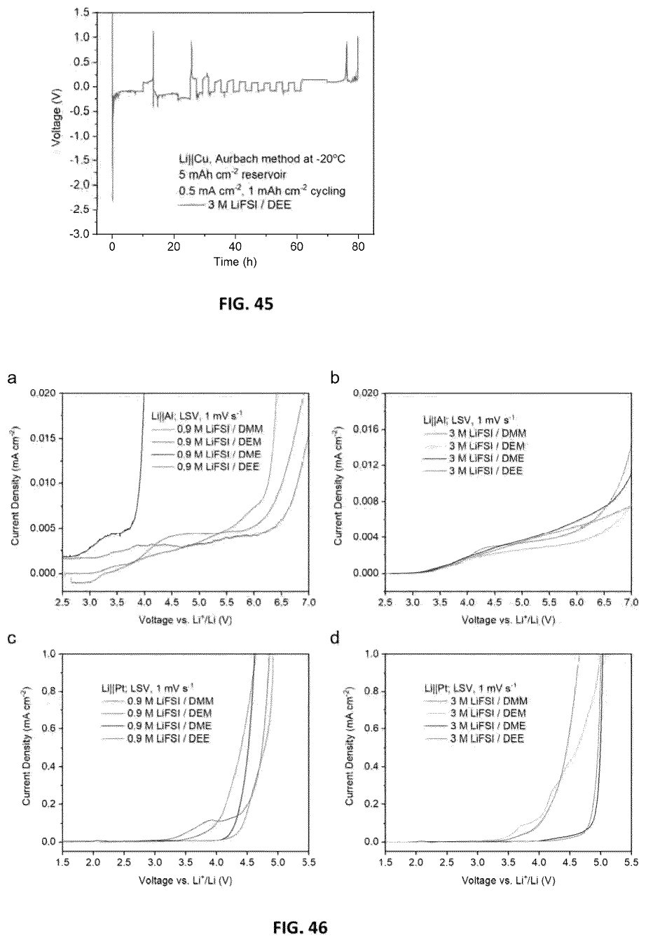

The CE were also measured by the modified Aurbach method (Id.) at room temperature (FIG. 37c, repeated data in FIG. 42). With 0.9 M LiFSI, DMM (99.3%) and DEM (99.2%) both slightly outperformed DEE (99.0%). With 3 M LiFSI, DMM (99.4%) and DEM (99.3%) showed similar CE as DEE (99.4%). The advantage of acetals compared to DEE was more apparent on bare Cu (FIGS. 37a-b), whereas cycling on top of excess Li obscured the difference (FIG. 37c). In addition, stable cycling (both plating and stripping) of Li at low temperatures is of great interest but rarely demonstrated. (Rustomji, C. S.; Yang, Y.; Kim, T. K.; Mac, J.; Kim, Y. J.; Caldwell, E.; Chung, H.; Meng, Y. S. Liquefied Gas Electrolytes for Electrochemical Energy Storage Devices. Science (80-.). 2017, 356 (6345). https://doi.org/10.1126/science.aa14263; Gao, Y.; Rojas, T.; Wang, K.; Liu, S.; Wang, D.; Chen, T.; Wang, H.; Ngo, A. T.; Wang, D. Low-Temperature and High-Rate-Charging Lithium Metal Batteries Enabled by an Electrochemically Active Monolayer-Regulated Interface. Nat. Energy 2020, 5 (7), 534-542. https://doi.org/10.1038/s41560-020-0640-7; Dong, X.; Lin, Y.; Li, P.; Ma, Y.; Huang, J.; Bin, D.; Wang, Y.; Qi, Y.; Xia, Y. High-Energy Rechargeable Metallic Lithium Battery at −70° C. Enabled by a Cosolvent Electrolyte. Angew. Chemie—Int. Ed. 2019, 58 (17), 5623-5627. https://doi.org/10.1002/anie.201900266). Therefore, we measured CE of 3 M LiFSI in DMM, DEM and DEE by the modified Aurbach method at 0° C. Both acetal electrolytes demonstrated stable CE above 99%, whereas the DEE electrolyte showed significant instability with a large initial overpotential of ˜300 mV and low CE of ˜90% (FIG. 43). Notably, such instability was not due to bulk ion transport since the ionic conductivities at 0° C. (with Celgard 2325) decreased in the order of DMM>DEE>DEM with 3 M LiFSI (FIG. 44). Therefore, the stable Li cycling in the DMM and DEM electrolytes at 0° C. could be attributed to the more facile de-solvation of Li⁺ as a result of the weakly solvating property of acetals. (Cai, G.; Holoubek, J.; Li, M.; Gao, H.; Yin, Y.; Yu, S.; Liu, H.; Pascal, T. A.; Liu, P. Solvent Selection Criteria for Temperature-Resilient Lithium-Sulfur Batteries. 2022, 1-9. https://doi.org/10.1073/pnas.2200392119/-/DCSupplemental.Published.) In addition, the CE were measured at −20° C. Compared to room temperature (FIG. 37c and FIG. 42), the CE were higher for both acetal electrolytes at −20° C. albeit slightly larger variations (FIG. 37d). The increase in CE was likely due to kinetically suppressed side reactions at low temperatures. In stark contrast, 3 M LiFSI in DEE failed to achieve stable cycling at −20° C. (FIG. 45) with a large initial overpotential (ca. −2.3 V) and spiky voltage. In addition to the slow charge transfer kinetics, the poor ion transport at −20° C. further aggravated instability (FIG. 44).

A major issue of imide-based salts is their side reaction with aluminum (Al) cathode current collector at high voltages. Previously, we demonstrated that a weakly solvating electrolyte allowed the buildup of a thick and fluorine-rich passivation layer on Al even when LiFSI was used. This was attributed to less dissolution of Al(FSI)x and other reaction products in a weakly solvating electrolyte. (Yamada, Y.; Chiang, C. H.; Sodeyama, K.; Wang, J.; Tateyama, Y.; Yamada, A. Corrosion Prevention Mechanism of Aluminum Metal in Superconcentrated Electrolytes. ChemElectroChem 2015, 2 (11), 1687-1694. https://doi.org/10.1002/celc.201500235.) Therefore, we predicted that the acetal electrolytes should be compatible with Al current collector. We performed linear scanning voltammetry (LSV) using Li∥Al cells. The acetal electrolytes showed no sharp increase in leakage current within the operating voltage window of common cathode materials (FIG. 37e), which indicates good stability with Al current collector. In comparison, the compatibility of DMM and DEM with Al was similar to DEE and significantly better than DME with 0.9 M LiFSI (FIG. 46a). With 3 M LiFSI, all four electrolytes showed similar stability with Al within the practical voltage range (FIG. 46b).

The oxidative stability of the acetal electrolytes was also characterized by Li∥Pt cells. The Pt working electrode is inert and non-reactive. Therefore, electrolyte oxidation can be captured without the passivation effect seen on Al electrode. The onset of rapid oxidation on Pt was around 4 V (versus Li⁺/Li) for 0.9 M and 3 M LiFSI in DMM, and was slightly lower for DEM electrolytes (FIG. 37f). Significant oxidation reactions occurred at a much lower voltage range on Pt compared to Al, which indicated limited anodic stability of the acetal electrolytes despite good passivation on Al. In comparison, the acetal electrolytes showed worse anodic stability compared to DME and DEE electrolytes with both 0.9 M and 3 M LiFSI (FIG. 46c-d). Therefore, the acetal electrolytes here are not compatible with high voltage cathodes (such as NMC) but rather more suitable with LFP and sulfur cathodes.

Considering the overall Li cycling stability and voltage tolerance, 3 M LiFSI in DMM and DEM appeared more suitable than the 0.9 M electrolytes for the stable operation of LMBs. Therefore, in the following sections, we focus our discussion on 3 M LiFSI in DMM and DEM. The acetal electrolytes were evaluated in comparison to 3 M LiFSI in DEE, which is the state-of-the-art organofluorine-free electrolyte.

I. C. 2. d. Li Morphology

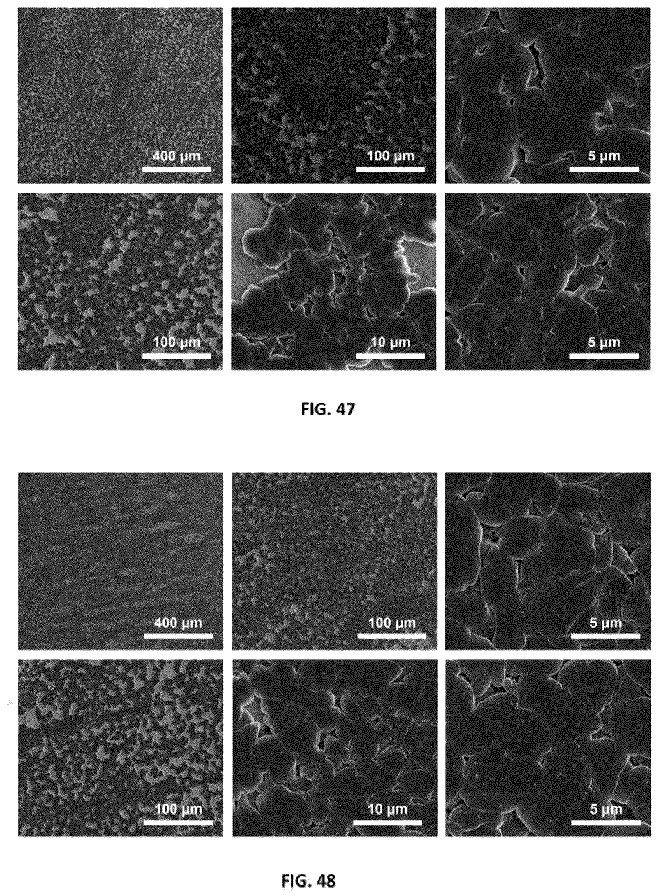

FIG. 38 illustrates SEM images of the initial Li deposition morphology in 3 M LiFSI in DMM, DEM and DEE. A small amount of Li (0.5 mAh cm⁻²) was plated onto Cu at 0.5 mA cm⁻² in uncycled Li∥Cu cells. Additional images are provided in FIGS. 47-49.

The initial Li deposition morphology was characterized by SEM. A small amount of Li (0.5 mAh cm⁻²) was plated onto Cu at 0.5 mA cm⁻² in uncycled Li∥Cu cells. All three electrolytes showed bulky Li growth without dendrite formation.

I. C. 2. e. Ion Transport Properties

Despite the recent progress in improving CE of Li anode by electrolyte designs, there are still major barriers to the practical application of LMBs. In particular, we would like to draw attention to the poor ion transport in many advanced electrolyte designs, which has two major consequences under practical current densities. First, the slow ion transport leads to high internal resistance and low capacity utilization, thereby reducing the actual energy density of LMBs. Second, the buildup of a large concentration gradient due to slow ion transport results in unfavorably Li deposition morphology and poor stability. (Louli, A. J.; Eldesoky, A.; deGooyer, J.; Coon, M.; Aiken, C. P.; Simunovic, Z.; Metzger, M.; Dahn, J. R. Different Positive Electrodes for Anode-Free Lithium Metal Cells. J. Electrochem. Soc. 2022, 169 (4), 040517. https://doi.org/10.1149/1945-7111/ac62c4; Louli, A. J.; Coon, M.; Genovese, M.; deGooyer, J.; Eldesoky, A.; Dahn, J. R. Optimizing Cycling Conditions for Anode-Free Lithium Metal Cells. J. Electrochem. Soc. 2021, 168 (2), 020515. https://doi.org/10.1149/1945-7111/abe089.) Therefore, it is crucial to design electrolytes that improve CE without sacrificing ion transport—an aspect that deserves more attention.

In non-fluorinated solvents, both Li anode and cathode stabilities generally benefit from elevated LiFSI concentrations (>1 M). However, a common concern is that the increased viscosity with concentration leads to unfavorable ion transport. (Yamada, Y.; Wang, J.; Ko, S.; Watanabe, E.; Yamada, A. Advances and Issues in Developing Salt-Concentrated Battery Electrolytes. Nat. Energy 2019, 4 (4), 269-280. https://doi.org/10.1038/s41560-019-0336-z). To the best of our knowledge, there is very limited understanding on the concentration-dependent ion transport in high-performance electrolytes for LMBs. Therefore, we carried out a detailed analysis on ion transport.

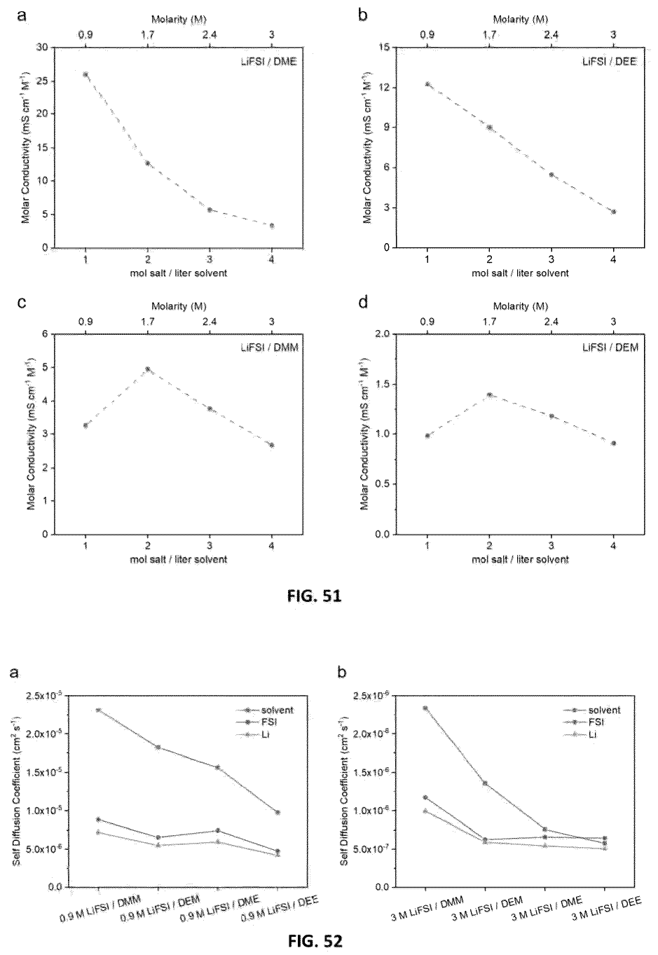

As the solvation ability of solvents weakens (DME >DEE>DMM>DEM), the ionic conductivity without separator (a) peaks at a higher LiFSI concentration (FIG. 50). In the battery literature, the peak in a with varying concentrations is typically explained by the opposing effects of increasing concentration and viscosity according to Nernst-Einstein and Stokes-Einstein equations. However, the molar conductivities (A) of acetal electrolytes peak at intermediate concentrations (FIG. 51). Interestingly, in both DMM and DEM, 1.7 M and 2.4 M electrolytes have higher A than 0.9 M electrolytes, which indicates that ion transport is faster despite higher viscosity, and that the initial increase in a is not solely due to increased concentration. Importantly, the observed trend in a and A indicates that ion transport is not necessarily slowed by increased ion aggregation in weakly solvating, high concentration electrolytes.

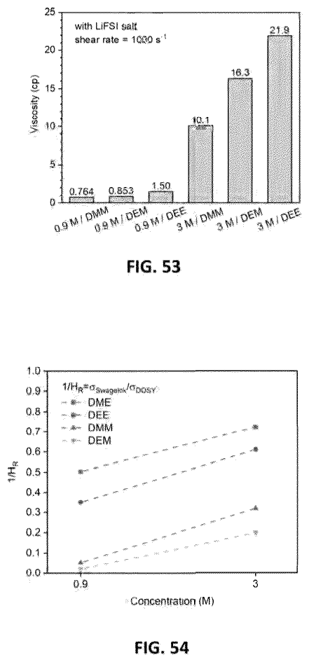

We further investigated ion transport in 0.9 M and 3 M electrolytes, which are representative of low and high concentrations. The self-diffusion coefficients (D_{self}) of solvents, Li⁺ and FSI⁻ were measured by diffusion ordered spectroscopy (DOSY) (FIG. 52). The general trend of D_{self} follows viscosity (FIG. 53) in the reverse order with some variations, which are likely due to deviation from Stokes-Einstein equation as a result of non-sphericality of ion clusters. From D_{self}, the inverse Haven ratios (1/H_{R}) can be calculated (FIG. 54 and Equation 1, 2), which increase with LiFSI concentration in all four solvents. Traditionally, 1/H_{R} was interpreted as the degree of ion dissociation (Hayamizu, K.; Aihara, Y.; Arai, S.; Martinez, C. G. Pulse-Gradient Spin-Echo 1H, 7Li, and 19F NMR Diffusion and Ionic Conductivity Measurements of 14 Organic Electrolytes Containing LiN(SO2CF3)2. J. Phys. Chem. B 1999, 103 (3), 519-524. https://doi.org/10.1021/jp9825664; Noda, A.; Hayamizu, K.; Watanabe, M. Pulsed-Gradient Spin-Echo 1H and 19F NMR Ionic Diffusion Coefficient, Viscosity, and Ionic Conductivity of Non-Chloroaluminate Room-Temperature Ionic Liquids. J. Phys. Chem. B 2001, 105 (20), 4603-4610. https://doi.org/10.1021/jp004132q; Ueno, K.; Tokuda, H.; Watanabe, M. Ionicity in Ionic Liquids: Correlation with Ionic Structure and Physicochemical Properties. Phys. Chem. Chem. Phys. 2010, 12 (8), 1649-1658. https://doi.org/10.1039/c001176m; Chintapalli, M.; Timachova, K.; Olson, K. R.; Mecham, S. J.; Devaux, D.; Desimone, J. M.; Balsara, N. P. Relationship between Conductivity, Ion Diffusion, and Transference Number in Perfluoropolyether Electrolytes. Macromolecules 2016, 49 (9), 3508-3515. https://doi.org/10.1021/acs.macromol.6b00412)—an ideal solution has 1/H_{R} of 1 and ion association leads to smaller 1/H_{R}. However, the trend observed here clearly contradicts with the previous interpretation as ion aggregation should increase with concentration. Therefore, ion transport in these electrolytes cannot be described by the general framework of vehicular transport. In another word, there is not a simple correlation between the individual ion movement and overall ion transport.

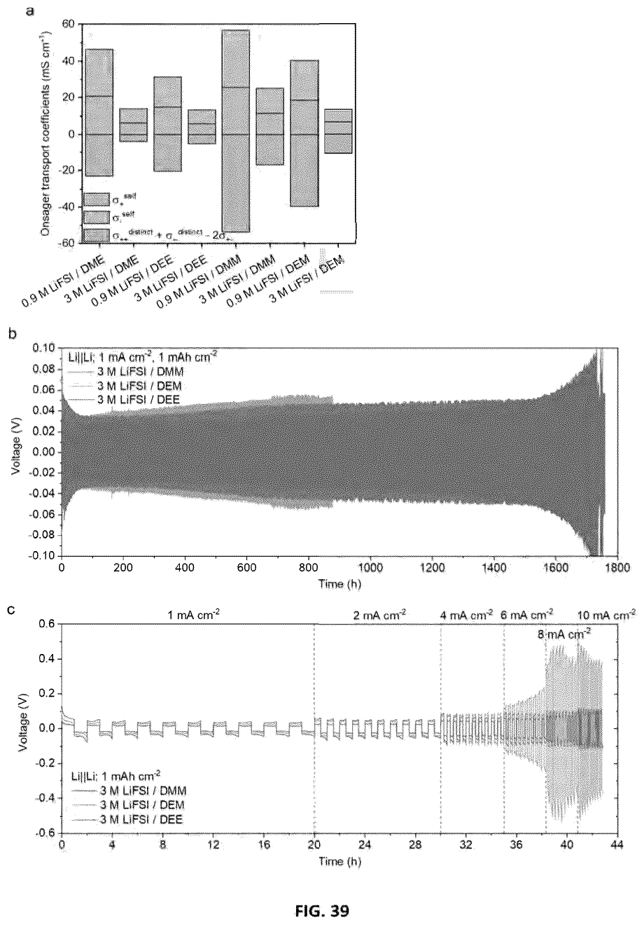

FIG. 39 provides an Analysis of ion transport: (a) Onsager transport coefficients calculated from experimental data (Supplementary Equation 3-5). (b-c) Voltage profiles of Li∥Li cells. (b) Long-term cycling at a low current density. The zoomed-in voltage curves are provided in FIG. 56. The data of 3 M LiFSI/DEE were reproduced from above. (c) Cycling at 1 to 10 mA cm⁻² with 10 cycles at each current density. The zoomed-in voltage curves of the later cycles are provided in FIG. 58.

Stefan-Maxwell (Grundy, L. S.; Shah, D. B.; Nguyen, H. Q.; Diederichsen, K. M.; Celik, H.; DeSimone, J. M.; McCloskey, B. D.; Balsara, N. P. Impact of Frictional Interactions on Conductivity, Diffusion, and Transference Number in Ether- and Perfluoroether-Based Electrolytes. J. Electrochem. Soc. 2020, 167(12), 120540. https://doi.org/10.1149/1945-7111/abb34e; Mistry, A.; Yu, Z.; Peters, B. L.; Fang, C.; Wang, R.; Curtiss, L. A.; Balsara, N. P.; Cheng, L.; Srinivasan, V. Toward Bottom-Up Understanding of Transport in Concentrated Battery Electrolytes. ACS Cent. Sci. 2022. https://doi.org/10.1021/acscentsci.2c00348) and Onsager (Fong, K. D.; Self, J.; McCloskey, B. D.; Persson, K. A. Ion Correlations and Their Impact on Transport in Polymer-Based Electrolytes. Macromolecules 2021, 54 (6), 2575-2591. https://doi.org/10.1021/acs.macromol.0c02545; Vargas-Barbosa, N. M.; Roling, B. Dynamic Ion Correlations in Solid and Liquid Electrolytes: How Do They Affect Charge and Mass Transport?ChemElectroChem 2020, 7(2), 367-385. https://doi.org/10.1002/celc.201901627) frameworks have been used to describe ion transport properties. The main advantage is to properly capture the effects of ion correlation on overall ion transport. We selected the Onsager framework due to its simple computation from molecular dynamics (MD) simulation. (Id.) There are five independent transport coefficients: σ₊^{self} and σ₋^{self} correspond to the individual uncorrelated ion movement, and are proportional to D_{self}(Supplementary Equation 3, 4); σ₊₊^{distinct}, σ₋^{distinct}, and σ₊₋ capture the cation-cation, anion-anion, and cation-anion correlations between two distinct particles of the same or different ion species. We present the transport coefficients in units of mS cm⁻¹ for simplicity. However, we stress that the transport coefficients are not conductivities. Instead, the combinations of them provide experimentally relevant properties such as conductivity.

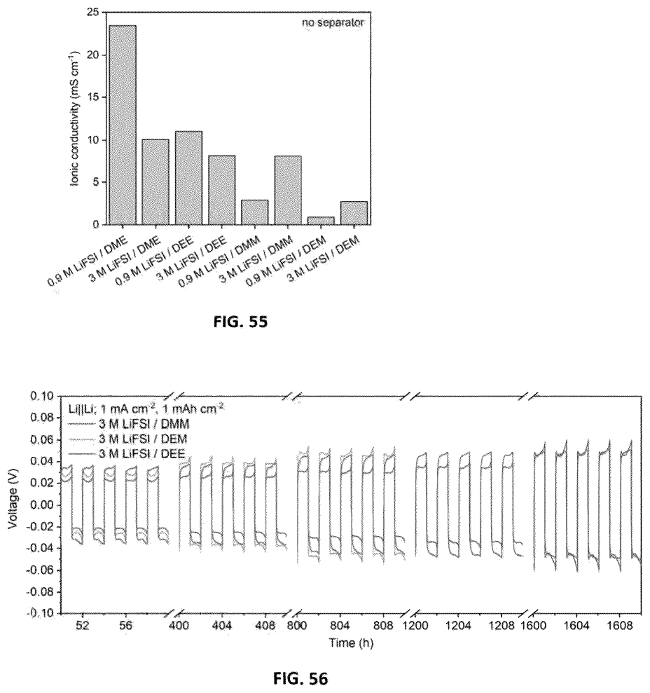

We first calculated σ₊^{self}, σ₋^{self} and (σ₊₊^{distinct}+σ₋^{distinct}−2σ₊₋) from experimental conductivities and self-diffusion coefficients (Supplementary Equation 3-5). In all four solvents, as LiFSI concentration increases from 0.9 M to 3 M, the σ₊^{self} and σ₋^{self} decrease (FIG. 39a) as a result of the more drastic decrease of D_{self} compared to the increase of ion concentration (Supplementary Equation 3, 4). Interestingly, the (σ₊₊^{distinct}+σ₋^{distinct}−2σ₊₋) decreases in magnitude from 0.9 M to 3 M as well. This is somewhat counterintuitive since it is well documented that increasing concentration leads to more ion-ion interactions. (Wang, Z.; Wang, H.; Qi, S.; Wu, D.; Huang, J.; Li, X.; Wang, C.; Ma, J. Structural Regulation Chemistry of Lithium Ion Solvation for Lithium Batteries. EcoMat 2022, No. January, 1-24. https://doi.org/10.1002/eom2.12200.) Overall, the relative change of σ₊^{self}, σ₋^{self} and (σ₊₊^{distinct}+σ₋^{distinct}−2σ₊₋) from 0.9 M to 3 M determines the change in ionic conductivity-DME and DEE electrolytes decrease in conductivity, whereas DMM and DEM electrolytes increase in conductivity (FIG. 55). As a result, 3 M LiFSI/DMM have similar ionic conductivity as 3 M LiFSI/DME and DEE despite that the DMM electrolyte is significantly more weakly solvating with more anion-rich Li⁺ solvation shells. In addition, FIG. 39a clearly demonstrates that individual ion movements are insufficient to describe ion transport, and that ion-ion correlations have quite significant contributions.

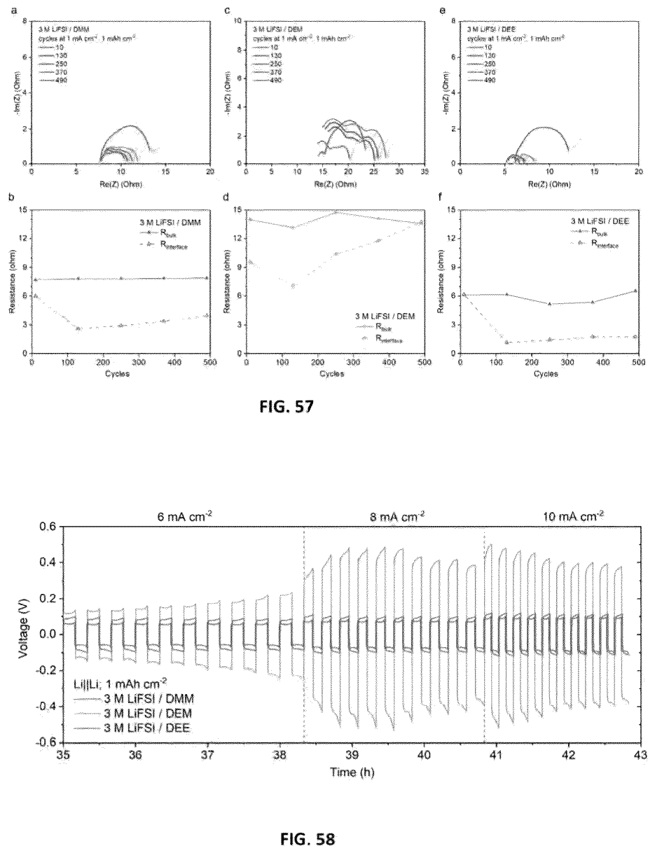

The overpotential of Li∥Li cells is often a simple and good indicator of ion transport. The cells were cycled at 1 mA cm⁻² for 1 mAh cm⁻² in each step (FIG. 39b and FIG. 56). The overpotential in 3 M LiFSI/DMM was significantly lower (˜22 mV after 50 cycles, −30 mV after 800 cycles, and −34 mV after 1200 cycles) than many reported high-CE electrolytes. (Xue, W.; Huang, M.; Li, Y.; Zhu, Y. G.; Gao, R.; Xiao, X.; Zhang, W.; Li, S.; Xu, G.; Yu, Y.; et al. Ultra-High-Voltage Ni-Rich Layered Cathodes in Practical Li Metal Batteries Enabled by a Sulfonamide-Based Electrolyte. Nat. Energy 2021, 6 (5), 495-505. https://doi.org/10.1038/s41560-021-00792-y). It is also lower than 3 M LiFSI/DEE⁹ despite the sudden increase after 1500 hours. The overpotential in 3 M LiFSI/DEM was the highest, and it increased more quickly than the DMM and DEE electrolytes. The contributions to overpotential were investigated by performing electrochemical impedance spectroscopy (EIS) on Li∥Li cells after every 120 cycles at 1 mA cm⁻² and 1 mAh cm⁻² (FIG. 57). Throughout cycling, the bulk resistance (R_{bulk}) remained stable in all three electrolytes. The interfacial resistance (R_{interface}) continuously increased in 3 M LiFSI/DEM, whereas R_{interface} remained stable in 3 M LiFSI/DMM and DEE after the initial decrease. Since the exchange current density of Li redox reaction is significantly higher than 1 mA cm⁻² at room temperature, the contribution from charge transfer resistance to R_{interface} should be small. (Boyle, D. T.; Kong, X.; Pei, A.; Rudnicki, P. E.; Shi, F.; Huang, W.; Bao, Z.; Qin, J.; Cui, Y. Transient Voltammetry with Ultramicroelectrodes Reveals the Electron Transfer Kinetics of Lithium Metal Anodes. ACS Energy Lett. 2020, 5 (3), 701-709. https://doi.org/10.1021/acsenergylett.0c0031.) Therefore, the quick increase in overpotential in 3 M LiFSI/DEM was attributed to SEI instability. Both R_{bulk} and R_{interface} were slightly higher in 3 M LiFSI/DMM than in 3 M LiFSI/DEE after cycling (FIGS. 57b, f) despite that the overpotential in 3 M LiFSI/DMM was lower. This was due to the significantly higher limiting current fraction (ρ₊) in 3 M LiFSI/DMM than in 3 M LiFSI/DEE (Supplementary Table S2). Notably, in 3 M LiFSI/DEM, the high p. was not sufficient to compensate for the large R_{bulk} and R_{interface} in the later cycles, leading to the highest overpotential.

In addition, the overpotential at 1 to 10 mA cm⁻² was evaluated in Li∥Li cells (FIG. 39c and FIG. 58). 3 M LiFSI/DMM showed <100 mV overpotential even at 10 mA cm⁻², which is extremely low compared to other high-CE electrolytes. 3 M LiFSI/DEE showed slightly higher overpotential than 3 M LiFSI/DMM. In contrast, 3 M LiFSI/DEM with the lowest conductivity showed a sharp increase in overpotential at >6 mA cm⁻², and the overpotential became unstable.

Based on the Li∥Li cell overpotential, 3 M LiFSI/DMM showed great promise for simultaneously achieving high CE and fast ion transport.

I. C. 2. f. Full Cell Performance

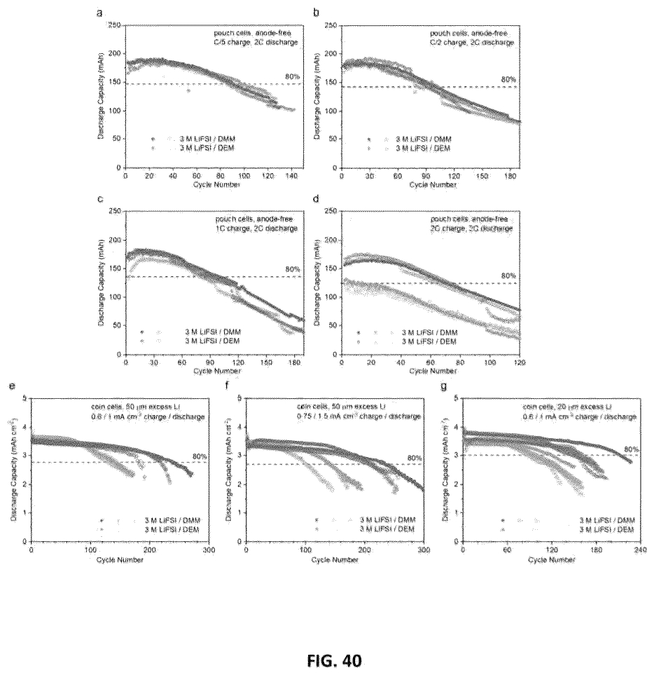

FIG. 40 illustrates LFP-based full cells cycled with 3 M LiFSI/DMM and 3 M LiFSI/DEM: (a-d) Anode-free Cu∥micro-LFP pouch cells (nominally ˜210 mAh, ˜2.1 mAh cm⁻², 2.5 to 3.65 V, 0.5 mL electrolyte, 1C=200 mA or 2 mA cm⁻²) cycled at various charge rates and 2 C discharge rate. The first-cycle charge rate was C/10. The 80% capacity retention line is based on the solid trace of 3 M LiFSI/DMM at the 2^{nd} cycle. The pouch cell parameters are provided in Supplementary Table S3. (e-g) Thin-Li∥micro-LFP coin cells (nominally 3.6 to 4 mAh cm⁻², 2.5 to 3.65 V, 40 μL electrolyte) with free-standing Li foil of 50 μm (e, f) and 20 μm (g) thickness cycled at various current densities. Two activation cycles were carried out at 0.3 mA cm⁻². The 80% capacity retention line is based on the solid trace of 3 M LiFSI/DMM at the 4^{th} cycle. The corresponding CE values are shown in Supplementary Figure S19.

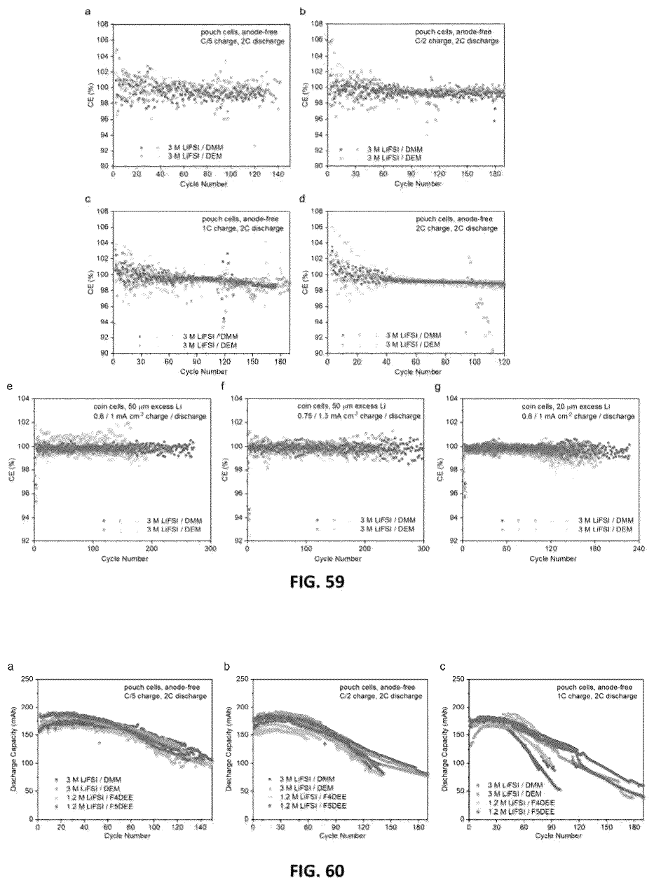

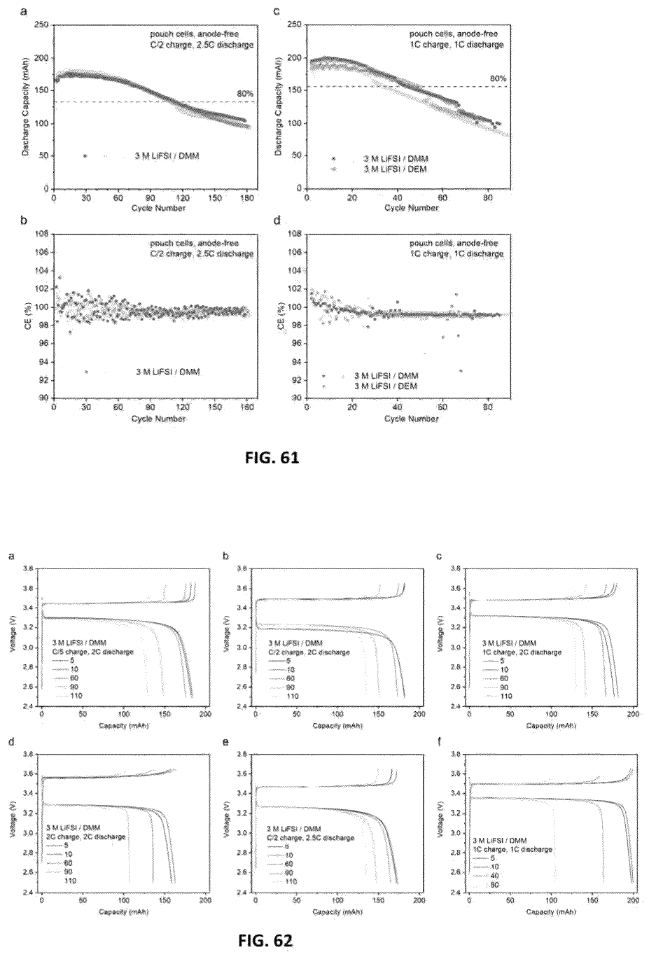

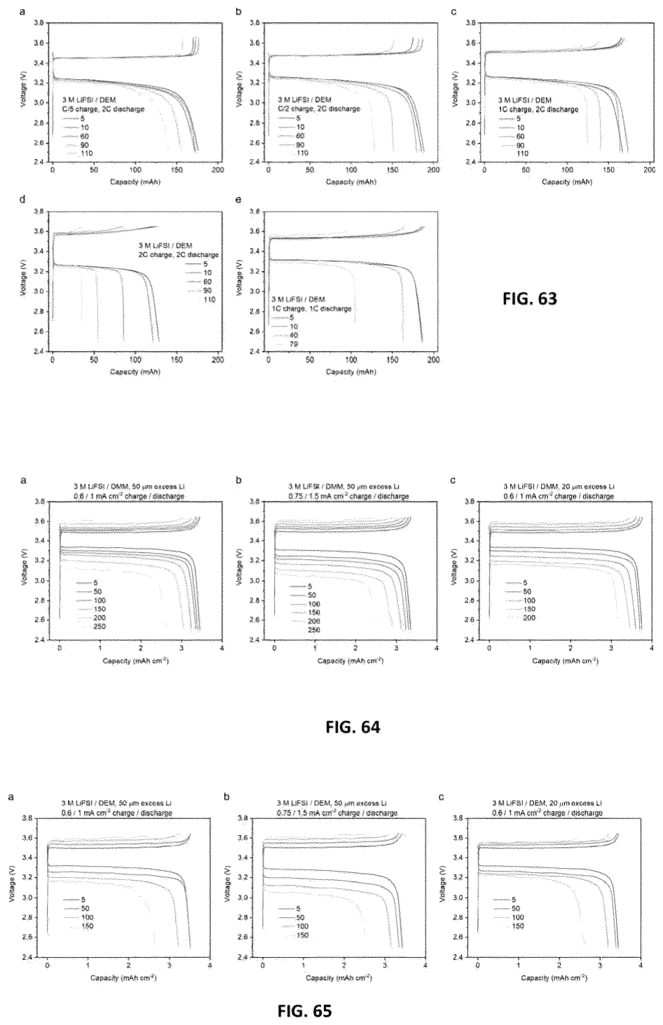

The fast activation of CE, high average CE, fast ion transport, and low overpotential make 3 M LiFSI/DMM a promising candidate for anode-free LMBs with high-rate capability. Commercial Cu∥micro-LFP dry pouch cells (Supplementary Table S3) were tested using both 3 M LiFSI/DMM and 3 M LiFSI/DEM electrolytes. Previously, almost identical pouch cells were used to test fluorinated DEE (F4DEE and F5DEE) electrolytes. For a direct comparison, the same charge rates (C/5, C/2 and 1C) and discharge rate (2C) as before were selected. Under the three different charge rates, both the DMM and DEM electrolytes achieved around 100 cycles before 80% capacity retention with good reproducibility (FIGS. 40a-c). The corresponding CE were above 99% with only small fluctuations (FIGS. 59a-c), indicating good cycling stability. In comparison to F4DEE and F5DEE, which were the best-performing electrolytes for high-rate anode-free LFP cells, the DMM and DEM electrolytes achieved similar cycle life with higher capacity utilization at C/5 charge and 2 C discharge rates (FIG. 60a). As the charge rate further increased to C/2 and 1C, the DMM and DEM electrolytes demonstrated better cycling stability than F4DEE and F5DEE (FIGS. 60b, c) due to the more apparent advantage of fast ion transport under high rates. At a very high 2 C charge rate, the capacity utilization was significantly higher in 3 M LiFSI/DMM than 3 M LiFSI/DEM (FIG. 40d) due to their difference in ion transport while CE remained stable in both electrolytes (FIG. 59d). Notably, despite the differences in bulk and interfacial ion transport, the capacity utilization was similar in 3 M LiFSI/DMM and 3 M LiFSI/DEM at ≤1 C charge rate (2 mA cm⁻²) due to the flat voltage curve of LFP cells—a cut-off voltage of 3.65 V was sufficient to accommodate the overpotential (FIGS. 62a-c and 63a-c). In contrast, at 2 C charge rate, the voltage divergence at the end of charge was obscured by overpotential, which led to the strong dependence of capacity utilization on overpotential (FIGS. 62d and 63d). Additional Cu∥micro-LFP pouch cells were cycled at C/2 charge, 2.5 C discharge and 1 C charge, 1 C discharge rates (FIGS. 61, 62e-f and 63e). In all anode-free pouch cells, cycle life was most likely limited by Li consumption since very little overpotential increase was observed (FIGS. 62 and 63).

The relatively short cycle life in anode-free cells obscured the difference in long-term stability. Therefore, thin-Li∥micro-LFP coin cells were also tested using high-loading cathode (nominally 3.6 to 4 mAh cm⁻²) and limited excess Li anode (50 or 20 μm thick) cycled at 0.6/1 mA cm⁻² or 0.75/1.5 mA cm⁻² charge/discharge current densities (FIGS. 40e-g and 59e-g). Under the three testing conditions, 3 M LiFSI/DMM showed significantly longer cycle life than 3 M LiFSI/DEM. With 50 μm thick Li, the cycle life was limited by increase in overpotential as evidence by the lack of voltage divergence at the end of charge of the 250^{th} and 150^{th} cycle (roughly corresponding to 80% capacity retention) in 3 M LiFSI/DMM and 3 M LiFSI/DEM respectively (FIGS. 64a-b and 65a-b). As a result, 3 M LiFSI/DMM with a slower increase in overpotential outperformed 3 M LiFSI/DEM. With 20 μm thick Li, the voltage divergence at the end of charge remained visible at the 200^{th} and 150^{th} cycle (roughly corresponding to 80% capacity retention) in 3 M LiFSI/DMM and 3 M LiFSI/DEM respectively (FIGS. 64c and 65c), which indicated that Li consumption likely limited cycle life. Consequently, 3 M LiFSI/DMM with higher initial CE and stabilized CE outperformed 3 M LiFSI/DEM.

### I. C. 3. Conclusions

By designing solvent coordination geometry, we were able to effective tune Li⁺ solvation structure and electrolyte reactivity. The non-linear molecular geometry of DMM and DEM enabled more weakly single-oxygen coordination with Li⁺, which leads to favorable Li⁺—FSI⁻ interaction and interfacial reactivity. At both 0.9 M and 3 M LiFSI concentrations, DMM and DEM demonstrated high CE >99%. In addition, the DMM electrolytes enabled fast activation of Li∥Cu cells to reach 99% CE within 3 to 5 cycles.

Ion transport is another crucial aspect to enable the practical application of LMBs. Due to similar ionic conductivity and higher limiting current fraction, 3 M LiFSI/DMM showed slightly lower overpotential than 3 M LiFSI/DEE in Li∥Li cells.

The fast activation of CE, high average CE, fast ion transport, and low overpotential make 3 M LiFSI/DMM a promising candidate for LMBs with high-rate capability.

### I. C. 4. Supplemental Information

FIG. 41 illustrates ¹J_{CH} coupling constants of anomeric —CH₂— of DMM and DEM with various concentrations of LiFSI. The corresponding molecular geometries for different ranges of ¹J_{CH} are shown on the right.

TABLE S1
Densities and concentrations of the various electrolytes
investigated in this work. The molarities were
calculated using electrolyte densities.

| | Electrolyte | Solvent | Molarity | Molality |
|---|---|---|---|---|
| | Density | Density | (M, | (m, |
| | (g/mL) | (g/mL) | mol/L) | mol/kg) |
| 1 mol LiFSI / liter DME | 0.975 | 0.867 | 0.93 | 1.15 |
| 4 mol LiFSI / liter DME | 1.242 | 0.867 | 3.08 | 4.61 |
| 1 mol LiFSI / liter DEE | 0.963 | 0.842 | 0.94 | 1.19 |
| 4 mol LiFSI / liter DEE | 1.216 | 0.842 | 3.06 | 4.75 |
| 1 mol LiFSI / liter DMM | 0.985 | 0.86 | 0.94 | 1.16 |
| 4 mol LiFSI / liter DMM | 1.240 | 0.86 | 3.08 | 4.65 |
| 1 mol LiFSI / liter DEM | 0.910 | 0.831 | 0.89 | 1.20 |
| 4 mol LiFSI / liter DEM | 1.172 | 0.831 | 2.97 | 4.81 |

FIG. 42 illustrates Repeated Li∥Cu CE measurement by a modified Aurbach method at room temperature (corresponds to FIG. 37c in the main text).

FIG. 43 illustrates Li∥Cu CE of 3 M LiFSI in DMM, DEM and DEE measured by the modified Aurbach method at 0° C. Two repeated cells are shown for each electrolyte.

FIG. 44 illustrates Temperature-dependent ionic conductivities of 3 M LiFSI in DMM, DEM and DEE with Celgard 2325 separator.

FIG. 45 illustrates Li∥Cu CE of 3 M LiFSI in DEE measured by the modified Aurbach method at −20° C.

FIG. 46 illustrates Oxidative stability of the electrolytes measured by LSV using Al (a-b) and Pt (c-d) as the working electrode. For comparison, the data of DME and DEE electrolytes are reproduced from Chen, Y.; Yu, Z.; Rudnicki, P.; Gong, H.; Huang, Z.; Kim, S. C.; Lai, J.-C.; Kong, X.; Qin, J.; Cui, Y.; et al. Steric Effect Tuned Ion Solvation Enabling Stable Cycling of High-Voltage Lithium Metal Battery. J. Am. Chem. SOC. 2021, 143 (44), 18703-18713. https://doi.org/10.1021/jacs.1c09006.

FIG. 47 illustrates Additional SEM images of the initial Li deposition morphology in 3 M LiFSI in DMM. A small amount of Li (0.5 mAh cm⁻²) was plated onto Cu at 0.5 mA cm⁻² in an uncycled Li∥Cu cell.

FIG. 48 provides Additional SEM images of the initial Li deposition morphology in 3 M LiFSI in DEM. A small amount of Li (0.5 mAh cm⁻²) was plated onto Cu at 0.5 mA cm⁻² in an uncycled Li∥Cu cell.

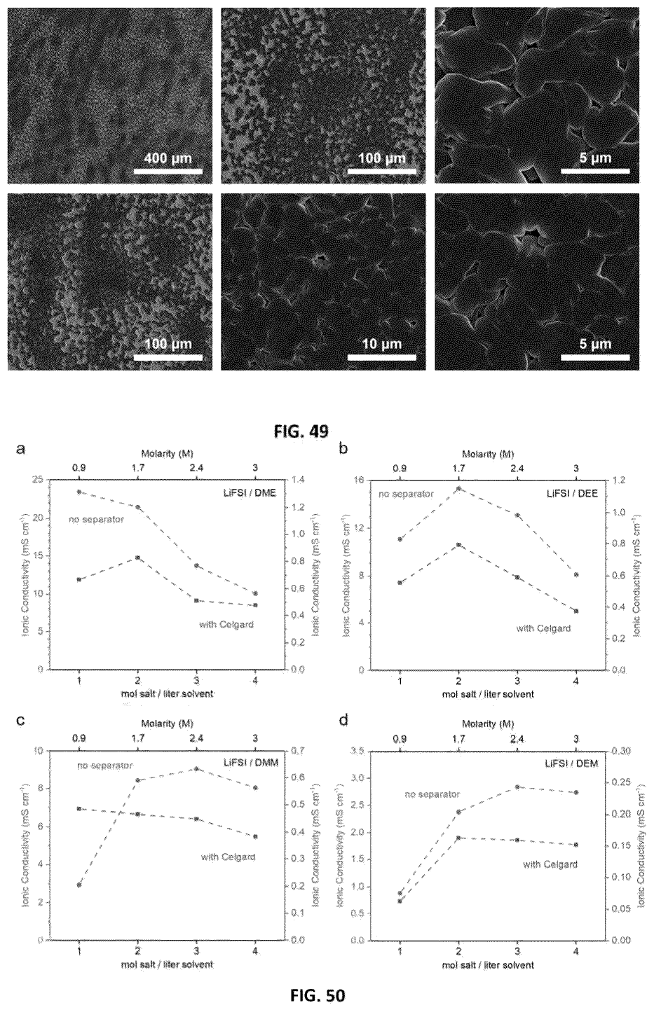

FIG. 49 provides Additional SEM images of the initial Li deposition morphology in 3 M LiFSI in DEE. A small amount of Li (0.5 mAh cm⁻²) was plated onto Cu at 0.5 mA cm⁻² in an uncycled Li∥Cu cell.

FIG. 50 illustrates Concentration-dependent ionic conductivities of LiFSI in DME (a), DEE (b), DMM (c) and DEM (d) with Celgard 2325 separator (blue, right axis) and without separator (green, left axis).

FIG. 51 illustrates Concentration-dependent molar conductivities of LiFSI in DME (a), DEE (b), DMM (c) and DEM (d) without separator.

FIG. 52 illustrates Self-diffusion coefficients of solvents, Li⁺ and FSI⁻ in 0.9 M and 3 M electrolytes measured by DOSY.

FIG. 53 illustrates Viscosity of 0.9 M and 3 M electrolytes.

FIG. 54 illustrates Inverse Haven ratios (1/H_{R}) of 0.9 M and 3 M electrolytes calculated using the equations below.

FIG. 55 illustrates Ionic conductivities of 0.9 M and 3 M electrolytes. The values were replotted from FIG. 50. The presentation here directly correlates to FIG. 39a.

FIG. 56 provides a Zoomed-in view of FIG. 39b showing overpotential at different stages of Li∥Li cycling.

FIG. 57 illustrates Impedance of Li∥Li cells over cycling with 3 M LiFSI in DMM (a, b), DEM (c, d) and DEE (e, f). (a, c, e) Nyquist plots and fitting curves. An equivalent circuit of (R1+C2/R2+C3/R3+C4/R4) was used. (b, d, f) The corresponding fitting values for each electrolyte, where R_{bulk}=R1 and R_{interface}=R2+R3+R4.

TABLE S2
Limiting current fraction measured by Vincent-Bruce method.

| | Limiting current |
|---|---|
| | fraction (ρ₊) |
| 0.9M LiFSI DMM | 0.48 |
| 3M LiFSI DMM | 0.46 |
| 0.9M LiFSI DEM | 0.71 |
| 3M LiFSI DEM | 0.57 |
| 3M LiFSI DEE | 0.30 |

FIG. 58 provides a Zoomed-in view of FIG. 39c showing overpotential under high current densities.

TABLE S3
Commercial pouch cell parameters

| Cu foil | 7 | μm |
|---|---|---|
| Separator | 12 μm PE coated with alumina | |
| Al foil | 12 | um |
| LFP:carbon:binder | 96.7:1.5:1.8 | |
| Nominal capacity | ~210 | mAh |
| Areal capacity | ~2.1 | mAh cm⁻² |
| Electrolyte | 0.5 | mL |
| Temperature | Uncontrolled room | |
| | temperature | |
| Pressure | ~1000 | kPa |

Note: the only difference between these pouch cells and the Cu∥LFP pouch cells used in Yu, Z.; Rudnicki, P. E.; Zhang, Z.; Huang, Z.; Celik, H.; Oyakhire, S. T.; Chen, Y.; Kong, X.; Kim, S. C.; Xiao, X.; et al. Rational Solvent Molecule Tuning for High-Performance Lithium Metal Battery Electrolytes. Nat. Energy 2022, 7 (1), 94-106. https://doi.org/10.1038/s41560-021-00962-y is the absence of 1 μm carbon coating on Cu.

FIG. 59 illustrates Corresponding CE values of cells in FIG. 40.

FIG. 60 provides Direct comparison of FDEE electrolytes with DMM and DEM electrolytes in Cu∥micro-LFP pouch cells. The data of fluorinated DEE were reproduced from Yu, Z.; Rudnicki, P. E.; Zhang, Z.; Huang, Z.; Celik, H.; Oyakhire, S. T.; Chen, Y.; Kong, X.; Kim, S. C.; Xiao, X.; et al. Rational Solvent Molecule Tuning for High-Performance Lithium Metal Battery Electrolytes. Nat. Energy 2022, 7 (1), 94-106. https://doi.org/10.1038/s41560-021-00962-y.

FIG. 61 illustrates Anode-free Cu∥micro-LFP pouch cells (nominally ˜210 mAh, ˜2.1 mAh cm⁻², 2.5 to 3.65 V, 0.5 mL electrolyte, 1C=200 mA) cycled at various rates (the first-cycle charge was at C/10) with 3 M LiFSI/DMM and 3 M LiFSI/DEM.

FIG. 62 provides Voltage curves of anode-free Cu∥micro-LFP pouch cells cycled at various charge and discharge rates in 3 M LiFSI/DMM.

FIG. 63 provides Voltage curves of anode-free Cu∥micro-LFP pouch cells cycled at various charge and discharge rates in 3 M LiFSI/DEM.

FIG. 64 provides Voltage curves of thin-Li∥micro-LFP coin cells cycled at various charge and discharge current densities in 3 M LiFSI/DMM.

FIG. 65 provides Voltage curves of thin-Li∥micro-LFP coin cells cycled at various charge and discharge current densities in 3 M LiFSI/DEM.

I. D. Designing Non-Fluorinated Solvents Via Alkoxy Chain Length Tuning for Stable. High-Voltage Lithium Metal Batteries

### I. D. 1. Abstract

Lithium metal batteries commonly use 1,2-dimethoxyethane (DME) as an electrolyte solvent. However, cell performance is constrained by DME's poor high-voltage stability at the cathode and inadequate Coulombic efficiency at the Li anode. Previously, it was shown that interfacial stability at both electrodes can be improved by substituting methoxy groups on DME with ethoxy groups. The resulting 1,2-diethoxyethane (DEE) features weakened solvation ability through steric hindrance effect, which induces stable anion-derived SEI on Li anode and which improves passivation of the aluminum cathode current collector. To further investigate the effects of steric hindrance of solvents on electrolyte performance, we fine tune the substituent size of a series of 1,2-dialkoxyethane solvents, including 1-ethoxy-2-n-propoxyethane (EtPrE), 1,2-di-n-propoxyethane (DnPE), and 1,2-di-n-butoxyethane (DnBE). With 4 mol lithium bis(fluorosulfonyl)imide (LiFSI) per liter of solvent, both EtPrE and DnPE exhibit good Li cycling stability, sufficient ionic conductivity, and superior oxidative stability compared to DME and DEE. However, the ionic conductivity decreases with substituent size. In the most extreme case of DnBE, the ion transport becomes too sluggish, which leads to low capacity utilization. When using 4 mol LiFSI per liter of DnPE or EtPrE, high-voltage full cells with 50 μm Li∥ca. 4 mAh cm⁻² NMC811 achieved more than 350 cycles at 80% capacity retention. This work demonstrates the fine tuning of steric hindrance as an effective strategy for designing non-fluorinated ether solvents for stable, high-voltage Li metal batteries.

### I. D. 2. Introduction

Lithium (Li) metal batteries are widely seen as the next step forward for energy storage applications in consumer electronics and electric mobility. As the specific energy of conventional lithium (Li)-ion batteries (LIBs) approaches its theoretical limit when using graphite-based anodes, Li metal anodes can enable vastly improved performance owing to its highest specific capacity (3,860 mAh/g) and lowest reduction potential (3.04 V versus standard hydrogen electrode [SHE]). (Chu, S., Cui, Y., & Liu, N. The path towards sustainable energy. Nat. Materials, 16, 16-22 (2016); Wang, H., et al. Liquid electrolyte: The nexus of practical lithium metal batteries, Joule, 6, 588-616 (2022); Liu, J. et al. Pathways for practical high-energy long-cycling lithium metal batteries. Nat. Energy. 4, 180-186 (2019).) However, Li metal batteries currently suffer from low Coulombic efficiency (CE) and poor cycle life, both of which arise from uncontrollable Li-electrolyte side reactions and large volume changes of the Li anode during cycling. (Wang, H. et al. Lithium Metal Anode Materials Design: Interphase and Host. Electrochem. Energ. Rev. 2, 509-517 (2019); Zhang, J. G., Xu, W., Xiao, J., Cao, X., Liu, J. Lithium Metal Anodes with Nonaqueous Electrolytes. Chemical Reviews. 120, 13312-13348 (2020).) Specifically, the protective solid electrolyte interphase (SEI) breaks down from the volume change of Li, promoting consumption of Li and electrolyte. (Peled., S., Menkin, S. Review-SEI. Past, Present, and Future. J. Electrochem. Soc. 164, A1703 (2017).) Non-uniform SEI formation further encourages dendritic Li growth and ‘dead Li’, contributing to higher cell overpotential, irreversible Li loss, and increased risk of internal short circuiting.

To combat these issues, ether-based electrolyte systems have seen a revival in interest and development. (Koch., V. R., Young, J. H. The stability of the secondary lithium electrode in tetrahydrofuran-based electrolytes. J. Electrochem. Soc. 125, 1371 (1978).) Compared to conventional carbonate-based electrolytes used in LIBs (Xu, K. et al. Nonaqueous Liquid Electrolytes for Lithium-Based Rechargeable Batteries. Chem. Rev. 104, 4303-4417 (2004)), ether-based electrolytes are able to form more stable SEI and increase CE of Li metal anode. A variety of liquid electrolyte engineering strategies for ether-based electrolytes have been developed, including high concentration electrolytes (HCEs) (Jeong, S.-K., Inaba, M., Iriyama, Y., Abe, T., Ogumi, Z. Interfacial reactions between graphite electrodes and propylene carbonate-based solutions: electrolyte concentration dependence of electrochemical lithium intercalation reaction. J. Power Sources. 175, 540-546 (2008); Ren, X. et al. High-Concentration Ether Electrolytes for Stable High-Voltage Lithium Metal Batteries. ACS Energy Lett. 4, 896-902 (2019); Qian, J. et al. Anode-free rechargeable lithium metal batteries. Adv. Funct. Mater. 26, 7094-7102 (2016); Yamada, Y., Wang, J., Ko, S., Watanabe, E., Yamada, A. Advances and issues in developing salt-concentrated battery electrolytes. Nat. Energy. 4, 269-280 (2019)), localized high concentration electrolytes (LCHEs) (Cao, X., Jia, H., Xu, W. Zhang, J.-G. Review—localized high-concentration electrolytes for lithium batteries. J. Electrochem. Soc. 168, 010522 (2021); Dokko, K. et al. Solvate ionic liquid electrolyte for Li—S batteries. J. Electrochem. Soc. 160, A1304-A1310 (2013); Ren, X. et al. Localized high-concentration sulfone electrolytes for high-efficiency lithium-metal batteries. Chem. 4, 1877-1892 (2018); Ren, X. et al. Enabling high-voltage lithium-metal batteries under practical conditions. Joule. 3, 1662-1676 (2019)), dual salt designs (Jiao, S. et al. Stable cycling of high-voltage lithium metal batteries in ether electrolytes. Nat. Energy. 3, 739-746 (2018); Qiu, F. et al. A concentrated ternary-salts electrolyte for high reversible Li metal battery with slight excess Li. Adv. Energy Mater. 9, 1803372 (2019)) and single-salt-single-solvent systems (Yu, Z. et al. Molecular design for electrolyte solvents enabling energy-dense and long-cycling lithium metal batteries. Nat. Energy. 5, 526-533 (2020); Wang, H. et al. Dual-solvent Li-ion solvation enables high-performance Li-metal batteries. Adv. Mater. 33, 2008619 (2021); Xue, W. et al. Ultra-high-voltage Ni-rich layered cathodes in practical Li metal batteries enabled by a sulfonamide-based electrolyte. Nat. Energy. 6, 495-505 (2021); Ma, P., Mirmira, P. Amanchukwu, C. V. Effect of building block connectivity and ion solvation on electrochemical stability and ionic conductivity in novel fluoroether electrolytes. ACS Cent. Sci. 7, 1232-1244 (2021). In particular, localized high concentration electrolytes (LCHEs) using lithium bis(fluorosulfonyl)imide (LiFSI) in 1,2-dimethoxyethane (DME) have found success thanks to excellent dendrite suppression, high Li CE, good salt solubility, and low viscosity. Nevertheless, shortcomings in DME—oxidative instability at high voltages (>4.2 V) and incompatibility with aluminum (Al) current collectors—have made DME a challenge to pair with high-voltage cathodes, such as layered transition metal oxides (NMC). To rectify this, fluorinated ether solvents such as 2,2,3,3-tetrafluoro-1,4-dimethoxybutane (FDMB) and several 1,2-di-(fluoroethoxy)ethane (FDEE) species (Yu, Z., Rudnicki, P. E., Zhang, Z. et al. Rational solvent molecule tuning for high-performance lithium metal battery electrolytes. Nat Energy 7, 94-106 (2022)) have demonstrated excellent high-voltage stability in addition to further improved Li metal performance. However, concerns over the environmental impact and high cost of fluorinated components necessitate the development of non-fluorinated ether solvents. (Flamme, B. et al. Guidelines to Design Organic Electrolytes for Lithium-Ion Batteries: Environmental Impact, Physicochemical and Electrochemical Properties. Green Chem. 19, 1828-1849 (2017)).

Based on performance and cost requirements, a practical electrolyte for Li metal batteries must be holistically designed for high CE for Li metal cycling, oxidative stability for high-energy cathodes, high ionic conductivity for practical cycling rates, cost-effectiveness using inexpensive ingredients, and environmental friendliness. To satisfy these conditions, our group has previously investigated single-salt, single-solvent electrolytes using 1,2-diethoxyethane (DEE) with LiFSI. (Chen, Y. et al. Steric Effect Tuned Ion Solvation Enabling Stable Cycling of High-Voltage Lithium Metal Battery. J. Am. Chem. Soc. 143, 18703-18713 (2021).) By substitution of the methoxy groups on DME with longer ethoxy groups, steric hindrance weakens solvation of LiFSI to promote both a stable, anion-derived SEI as well as improved oxidative stability against NMC811 cathode and Al cathode current collector. Full cells with 4 M LiFSI/DEE electrolyte were found to operate above 80% capacity retention for 182 cycles. The use of DEE-based electrolytes introduces a new design strategy wherein structural changes of solvent molecules can sterically alter solvation ability and interfacial stability with both the anode and cathode.

In this subsection of the disclosure, we utilize the structural diversity of 1,2-dialkoxyethane to introduce a series of molecular analogues to DEE with varying alkoxy chain lengths, namely 1-ethoxy-2-n-propoxyethane (EtPrE), 1,2-di-n-propoxyethane (DnPE), and 1,2-di-n-butoxyethane (DnBE). The electrochemical stabilities and ion transport properties of the corresponding electrolytes were characterized. When paired with 4 mol LiFSI per liter of solvent, EtPrE and DnPE were found to have high Li CE, sufficient ionic conductivity, and improved oxidative ability at Al current collector versus DME and DEE. On the other hand, additional increases in alkoxy chain length significantly impedes ion transport at practical current densities, as seen with DnBE. We assessed high-voltage (4.4 V) full-cell performance of these electrolytes with high-loading NMC811 (ca. 4 mAh cm⁻²) and thin Li (50 μm thickness). With 4 mol LiFSI per liter of DnPE or EtPrE, full cells were able to achieve more than 350 cycles at 80% capacity retention. Our investigation sheds light on how steric hindrance effects of DEE can be applied with a promising class of DEE analogues to tune solvation ability and achieve stable, high-voltage Li metal battery performance.

### I. D. 3. Results and Discussion

I. D. 3. a. Ion Transport Properties

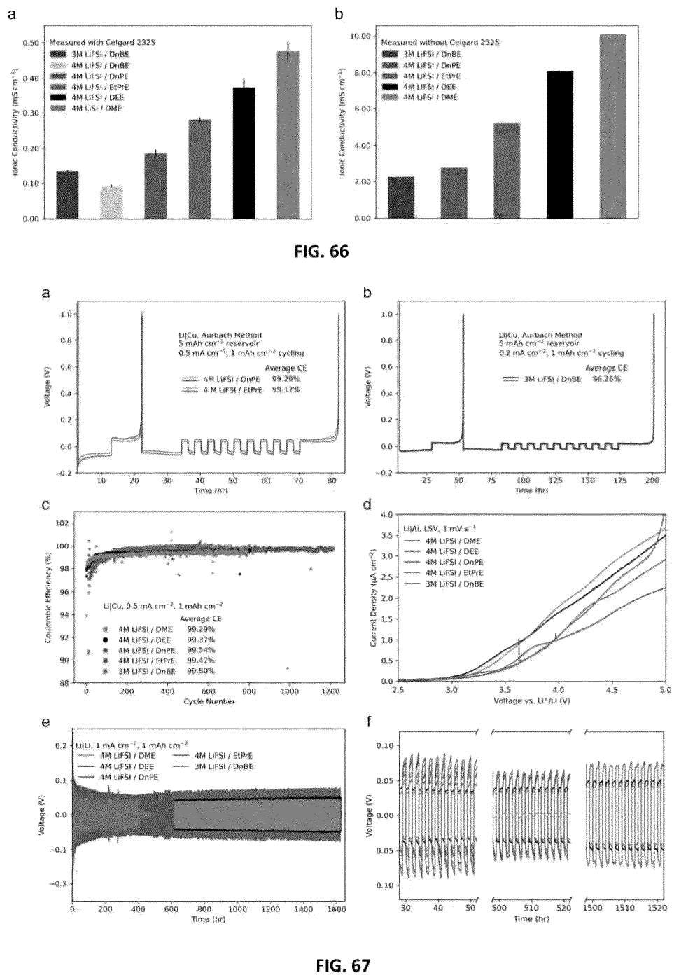

FIG. 66 illustrates Ionic conductivities of evaluated electrolytes with (a) and without (b) separators. Bar values in (a) represent the mean of multiple ionic conductivity measurements.

High electrolyte ionic conductivity is a key challenge for practical cycling of Li metal batteries. In particular, HCEs are prone to low ionic conductivity from weak solvation that causes ion clustering and disrupts ion mobility. (Qian, J. et al. High rate and stable cycling of lithium metal anode. Nat. Commun. 6, 6362 (2015).) Accounting for weaker solvation caused by steric effects of DEE compared to DME, we expect electrolytes with longer-chain solvents to have lower conductivities. Measured ionic conductivities with separators (FIG. 66a) reflected this expectation with 4M LiFSI/DnBE displaying the lowest conductivity and 4M LiFSI/DME displaying the highest. Anticipating issues with electrode wetting and poor ion transport of 4M LiFSI/DnBE, 3M LiFSI/DnBE was also measured and selected as an electrolyte candidate instead. Ionic conductivity trends were identical when measured without separator (FIG. 66b)—note that the lack of separator increases the conductivity values by an order of magnitude.

I. D. 3. b. Electrochemical Stability

FIG. 67 illustrates Electrochemical stability of 4 M LiFSI/EtPrE, 4 M LiFSI/DnPE, and 3 M LiFSI/DnBE electrolytes: (a) Modified Aurbach measurements of Li CEs for LiFSI/EtPrE and LiFSI/DnPE. (b) LiFSI/DnBE was evaluated at a lower rate. (c) Li CEs from Li∥Cu long term cycling, with average stabilized CEs calculated from the 200^{th} to 400^{th} cycle. (d) Oxidative stability on Al current collector. (e) Long-term cycling of Li∥Li symmetric cells. (f) Zoomed-in voltage curves during varying stages of Li∥Li cycling.

The electrolytes were assessed for stability at both the Li anode and at the Al cathode current collector. First, we evaluated electrolyte stability at the Li anode. Li CEs of Li∥Cu cells were calculated using a modified Aurbach method. (Adams, B. D., Zheng, J., Ren, X., Xu, W., Zhang, J. G. Accurate Determination of Coulombic Efficiency for Lithium Metal Anodes and Lithium Metal Batteries. Adv. Energy Mater. 8, 1702097 (2018).) At 4 M LiFSI, DnPE and EtPrE showed good Li CE (99.29% and 99.17%, respectively), with DnPE exhibiting better anode stability given its higher CE (FIG. 67a). On the other hand, cycling via the Aurbach method at 0.5 mA cm⁻² was unstable with 3 M LiFSI/DnBE. Even at a lower current density of 0.2 mA cm⁻², the measured Li CE (96.26%) was remarkably lower compared to DnPE and EtPrE (FIG. 67b).

To substantiate Li CE values from the Aurbach method, long-term cycling of Li∥Cu cells was performed (FIG. 67c). Average CEs for all electrolytes exceeded 99.2% with stable cycling. Compared to 4 M LiFSI/DME (99.29%) and 4 M LiFSI/DEE (99.37%), electrolytes with longer-chain solvents exhibited higher stabilized Li CEs (calculated from the 200^{th} to 400^{th} cycle) of 99.47%, 99.54%, and 99.80% for 4 M LiFSI/EtPrE, 4 M LiFSI/DnPE, and 3 M LiFSI/DnBE, respectively. Solvents with longer alkoxy chain lengths appeared to contribute to higher Li CEs, as seen with the relatively higher CE of DnBE and DnPE electrolytes. Values measured with Li∥Cu long-term cycling varied slightly from Aurbach method measurements due to the different substrates during Li plating and stripping between the two methods. In addition, the discrepancy between measured 3 M LiFSI/DnBE CE from long-term cycling versus Aurbach method may arise from the Aurbach method's measurement of only initial cycles. It may also come from the difficulty for DnBE to handle the Aurbach method's larger plating capacity (5 mAh cm⁻²) compared to that of long-term cycling (1 mAh cm⁻²).

Li∥Li symmetric cells were also built to verify long-term stability and investigate electrolyte overpotential (FIGS. 67e, f). For long-term stability, electrolytes composed of DnPE or DnBE solvents performed poorly at the 1 mA cm⁻² rate used. The importance of ion transport was clear here, as more ionically conductive electrolytes such as 4 M LiFSI/EtPrE fared much better in long-term performance. Contrary to expectations, Li∥Li overpotential for EtPrE, DnPE, and DnBE electrolytes did not follow trends of ionic conductivity, where overpotential should decrease with conductivity. It is possible that Li⁺ transference number may be higher for electrolytes with longer alkoxy chain length solvents, resulting in smaller overpotential. Differences in interfacial resistance may also be responsible for the deviation from expected trends and can be further investigated with EIS throughout Li∥Li cycling.

In addition to electrolyte stability at the Li anode, the oxidative stability of electrolytes was characterized with linear sweep voltammetry (LSV) on Li∥Al cells (FIG. 67d), where Al simulated the presence of an Al current collector in a practical full cell scenario. In LiFSI-based electrolytes, Al corrosion at high voltages typically limits the oxidative stability. (McOwen, D. et al. Concentrated electrolytes: decrypting electrolyte properties and reassessing Al corrosion mechanisms. Energy Environ. Sci. 7, 416-426 (2014).) Fast increases in leakage current during LSV indicate corrosion of the Al current collector. We observe no significant leakage current spikes under 5 V, indicating little corrosion from all electrolytes and good overall oxidative stability within the typical cathode voltage range. Furthermore, the results suggested that oxidative stability improved with weakened solvation ability, since at a given voltage the leakage current generally decreased with increasing alkoxy chain length.

I. D. 3. c. Full Cell Performance

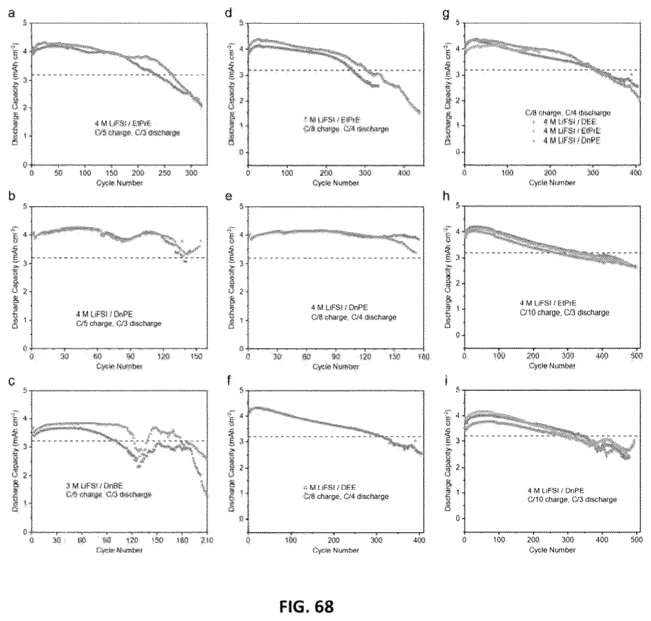

FIG. 68 illustrates Discharge capacities of Li∥NMC811 full cells consisted of NMC811 (ca. 4 mAh cm⁻²), thin Li (50 μm thick, N/P˜2.5), and relatively lean electrolyte amount (E/C˜10 mL Ah⁻¹). The cells were cycled between 2.8 and 4.4 V. Two formation cycles were conducted at C/10 charge and discharge (1C=4 mA cm⁻²). Long-term cycling was carried out at C/5 charge and C/3 discharge (a-c), C/8 charge and C/4 discharge (d-g), C/10 charge and C/3 discharge (h-i). The electrolytes are labeled in each figure. The dash line corresponds to 80% retention (3.2 mAh cm⁻²) of nominal discharge capacity.

We further tested full coin cells to demonstrate electrolyte performance in realistic cycling conditions. NMC811 cathode with ca. 4 mAh cm⁻² nominal capacity was chosen with a high cutoff voltage of 4.4 V to impose high-voltage, high specific capacity conditions and deep cycling of Li anode. Thin Li foil (50 μm thickness, N/P˜2.5) and a relatively lean electrolyte amount (E/C˜10 mL Ah⁻¹) were used.

We first carried out cycling at C/5 charge and C/3 discharge rates. The cells with 4 M LiFSI/EtPrE sustained about 235 to 265 cycles before the 80% retention (3.2 mAh cm⁻²) of nominal discharge capacity (FIG. 68a). The cells with 4 M LiFSI/DnPE suddenly failed by short circuit after about 150 cycles (FIG. 68b). The cells with 3 M LiFSI/DnBE became unstable after about 120 cycles (FIG. 68c). The cycle life appeared to follow the trend of ionic conductivity. The low conductivities of 4 M LiFSI/DnPE and 3 M LiFSI/DnBE likely limited the cycle life. In addition, due to the low conductivity of 3 M LiFSI/DnBE, the cells exhibited obvious loss of capacity utilization (FIG. 68c).

We further tested 4 M LiFSI/EtPrE and DnPE at slower charge (C/8) and discharge (C/4) rates. A slight improvement in cycle life was observed in 4 M LiFSI/EtPrE cells, which sustained about 265 to 300 cycles before 80% capacity retention (FIG. 68d). The 4 M LiFSI/DnPE cells showed a more significant improvement with more stable discharge capacity (FIG. 68e). However, it still failed by short circuit after about 170 cycles. For comparison, we also tested 4 M LiFSI/DEE under the same condition (FIG. 68f). The three electrolytes were directly compared in FIG. 68g. 4 M LiFSI/EtPrE showed slower initial decay in capacity compared to 4 M LiFSI/DEE although both electrolytes exhibited similar cycle life to 80% retention. In contrast, 4 M LiFSI/DnPE significantly underperformed.

Finally, 4 M LiFSI/EtPrE and DnPE were tested at C/10 charge and C/3 discharge. For 4 M LiFSI/EtPrE cells, a minimal improvement in cycle life (260 to 320 cycles before 80% retention) was observed compared to C/8 charge and C/4 discharge, which indicated that ionic conductivity was not limiting the cycling stability. Interestingly, 4 M LiFSI/DnPE showed a significant improvement in cycle life (280 to 340 cycles before 80% retention) compared to faster rates. This supported our hypothesis that poor rate capability of 4 M LiFSI/DnPE limited its cycle life at C/5, C/3 and C/8, C/4 rates due to its low ionic conductivity.

### I. D. 4. Conclusion

The common Li metal electrolytes such as LiFSI/DME suffer from anode and oxidative instability. Previously, we demonstrated solvation tuning via steric hindrance using DEE as a solvent. Building on the molecular design of DEE to make sterically hindered, weakly solvating electrolytes, our work extended the steric hindrance effect seen in DEE to a new series of electrolytes—EtPrE, DnPE, and DnBE—for improved stability in high-voltage Li metal batteries. All three electrolytes exhibited superior long-term Li∥Cu CE and oxidative stability compared to DEE, a consequence of improved SEI quality and Al passivation. However, ionic conductivity decreased with the chain length of the substituent. The poor ionic conductivity led to low capacity utilization of 3 M LiFSI/DnBE cells as well as poor cycle life of 4 M LiFSI/DnPE and 3 M LiFSI/DnBE cells at higher charge and discharge rates. Therefore, improved stability from longer alkoxy chain lengths must be balanced with sufficient ionic conductivity. In strict full cell cycling conditions with ca. 4 mAh cm⁻² NMC811 and 50 μm Li at 2.8 to 4.4 V, 4 M LiFSI/EtPrE and DnPE sustained about 300 cycles before 80% capacity retention at C/10 charge and C/3 discharge. Overall, we demonstrated the generality of steric hindrance effect for designing non-fluorinated ether solvents for high-voltage LMBs.

### I. D. 5. Experimental Section

I. D. 5. a. Materials

Ethylene glycol diethyl ether (DEE, 99%, anhydrous) was purchased from Fisher Scientific. Ethylene glycol dibutyl ether (DnBE, 98%) was purchased from TCI. Ethylene glycol monopropyl ether and 1-Iodopropane were purchased from Sigma Aldrich. LiFSI was purchased from Arkema. Celgard 2325 separator (25 μm thick, polypropylene/polyethylene/polypropylene) was purchased from Celgard. The Cu current collector (25 μm thick) was purchased from Alfa Aesar. Thin Li foil (50 μm, free standing) and lithium chips (700 μm) were purchased from MSE Supplies. CR2032 battery casings, stainless steel spacers, springs, and Al-clad coin cell cases were purchased from MTI. NMC811 cathode sheets (ca. 4 mAh cm⁻²) were purchased from Targray.

I. D. 5. b. Synthesis of DnPE and EtPrE

To a 1 L round bottom flask, ethylene glycol monopropyl ether (50 mL, 1 eqv.) was mixed with 600 mL of anhydrous THF. The solution was cooled in an ice bath. NaH (21 g, 60% in paraffin, 1.2 eqv.) was added slowly to the solution. The mixture was stirred for an hour at room temperature. 1-Iodopropane (51 mL, 1.2 eqv.) was added in one portion and the mixture was stirred for an hour, followed by refluxing at 60 C overnight. The reaction mixture was filtered and THF was removed on the rotavap. The DnPE crude product was purified by vacuum distillation three times. A small amount of NaH was added before the last two distillations to remove water. The synthesis of EtPrE is similar, expect iodoethane was used instead of 1-iodopropane.

I. D. 5. c. Electrolyte Preparation

All solvents were stored in the Ar glovebox. A piece of fresh Li was added to remove any trace amount of water. Electrolytes were prepared by dissolving 4 mol of LiFSI per liter of EtPrE, 4 mol of LiFSI per liter of DnPE, and 3 or 4 mol of LiFSI per liter of DnBE.

I. D. 5. d. Electrochemical Measurements

Battery fabrication was performed in an Ar-filled glovebox. Unless otherwise specified, CR2032 coin cells were used for all electrochemical measurements and were cycled under ambient conditions.

Electrolyte ionic conductivities were measured by electrochemical impedance spectroscopy (Biologic VSP) on stainless steel symmetric electrodes and electrolyte soaked Celgard 2325 separator. Swagelok cells were also used to measure ionic conductivities without the presence of a separator.

To characterize Li metal anode stability, Li CEs were measured with a modified Aurbach method on Li∥Cu half cells. Cu surface was first conditioned by plating 5 mAh cm⁻² of Li and stripping to 1 V at 0.5 mA cm⁻². A Li reservoir of 5 mAh cm⁻² was subsequently plated onto Cu, followed by 10 cycles of Li plating and stripping at 1 mAh cm⁻² and 0.5 mA cm⁻². After 10 cycles, all Li on Cu was stripped to 1 V at 0.5 mA cm⁻². For 3 M LiFSI/DnBE, 0.2 mA cm⁻² was used due to poor ion transport. For Li CE measurements with long-term cycling of Li∥Cu half cells, Cu surface was first conditioned with a 0.01 V hold for 5 h, followed by 10 cycles between 0 and 1 V at 0.2 mA cm⁻². Cycling consisted of plating 1 mAh cm⁻² of Li onto Cu and then stripping to 1 V at 0.5 mA cm⁻². To investigate overpotential and long-term stability, Li∥Li symmetric cells were cycled at 1 mA cm⁻² for 1 mAh cm⁻².

Electrolyte oxidative stability was measured with linear sweep voltammetry (LSV) on Li∥Al cells using a Biologic VSP300. The voltage swept from open-circuit voltage to 7 V vs Li⁺/Li at a rate of 1 mV s⁻¹. The leakage current density was calculated based on an electrode area of 2.11 cm⁻² for Al.

Li∥NMC811 full cells were fabricated with 50 μm thin Li (ca. 10 mAh cm⁻²) and NMC811 cathode (ca. 4 mAh cm⁻²), with relatively lean electrolyte volume (40 μL). Al-clad cathode cases were used for high voltage. A piece of Al foil between cathode and cathode casing was used to avoid defects in Al cladding. Full cells were cycled between 2.8 and 4.4 V. Two formation cycles were performed at 0.4 mA cm⁻² charge and discharge current densities. For long-term cycling, cells were charged at 0.5 mA cm⁻² and discharged at 1 mA cm⁻².

### II. Tuning Fluorination Degree of Ether and Carbonate Based Electrolyte Solvents for Lithium Metal and Lithium Ion Batteries

### II. A. Abstract

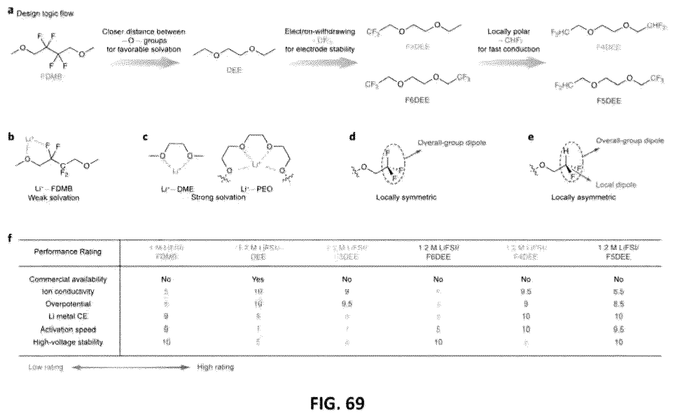

The present embodiments of this subsection relate to a family of fluorinated-1,2-diethyoxyethane (fluorinated-DEE) molecules that are readily synthesized in large scales to use as the electrolyte solvents. Selected positions on 1,2-diethyoxyethane (DEE, distinct from the diethyl ether previously reported (Holoubek, J. et al. Tailoring electrolyte solvation for Li metal batteries cycled at ultra-low temperature. Nat. Energy 6, 303-313 (2021)) are functionalized with various numbers of fluorine atoms through iterative tuning, to reach a balance between CE, oxidative stability, and ionic conduction (FIG. 69a). Paired with 1.2 M lithium bis(fluorosulfonyl)imide (LiFSI), these fluorinated-DEE-based, single-salt single-solvent electrolytes are thoroughly characterized. Their Li⁺-solvent binding energies and geometries (from density functional theory [DFT] calculations), solvation environments (from solvation free energy measurements, 7Li-nuclear magnetic resonance [NMR], molecular dynamics [MD] simulations and diffusion-ordered spectroscopy [DOSY](Su, C.-C. et al. Principle in developing novel fluorinated sulfone electrolyte for high voltage lithium-ion batteries. Energy Environ. Sci. 14, 3029-3034 (2021))), and results in batteries (measured ion conductivities and cell overpotentials) are found to be tightly correlated with each other. The above studies lead to an unexpected finding: partially-fluorinated, locally-polar —CHF₂ group results in higher ionic conduction than fully-fluorinated —CF₃ while still maintaining excellent electrode stability. Specifically, the best-performing F4DEE and F5DEE solvents both contain —CHF₂ group(s). In addition to high ionic conductivity and low, stable overpotential, they achieve ˜99.9% average CE for Li metal anode as well as fast activation, i.e., the CEs of the Li∥copper (Cu) half cells reach >99.3% from the second cycle. Aluminum (Al) corrosion was also significantly suppressed due to the oxidative stability that originated from suitable amount of fluorination. These features enabled ˜270 cycles in thin-Li (50-μm-thick)∥high-loading-NMC811 (LiNi_{0.8}Mn_{0.1}Co_{0.1}O₂, ˜4.9 mAh cm⁻²) full batteries and >140 cycles in fast-cycling anode-free Cu II microparticle-LFP (LiFePO₄, ˜2.1 mAh cm⁻²) pouch cells, both of which stand among the state-of-the-art performances. It is worth noting that anode-free cells based on microparticle-LFP are rarely studied (Sripad, S., Bills, A. & Viswanathan, V. The Iron Age of Automotive Batteries: Techno-economic assessment of batteries with lithium metal anodes paired with iron phosphate cathodes. ECSarXiv Prepr. (2021) doi:10.1149/osf.io/fx4p9; Eftekhari, A. LiFePO4/C nanocomposites for lithium-ion batteries. J. Power Sources 343, 395-411 (2017)) due to its low conductivity and limited-excess Li inventory compared to NMC (lithium nickel manganese cobalt oxide) cells. The long-cycling, high-rate Cu∥microparticle-LFP pouch cells demonstrated in this work thus fill the gap and allow for opportunities for low-cost Li metal batteries. The rational design process behind the electrolyte family presented in our work and our comprehensive investigation of its properties can be used to further develop the electrolytes towards practical Li metal batteries and fast cycling anode-free cells.

### II. A. 2. Introduction

Lithium (Li) metal battery is highly pursued as the next-generation power source (Liu, J. et al. Pathways for practical high-energy long-cycling lithium metal batteries. Nat. Energy 4, 180-186 (2019); Cao, Y., Li, M., Lu, J., Liu, J. & Amine, K. Bridging the academic and industrial metrics for next-generation practical batteries. Nat. Nanotechnol. 14, 200-207 (2019)). However, the implementation of Li metal anode is hindered by poor cycle life, which originates from uncontrollable Li/electrolyte side reactions, and the resulting unstable and fragile solid-electrolyte interphase (SEI). Subsequently, the notorious issues such as cracking of SEI, dendritic Li growth, and ‘dead Li’ formation generate a vicious cycle, irreversible Li consumption and finally battery failure. (Tikekar, M. D., Choudhury, S., Tu, Z. & Archer, L. A. Design principles for electrolytes and interfaces for stable lithium-metal batteries. Nat. Energy 1, 16114 (2016); Cheng, X.-B. et al. A Review of Solid Electrolyte Interphases on Lithium Metal Anode. Adv. Sci. 3, 1500213 (2016); Lin, D., Liu, Y. & Cui, Y. Reviving the lithium metal anode for high-energy batteries. Nat. Nanotechnol. 12, 194-206 (2017).)

Liquid electrolyte engineering is regarded as a cost-effective and pragmatic approach (Flamme, B. et al. Guidelines to design organic electrolytes for lithium-ion batteries: Environmental impact, physicochemical and electrochemical properties. Green Chem. 19, 1828-1849 (2017); Aspern, N., Röschenthaler, G.-V., Winter, M. & Cekic-Laskovic, I. Fluorine and Lithium: Ideal Partners for High-Performance Rechargeable Battery Electrolytes. Angew. Chemie Int. Ed. 58, 15978-16000 (2019); Jie, Y., Ren, X., Cao, R., Cai, W. & Jiao, S. Advanced Liquid Electrolytes for Rechargeable Li Metal Batteries. Adv. Funct. Mater. 30, 1910777 (2020); Fan, X. & Wang, C. High-voltage liquid electrolytes for Li batteries: progress and perspectives. Chem. Soc. Rev. 50, 10486-10566 (2021); Hobold, G. M. et al. Moving beyond 99.9% Coulombic efficiency for lithium anodes in liquid electrolytes. Nat. Energy 6, 951-960 (2021)). to address the root cause, i.e., uncontrollable parasitic reactions between Li metal anodes and electrolytes. By fine-tuning electrolyte components, the SEI chemistry and Li morphology can be regulated to improve Li metal cyclability. Several promising strategies have been investigated, including high concentration electrolytes (Yamada, Y., Wang, J., Ko, S., Watanabe, E. & Yamada, A. Advances and issues in developing salt-concentrated battery electrolytes. Nat. Energy 4, 269-280 (2019)), localized high concentration electrolytes (Cao, X., Jia, H., Xu, W. & Zhang, J.-G. Review-Localized High-Concentration Electrolytes for Lithium Batteries. J. Electrochem. Soc. 168, 010522 (2021); Ren, X. et al. Enabling High-Voltage Lithium-Metal Batteries under Practical Conditions. Joule 3, 1662-1676 (2019)), mixed solvents (Chen, J. et al. Electrolyte design for Li metal-free Li batteries. Mater. Today 39, 118-126 (2020); Holoubek, J. et al. An All-Fluorinated Ester Electrolyte for Stable High-Voltage Li Metal Batteries Capable of Ultra-Low-Temperature Operation. ACS Energy Lett. 5, 1438-1447 (2020); Wang, H. et al. Dual-Solvent Li-Ion Solvation Enables High-Performance Li-Metal Batteries. Adv. Mater. 33, 2008619 (2021)), additive tuning (Zhang, H. et al. Electrolyte Additives for Lithium Metal Anodes and Rechargeable Lithium Metal Batteries: Progress and Perspectives. Angew. Chemie Int. Ed. 57, 15002-15027 (2018)), liquified gas electrolytes (Yang, Y. et al. Liquefied gas electrolytes for wide-temperature lithium metal batteries. Energy Environ. Sci. 13, 2209-2219 (2020)), dual-salt-dual-solvent electrolytes (Weber, R. et al. Long cycle life and dendrite-free lithium morphology in anode-free lithium pouch cells enabled by a dual-salt liquid electrolyte. Nat. Energy 4, 683-689 (2019); Louli, A. J. et al. Diagnosing and correcting anode-free cell failure via electrolyte and morphological analysis. Nat. Energy 5, 693-702 (2020)), and single-salt-single-solvent electrolytes (Yu, Z. et al. Molecular design for electrolyte solvents enabling energy-dense and long-cycling lithium metal batteries. Nat. Energy 5, 526-533 (2020); Amanchukwu, C. V et al. A New Class of Ionically Conducting Fluorinated Ether Electrolytes with High Electrochemical Stability. J. Am. Chem. Soc. 142, 7393-7403 (2020); Xue, W. et al. Ultra-high-voltage Ni-rich layered cathodes in practical Li metal batteries enabled by a sulfonamide-based electrolyte. Nat. Energy 6, 495-505 (2021); Holoubek, J. et al. Tailoring electrolyte solvation for Li metal batteries cycled at ultra-low temperature. Nat. Energy 6, 303-313 (2021); Ma, P., Mirmira, P. & Amanchukwu, C. V. Effect of Building Block Connectivity and Ion Solvation on Electrochemical Stability and Ionic Conductivity in Novel Fluoroether Electrolytes. ACS Cent. Sci. 7, 1232-1244 (2021)). These approaches functioned well in Li metal batteries with limited Li inventory and even in anode-free cells with zero Li excess.

Specifically, the concept of anode-free cells only emerged recently for maximizing the energy density of Li metal batteries; however, they suffer from short cycle life since no Li inventory is present at the original anode (Nanda, S., Gupta, A. & Manthiram, A. Anode-Free Full Cells: A Pathway to High-Energy Density Lithium-Metal Batteries. Adv. Energy Mater. 11, 2000804 (2021); Park, S. H., Jun, D., Lee, G. H., Lee, S. G. & Lee, Y. J. Toward high-performance anodeless batteries based on controlled lithium metal deposition: a review. J. Mater. Chem. A 9, 14656-14681 (2021); Qian, J. et al. Anode-Free Rechargeable Lithium Metal Batteries. Adv. Funct. Mater. 26, 7094-7102 (2016); Sripad, S., Bills, A. & Viswanathan, V. The Iron Age of Automotive Batteries: Techno-economic assessment of batteries with lithium metal anodes paired with iron phosphate cathodes. ECSarXiv Prepr. (2021) doi:10.1149/osf.io/fx4p9). Unlike Li-ion batteries where the graphite anode can be quickly activated, Li metal anode usually takes hundreds of cycles to reach optimum Coulombic efficiency (CE) due to initial SEI stabilization and electrode activation (Xiao, J. et al. Understanding and applying coulombic efficiency in lithium metal batteries. Nat. Energy 5, 561-568 (2020)). Therefore, the anode-free cell design requires high Li metal CE over the whole cycling life, particularly during the initial activation cycles.

To enable practical Li metal or anode-free batteries, several key requirements, as proposed by the community, should be simultaneously fulfilled for a promising electrolyte: (1) high CE including the initial cycles, i.e., fast activation of Li metal anode, as illustrated above, (2) anodic stability to avoid cathode corrosion, (3) low electrolyte consumption under practical operating conditions such as lean electrolyte and limited Li inventory, (4) moderate Li salt concentration for cost effectiveness and (5) high boiling point and the absence of gassing issue to ensure processability and safety.

Beyond these requirements, high ionic conductivity is another critical parameter for realistic cycling rates. Several papers (Chen, Y. et al. Steric Effect Tuned Ion Solvation Enabling Stable Cycling of High-Voltage Lithium Metal Battery. J. Am. Chem. Soc. (2021) doi:10.1021/jacs.1c09006; Pham, T. D. & Lee, K. Simultaneous Stabilization of the Solid/Cathode Electrolyte Interface in Lithium Metal Batteries by a New Weakly Solvating Electrolyte. Small 17, 2100133 (2021); Xu, R. et al. Designing and Demystifying the Lithium Metal Interface toward Highly Reversible Batteries. Adv. Mater. (2021) doi:10.1002/adma.202105962) reported improved Li metal stability using weakly solvating solvents. However, insufficient solvation will lead to ion clustering, poor ion motion, and low solubility of salts, leading to low ionic conductivity. Therefore, fine-tuning of the solvation capability (Chen, X. & Zhang, Q. Atomic Insights into the Fundamental Interactions in Lithium Battery Electrolytes. Acc. Chem. Res. 53, 1992-2002 (2020)) of the solvent is necessary in order to simultaneously achieve Li metal cyclability, oxidative stability, and ionic conductivity of the electrolyte.

In this work, we systematically investigate a family of fluorinated-1,2-diethyoxyethane (fluorinated-DEE) molecules that are readily synthesized in large scales to use as the electrolyte solvents. Selected positions on 1,2-diethyoxyethane (DEE, distinct from the diethyl ether previously reported) are functionalized with various numbers of fluorine atoms through iterative tuning, to reach a balance between CE, oxidative stability, and ionic conduction (FIG. 69a). Paired with 1.2 M lithium bis(fluorosulfonyl)imide (LiFSI), these fluorinated-DEE-based, single-salt-single-solvent electrolytes are thoroughly characterized. Their Li⁺-solvent binding energies and geometries (from density functional theory [DFT] calculations), solvation environments (from solvation free energy measurements, Li-nuclear magnetic resonance [NMR], molecular dynamics [MD] simulations and diffusion-ordered spectroscopy [DOSY](Su, C.-C. et al. Principle in developing novel fluorinated sulfone electrolyte for high voltage lithium-ion batteries. Energy Environ. Sci. 14, 3029-3034 (2021)), and results in batteries (measured ion conductivities and cell overpotentials) are found to be tightly correlated with each other. The above studies lead to an unexpected finding: partially-fluorinated, locally-polar —CHF₂ group results in higher ionic conduction than fully fluorinated —CF₃ while still maintaining excellent electrode stability. Specifically, the best-performing F4DEE and F5DEE solvents both contain —CHF₂ group(s). In addition to high ionic conductivity and low, stable overpotential, they achieve ˜99.9% average CE for Li metal anode as well as fast activation, i.e., the CEs of the Li∥copper (Cu) half cells reach >99.3% from the second cycle. Aluminum (Al) corrosion was also significantly suppressed due to the oxidative stability that originated from suitable amount of fluorination. These features enabled ˜270 cycles in thin-Li (50-μm-thick) II high loading-NMC811 (LiNi_{0.8}Mn_{0.1}Co_{0.1}O₂, ˜4.9 mAh cm⁻²) full batteries and >140 cycles in 1 fast-cycling anode-free Cu II microparticle-LFP (LiFePO₄, ˜2.1 mAh cm⁻²) pouch cells, both of which stand among the state-of-the-art performances. It is worth noting that anode-free cells based on microparticle-LFP are rarely studied (Eftekhari, A. LiFePO4/C nanocomposites for lithium-ion batteries. J. Power Sources 343, 395-411 (2017)) due to its low conductivity and limited-excess Li inventory compared to NMC (lithium nickel manganese cobalt oxide) cells. The long-cycling, high-rate Cu II microparticle-LFP pouch cells demonstrated in this work thus fill the gap and allow for opportunities for low-cost Li metal batteries. The rational design process behind the electrolyte family presented in our work and our comprehensive investigation of its properties can be used to further develop the electrolytes towards practical Li metal batteries and fast cycling anode-free cells.

### II. A. 3. Design Logic of Fluorinated-DEE Molecular Family

Despite its high stability towards Li metal anodes and high-voltage cathodes, our previously reported FDMB solvent (FIG. 69a) was found to have the drawbacks of poor ionic conductivity and large overpotential (Wang, H. et al. Efficient Lithium Metal Cycling over a Wide Range of Pressures from an Anion-Derived Solid-Electrolyte Interphase Framework. ACS Energy Lett. 6, 816-825 (2021)), which stem from the weak solvation ability of FDMB molecules (FIG. 69b). Such a feature hindered ion diffusion due to the formation of ionic clusters as the majority of electrolyte solvates, while, on the other hand, benefiting Li metal anode stability (Liu, X. et al. Enhanced Li+ Transport in Ionic Liquid-Based Electrolytes Aided by Fluorinated Ethers for Highly Efficient Lithium Metal Batteries with Improved Rate Capability. Small Methods 9, 2100168 (2021)). To address this issue, we rationalize that ethylene oxide (EO) structure may be desirable as it is a known and widely-used segment (Halat, D. M. et al. Modifying Li+ and Anion Diffusivities in Polyacetal Electrolytes: A Pulsed-Field-Gradient NMR Study of Ion Self-Diffusion. Chem. Mater. 33, 4915-4926 (2021)) for good solvation and separating Li⁺ and anion. The ether groups in the EO segment, separated by two methylene groups, can form a stable five-member ring with Li⁺ (FIG. 69c), thus enhancing cation-anion separation. Such a chelating structure has been commonly observed in liquid electrolytes containing 1,2-dimethoxyethane (DME) and in solid polymer electrolytes (Id.) using polyethylene oxide (PEO). However, we herein select DEE (FIG. 69a) instead of DME as the starting backbone for the following additional reasons:
(i) The DEE electrolyte has been inadequately studied in the community despite recent reports on its superior high-rate performance than DME for Li metal and silicon (Ando, H. et al. Mixture of monoglyme-based solvent and lithium Bis(trifluoromethanesulfonyl)amide as electrolyte for lithium ion battery using silicon electrode. Mater. Chem. Phys. 225, 105-110 (2019)) anodes;
(ii) The ethyl terminal groups of DEE provide more structural tunability than DME and suitable β-fluorination (Sasaki, Y., Shimazaki, G., Nanbu, N., Takehara, M. & Ue, M. Physical and Electrolytic Properties of Partially Fluorinated Organic Solvents and Its Application to Secondary Lithium Batteries: Partially Fluorinated Dialkoxyethanes. ECS Trans. 16, 23-31 (2019); Yue, Z., Dunya, H., Aryal, S., Segre, C. U. & Mandal, B. Synthesis and electrochemical properties of partially fluorinated ether solvents for lithium-sulfur battery electrolytes. J. Power Sources 401, 271-277 (2018)) is expected to endow DEE with both stability and high conductivity.

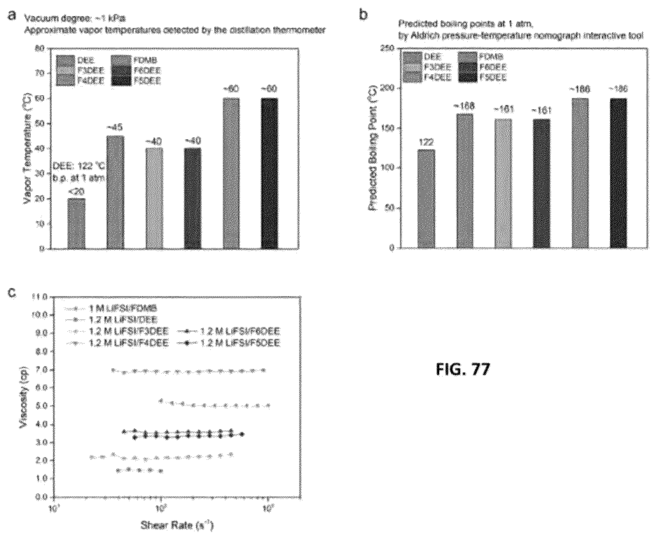

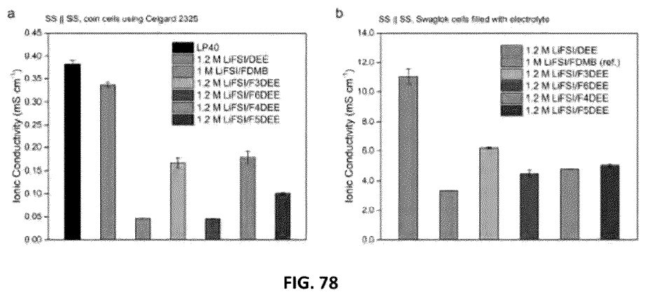

As will be elaborated in the following sections, the Li metal CE and oxidative stability of unmodified DEE still fall short when tested under strict full-cell conditions, albeit performing slightly better than DME. Therefore, starting from DEE structure, we first incorporate the electron-withdrawing —CF₃ groups (Zhang, Y. & Viswanathan, V. Design Rules for Selecting Fluorinated Linear Organic Solvents for Li Metal Batteries. J. Phys. Chem. Lett. 12, 5821-5828 (2021)) in the P-position of DEE, to enhance both Li metal and oxidative stability while retaining its solvation ability of —O— groups (FIGS. 69a and d). The two obtained electrolyte solvents, F3DEE and F6DEE (FIG. 69a), were found to outperform their DEE counterpart in Li metal batteries, although over-fluorination decreases the ionic conductivity of F6DEE. Next, we further finely tune the degree of fluorination, i.e., changing from —CF₃ groups to —CHF₂, to obtain more ionically conductive and stable solvents, F4DEE and F5DEE (FIG. 69a). The partially-fluorinated, asymmetric —CHF₂ group, as will be discussed in detail later, contains a local dipole (FIG. 69e) that enables strong intermolecular interactions in F4DEE and F5DEE and better Li⁺ solvation than its all-fluorinated, symmetric counterpart, —CF₃ (FIG. 69d). The stronger intermolecular interaction was also evidenced by the high boiling points and viscosities measured for F4DEE and F5DEE (FIGS. 77 and 78, Table 1). The iteratively designed molecules F4DEE and F5DEE integrate several desired properties, including fast ion conduction, low and stable cell overpotential, high Li metal efficiency, fast activation, and oxidative stability (FIG. 69f).

None of the designed molecules are commercially available, and they were obtained by two-step syntheses at large scales (Methods Syntheses). After purification by distillation, the general physicochemical properties of this molecular family were determined, and they were further prepared as 1.2 M LiFSI electrolytes (Supplementary Table 1), to systematically study the structure-performance relationships.

### II. A. 4. Improved Ionic Transport by Experimental Results

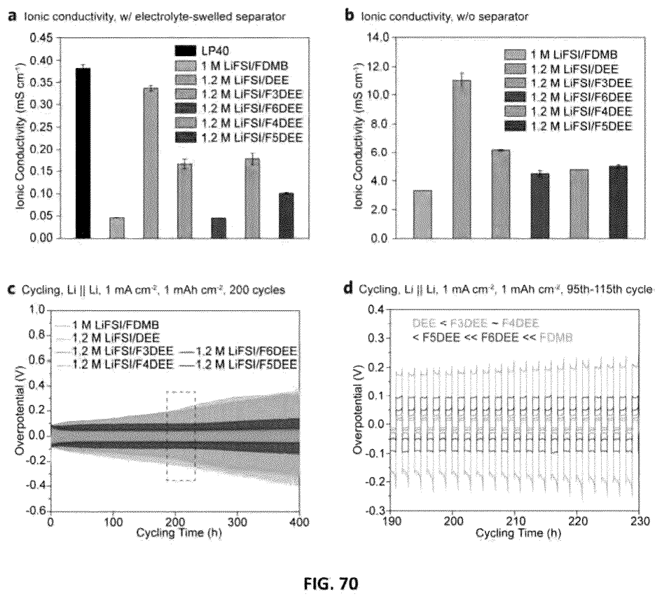

The critical targets in this work are to improve the ionic conductivity and interfacial transport issues of the already high-performing FDMB electrolyte. Conventional battery separators (Celgard, 25-μm-thick polypropylene-polyethylene-polypropylene trilayer membrane) were wetted by conventional carbonate electrolyte LP40 (1 M LiPF₆ in ethylene carbonate/diethyl carbonate), 1 M LiFSI/FDMB and 1.2 M LiFSI in fluorinated-DEEs, respectively, followed by sandwiching between two stainless steel (SS) electrodes to imitate the practical battery structure. The 1 M LiFSI/FDMB electrolyte was used to maintain consistency with our previous reports while 1.2 M LiFSI was dissolved in fluorinated-DEEs for optimized conductivity. The ionic conductivities measured by this setup followed the trend of LP40˜DEE>>F4DEE˜F3DEE>F5DEE>>F6DEE˜FDMB (FIG. 70a), which is fully consistent with our rationales. Those measured without separators by Swagelok cells showed a similar trend (FIG. 70b, FIG. 78 and Table 1), although the values are higher due to the absence of the separator.

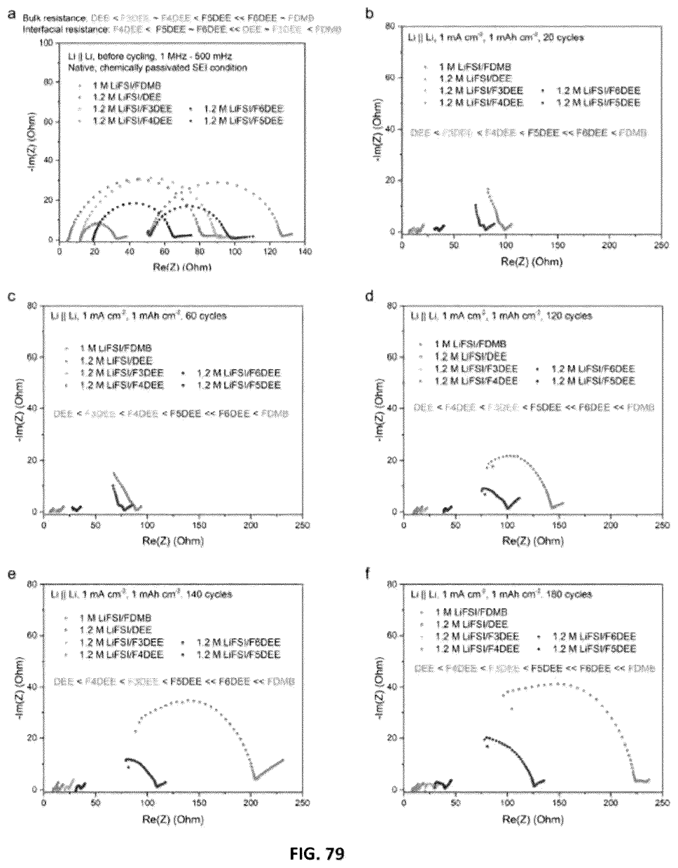

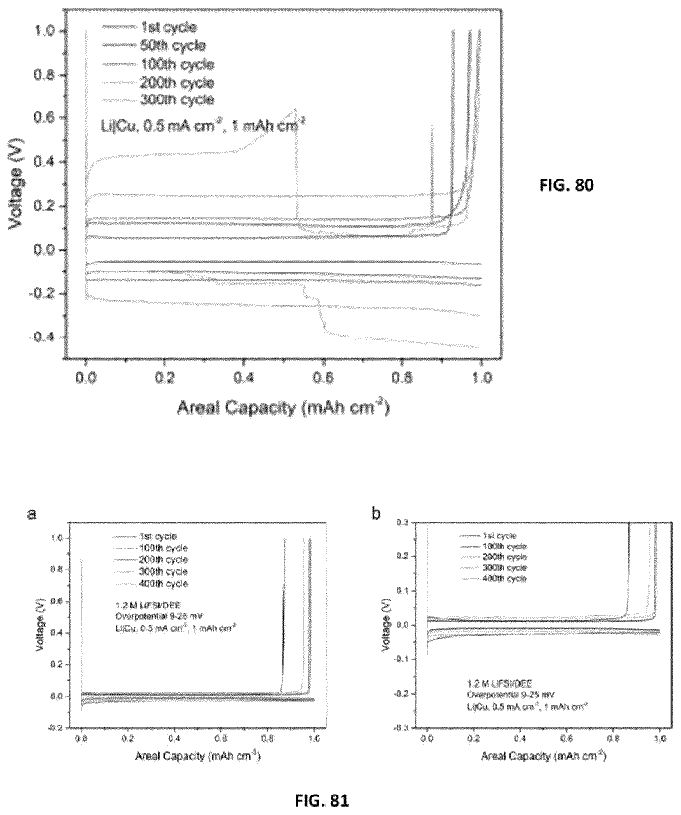

Li∥Li symmetric cells were used to evaluate the overall ionic transport, especially the dominating interfacial conduction. As shown in FIG. 70c, the overpotential of 1 M LiFSI/FDMB cell vastly increased with cycling; by contrast, the cells using fluorinated-DEE electrolytes maintained stable and low overpotentials. The electrochemical impedance spectra (EIS) of Li∥Li cells at different cycle numbers confirmed these cycling observations (FIG. 79). Additionally, the voltage plateau of Li∥Cu cells with cycling showed a much higher and continuously increasing overpotential for FDMB but stable and low values for almost all fluorinated-DEE electrolytes (FIGS. 80-84). Although the large overpotential increase in the FDMB electrolyte caused only a small capacity drop in full cells according to our previous reports, the excellent maintenance of low overpotential in fluorinated-DEE electrolytes is required for realistic batteries. The zoomed-in plot of Li∥Li cycling overpotentials shows the trend of DEE<F3DEE˜F4DEE<F5DEE<<F6DEE<<FDMB (FIG. 70d), which is in accord with the inverse of the ion conductivity trends mentioned above.

### II. A. 5. Rationales for Improved Ionic Conduction

In addition to the experimental observations, we here rationalize the improvements of ionic transport in fluorinated-DEE electrolytes via thorough theoretical studies, and correlate both theoretical and experimental results for better understanding the structure-property relationships.

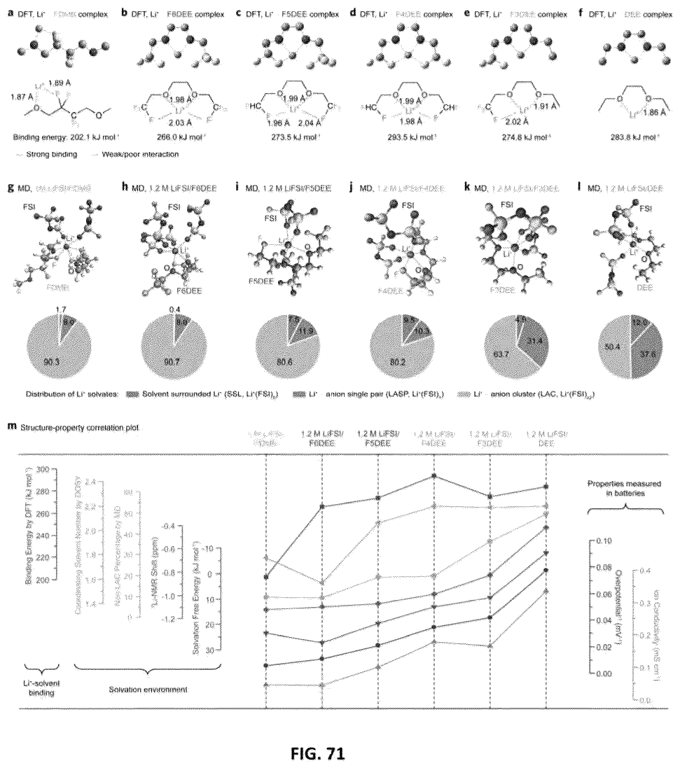

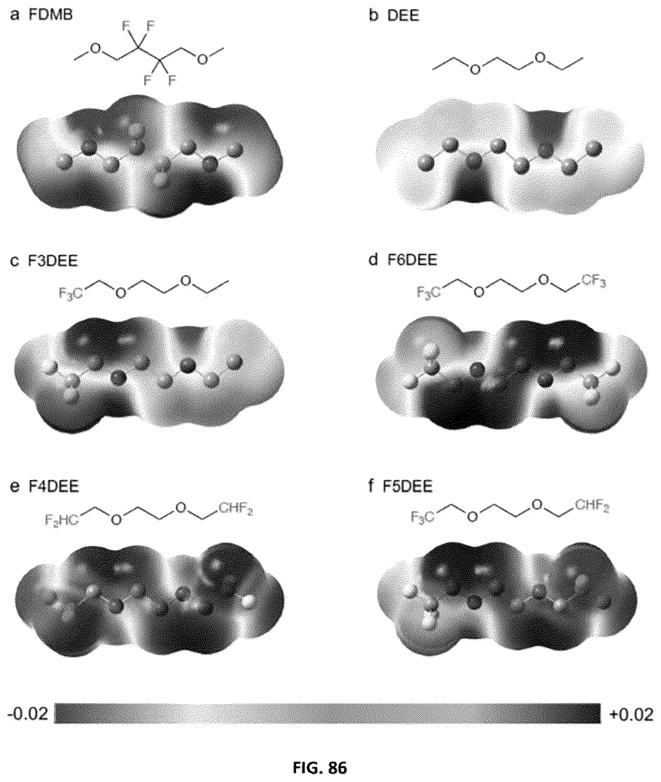

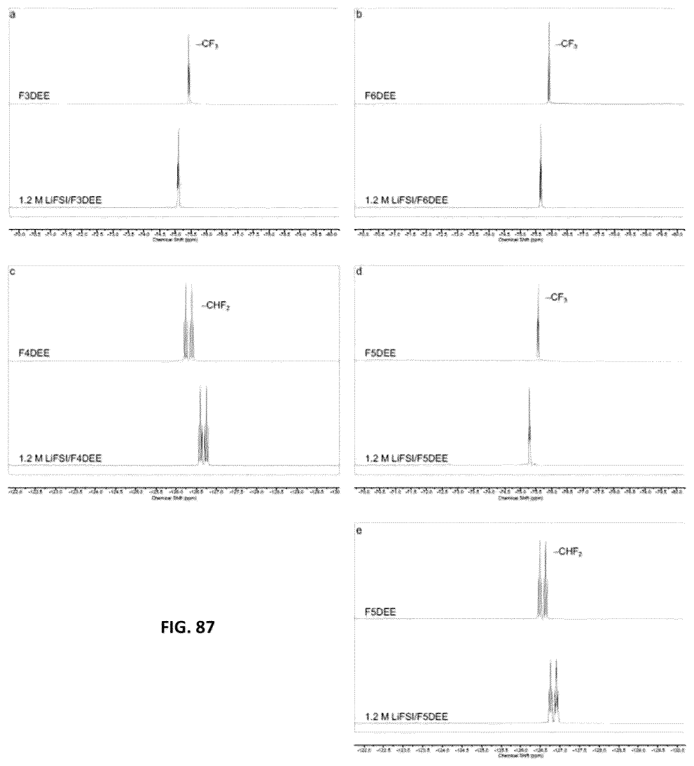

We first used DFT to determine optimized binding configurations between Li⁺ and each type of solvent molecule (FIGS. 71a-f). While the coordination structure of Li⁺-FDMB and Li⁺-DEE matched with those in the previous report, the Li⁺ ions all showed tripod or tetrapod coordination geometry with fluorinated-DEEs whose F atoms interacted with Li⁺ ions. The Li⁺ showed stronger interaction (i.e., shorter Li—F distance) with —CHF₂ than —CF₃. Taking Li⁺-F5DEE as a representative example (FIG. 71f), the Li—F (on —CHF₂) distance was 1.96 A versus 2.04 A for —CF₃. The nonparticipation of —CF₃ in Li⁺ solvation was also proved by Amanchukwu et al. recently. Such a stronger interaction between Li⁺ and the —CHF₂ group can be rationalized by the fact that —CHF₂ group is locally polar and more negatively charged than —CF₃ in the calculated electrostatic potentials (FIG. 69e and FIG. 86). The upfield shift of —CHF₂ signals detected by 19F-NMR spectra of fluorinated-DEE electrolytes also supports this Li—F interaction trend (Yu, Z. et al. A Dynamic, Electrolyte-Blocking, and Single-Ion-Conductive Network for Stable Lithium-Metal Anodes. Joule 3, 2761-2776 (2019); Jia, M. et al. Fluorinated Bifunctional Solid Polymer Electrolyte Synthesized under Visible Light for Stable Lithium Deposition and Dendrite-Free All-Solid-State Batteries. Adv. Funct. Mater. 31, 2101736 (2021)) (FIG. 87).

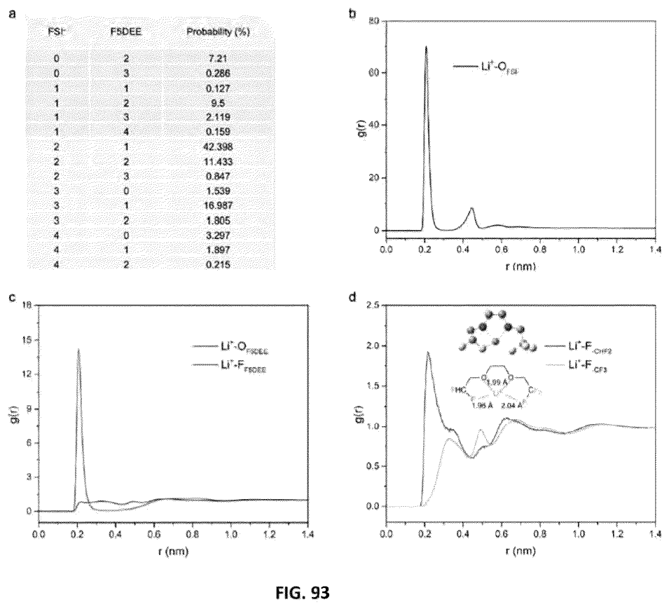

MD simulations were conducted to further investigate the Li⁺ solvation sheath and determine the distribution of Li⁺ solvates (FIGS. 71g-1 and 88-93). The functional groups tightly interacting with Li⁺ in the first solvation sheath were generally similar to those in the aforementioned DFT results (e.g., —CHF₂ on F4DEE and F5DEE preferentially coordinated with Li⁺ rather than —CF₃, FIGS. 71i and j). Particularly, the Li—F radial distribution functions (RDFs) of simulated 1.2 M LiFSI/F5DEE clearly demonstrated more F atoms on —CHF₂ participating in Li⁺ solvation than those on —CF₃ (FIG. 93). More information was provided by the distribution of Li⁺ solvates, i.e., percentages of solvent surrounded Li⁺ (SSL), Li⁺-anion single pair (LASP), and Li⁺-anion cluster (LAC), each of which has a distinct number of Li⁺ coordinating anions of 0, 1 and >2 in the primary solvation sheath, respectively. It is noteworthy that the classification of these Li⁺ solvates is slightly different from the conventional definition of solvent separated ion pair, contact ion pair, or aggregate1. The later ones use anion as the center to count the coordinating Li⁺ number; instead, the SSL, LASP and LAC herein are proposed based on Li⁺ solvation structures. In all electrolytes, LAC dominated the Li⁺ solvate species but the content of SSL and LASP (both classified as non-LAC) varied dramatically from one electrolyte to another, indicating significant difference in ion dissociation degree. While almost no SSL and only a small proportion of LASP was observed in FDMB or F6DEE electrolytes, the non-LAC increased in the order of F5DEE (7.5% SSL+11.9% LASP), F4DEE (9.5% SSL+10.3% LASP), F3DEE (4.9% SSL+31.4% LASP), and DEE (12.0% SSL+37.6% LASP).

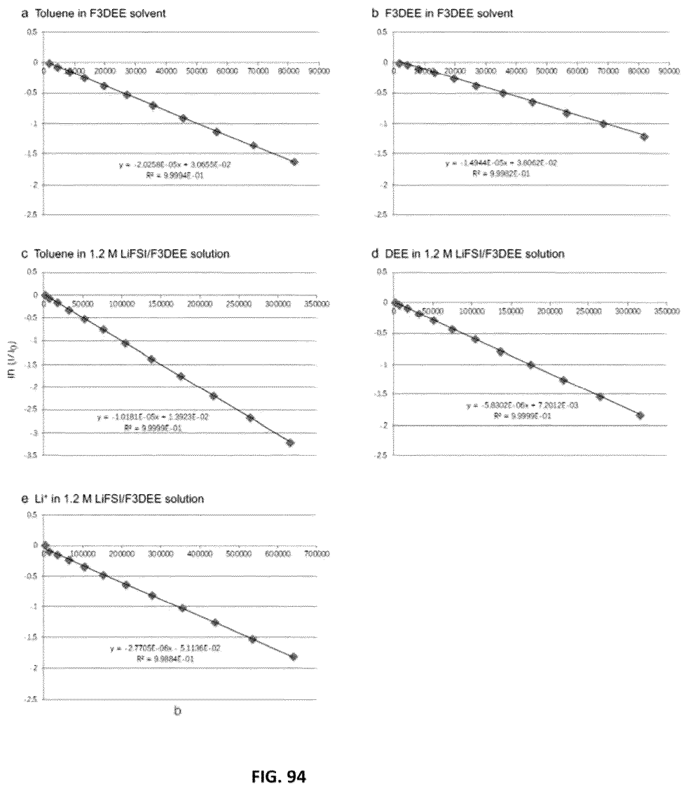

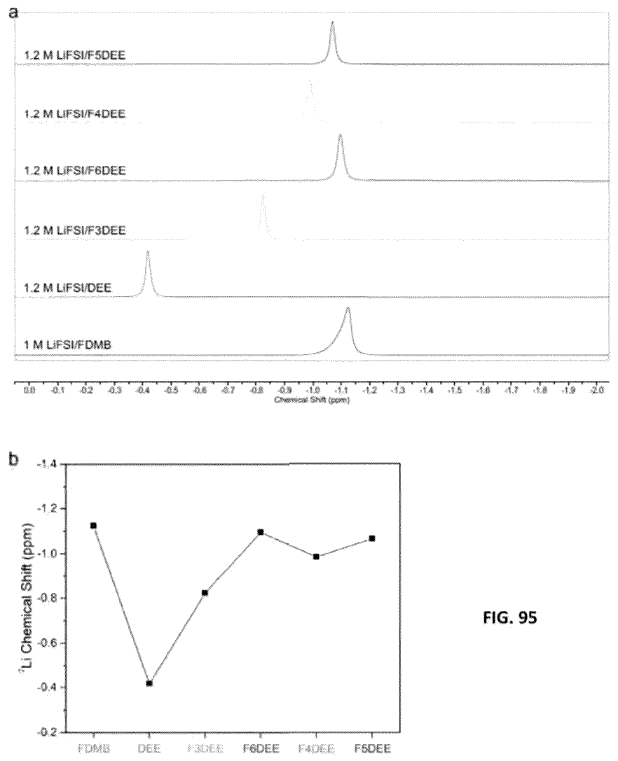

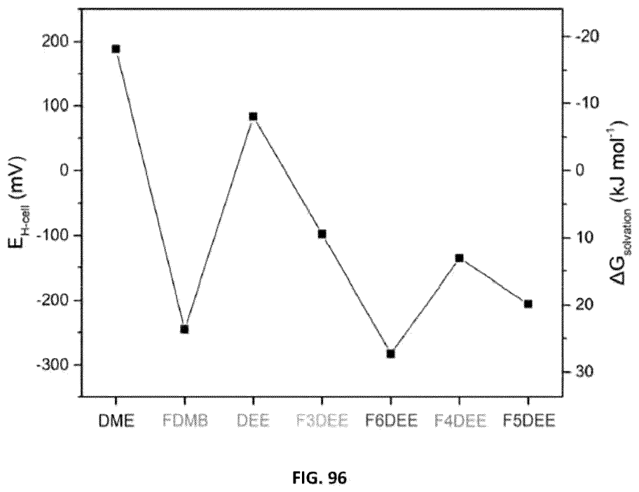

To elucidate structure-property correlations in depth, the following parameters/properties were leveraged to cross-validate the Li⁺-solvent interaction, solvation environments, and properties measured in batteries: (1) Li⁺-solvent binding energies from DFT (FIGS. 71a-f), (2) coordinating solvent numbers calculated from DOSY-NMR, a method developed by Amine et al. (Supplementary Table 2 and FIG. 94), (3) non-LAC percentages from MD simulations (FIGS. 71g-1 and 88-93), (4) chemical shifts of 7Li-NMR (FIG. 95), (5) solvation free energies measured according to our recent work (Kim, S. C. et al. Potentiometric Measurement to Probe Solvation Energy and Its Correlation to Lithium Battery Cyclability. J. Am. Chem. Soc. 143, 10301-10308 (2021)) (FIG. 96), (6) ionic conductivities shown in FIG. 70a, and (7) overall cycling overpotentials of Li∥Li cell extracted from FIG. 70c (converted to inversed overpotentials to better represent conduction property).

As plotted in FIG. 71m, these parameters follow similar trends against the choice of electrolytes. The main logic and rationales are as follows:
(i) More solvent molecules participating in the Li⁺ solvation sheath, i.e., higher coordination numbers calculated by DOSY and more non-LAC solvates shown in MD simulations, indicate greater binding ability and stronger Li⁺-solvent interaction regardless of minor deviations (Zou, Y. et al. Interfacial Model Deciphering High-Voltage Electrolytes for High Energy Density, High Safety, and Fast-Charging Lithium-Ion Batteries. Adv. Mater. (2021) doi:10.1002/adma.202102964); meanwhile, more coordinating solvents dispel electron-dense FSI− anions near Li⁺ and cause downfield (less negative) shift of 7Li-NMR peak.
(ii) Solvation free energy is an overall estimation of the solvation environment46 (and the extent of Gibbs free energy decrease) between Li⁺ ions and surrounding species including both solvents and anions. Since the anion was fixed as FSI− in this work, stronger binding solvents will lead to more negative solvation energies.
(iii) At moderate concentrations, i.e., 1 or 1.2 M solutions with low viscosities (Supplementary Table 1) where the vehicular mechanism dominates Li⁺ transport, strong binding solvents and good solvation reduces severe Li⁺-FSI− clustering (revealed by increasing non-LAC percentage and downfield 7Li shift), and result in separated, mobile Li⁺ charge carriers, which are responsible for the higher ionic conductivity and lower overall overpotential (higher inversed overpotential value) obtained in batteries.
(iv) It is worth noting that all the fluorinated-DEEs should still be classified as weakly-solvating solvents; however, fine-tuning of fluorination enables sufficient solvation for fast transport while retaining electrode stabilities.

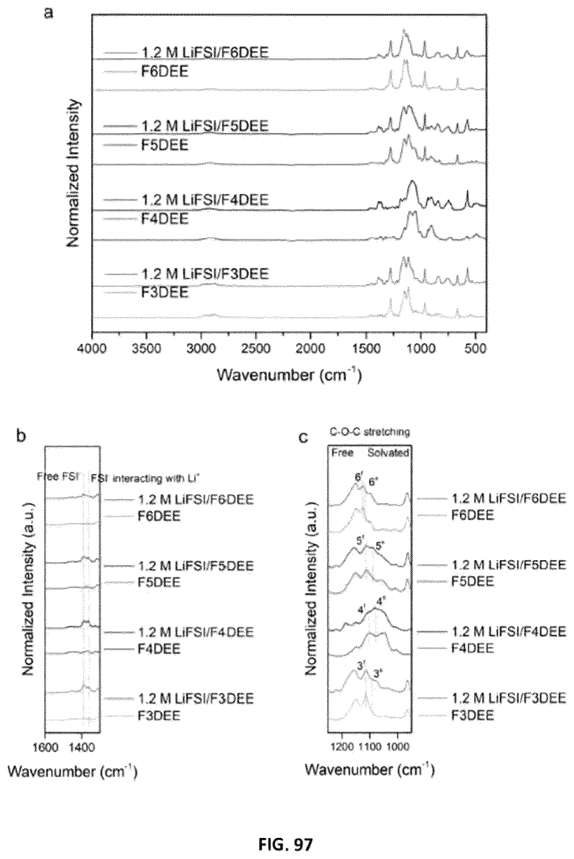

These arguments can be further cross-validated by attenuated total reflection Fourier transform infrared spectroscopy (ATR-FTIR) results, which showed more solvating ether groups in F3DEE, F4DEE and F5DEE electrolytes compared to the poorly solvating F6DEE one (FIG. 97). All these factors and their correlations are consistent with each other and fill a broad spectrum of scales ranging from molecular-level structure to mesoscopic Li⁺ solvation cluster statistics to bulk electrolyte properties, and finally to battery performance.

### II. A. 6. Enhanced Li Metal and Oxidative Stability

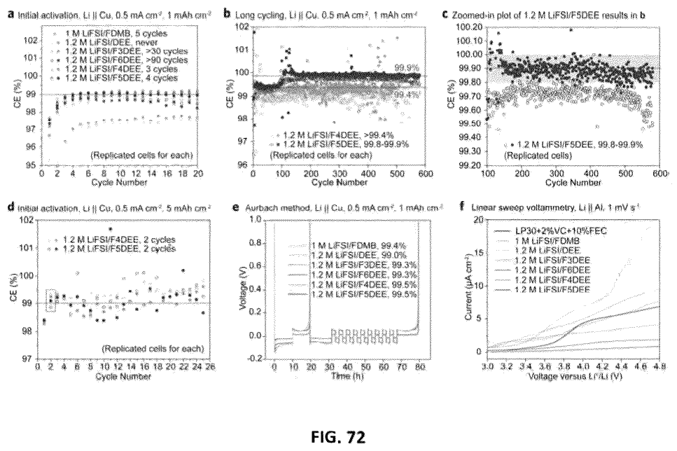

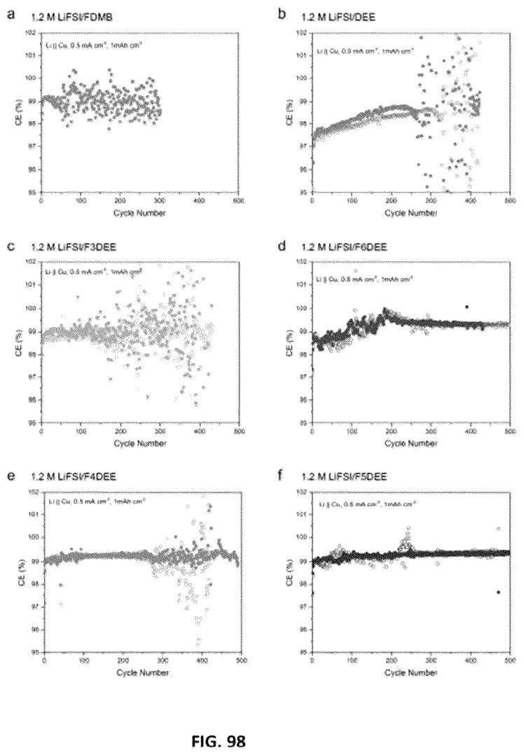

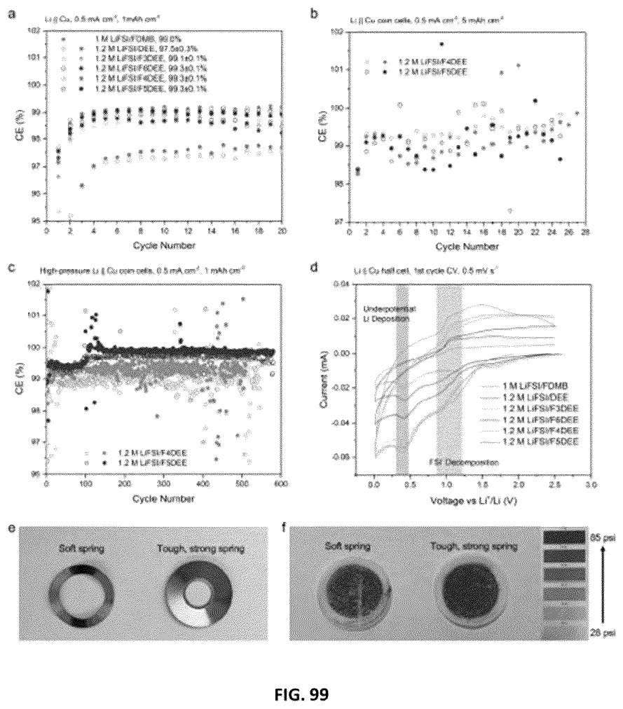

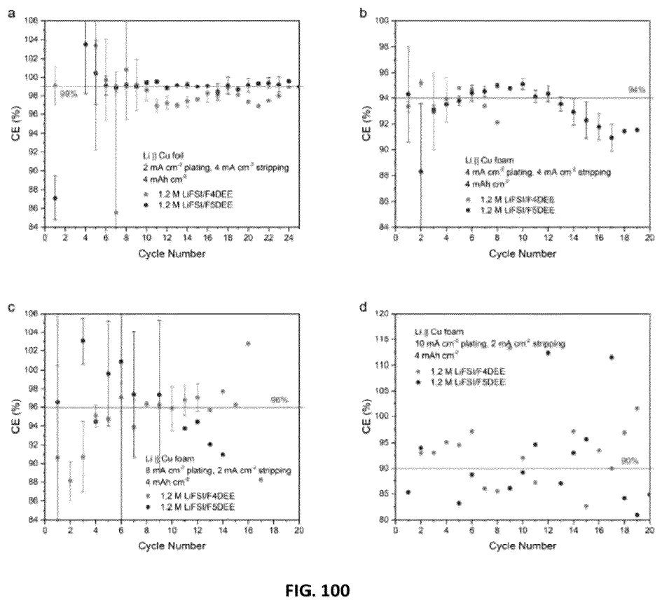

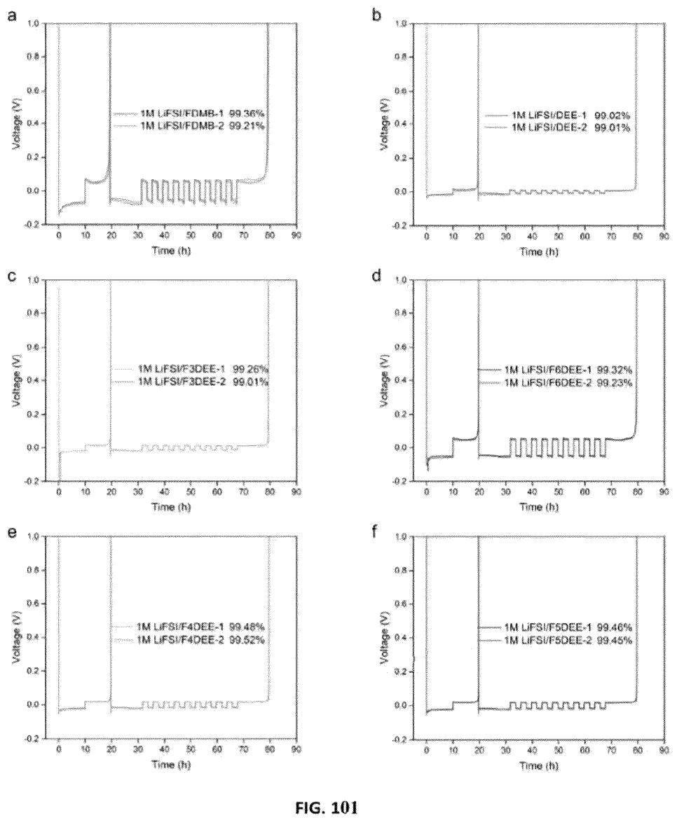

Next, we investigated the electrolyte stability at Li metal anode and at high voltage separately. The Li∥Cu half-cell setup is commonly used to examine Li metal efficiency, and here we first focus on the activation of Li metal CE in the initial cycles, which is defined as the cycle number needed to reach >99% CE in Li∥Cu half cells. Initial Li consumption will be detrimental to practical Li metal batteries requiring limited- or zero-excess Li inventory. As shown in FIG. 72a, activation during initial cycles was tested under 0.5 mA cm⁻² current density and 1 mAh cm⁻² areal capacity. The 1 M LiFSI/FDMB showed a five-cycle activation before ramping up to 99% CE21; while the DEE electrolyte never reached a CE of 99% (FIG. 98). This confirms the argument above that DEE possesses fast ion conduction but sacrifices Li metal stability. In accord with our design, F3DEE and F6DEE solvents showed a substantial improvement over DEE, with activation periods measured to be 30 and 90 cycles, respectively (FIGS. 72a and 98). Although the initial CEs of F3DEE and F6DEE electrolytes were stabilized at ˜98.5%, they were significantly better than that of DEE (97-97.5%) and finally reached >99% in later cycles (FIG. 98), confirming the benefit of fluorination. However, tens of activation cycles are still far from ideal case. The partially-fluorinated electrolytes that contain —CHF₂ groups (FIG. 69a) performed much better, as the Li metal anode in F4DEE and F5DEE was activated within only 3 and 4 cycles, respectively. Long cycling of Li∥Cu half cells showed high CEs of 99.3±0.1% for both F4DEE and F5DEE electrolytes (FIG. 98), and the CE of 1.2 M LiFSI/F5DEE was further boosted to ˜99.9% when tough spring was implemented in coin cells since high pressure was known to improve Li metal efficiency (FIGS. 72b and 99). Such a high average CE of 1.2 M LiFSI/F5DEE is reliable since the fluctuation range is about ±0.1% from the 100th to 580th cycle even under ambient conditions (FIG. 72c and Source Data of FIG. 72). When the cycling areal capacity was increased to a more practical value, i.e., 5 mAh cm⁻², the CE rapidly reached ˜99.5% and the activation could even be completed by the second cycle (the second cycle CE >99.3%), which is one of the fastest among the state-of-the-art electrolytes (FIG. 72d). At high current densities (>4 mA cm⁻²), the CE of Li∥Cu cells showed slight decrease and fluctuation (FIG. 100). It is worth noting that fluctuation of CE >100% occasionally happened (FIGS. 72b-d), which may be attributed to uncontrolled temperature fluctuation or re-activation of initial dead Li. The benefit of fluorinated-DEE electrolytes was further validated by Aurbach CE measurements (Aurbach, D., Gofer, Y. & Langzam, J. The Correlation Between Surface Chemistry, Surface Morphology, and Cycling Efficiency of Lithium Electrodes in a Few Polar Aprotic Systems. J. Electrochem. Soc. 136, 3198-3205 (1989); Adams, B. D., Zheng, J., Ren, X., Xu, W. & Zhang, J.-G. Accurate Determination of Coulombic Efficiency for Lithium Metal Anodes and Lithium Metal Batteries. Adv. Energy Mater. 8, 1702097 (2018)), in which F4DEE and F5DEE showed higher average CEs than other electrolytes (FIGS. 72e and 101).

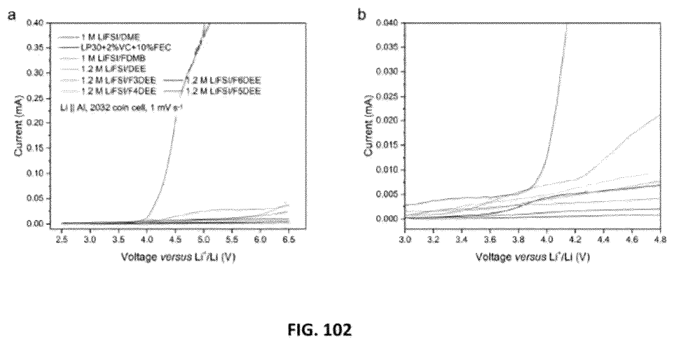

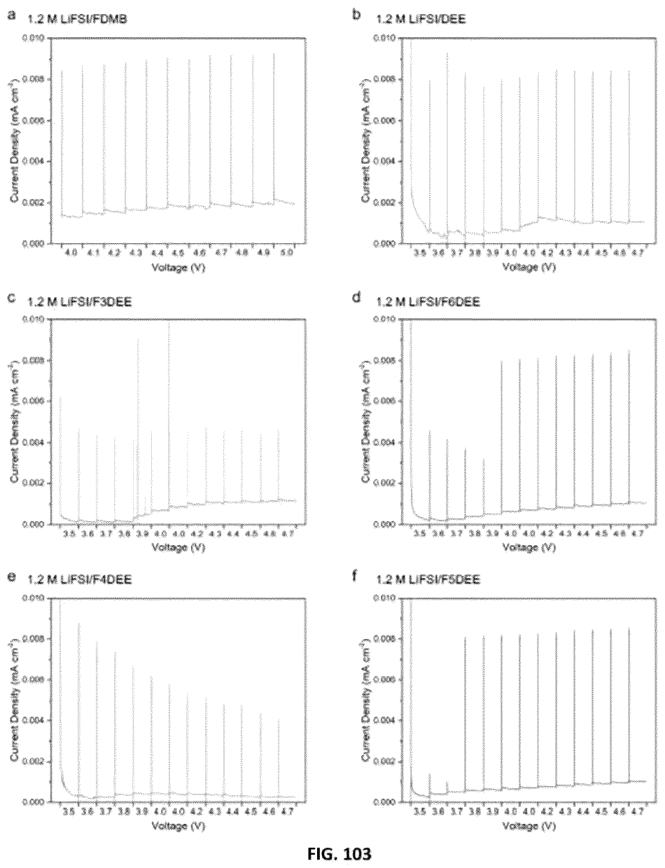

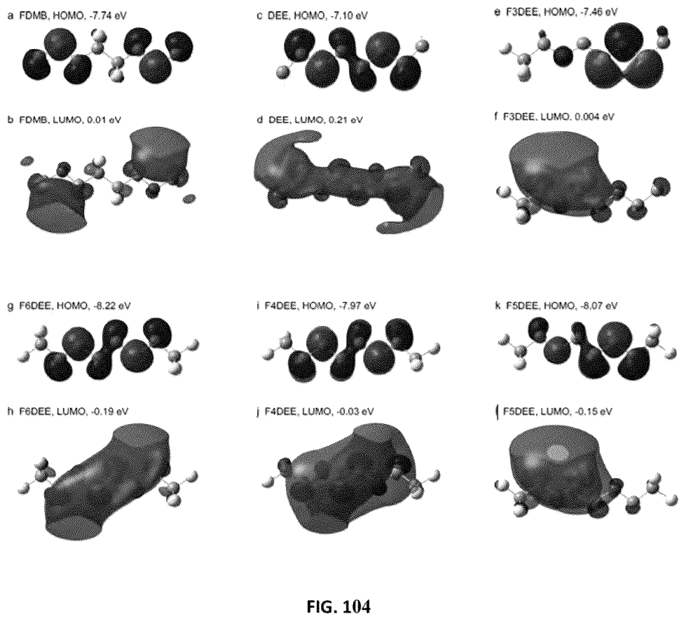

The anodic stability was evaluated by linear sweep voltammetry (LSV) of Li∥Al half cells, where the leakage current is a good metric to evaluate the corrosion of Al current collectors for realistic battery cathodes. As shown in FIG. 72f, the DEE electrolyte was the most vulnerable at high voltage among these electrolytes; however, it was still far more stable against oxidation than DME (FIG. 102). The leakage current evolution of FDMB21 under a high voltage scan was similar to that of a conventional carbonate electrolyte (1 M LiPF₆ in ethylene carbonate/dimethyl carbonate [1/1] with 2% vinylene carbonate and 10% fluoroethylene carbonate, denoted as LP30+2% VC+10% FEC), indicating reasonable high voltage stability. As expected, the anodic stability of fluorinated-DEE electrolytes generally followed the trend of fluorination: F5DEE˜F6DEE>F4DEE>>F3DEE. Potentiostatic polarization tests at high voltage and molecular orbital energy level calculations provided similar trends (FIGS. 103 and 104).

### II. A. 7. Performance of Li 1 Metal and Anode-Free Full Cells

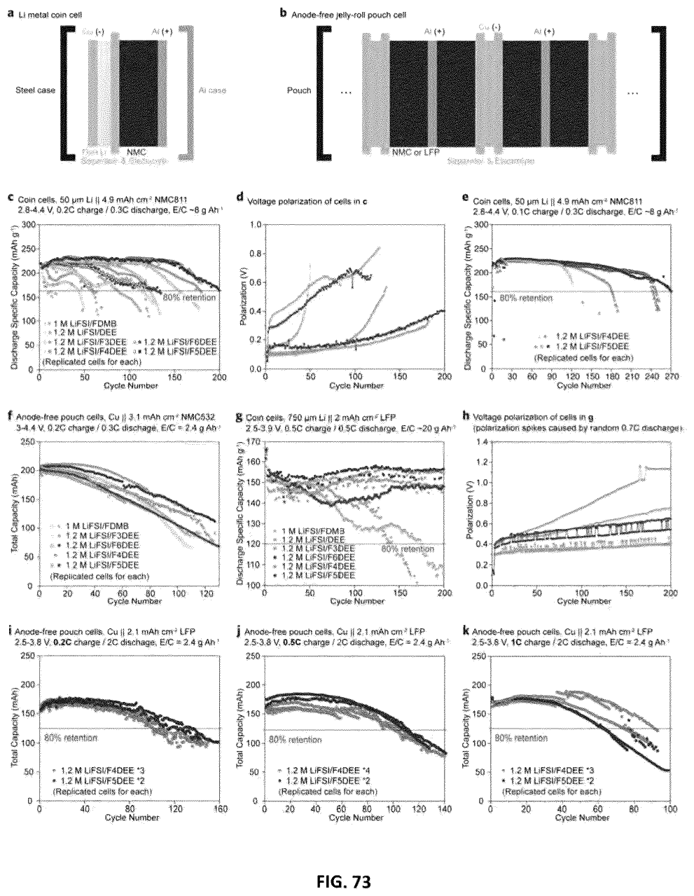

After half-cell screening, we proceeded to Li metal full cells to test the practicality of these developed electrolytes in realistic batteries. Two types of Li metal batteries are examined in this work: Li metal full cells using thin Li foil (FIG. 81a) and industrial anode-free jelly-roll pouch cells (FIG. 73b, Supplementary Table 3).

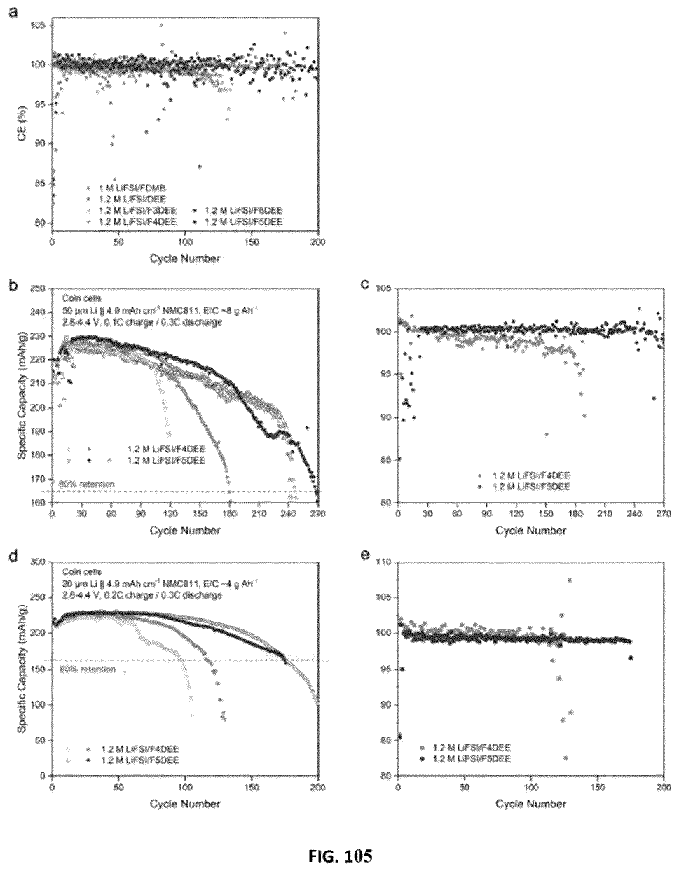

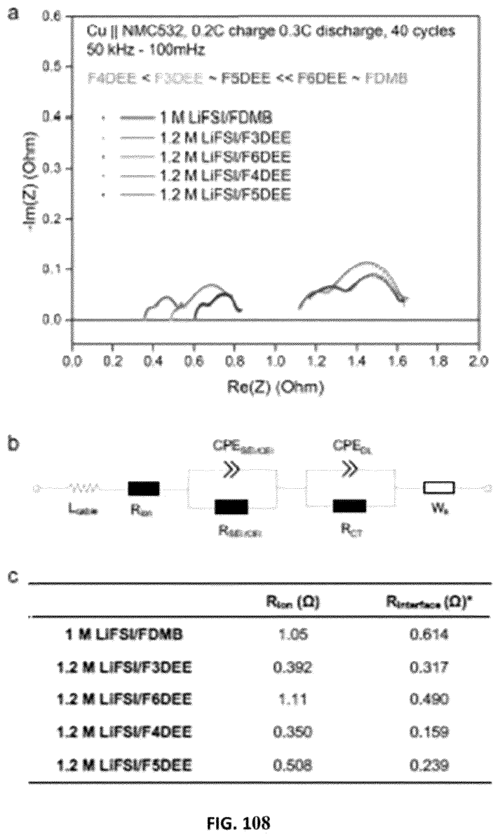

We first constructed Li metal full cells by pairing thin Li foil (50 μm thick, ˜10 mAh cm⁻²) with an industrial high-loading NMC811 cathode (˜4.9 mAh cm⁻²). Using the electrolyte-to-cathode (E/C) ratio of ˜8 g Ah⁻¹, these coin cells were cycled at 0.2 C charge and 0.3 C discharge. These battery conditions are harsh among the state-of-the-art cells. The cycle life which is defined as the cycle number before reaching 80% capacity retention followed the trend of F5DEE>F4DEE>>F6DEE˜F3DEE>FDMB>>DEE (FIG. 73c), and all the cells showed high and stable full-cell CEs before failure (FIG. 105a). The cycle life can be further correlated with voltage polarization50, which is defined as the average voltage gap between charge and discharge. As shown in FIGS. 73d, 106 and 107, the poorly performing DEE showed drastic polarization increase with cycling; while the FDMB and F6DEE showed high yet slowly evolving overpotentials. The polarization of the F3DEE cell sharply increased at ˜100 cycles, coinciding with when the cell suffered significant capacity loss. Consistent with our expectation, the overpotentials of the long-cycling F4DEE and F5DEE full cells were low and stably controlled throughout the whole cycle life. Using the best-performing electrolyte 1.2 M LiFSI/F5DEE, 50-μm-thick Li∥4.9 mAh cm⁻² NMC811 full cells maintained stable capacity for 270 cycles at a slow charging rate of 0.1C, which are among the best high-loading Li metal full-cell performances (FIG. 73e and Supplementary Table 4). Similar to Li metal coin cells, the industrial anode-free pouch cells using single-crystal NMC532 (LiNi_{0.5}Mn_{0.3}Co_{0.2}O₂, ˜3.1 mAh cm⁻²) showed the same trend of cycle life and impedance (FIGS. 73f and 108). Other types of cells (e.g., industrial Li metal pouches, coin cells with different Li foil thicknesses and cathode loadings) or different cycling conditions also supported these conclusions (FIGS. 105, 109-111). These facts confirmed that the fine-tuning of fluorination yields a highly conductive, Li-metal and high-voltage compatible electrolyte system.

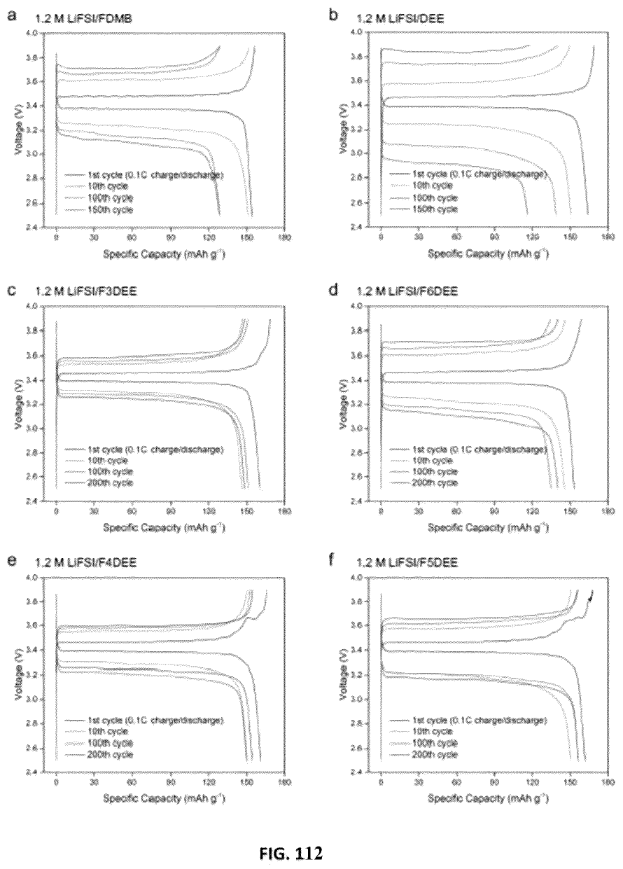

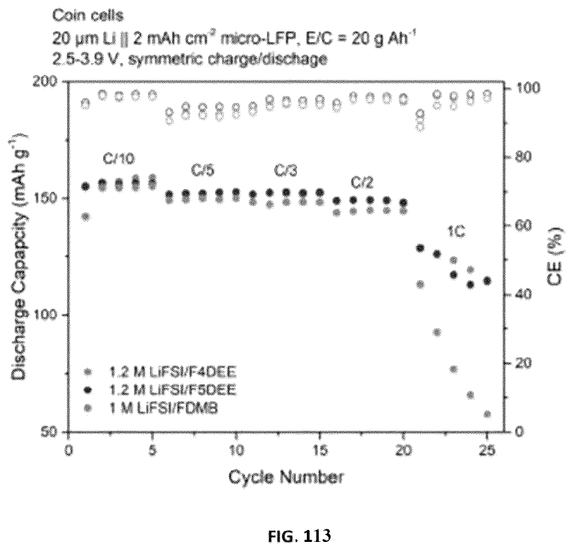

To better evaluate the effect of fast ionic transport on full-cell performance, we further selected microparticle-LFP, a known poorly-conductive yet cost-effective and recently-popular cathode material. The achievement of high-rate capability using such a poorly-conductive cathode is meaningful. We started the investigation with thick-Li∥LFP half cells at a slightly higher cycling rate (0.5 C charge, 0.5 C discharge with random 0.7 C discharge caused by instrument error). As demonstrated in FIG. 73g, the highly conductive electrolytes, F3DEE, F4DEE, and F5DEE, resulted in stable cycling with high capacities. The half cell using less conductive yet Li metal compatible F6DEE electrolyte delivered slightly lower specific capacity. Although the capacity of both DEE and FDMB cells gradually diminished, we ascribed this to different mechanisms (Niu, C. et al. Balancing interfacial reactions to achieve long cycle life in high-energy lithium metal batteries. Nat. Energy 6, 723-732 (2021)): for DEE, oxidation still happened at the charge voltage cut-off and the accumulation of side products sharply increased the cell polarization (FIGS. 73h, 112), leading to capacity loss; for FDMB, its slow ionic conduction and continuously increasing overpotential due to residue SEI accumulation were responsible for its steady capacity decay, which was similar to the thin-Li∥NMC811 case (FIGS. 73c-e and 106). The benefit of stable and low overpotentials using F4DEE and F5DEE (FIGS. 73h and 112) was further validated by the rate capability tests of LFP half cells, in which these developed electrolytes outperformed FDMB (FIG. 113).

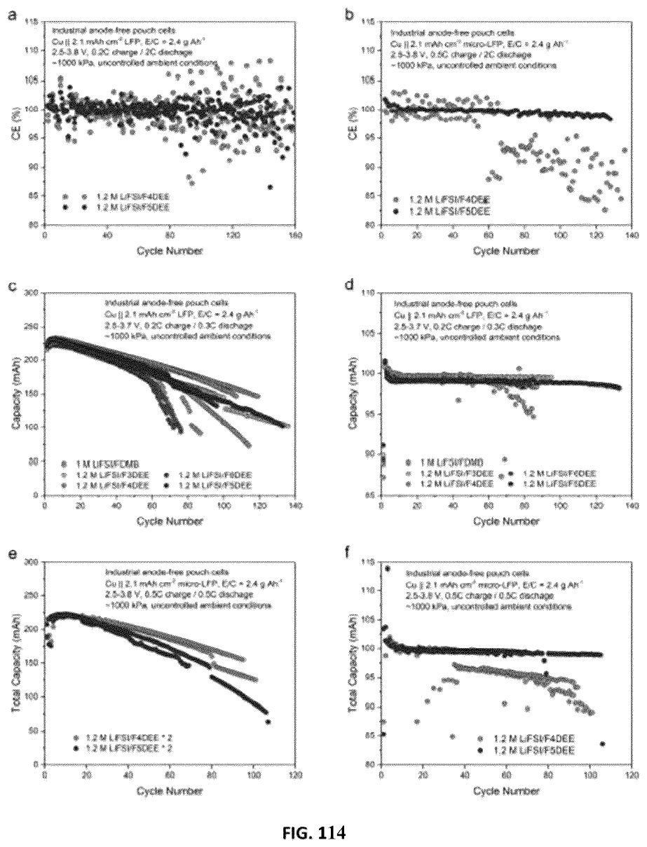

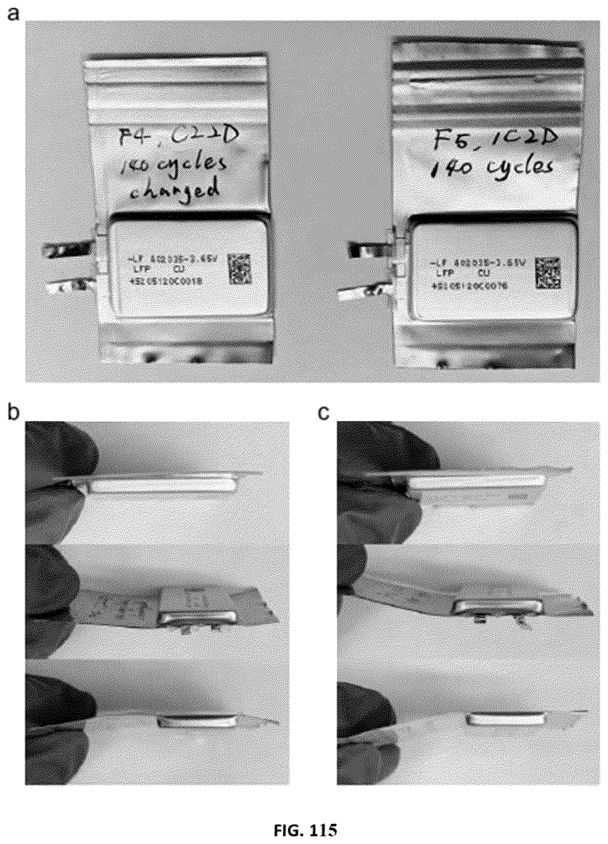

Industrial anode-free multilayer pouch cells using microparticle-LFP (with a practical loading of 1 2.1 mAh cm⁻²) without conductive carbon coating were cycled at high rates to examine the limit of the developed electrolytes under stringent conditions. Compared to the NMC cathodes in anode-free cells, the LFP cathode provides less Li excess inventory on the anode side during the first charging and consequently the cycle life will be shorter. Due to this material limitation, LFP-based anode-free batteries have seldom been studied in the community, but it is an ideal platform to examine the influence of electrolyte efficiency and ionic transport on cell performance. As shown in FIG. 73i, at slow charge (0.2C) and fast discharge (2C) rate, the F4DEE and F5DEE electrolyte maintained ˜110 and ˜140 cycles respectively before reaching 80% capacity. Faster charging rates were applied to Cu∥microparticle-LFP anode-free cells. At 0.5 C charge and 2 C discharge rate, ˜110 cycles were achieved for both F4DEE and F5DEE (FIG. 73j). When the charge rate was further boosted to 1C, the faster conducting F4DEE electrolyte outperformed F5DEE, enabling 80-90 cycles before fading (FIG. 73k). These fast-cycling conditions, to our best knowledge, are the first attempt in low-cost microparticle-LFP based anode-free pouch cells, and the cycle lives are believed to be among the state-of-the-art (Supplementary Table 5). Performance of anode-free cells under other cycling conditions also supported our arguments (FIG. 114). Moreover, no gassing issue was observed for these pouch cells after fast cycling even though no degassing procedure was implemented, indicating high safety and ease of manufacturing (FIG. 115).

### II. A. 8. Li Morphology, SEI Structure and Cathode Characterization

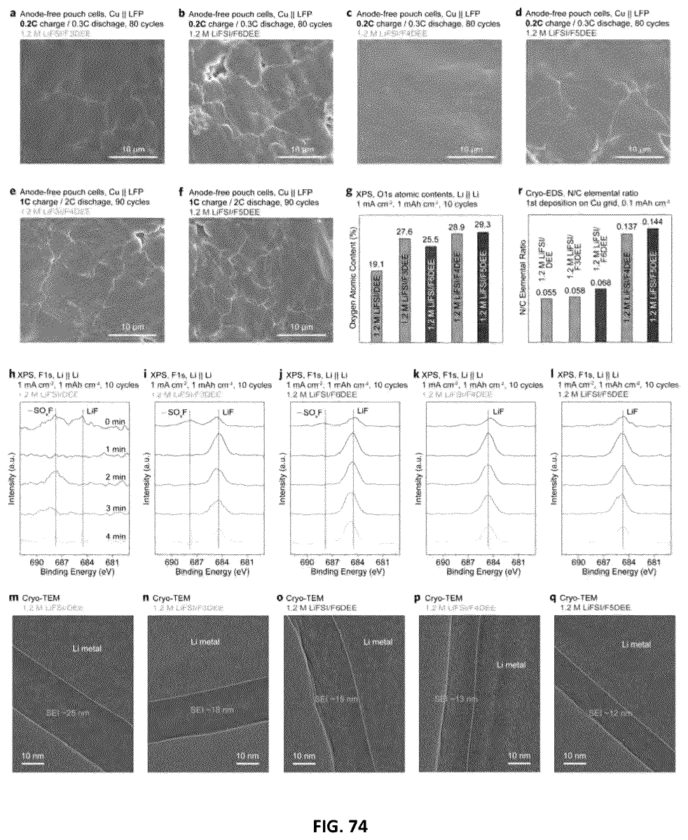

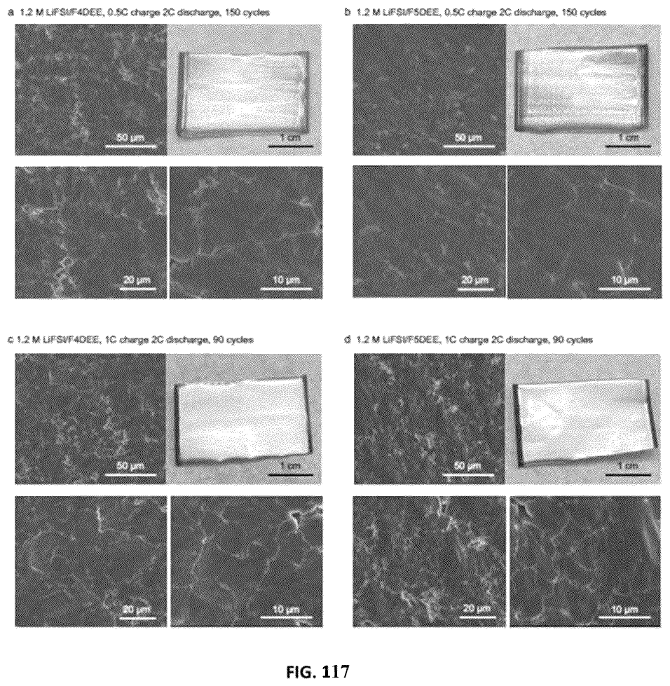

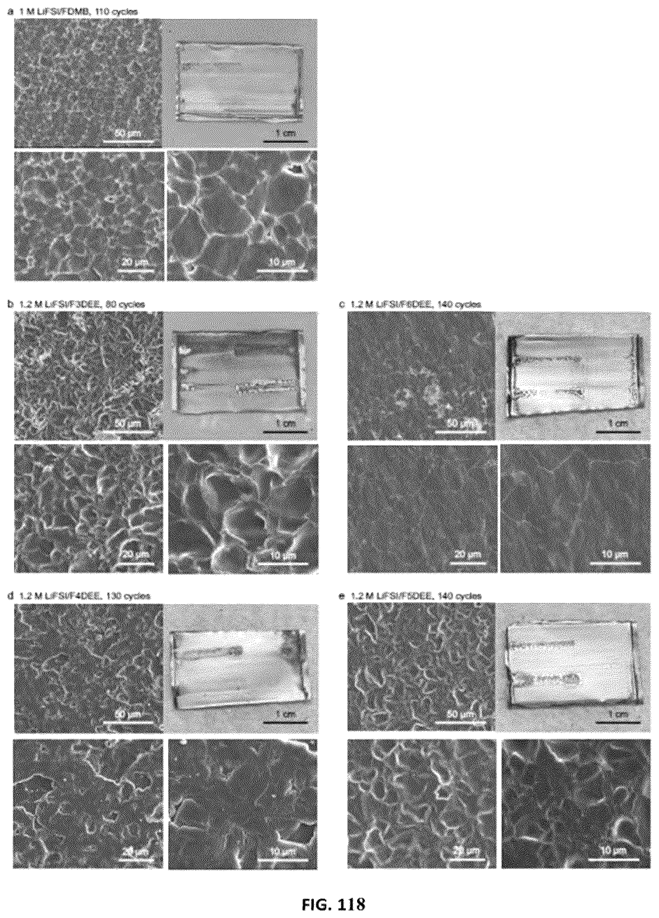

Li metal morphology and SEI properties are crucial factors that correlate with battery performance. Anode-free pouch cells after cycling were chosen here for scanning electron microscope (SEM) examination since they generated the Li morphologies under realistic full-cell conditions. We first investigated the images after slow cycling. After 80 cycles at 0.2 C charge and 0.3 C discharge, the Cu∥LFP pouch cells were charged to the upper cut-off voltage, i.e., Li⁺ ions in LFP cathode were fully deposited as metallic Li on the anode. As shown in FIGS. 74a-d, 116, 117, chunky and desired Li deposits were observed in all fluorinated-DEE electrolytes. However, careful examination revealed more favorable Li deposition in F4DEE and F5DEE electrolytes where the Li deposits had characteristic length scales much larger than 10 μm (FIGS. 74c and d). In particular, the Li deposits in the F4DEE electrolyte were almost flat with few grain boundaries, and such morphology was consistent with its long cycle life in anode-free cells. The diameters of Li deposits in F3DEE and F6DEE, in contrast, were slightly lower than 10 μm (FIGS. 74a and b). Under fast cycling condition (1 C charge 2 C discharge), F4DEE and F5DEE maintained chunky Li morphology, which matched well with their outstanding cycle life at high rate (FIGS. 74e, f, and 117). The SEM images taken under other cycling conditions (FIG. 117) or with Cu∥NMC532 pouch cells (FIG. 118) exhibited similar features.

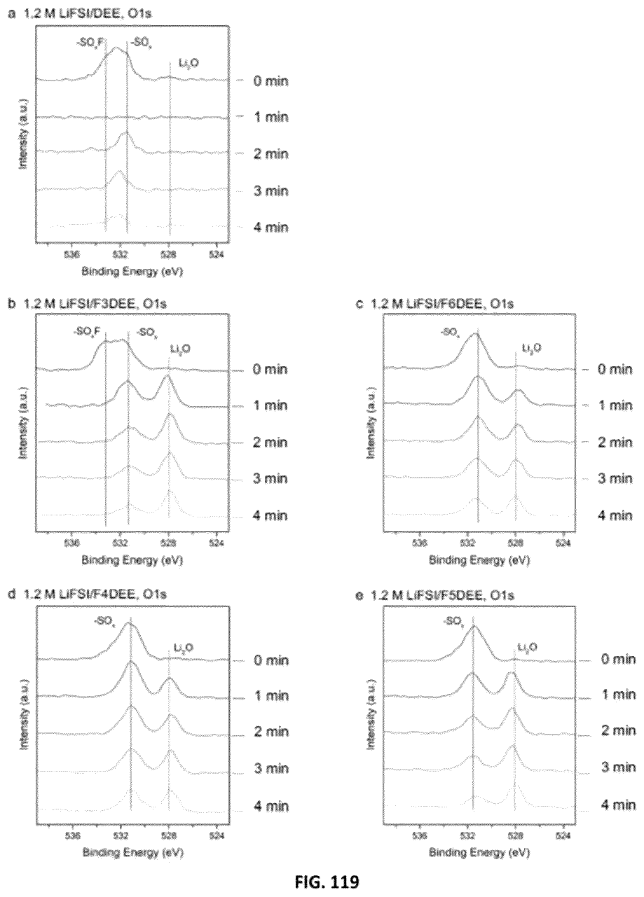

Next, X-ray photoelectron spectroscopy (XPS) was used to examine the SEI compositions. The O1s spectra showed that Li₂O and —SOₓ species were present (FIG. 119) and the oxygen content was higher in the fluorinated-DEE electrolytes especially in the best performing F4DEE and F5DEE, indicating an oxygen-rich SEI on Li metal surface (FIG. 74g). Such a robust SEI was reported to be beneficial to Li metal efficiency as well as interfacial Li⁺ ion transport (Guo, R. & Gallant, B. M. Li₂O Solid Electrolyte Interphase: Probing Transport Properties at the Chemical Potential of Lithium. Chem. Mater. 32, 5525-5533 (2020); May, R., Fritzsching, K. J., Livitz, D., Denny, S. R. & Marbella, L. E. Rapid Interfacial Exchange of Li Ions Dictates High Coulombic Efficiency in Li Metal Anodes. ACS Energy Lett. 6, 1162-1169 (2021)), and was consistent with the integration of high Li metal CE, fast activation and low overpotential/polarization observed in F4DEE and F5DEE electrolytes. In addition to the overall elemental content information, the species distribution through depth profiling is critical as well. The XPS F1s spectra with sputtering showed a distinct difference between DEE and fluorinated-DEEs, in which the latter contained clear LiF species while the former only showed trivial signal for this species (FIGS. 74h-l). Careful scrutiny revealed small differences in the depth profiles between F3DEE/F6DEE and F4DEE/F5DEE.

Although uniformly distributed LiF throughout depth profiling dominated the surface fluorine species in all fluorinated-DEEs, the anion species —SOₓF remained on the top surface of Li metal in F3DEE and F6DEE electrolytes, indicating incomplete anion decomposition or passivation. The LiF-rich, vertically homogeneous SEI in F4DEE and F5DEE corroborates with their outstanding Li metal efficiency (FIGS. 72a-d). Depth profiles of other representative elements demonstrated similar observations (FIGS. 119-121). Such a fine difference agreed well with our careful design rationales evolving from F3DEE/F6DEE to F4DEE/F5DEE, as elaborated earlier.

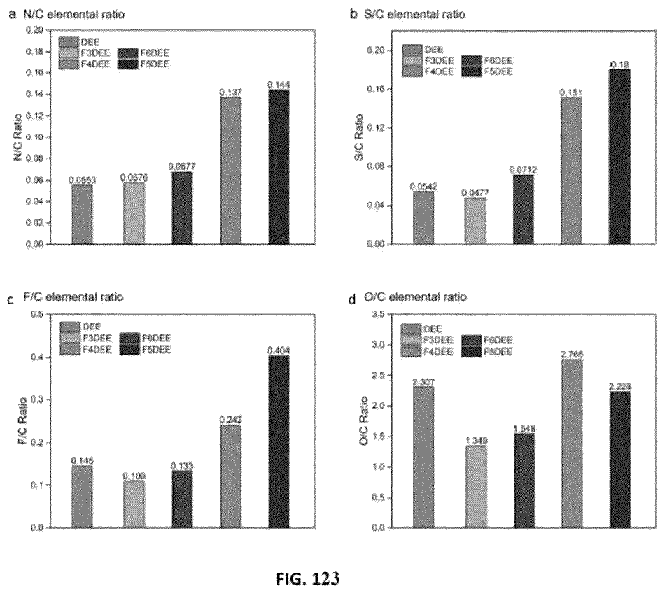

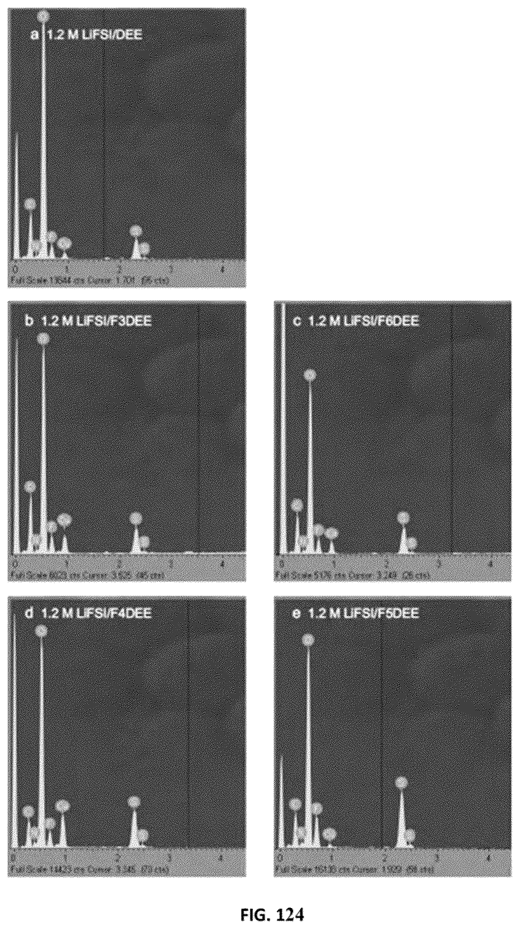

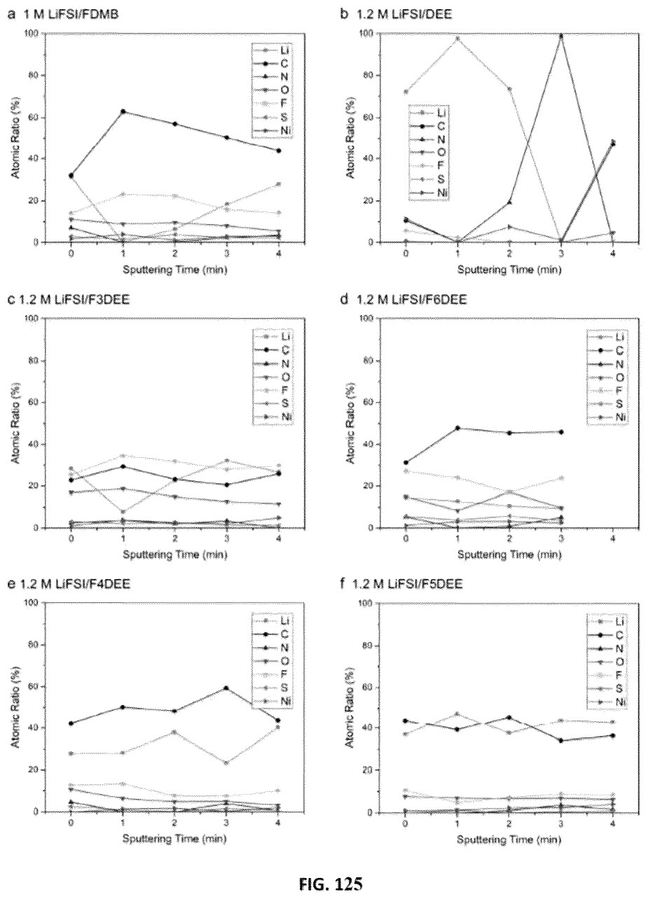

We further performed cryogenic transmission electron microscopy (cryo-TEM) and cryogenic transmission electron microscopy energy-dispersive X-ray spectroscopy (cryo-TEM EDS or cryo-EDS) to unveil the fine structural and local chemical information of compact, direct SEIs (Huang, W., Wang, H., Boyle, D. T., Li, Y. & Cui, Y. Resolving Nanoscopic and Mesoscopic Heterogeneity of Fluorinated Species in Battery Solid-Electrolyte Interphases by Cryogenic Electron Microscopy. ACS Energy Lett. 5, 1128-1135 (2020)) on Li metal surface. All compact SEIs in these electrolytes exhibited thin, uniform and amorphous nanostructure under cryo-TEM (FIGS. 74m-q and 122); however, the SEI in F4DEE and F5DEE showed the thinnest thickness, corroborating with their high CE and fast activation. The nitrogen-to-carbon (N/C) ratio by cryo-TEM EDS served as an indicator of anion-derived favorable SEIs since FSI− is solely the source of N element in these electrolytes (FIG. 74r). Similar to the results of XPS elemental contents, the SEIs in F4DEE and F5DEE showed much higher N/C ratios corresponding to a more anion-derived SEI compared to others. These facts were cross validated by other elemental ratios, especially the sulfur-to-carbon (S/C) and fluorine-to-carbon (F/C) ratios (FIGS. 123 and 124), again indicating anion-derived, inorganics-rich SEIs.

Robust cathode-electrolyte interphase (CEI) and suppression of cathode cracking are also critical for stable cell operation. We analyzed the elemental composition of CEI by XPS and found that high C and F content yet negligible Ni species were observed on the cathode surface when using FDMB and fluorinated-DEE electrolytes, confirming their cathode protection effect (FIG. 124). Furthermore, NMC811 particles showed limited intergranular cracking after cycling, again indicating the stability of cathode towards these developed electrolytes (FIG. 125).

### II. A. 9. Overall Evaluation of 1 Fluorinated-DEE Electrolytes

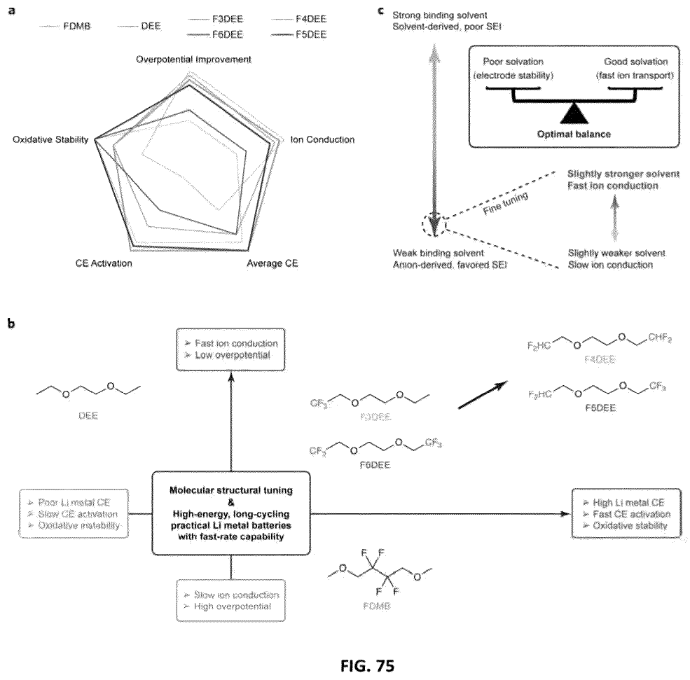

We further categorize the critical factors of the electrolytes studied in this work: bulk ionic conduction, overpotential/polarization improvement, Li metal CE, activation, and oxidative stability. Semi-quantification of these factors is presented in the radar plot (FIG. 75a). The DEE electrolyte exhibits advantageous ionic conduction but poor Li metal CE, activation, and oxidative stability; conversely, FDMB shows significant improvement over all the above parameters except for worse ionic conduction. The fluorinated-DEEs all show more balanced behavior; however, F4DEE and F5DEE outperform F3DEE and F6DEE, which confirms our design logic (FIG. 75b). Our study suggests that the strongest binding solvents are not necessarily desirable; instead, a balance needs to be achieved by finely modulating the molecular structure of weakly binding solvents, which ensures both electrode stability and sufficient solvation for fast transport (FIG. 75c). This work shows that fine-tuning fluorination content is an effective way to identify function-balanced solvent molecules.

### II. A. 10. Conclusions

In summary, we investigated a family of fluorinated-DEE based electrolytes for Li metal batteries, in which the partially fluorinated —CHF₂ group was identified and rationalized as the designer choice. The obtained electrolytes, especially F4DEE and F5DEE, simultaneously possess high ionic conductivity, low and stable interfacial transport, reproducibly high Li metal efficiency (up to 99.9% with only ±0.1% fluctuation for 1.2 M LiFSI/F5DEE in Li∥Cu half cells), record-fast activation (CE >99.3% within from the second cycle in Li∥Cu half cells) and high-voltage stability. These features enable ˜270 cycles in thin-Li (50 μm thick)∥high-loading-NMC811 (˜4.9 mAh cm⁻²) full batteries and >140 cycles in fast-cycling industrial anode-free Cu∥microparticle-LFP pouch cells under lean electrolyte and realistic testing conditions. Thorough morphological characterization and SEI examination revealed flat Li deposition as well as an ideal anion-derived SEI, which enable outstanding full-cell cycling performance. We additionally conducted a systematic study on the structure performance relationship in these electrolytes via multiple theoretical and experimental tools. Crucial properties including Li⁺-solvent coordination, solvation structure, and battery performance were cross-validated and their correlations were thoroughly explained. Our work emphasizes the critical yet less-studied direction, fast ion conduction, in the Li metal battery electrolyte research. It is critical to achieve a balance between fast ion conduction and electrode stability through fine-tuning the solvation ability of the solvent, and molecular design and synthetic tools will thus play important roles. We believe that rational molecular-level design and chemical synthesis can endow the electrolyte field with more opportunities in the future.

### II. A. 11. Methods

General materials: 2,2,3,3-Tetrafluoro-1,4-butanediol, 2-(2,2,2-trifluoroethoxy)ethanol, 2,2-difluoroethanol, ethyl p-toluenesulfonate, 2,2,2-trifluoroethyl p-toluenesulfonate, 2,2-difluoroethyl p-toluenesulfonate were purchased from SynQuest. Ethylene carbonate (98%), sodium hydride (60% in mineral oil) and other general reagents were purchased from Sigma-Aldrich or Fisher Scientific. All chemicals were used without further purification. LiFSI was obtained from Guangdong Canrd New Energy Technology and Arkema. DME (99.5% over molecular sieves) and DEE (also denoted as ethylene glycol diethyl ether, 99%) were purchased from Acros. Anhydrous VC and FEC were purchased from Sigma-Aldrich. The commercial carbonate electrolytes LP30 and LP40 were purchased from Gotion. The commercial Li battery separator Celgard 2325 (25 μm thick, polypropylene/polyethylene/polypropylene) was purchased from Celgard and used in all coin cells. Thick Li foil (˜750 μm thick) and Cu current collector (25 μm thick) were purchased from Alfa Aesar.

Thin Li foils (˜50 μm and −20 μm thick, supported on Cu substrate) were purchased from China Energy Lithium.

Commercial LFP and NMC532 cathode sheets were purchased from MTI, and NMC811 cathode sheets were purchased from Targray (˜2.2 mAh cm⁻² and ˜4.9 mAh cm⁻² areal capacity). Industrial dry Cu∥NMC532 and Cu∥LFP pouch cells were purchased from Li-Fun Technology. Other battery materials, such as 2032-type coin-cell cases, springs and spacers, were all purchased from MTI.

Syntheses. FDMB was synthesized according to our previous report.

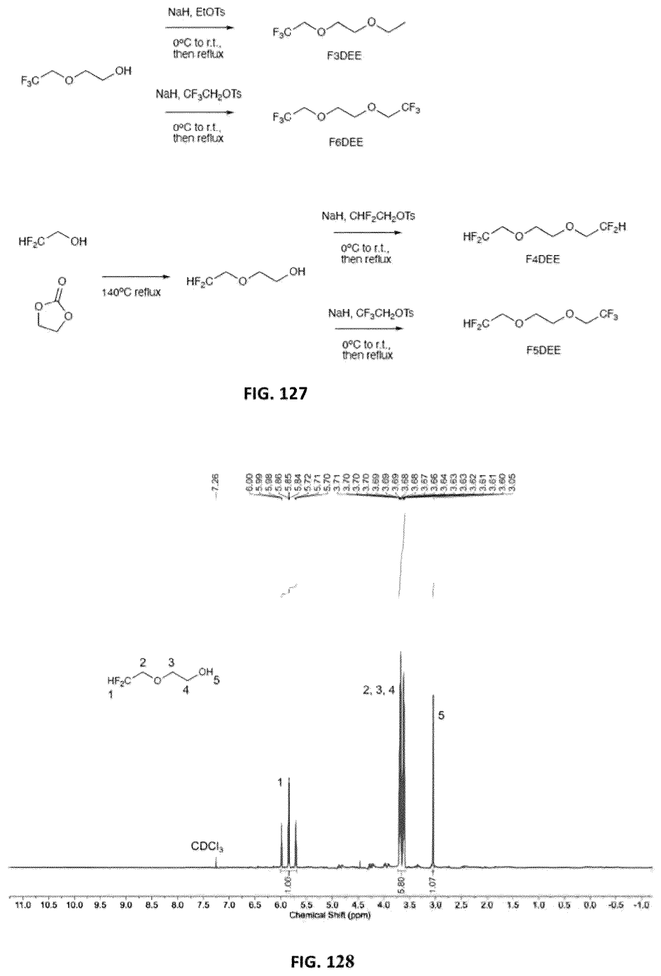

2-(2,2-difluoroethoxy)ethanol (FIGS. 127-130): In a 1000 mL round bottom flask were added 150 g 2,2-difluoroethanol, 140 g ethylene carbonate, 8 g NaOH and 200 mL tetraglyme. Under nitrogen atmosphere, the suspension was heated to 140° C. to stir for 48 h. The suspension was then distilled under vacuum (˜65° C. under ˜1 kPa) for three times to yield ˜100 g colorless liquid as the product. Yield ˜43%. 1H-NMR (400 MHz, CDCl3, δ/ppm): 6.00-5.70 (tt, 2H), 3.71-3.60 (m, 6H), 3.05 (s, 1H). 13C-NMR (100 MHz, CDCl3, δ/ppm): 116.96˜112.17, 73.63, 70.74˜70.20, 61.67. 19F-NMR (376 MHz, CDCl3, δ/ppm): −125.74˜−125.96 (dt, 4F).

F3DEE (FIGS. 127, 131-134): In a 1000 mL round bottom flask were added 22 g NaH (60% in mineral oil) and 400 mL anhydrous tetrahydrofuran, and the suspension was cooled to 0° C. by ice bath to stir for 10 min. Then 56 g 2-(2,2,2-trifluoroethoxy)ethanol was added into the cooled suspension dropwise. After addition, the suspension was further stirred at 0° C. for 30 min. 93 g ethyl p-toluenesulfonate was added in batches and then the ice bath was removed 1 to allow the suspension to warm up to room temperature. After stirring at room temperature for 2 h, the flask was slowly heated up to 60° C. to reflux overnight. After the completion of reaction, the flask was allowed to cool down to room temperature and 200 mL deionized water was slowly added into the suspension to dissolve all solids. The remaining tetrahydrofuran in the resulting solution was removed under vacuum, and then the solution was extracted with 500 mL dichloromethane for three times. The dichloromethane layer was washed with brine, dried by anhydrous MgSO₄, and the solvents were removed under vacuum. The crude product underwent vacuum distillation (˜40° C. under ˜1 kPa) for three times to yield ˜43 g colorless liquid as the product. Yield ˜64%. 1H-NMR (400 MHz, CDCl3, δ/ppm): 3.94˜3.87 (q, 2H), 3.77-3.59 (m, 4H), 3.55 ˜3.50 (q, 2H), 1.23˜1.19 (3H). 13C-NMR (100 MHz, CDCl3, δ/ppm): 128.44˜120.10, 72.25, 70.06, 69.48˜68.47, 67.00, 15.34. 19F-NMR (376 MHz, CDCl3, δ/ppm): −74.66˜˜74.71 (t, 3F). Electrospray ionization mass spectrometry (ESI-MS) calculated [M⁺H⁺]: 173.16; found: 173.32.

F6DEE (FIGS. 127, 134-136): The same procedure as for F3DEE synthesis was adopted, except that 93 g ethyl p-toluenesulfonate was replaced by 120 g 2,2,2-trifluoroethyl p-toluenesulfonate. The crude product underwent vacuum distillation (˜40° C. under ˜1 kPa) for three times to yield ˜50 g colorless liquid as the product. Yield ˜57%. 1H-NMR (400 MHz, CDCl₃, δ/ppm): 3.92˜3.86 (q, 4H), 3.80 (s, 4H). 13C-NMR (100 MHz, CDCl3, δ/ppm): 128.28˜119.95, 72.14, 69.53˜68.52. 19F-NMR (376 MHz, CDCl3, δ/ppm): −74.97˜−75.01 (t, 6F). ESI-MS calculated [M⁺H⁺]: 227.13; found: 227.20.

F4DEE (FIGS. 127, 137-139): The same procedure as for F3DEE synthesis was adopted, except that 56 g 2-(2,2,2-trifluoroethoxy)ethanol was replaced by 50 g 2-(2,2-difluoroethoxy)ethanol, and 93 g ethyl p-toluenesulfonate was replaced by 110 g 2,2-difluoroethyl p-toluenesulfonate. The crude product underwent vacuum distillation (˜60° C. under ˜1 kPa) for three times to yield ˜45 g colorless liquid as the product. Yield ˜60%. 1H-NMR (400 MHz, CDCl3, δ/ppm): 6.00-5.70 (tt, 2H), 3.73-3.68 (td, 4H), 3.69 (s, 4H). 13C-NMR (100 MHz, CDCl3, δ/ppm): 116.80-112.01, 71.35, 70.74-70.20. 19F-NMR (376 MHz, CDCl3, δ/ppm): −125.35˜−125.57 (dt, 4F). ESI-MS calculated [M⁺H⁺]: 191.15; found: 191.22.

F5DEE (FIGS. 127, 140-142): The same procedure as for F3DEE synthesis was adopted, except that 56 g 2-(2,2,2-trifluoroethoxy)ethanol was replaced by 50 g 2-(2,2-difluoroethoxy)ethanol, and 93 g ethyl p-toluenesulfonate was replaced by 120 g 2,2-difluoroethyl p-toluenesulfonate. The crude product underwent vacuum distillation (˜60° C. under ˜1 kPa) for three times to yield ˜62 g colorless liquid as the product. Yield ˜75%. 1H-NMR (400 MHz, CDCl3, δ/ppm): 6.01-5.71 (tt, 1H), 3.92-3.85 (td, 2H), 3.79-3.67 (m, 6H). 13C-NMR (100 MHz, CDCl3, δ/ppm): 128.09˜119.74, 116.74˜111.94, 71.83, 71.41, 70.82˜70.28, 69.21˜68.19. 19F-NMR (376 MHz, CDCl3, δ/ppm): −74.53˜−74.58 (t, 3F), −125.37˜−125.59 (dt, 2F). ESI-MS calculated [M⁺H⁺]: 209.14; found: 209.31.

Electrolyte preparation. LiFSI (2,244 mg) was dissolved in 10 mL DEE or fluorinated-DEEs to obtain the respective 1.2 M LiFSI electrolyte. LiFSI (1,122 mg) was dissolved in 6 mL DME or FDMB to obtain 1 M LiFSI/DME and 1 M LiFSI/FDMB, respectively. All the electrolytes were prepared and stored in argon-filled glovebox (Vigor, oxygen <0.5 ppm, water <0.1 ppm) at room temperature.

Theoretical calculations. DFT: The molecular geometries for the ground states were optimized by DFT at the B3LYP/6-311G+(d, p) level, and then the energy, orbital levels and ESPs of molecules were evaluated at the B3LYP/6-311G+(d, p) level as well. All DFT calculations were carried out with Gaussian 16 on Sherlock server at Stanford University.

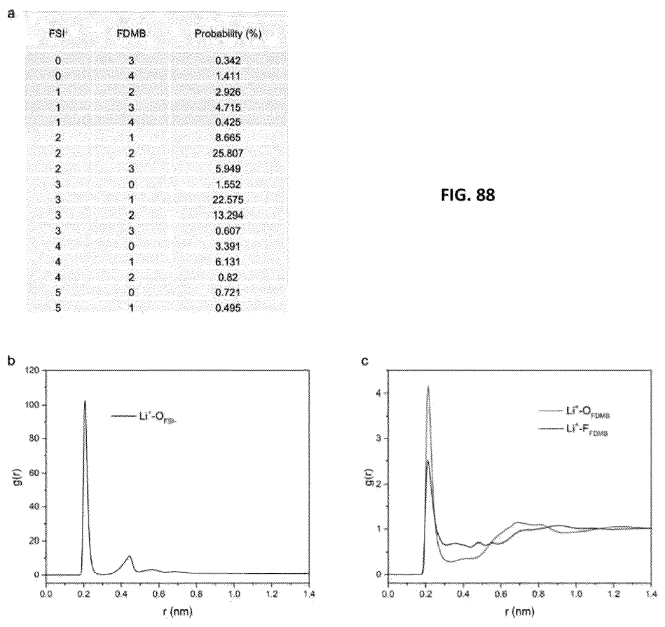

MD: MD simulations were carried out using Gromacs 2018 program (Abraham, M. J. et al. GROMACS: High performance molecular simulations through multi-level parallelism from laptops to supercomputers. SoftwareX 1-2, 19-25 (2015)), with electrolyte molar ratios taken from experimental results. Molecular forces were calculated using the Optimized Potentials for Liquid Simulations all atom (OPLS-AA) force field (Jorgensen, W. L., Maxwell, D. S. & Tirado-Rives, J. Development and Testing of the OPLS All-Atom Force Field on Conformational Energetics and Properties of Organic Liquids. J. Am. Chem. Soc. 118, 11225-11236 (1996)). Topology files and bonded and Lennard-Jones parameters were generated using the LigParGen server (Dodda, L. S., Cabeza de Vaca, I., Tirado-Rives, J. & Jorgensen, W. L. LigParGen web server: an automatic OPLS-AA parameter generator for organic ligands. Nucleic Acids Res. 45, W331-W336 (2017)). Atomic partial charges were calculated by fitting the molecular electrostatic potential at atomic centers in Gaussian 16 using the Meller-Plesset second-order perturbation method with a cc-pVTZ basis set (Sambasivarao, S. V. & Acevedo, O. Development of OPLS-AA Force Field Parameters for 68 Unique Ionic Liquids. J. Chem. Theory Comput. 5, 1038-1050 (2009)). Due to the use of a non-polarizable force field, partial charges for charged ions were scaled by 0.8 to account for electronic screening, which has been shown to improve predictions of interionic interactions (Self, J., Fong, K. D. & Persson, K. A. Transport in Superconcentrated LiPF₆ and LiBF4/Propylene Carbonate Electrolytes. ACS Energy Lett. 4, 2843-2849 (2019)). The simulation procedure consisted of an energy minimization using the steepest descent method followed by an 8 ns equilibration step using a Berendsen 1 barostat and a 40 ns production run using a Parrinello-Rahman barostat, both at a reference pressure of 1 bar with timesteps of 2 fs. A Nose—Hoover thermostat was used throughout with a reference temperature of 300 K. The particle mesh Ewald method was used to calculate electrostatic interactions, with a real space cutoff of 1.2 nm and a Fourier spacing of 0.12 nm. The Verlet cutoff scheme was used to generate pairlists. A cutoff of 1.2 nm was used for non-bonded Lennard-Jones interactions. Periodic boundary conditions were applied in all directions. Bonds with hydrogen atoms were constrained. Convergence of the system energy, temperature, and box size were checked to verify equilibration. The final 30 ns of the production run were used for the analysis. Density profiles and RDFs were generated using Gromacs, while visualizations were generated with VMD (Humphrey, W., Dalke, A. & Schulten, K. VMD: Visual molecular dynamics. J. Mol. Graph. 14, 33-38 (1996)). Solvation shell statistics were calculated using the MDAnalysis Python package (Michaud-Agrawal, N., Denning, E. J., Woolf, T. B. & Beckstein, O. MDAnalysis: A toolkit for the analysis of molecular dynamics simulations. J. Comput. Chem. 32, 2319-2327 (2011)) by histogramming the observed first solvation shells for Li⁺ ions during the production simulation, using a method similar to our previous work. The cutoff distance for each species in the first solvation shell was calculated from the first minimum occurring in the RDF (referenced to Li⁺ ions) after the initial peak. The SSL, LASP and LAC each has a distinct number of Li⁺ coordinating anions of 0, 1 and ≥2 (2-5 in this work), respectively (FIGS. 88-93), in the first solvation sheath, and the percentage of each was counted based on this criterion.

General material characterizations. 1H-, 13C- and 19F-NMR spectra were recorded on a Varian Mercury 400 MHz NMR spectrometer and 7Li-NMR spectra were recorded on a UI 500 MHz NMR spectrometer at room temperature. Solvation free energies were measured according to our recent work. ATR-FTIR spectra were measured using a Nicolet iS50 with a diamond attenuated total reflectance attachment. FEI Magellan 400 XHR and Thermo Fisher Scientific Apreo S LoVac were used for taking SEM images. Ion milling was done by Fischione Model 1061 Ion Mill. For XPS measurements, each Li foil (after ten Li∥Li cell cycles) or NMC811 cathode (after thirty Li∥NMC811 cell cycles) was washed with DME for 30 s to remove the remaining electrolytes. The samples were transferred and sealed into the XPS holder in the argon-filled glovebox. The XPS profiles were collected with a PHI VersaProbe 1 scanning XPS microprobe. Viscosity measurements were carried out using an Ares G2 rheometer (TA Instruments) with an advanced Peltier system at 25.0° C.

Cryo-TEM and cryo-TEM EDS. A Thermo Fisher Titan 80-300 environmental transmission electron microscope at an accelerating voltage of 300 kV and a Gatan 626 side-entry holder were used for cryo-TEM and cryo-TEM EDS experiments. Cryo-TEM sample preparations prevent air and moisture exposure and reduce electron beam damage, as described previously. The TEM is equipped with an aberration corrector in the image-forming lens, which was tuned before imaging.

Cryo-TEM images were acquired by a Gatan K3 IS direct-detection camera in the electron-counting mode. Cryo-TEM images were taken with an electron dose rate of around 100 e-Å-2 s⁻¹, and a total of five frames were taken with 0.1 s per frame for each image.

DOSY-NMR. Sample preparation: Benzene-d6 was placed in an external coaxial insert and the 1H chemical shifts were referenced to it at 7.16 ppm. In an argon glovebox, 20 μL anhydrous toluene was mixed into 300 μL sample solution and then added into the NMR tube. The cap of NMR tube was sealed by parafilm to avoid moisture penetration during the DOSY-NMR experiment.

Measurement methods and parameters: All DOSY-NMR experiments were carried out using a 500 MHz Bruker Avance I spectrometer equipped with a z-axis gradient amplifier and a 5 mm BBO probe with a z-axis gradient coil that is capable of a maximum gradient strength at 0.535 T m⁻¹. The spectrometer frequencies for 1H- and 7Li-experiments were 500.23 MHz and 194.41 MHz, respectively. 1H- and 7Li-pulsed field gradient (PFG) measurements were performed to determine the diffusion coefficients for the solvents and electrolytes in this work. Both 1H- and 7Li-PFG measurements were performed at 298 K using the standard dstebpgp3s Bruker pulse program, employing a double stimulated echo sequence, bipolar gradient pulses for diffusion, and three spoil gradients. Apparent diffusion coefficients were calculated by fitting peak integrals to the Stejskal-Tanner equation modified for the dstebpgp3s pulse sequence (Sinnaeve, D. The Stejskal-Tanner equation generalized for any gradient shape—an overview of most pulse sequences measuring free diffusion. Concepts Magn. Reson. Part A 40A, 39-65 (2012)), and the signal attenuation due to diffusion as a function of gradient strength was in good agreement with the numerical fits for all data sets (Supplementary Table 2 and FIG. 95). The sample temperature was calibrated to 298 K using the 1H chemical shifts of the ethylene glycol sample (Ammann, C., Meier, P. & Merbach, A. A simple multinuclear NMR thermometer. J. Magn. Reson. 46, 319-321 (1982)). Similarly, the performance for the PFGs was calibrated at 298 K using dstebpgp3s sequence and the ethylene glycol sample (Spees, W. M., Song, S.-K., Garbow, J. R., Neil, J. J. & Ackerman, J. J. H. Use of ethylene glycol to evaluate gradient performance in gradient-intensive diffusion MR sequences. Magn. Reson. Med. 68, 319-324 (2012)). The PFG experiments were conducted using the following set of parameters. 1H-PFG of solvents: diffusion delay (Δ, d20)=40 ms, gradient pulse duration (8, 2*p30)=2 ms, gradient recovery delay (d16)=200 μs, array of gradient strength (gpz6)=5% to 80% with 12 linear increments, recycling delay (d1)=2 s, high power 90° pulse (p1)=9 μs. 1H-PFG of electrolytes: diffusion delay (A, d20)=150 ms, gradient pulse duration (8, 2*p30)=2 ms, gradient recovery delay (d16)=200 μs, array of gradient strength (gpz6)=5% to 80% with linear 12 increments, recycling delay (d1)=2 s, high power 90° pulse (p1)=9 μs. 7Li-PFG of electrolytes: diffusion delay (A, d20)=500 ms, gradient pulse duration (8, 2*p30)=4 ms, gradient recovery delay (d16)=200 μs, array of gradient strength (gpz6)=5% to 80% with linear 12 increments, recycling delay (d1)=2 s, high power 90° pulse (p1)=13 μs.

Electrochemical measurements. All battery components used in this work were commercially available and all electrochemical tests were carried out in a Swagelok-cell, 2032-type coin-cell or pouch-cell configuration. All cells were fabricated in an argon-filled glovebox, and one layer of Celgard 2325 was used as a separator. The EIS, Li⁺ transference number (LTN), LSV and pouch-cell cycling were carried out on a Biologic VMP3 system. The cycling tests for coin cells and some pouch cells were carried out on an Arbin instrument. The EIS measurements were taken over a frequency range of 1 MHz to 100 mHz. For the LTN measurements, 10 mV constant voltage bias was applied to Li∥Li cells. The cathodic cyclic voltammetry tests were carried out over a voltage range of −0.1 to 2 V for one cycle in Li∥Cu cells, while the anodic LSV tests were over a voltage range of 2.5 to 6.5 V in Li∥Al cells. For Li∥Li symmetric-cell cycling, 1 mA cm⁻² current density and 1 mAh cm⁻² areal capacity were applied. For Li∥Cu half-cell CE tests, ten pre-cycles between 0 and 1 V were initialized to clean the Cu electrode surface, and then cycling was done by depositing 1 (or 5) mAh cm⁻² of Li onto the Cu electrode followed by stripping to 1 V. The average CE is calculated by dividing the total stripping capacity by the total deposition capacity after the formation cycle. For the Aurbach CE test[48,49], a standard protocol was followed: (1) perform one initial formation cycle with Li deposition of 5 mAh cm⁻² on Cu under 0.5 mA cm⁻² current density and stripping to 1 V; (2) deposit 5 mAh cm⁻² Li on Cu under 0.5 mA cm⁻² as a Li reservoir; (3) repeatedly strip/deposit Li of 1 mAh cm⁻² under 0.5 mA cm⁻² for 10 cycles; (4) strip all Li to 1 V. The Li∥NMC and Cu∥NMC full cells were cycled with the following method (unless specially listed): after the first two activation cycles at 0.1 C charge/discharge (or 0.1 C charge 0.3 C discharge for anode-free pouch cells), the cells were cycled at different rates. Then a constant-current-constant-voltage protocol was used for cycling: cells were charged to top voltage and then held at that voltage until the current dropped below 0.1 C. The NMC811 coin cells were cycled between 2.8 and 4.4 V and the single-crystal NMC532 pouch cells were cycled between 3.0 and 4.4 V. The Li∥LFP and Cu∥LFP full cells were cycled with the following method (unless specially listed): after the first two activation cycles at 0.1 C charge/discharge (or 0.1 C charge 2 C discharge for anode-free pouch cells), the cells were cycled at different rates. The LFP coin cells were cycled between 2.5 and 3.9 V and the LFP pouch cells were cycled between 2.5 and 3.8 V, or between 2.5 and 3.7 V. All cells were clamped in woodworking vises to a rough pressure of ˜1,000 kPa and cycled under ambient conditions without temperature control.

### II. A. 12. Chemical Structures

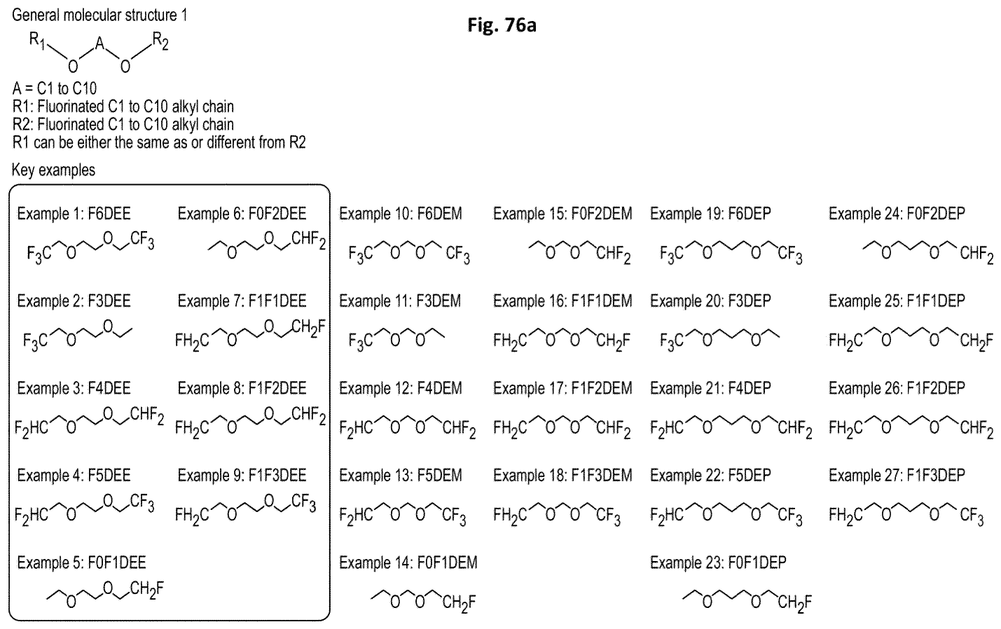

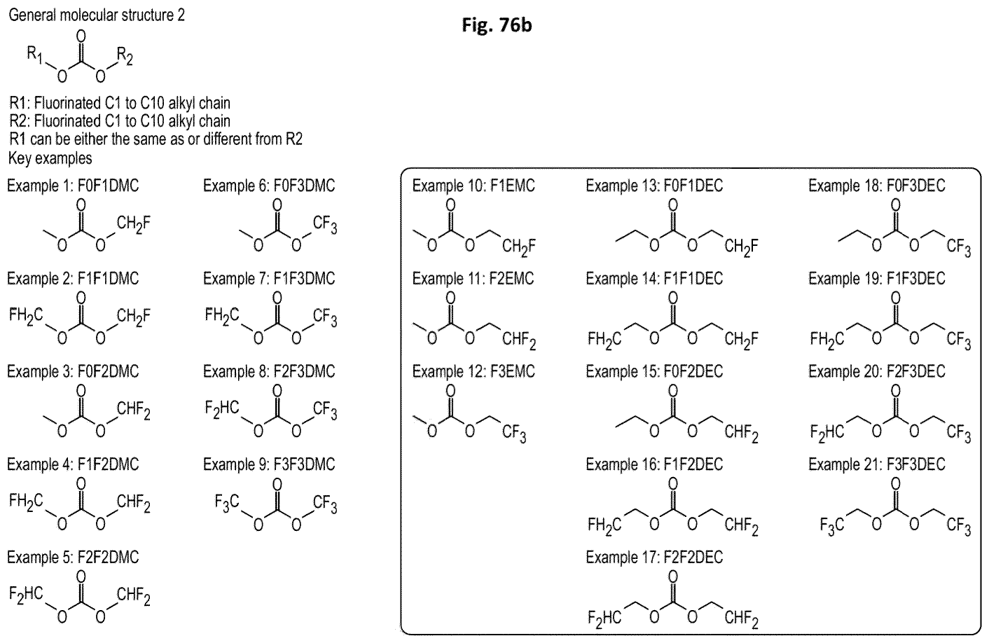

In some embodiments, a solvent for an electrolyte of a battery is a compound represented by the chemical formulas that are circled in FIGS. 76A and 76B, in which FIG. 76A contains the family of fluorinated-1,2-diethyoxyethanes (fluorinated-DEEs), fluorinated-1,1-diethyoxymethanes (fluorinated-DEMs), and fluorinated-1,3-diethyoxypropanes (fluorinated-DEPs), while FIG. 76B contains the family of fluorinated carbonates (fluorinated ethyl methyl carbonates, fluorinated dimethyl carbonates, and fluorinated diethyl carbonates).

In additional embodiments a solvent for an electrolyte of a battery is a mixture of one or more of the above-embodied fluoro-compounds and at least one of ethylene carbonate (EC), propylene carbonate (PC), dimethyl carbonate (DMC), diethyl carbonate (DEC), ethyl methyl carbonate (EMC), vinyl carbonate (VC), fluoroethylene carbonate (FEC), difluoroethylene carbonate (DFEC), 3,3,3-trifluoropropylene carbonate (TFPC), trifluoroethyl methyl carbonate (FEMC), bis(2,2,2-trifluoroethyl) carbonate (TFEC), 1,2-dimethyoxylethane (DME), 1,3-dioxolane (DOL), 1,4-dioxane (DOX), tetrahydrofuran (THF), 1,3,2-dioxathiolane-2,2-dioxide (DTD), 1,3-propanesultone (PS), acetonitrile (AN), ethyl acetate (EA), methyl acetate (MA), methyl propanoate (MP), succinonitrile (SN), trimethyl phosphate (TMP), triethyl phosphate (TEP); tris(trimethylsilyl)phosphate (TTSP), tris(2,2,2-trifluoroethyl) phosphate (TFEPa), tris(2,2,2-trifluoroethyl) phosphite (TFEPi), prop-i-ene-1,3-sultone (PES), ethylene sulfite (ES), 1,4-butane sultone (BS), dimethyl sulfoxide (DMSO), methylene methanedisulfonate (MMDS), N,N-Dimethylformamide (DMF), and gamma-butyrolactone (BL). In some embodiments, the mixture comprises two, three or four compounds from those listed above.

In some embodiments, the one or more of the above-embodied fluoro-compounds comprise at least 5 wt. %, 10 wt. %, 15 wt. %, 20 wt. %, 25 wt. %, 30 wt. %, 35 wt. %, 40 wt. %, 45 wt. %, 50 wt. %, 55 wt. %, 60 wt. %, 65 wt. %, 70 wt. %, 75 wt. %, 80 wt. %, 85 wt. %, 90 wt. %, 95 wt. %, 98 wt. %, 99 wt. %, 99 wt. %, 99.5 wt. %, or 100 wt. % of the solvent.

In additional embodiments, an electrolyte of a battery includes the solvent of any of the foregoing embodiments, and a salt. In some embodiments, the salt is a lithium salt, potassium salt, sodium salt, or a mixture thereof. For example, in some embodiments, the salt includes one or more of lithium bis(fluorosulfonyl)imide (LiFSI); lithium bis(trifluoromethanesulfonyl)imide (LiTFSI); lithium hexafluorophosphate (LiPF6); lithium hexafluoroarsenate (LiAsF6); lithium tetrafluoroborate (LiBF4); lithium bis(oxalato)borate (LiBOB); lithium difluoro(oxalato)borate (LiDFOB); lithium difluorophosphate (LiDFP); lithium nitrate (LiNO3); lithium perchlorate (LiClO4); lithium triflate (LiTf); lithium trifluoroacetate (LiTFA); lithium 4,5-dicyano-2-(trifluoromethyl)imidazole (LiTDI); sodium bis(fluorosulfonyl)imide (NaFSI); sodium bis(trifluoromethanesulfonyl)imide (NaTFSI); potassium bis(fluorosulfonyl)imide (KFSI); and potassium bis(trifluoromethanesulfonyl)imide (KTFSI).

In additional embodiments, an electrolyte of a battery includes the solvent of any of the foregoing embodiments, and a salt of any of the foregoing embodiments (e.g., a lithium salt). In some embodiments, the electrolyte includes a mixture of two or more solvents of the foregoing embodiments, and the salt (e.g., lithium salt). In some embodiments, an amount of the solvent (or the mixture of solvents) in the electrolyte is at least about 60% by weight of a total weight of the electrolyte, such as at least about 65% by weight, at least about 70% by weight, at least about 75% by weight, or at least about 80% by weight. In some embodiments, the electrolyte consists essentially of the solvent (or the mixture of solvents) and the salt (e.g., lithium salt). In some embodiments, the electrolyte includes (i) a mixture of one or more solvents of the foregoing embodiments and one or more additional solvents, such as selected from ethers and carbonates, and (ii) the salt (e.g., lithium salt). Examples of the lithium salt include lithium bis(fluorosulfonyl)imide, lithium bis(trifluoromethanesulfonyl)imide, lithium hexafluorophosphate, lithium hexafluoroarsenate, lithium tetrafluoroborate, lithium perchlorate, and lithium triflate.

In additional embodiments, a battery includes (1) an anode structure including an anode current collector, (2) a cathode structure including a cathode current collector and a cathode material disposed on the cathode current collector, and (3) the electrolyte of any of the foregoing embodiments disposed between the anode structure and the cathode structure. In some embodiments, the anode structure further includes an anode material disposed on the anode current collector. In some embodiments, the anode material comprises lithium metal, graphite, silicon, or a graphite/silicon (silicon can be Si, SiOₓ, SiC, or Si₃N₄) composite anode. In some embodiments, the graphite/silicon (silicon can be Si, SiOₓ, SiC, or Si₃N₄) composite anode includes a weight ratio of graphite/silicon of about 5:95 10:90, 20:80, 30:70, 40:60, 50:50, 60:40, 70:30, 20:80, 90:10, or 95:5. In some embodiments, the cathode material comprises a sulfur-based cathode or an air cathode (e.g., a Li—S, Li-SPAN, or a Li-air battery). In some embodiments, the cathode material comprises a lithium nickel manganese cobalt oxide (e.g., NMC111, NMC532, NMC622, NMC811, NMC900505, NMC95025025, etc.), a lithium nickel cobalt aluminum oxide (NCA), a lithium nickel manganese aluminum oxide (NMA), a lithium nickel manganese cobalt aluminum oxide (NMCA), a lithium nickel oxide (LNO), a lithium nickel manganese oxide (NM), a lithium cobalt ocide (LCO), a lithium manganese oxide (LMO), a lithium and manganese rich cathode (LMR or LLMO), a lithium iron phosphate (LFP), a lithium cobalt phosphate (LCP), a lithium manganese phosphate (LMP), a lithium manganese iron phosphate (LMFP), a transition metal sulfide (e.g., FeS, FeS₂, CuS, MoS₂, MoS₃, TiS₂, TiS₄, etc.), or any mixture combination of above cathode materials.

### II. A. 13. Supplemental Information

Supplementary TABLE 1
Physicochemical properties of developed solvents and electrolytes.

| | DEE | F3DEE | F6DEE | F4DEE | F5DEE |
|---|---|---|---|---|---|
| Molecular weight | 118.17 | 172.15 | 226.12 | 190.14 | 208.13 |
| (g mol⁻¹) | | | | | |
| Density (g mL⁻¹) | 0.842 | 1.07 | 1.40 | 1.24 | 1.29 |
| Boiling point | <20 | ~40 | ~40 | ~60 | ~60 |
| at~1 kPa (° C.) | | | | | |
| Boiling point | 122 | ~161 | ~161 | ~186 | ~186 |
| at 1 atm (° C.)* | | | | | |
| Closed-cup flash | <25 | 31 | 63 | >70 | 60-70 |
| point (° C.) | | | | | |

| | 1.M LiFSI/ | 1.M LiFSI/ | 1.M LiFSI/ | 1.M LiFSI/ | 1.M LiFSI/ |
|---|---|---|---|---|---|
| | DEE | F3DEE | F6DEE | F4DEE | F5DEE |
| Density (g mL [symbol] ) | 0.972 | 1.21 | 1.54 | 1.38 | 1.42 |
| Viscosity | 1.46 | 2.21 | 3.61 | 6.97 | 3.39 |
| (cp at 25° C.) | | | | | |
| Conductivity (mS | 11.0 ± 0.5 | 6.18 ± 0.04 | 4.48 ± 0.21 | 4.76 ± 0.007 | 5.01 ± 0.09 |
| cm [symbol] ) without separator | | | | | |
| Conductivity (mS | 0.336 ± 0.006 | 0.166 ± 0.011 | 0.045 ± 0.0004 | 0.178 ± 0.014 | 0.101 ± 0.002 |
| cm [symbol] ) with swelled | | | | | |
| Celgard separator | | | | | |
| LTN** | 0.223 | 0.316 | 0.483 | 0.234 | 0.398 |
| Li⁺ conductivity (mS | 2.45 | 1.95 | 2.16 | 1.11 | 1.99 |
| cm [symbol] ) = conductivity | | | | | |
| without separator × | | | | | |
| LTN | | | | | |

*Boiling points at 1 atm were estimated from those under vacuum using Sigma-Aldrich Pressure-Temperature Nomograph Interactive Tool ( [symbol] ).
**LTN. Li⁺ transference number measured using Li || Li symmetric cells under potentiostatic polarization¹. [symbol]
indicates data missing or illegible when filed

Ref 1: Yu, Z. et al. Molecular design for electrolyte solvents enabling energy-dense and long-cycling lithium metal batteries. Nat. Energy 5, 526-533 (2020). Supplementary Table 2. Fitting and calculation results from DOSY NMR (Su, C.-C. et al. Principle in developing novel fluorinated sulfone electrolyte for high voltage lithium-ion batteries. Energy Environ. Sci. 14, 3029-3034 (2021); Su, C.-C. et al. Solvating power series of electrolyte solvents for lithium batteries. Energy Environ. Sci. 12, 1249-1254 (2019).

| | | | | | Coordinating |
|---|---|---|---|---|---|
| Solution | D [symbol] | D [symbol] | D [symbol] | [symbol] | solvent number** |
| FDMB | 12.37 | 8.810 | N/A | N/A | N/A |
| 1M LiPSI/FDMB | 7.049 | 4.060 | 1.613 | 0.281 | 1.77 |

| | | | | | Coordinating |
|---|---|---|---|---|---|
| Solution | D [symbol] | D [symbol] | D [symbol] | [symbol] | solvent number** |
| DEE | 24.87 | 21.20 | N/A | N/A | N/A |
| 1.2M LiFSI/DEE | 11.86 | 8.269 | 3.511 | 0.306 | 2.20 |

| | | | | | Coordinating |
|---|---|---|---|---|---|
| Solution | D [symbol] | D [symbol] | D [symbol] | [symbol] | solvent number** |
| F3DEE | 20.26 | 14.94 | N/A | N/A | N/A |
| 1.2M LiPSI/F3DEE | 10.18 | 5.830 | 2.770 | 0.354 | 2.19 |

| | | | | | Coordinating |
|---|---|---|---|---|---|
| Solution | D [symbol] | D [symbol] | D [symbol] | [symbol] | solvent number** |
| F6DEE | 15.29 | 9.779 | N/A | N/A | N/A |
| 1.2M LiFSI/F6DEE | 8.239 | 4.280 | 1.361 | 0.253 | 1.57 |

| | | | | | Coordinating |
|---|---|---|---|---|---|
| Solution | D [symbol] | D [symbol] | D [symbol] | [symbol] | solvent number** |
| F4DEE | 9.028 | 5.765 | N/A | N/A | N/A |
| 1.2M LiFSI/F4DEE | 4.290 | 2.193 | 1.122 | 0.337 | 2.20 |

| | | | | | Coordinating |
|---|---|---|---|---|---|
| Solution | D [symbol] | D [symbol] | D [symbol] | [symbol] | solvent number** |
| F5DEE | 12.06 | 8.035 | NA | N/A | N/A |
| 1.2M LiFSI/F5DEE | 6.098 | 3.137 | 1.289 | 0.333 | 2.06 |

*Diffusion coefficients are in the unit of 10 [symbol] cm [symbol] .
**Coordinating solvent number was calculated by multiplying the coordination ratio ( [symbol] ) with the molar number of the solvent. [symbol]
indicates data missing or illegible when filed

Supplementary TABLE 3
Information about industrial pouch cells.

| Information | Cu \|\| single-crystal NMC532 | | Cu \|\| micro-LFP | Li \|\| poly-crystal NMC811 |
|---|---|---|---|---|
| Cu foil thickness | 8 | μm Cu | 7 μm Cu with 1 μm | No Cu current collector, |
| | | | carbon coating | Pristine 50 μm free- |
| | | | | standing Li foil towards |
| | | | | double-sided NMC811 |
| Al foil thickness | 12 μm | | | |
| Separator thickness | 12 μm thick PE coated with alumina | | | |
| Package foil thickness | ~80 μm | | | |
| Active material:Carbon:Binder | 94.0:4.0:2.0 | | 96.7:1.5:1.8 | 95.5:2.7:1.8 |
| Areal capacity* | ~3.1 | mAh cm [symbol] | ~2.1 mAh cm [symbol] | 3.8 mAh cm [symbol] |
| Total capacity* | 200 mAh | | 210 mAh | 120 mAh |
| | (0.3 C discharge) | | (0.3 C discharge) | (0.5 C discharge) |
| | | | 170 mAh | |
| | | | (2 C discharge) | |
| Electrolyte/Cathode ratio | 2.4 | | 2.4 | 2.5 |
| E/C, g Ah [symbol] ) | | | | |

| | |
|---|---|
| External temperature | Uncontrolled room temperature (18-25° C.) |
| Pressure | ~1000 kPa |

*The capacities were based on the corresponding cycling ranges: Cu || NMC532: 3.0-4.4 V; Cu || LFP: 2.5-3.8 V; Li || NMC811: 2.8-4.4 V. [symbol]
indicates data missing or illegible when filed

Supplementary TABLE 4
Comparison of the state-of-the-art high-voltage Li metal full battery performances.

| | Electrolyte | | | Cycling Condition &Capacity |
|---|---|---|---|---|
| Electrolyte | Amount | | Cell Condition | Retention |
| 7M LiFSI in FEC (ref. [symbol] ) | Not | | 2.5 mAh cm [symbol] Li \|\| 1.83 | 70% after 140 cycles |
| | mentioned | | mAh cm [symbol] LNMO | |
| | | | N/P = 1.37 | |
| 1LiFSI-3TMS-3TTE (ref. [symbol] ) | 40 | mL Ah [symbol] | 50 μm Li \|\| 1.5 mAh cm [symbol] | C/3 cycling, 80% after 300 cycles |
| | | | NMC111 | |
| | | | N/P = 6.67 | |
| 1.2M LiFSI/DMC-BTFE | 80 | mL Ah [symbol] | 450 μm Li \|\| 2 mAh cm [symbol] | 0.5 C charge 2 C discharge, 80% after 700 |
| (ref. [symbol] ) | | | NMC111 | cycles |
| | | | N/P = 45 | |
| 1M LiPF [symbol] /FEC-FEMC-HFE | 50 | g Ah [symbol] | 2 mAh cm⁻² Li \|\| 2 mAh | C/2 cycling, 95% after 120 cycles |
| (ref. [symbol] ) | | | cm [symbol] NMC811 coin cell | |
| | | | N/P = 1 | |
| 1LiFSI-1.2DME-3TTE (ref. [symbol] ) | 3 | g Ah [symbol] | 50 μm Li \|\|~4.2 mAh cm [symbol] | C/3 cycling, 80% after 155 cycles |
| | | | NMC811 coin cell | |
| | | | N/P = 2.38 | |
| 1M LiFSI/DME/TFEO (ref. [symbol] ) | 50 | g Ah [symbol] | 50 μm Li \|\|~1.5 mAh cm [symbol] | C/3 cycling, 80% after 300 cycles |
| | | | NMC811 coin cell | |
| | | | N/P = 6.67 | |
| 1LiFSI-1.3DME-2TFEO | ~4.5 | g Ah [symbol] | 50 μm Li \|\|~4.2 mAh cm [symbol] | 0.1 C charge 0.3 C discharge, 80% after |
| (ref. [symbol] ) | | | NMC811 coin cell | ~210 cycles |
| | | | N/P = 2.38 | |
| 1M LiFSI/FSA (ref. [symbol] ) | 25 | mL Ah [symbol] | 60 μm Li \|\| 1.6 mAh cm [symbol] | 89% after 200 cycles |
| | | | NMC622 | |
| | | | N/P = 7.5 | |
| 1m LiFSI in DMTMSA | ~12 | mL Ah [symbol] | 60 μm Li \|\|~1.7 mAh cm [symbol] | 4.7 V, 0.5 C cycling, 88% after 100 cycles |
| (ref. [symbol] ) | | | NMC811 | |
| | | | N/P = 7.06 | |
| 1M LiPF [symbol] /EC-DEC with 10 | 8.4 | g Ah [symbol] | 50 μm Li \|\|~4.3 mAh cm [symbol] | 0.3 C charge 0.5 C discharge, 80% after 160 |
| mM In(OTf) [symbol] and 0.5M | | | NMC811 coin cell | cycles |
| LiNO [symbol] (ref. [symbol] ) | | | N/P = 2.33 | |
| 1M LiPF [symbol] /FEC-EMC with 3 | 3.4 | g Ah [symbol] | 50 μm Li \|\|~3.4 mAh cm [symbol] | 0.1 C charge 0.3 C discharge, 80% after 140 |
| wt % LiNO [symbol] and 1 wt % | | | NMC811 coin cell | cycles |
| TPFPB (ref. [symbol] ) | | | N/P = 2.94 | |

[symbol]

indicates data missing or illegible when filed

The references in the above Table are as follows:
Ref 4: Suo, L. et al. Fluorine-donating electrolytes enable highly reversible 5-V-class Li metal batteries. Proc. Natl. Acad. Sci. 115, 1156-1161 (2018).
Ref 5: Ren, X. et al. Localized High-Concentration Sulfone Electrolytes for High-Efficiency Lithium-Metal Batteries. Chem 4, 1877-1892 (2018).
Ref 6: Chen, S. et al. High-Voltage Lithium-Metal Batteries Enabled by Localized High-Concentration Electrolytes. Adv. Mater. 30, 1706102 (2018).
Ref 7: Fan, X. et al. Non-flammable electrolyte enables Li-metal batteries with aggressive cathode chemistries. Nat. Nanotechnol. 13, 715-722 (2018).
Ref 8: Ren, X. et al. Enabling High-Voltage Lithium-Metal Batteries under Practical Conditions. Joule 3, 1662-1676 (2019).
Ref 9: Cao, X. et al. Monolithic solid-electrolyte interphases formed in fluorinated orthoformate-based electrolytes minimize Li depletion and pulverization. Nat. Energy 4, 796-805 (2019).
Ref 10: Cao, X. et al. Optimization of fluorinated orthoformate based electrolytes for practical high-voltage lithium metal batteries. Energy Storage Mater. 34, 76-84 (2021).
Ref 11: Xue, W. et al. FSI-inspired solvent and “full fluorosulfonyl” electrolyte for 4 V class lithium-metal batteries. Energy Environ. Sci. 13, 212-220 (2020).
Ref 12: Xue, W. et al. Ultra-high-voltage Ni-rich layered cathodes in practical Li metal batteries enabled by a sulfonamidebased electrolyte. Nat. Energy 6, 495-505 (2021).
Ref 13: Zhang, W. et al. Engineering Wavy-Nanostructured Anode Interphases with Fast Ion Transfer Kinetics: Toward Practical Li-Metal Full Batteries. Adv. Funct. Mater. 30, 2003800 (2020).
Ref 14: Li, S. et al. Synergistic Dual-Additive Electrolyte Enables Practical Lithium-Metal Batteries. Angew. Chemie 132, 15045-15051 (2020).

| | | | |
|---|---|---|---|
| 1M LiFSI/FDMB (ref. [symbol] ) | ~6 g Ah [symbol] | 20 μm Li \|\| 1.6 mAh cm [symbol] | C/3 cycling, 100%% after 210 cycles |
| | | NMC532 coin cell | |
| | | N/P = 2.5 | |
| This work: | ~2.5 g Ah [symbol] | 25 μm Li \|\| 3. [symbol] mAh cm [symbol] | 0.2 C charge 0.5 C discharge |
| 1.2M LiFSI/F3DEE | | NMC811 industrial | F3DEE, 80% after 135 cycles |
| 1.2M LiFSI/F6DEE | | pouch cells | F6DEE, 80% after 110 cycles |
| 1.2M LiFSI/F4DEE | | N/P = 1.32 | F4DEE, 80% after 105 cycles |
| 1.2M LiFSI/F5DEE | | | F5DEE, 80% after 150 cycles |
| This work: | ~8 g Ah [symbol] | 50 μm Li \|\| 4.9 mAh cm [symbol] | 0.2 C charge 0.3 C discharge |
| 1.2M LiFSI/F3DEE | | NMC811 coin cells | F3DEE, 80% after 125 cycles |
| 1.2M LiFSI/F6DEE | | N/P = 2.04 | F6DEE, 80% after 130 cycles |
| 1.2M LiFSI/F4DEE | | | F4DEE, 80% after 185 cycles |
| 1.2M LiFSI/F5DEE | | | F5DEE, 80% after >200 cycles |
| This work: | ~8 g Ah [symbol] | 50 μm Li \|\| 4.9 mAh cm [symbol] | 0.1 C charge 0.3 C discharge |
| 1.2M LiFSI/F4DEE | | NMC811 coin cells | F4DEE, 80% after 180 cycles |
| 1.2M LiFSI/F5DEE | | N/P = 2.04 | F5DEE, 80% after 270 cycles |

[symbol]

indicates data missing or illegible when filed

Supplementary TABLE 5
Comparison of anode-free Li metal battery (zero Li excess) performances.

| | Electrolyte | | | Cycling Condition & Capacity |
|---|---|---|---|---|
| Electrolyte | Amount | | Cell Condition | Retention |
| 4M LiPSI in DME (ref. [symbol] ) | ~44 | g Ah [symbol] | Cu \|\| LFP coin cell, | ~C/8 charge discharge, |
| | | | 1.71 mAh cm [symbol] | 60% after 50 cycles |
| 1M LiIFSI + 2M LiFSI + 3 | Not | | Cu \|\| LFP coin cell, | 39% after 100 cycles |
| wt % LiNO [symbol] in DME/DOL | mentioned | | 0.85 mAh cm [symbol] | |
| (ref. [symbol] ) | | | | |
| 1.7M LiFSI in MeTHF/TTE | 4.0 | g Ah [symbol] | Cu \|\| LFP pouch cell, | 0.3 C charge 0.5 C discharge, |
| (ref. [symbol] ) | | | 2.7 mAh cm [symbol] , 560 mAh | 41.6% after 150 cycles |
| 7M LiFSI in FEC (ref. [symbol] ) | Not | | Cu \|\| LNMO coin cell, | 54% after 50 cycles |
| | mentioned | | 1.43 mAh cm [symbol] | |
| 2M LiPF [symbol] in EC/DEC + 50% | Not | | Cu \|\| NMC111 coin cell, | ~C/8 charge/discharge, |
| FEC (ref. [symbol] ) | mentioned | | ~1.6 mAh cm [symbol] | 40% after 50 cycles |
| 1M LiPF [symbol] in | ~47 | g Ah [symbol] | Cu \|\| NMC811 coin cell, | ~C/4 charge/discharge, |
| FEC/FEMC/HFE (ref. [symbol] ) | | | ~2.0 mAh cm [symbol] | 50% after 30 cycles |
| 1LiFSI-1.2DME-3TTE (ref. [symbol] ) | 3 | g Ah [symbol] | Cu \|\| NMC811 coin cell, | C/10 charge C/3 discharge, |
| | | | ~4.2 mAh cm [symbol] | 77% after 70 cycles |
| 0.6 or 1M LiDFOB + 0.6 or | ~2 | g Ah [symbol] | Cu \|\| NMC532 pouch cell, | ~80% DOD (depth of discharge), C/5 |
| 0.2M LiBF [symbol] in FEC/DEC | | | ~245 mAh | charge C/2 discharge, 40° C., 80% after 80 |
| (ref. [symbol] ) | | | | or 90 cycles (low pressure) |
| High concentration, 1.8M | ~2 | g Ah [symbol] | Cu \|\| NMC532 pouch cell, | ~70% DOD, C/5 charge C/2 discharge, 40° |
| LiDFOB + 0.4M LiBF [symbol] in | | | ~210 mAh | C. hot formation, 80% after 90 cycles (low |
| FEC/DEC (ref. [symbol] ) | | | | pressure)/80% after 195 cycles (high |
| | | | | pressure) |
| 0.6M LiDFOB + 0.6M | ~2 | g Ah [symbol] | Cu \|\| NMC532 pouch cell, | ~70% DOD, C/5 charge C/2 discharge, |
| LiBF4 in FEC/DEC (ref. [symbol] ) | | | ~210 mAh | controlled 20° C., 80% after ~16 cycles |
| | | | | (low pressure)/80% after 50-60 cycles |
| | | | | (high pressure) |
| High concentration, 2.0M | ~2.6 | g Ah [symbol] | Cu \|\| NMC532 pouch cell, | ~70% DOD, C/5 charge C/2 discharge, |
| LiDFOB and 1.4M LiBF [symbol] in | | | ~210 mAh | controlled 20° C., 80% after 200 cycles |
| FEC/DEC (ref. [symbol] ) | | | | (high pressure) |
| 1m LiFSI & N,N- | ~3 | g Ah [symbol] | Cu \|\| NMC811 coin cell, | 100% DOD (3-4.5 V), C/10 charge C/3 |
| dimethyltrifluoromethane- | | | 4.5 mAh cm [symbol] | discharge, 73% after 65 cycles |
| sulfonamide (DMTMSA) | | | | |
| (ref. [symbol] ) | | | | |
| 1M LiFSI/FDMB (ref. [symbol] ) | ~2 | g Ah [symbol] | Cu \|\| NMC532, Cu \|\| | 100% DOD, C/5 charge C/3 discharge, |
| | | | NMC622, and Cu \|\| | uncontrolled room temperature (18-25° C.), |
| | | | NMC811 pouch cells, | 80% after 100 cycles (low pressure) |
| | | | 200-250 mAh | |
| This work: | ~2.4 | g Ah [symbol] | Cu \|\| micro-LFP pouch | Fast cycling: |
| 1.2M LiFSI/F4DEE | | | cells that have never been | 100% DOD, uncontrolled room |
| 1.2M LiFSI/F5DEE | | | tried in the field, ~170 | temperature (18-25° C.), |
| | | | mAh at 2 C discharge | 0.2 C charge 2 C discharge, 80% after |
| | | | | 140 cycles (high presure)/ |
| | | | | 0.5 C charge 2 C discharge, 80% after |
| | | | | 110 cycles (high pressure)/ |
| | | | | 1 C charge 2 C discharge, 80% after 90 |
| | | | | cycles (high pressure) |

[symbol]

indicates data missing or illegible when filed

The references in the above table are as follows:
Ref 15. Qian, J. et al. Anode-Free Rechargeable Lithium Metal Batteries. Adv. Funct. Mater. 26, 7094-7102 (2016).
Ref 16. Qiu, F. et al. A Concentrated Ternary-Salts Electrolyte for High Reversible Li Metal Battery with Slight Excess Li. Adv. Energy Mater. 9, 1803372 (2019).
Ref 17. Xu, R. et al. Design and Demystify the Lithium Metal Interface towards Highly Reversible Batteries. Adv. Mater. (2021) doi:10.1002/adma.202105962.
Ref 18. Hagos, T. T. et al. Locally Concentrated LiPF6 in a Carbonate-Based Electrolyte with Fluoroethylene Carbonate as a Diluent for Anode-Free Lithium Metal Batteries. ACS Appl. Mater. Interfaces 11, 9955-9963 (2019).
Ref 19. Weber, R. et al. Long cycle life and dendrite-free lithium morphology in anode-free lithium pouch cells enabled by a dual-salt liquid electrolyte. Nat. Energy 4, 683-689 (2019).
Ref 20. Genovese, M. et al. Hot Formation for Improved Low Temperature Cycling of Anode-Free Lithium Metal Batteries. J. Electrochem. Soc. 166, A3342-A3347 (2019).
Ref 21. Louli, A. J. et al. Diagnosing and correcting anode-free cell failure via electrolyte and morphological analysis. Nat. Energy 5, 693-702 (2020).

FIG. 77(a,b) Boiling points of synthesized fluorinated-DEEs: vapor temperatures measured during vacuum distillation (a) and estimated boiling points at 1 atm (b). (c) Viscosities of 1.2 M LiFSI in fluorinated-DEEs versus shear rate, measured by rheology.

Note: The same as those in Supplementary Table 1, boiling points at 1 atm were estimated from those under vacuum using Sigma-Aldrich Pressure-Temperature Nomograph Interactive Tool (https://www.sigmaaldrich.com/chemistry/solvents/learning-center/nomograph.html?gclid=CjwKCAjw9r-DBhBxEiwA9qYUpctOuvLP40XzFVLCoWHjYI6vEho6xQ1V2uNm3QzJUdEsakSbSvpu OxoCsFoQAvD_BwE).

FIG. 78 illustrates Ionic conductivities of developed electrolytes and control electrolytes measured with (a) and without (b) Celgard 2325 separators.

Note: Swagelok cells measure the conductivities of pure electrolyte liquids while coin cells measure the Celgard 2325 separators swelled by the electrolytes. The latter ones mimic the situation in realistic cells. The 1 M LiFSI/FDMB data in (b) was extracted from ref.1. From (a), we can see that the ion conductivity of 1.2 M LiFSI/DEE is similar to that of LP40 (1 M LiPF₆ in EC/DEC [1/1]) electrolyte, while that of F3DEE or F4DEE was as ˜60% high as the DEE one. The conductivity of 1.2 M LiFSI/F5DEE was ˜40% that of 1.2 M LiFSI/DEE, but 1.2 M LiFSI/F6DEE and 1 M LiFSI/FDMB were similarly low.

FIG. 79 illustrates EIS plots of Li∥Li symmetric cells with cycling: before cycling (a), and after 20 cycles (b), 60 cycles (c), 120 cycles (d), 140 cycles (e), 180 cycles (f).

Note: Generally, the impedance evolution of Li∥Li cells with cycling follows the overpotential trend. The overall impedances of DEE, F3DEE, F4DEE, and F5DEE cells were maintained to be low, while those of F6DEE and FDMB cells increased vastly with cycling.

FIG. 80 provides Voltage profiles of Li∥Cu half cell using 1 M LiFSI/FDMB at different cycle numbers.

FIG. 81 provides Voltage profiles of Li∥Cu half cell using 1.2 M LiFSI/DEE at different cycle numbers.

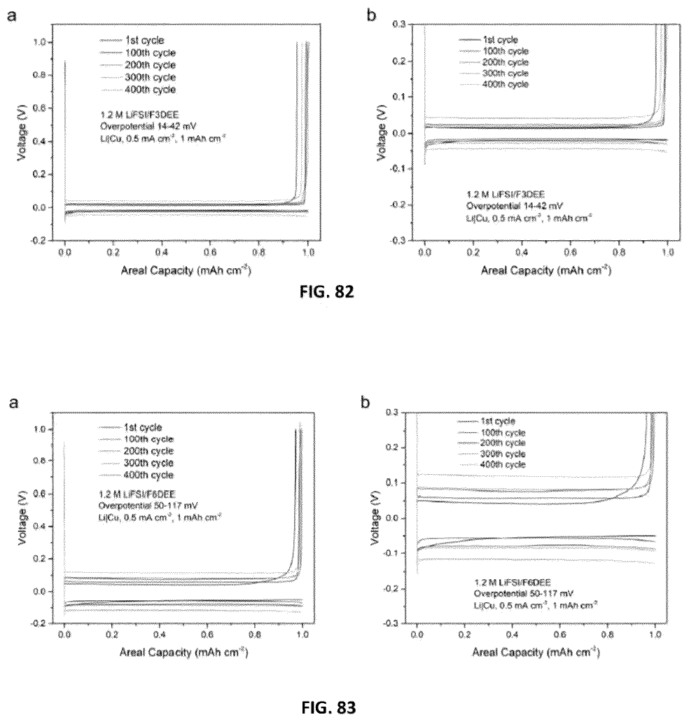

FIG. 82 provides Voltage profiles of Li∥Cu half cell using 1.2 M LiFSI/F3DEE at different cycle numbers.

FIG. 83 provides Voltage profiles of Li∥Cu half cell using 1.2 M LiFSI/F6DEE at different cycle numbers.

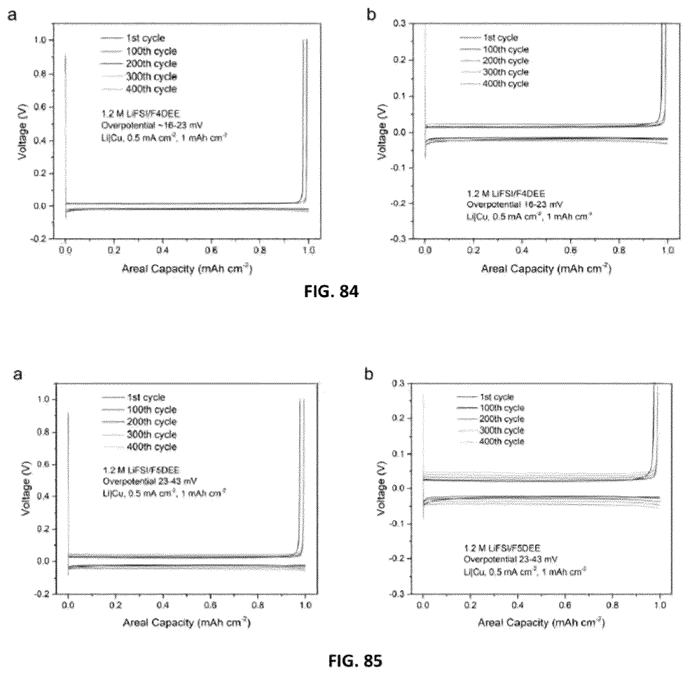

FIG. 84 provides Voltage profiles of Li∥Cu half cell using 1.2 M LiFSI/F4DEE at different cycle numbers.

FIG. 85 provides Voltage profiles of Li∥Cu half cell using 1.2 M LiFSI/F5DEE at different cycle numbers.

FIG. 86 provides Electrostatic potential (ESP) of different solvent molecules.

Note: Generally, the negative charge was more located on O and F atoms of these molecules. However, fine difference can be observed when comparing the ESP of —CF₃ and —CHF2. At the same isopotential scale, the —CHF2 group showed more concentrated negative charge (darker red color) while the symmetric —CF₃ group showed slightly less negative charge (more yellowish color), especially when one compares the —CF₃ and —CHF₂ in F5DEE, or compares F4DEE and F6DEE. This observation is consistent with the stronger coordination capability of —CHF₂ than —CF₃, as elaborated in the manuscript.

FIG. 87 provides 19F-NMR (376 MHz) spectra of pure fluorinated-DEEs and 1.2 M LiFSI in fluorinated-DEES. (a) —CF₃ on F3DEE and 1.2 M LiFSI/F3DEE. (b) —CF₃ on F6DEE and 1.2 M LiFSI/F6DEE. (c) —CHF2 on F4DEE and 1.2 M LiFSI/F4DEE. (d) —CF₃ on F5DEE and 1.2 M LiFSI/F5DEE. (e) —CHF2 on F5DEE and 1.2 M LiFSI/F5DEE.

Note: All —CF₃ groups on fluorinated-DEEs showed downfield shift while the —CHF2 ones showed upfield shift. The upfield shift was recognized as an indication of strong Li—F interaction (Yu, Z. et al. A Dynamic, Electrolyte-Blocking, and Single-Ion-Conductive Network for Stable Lithium-Metal Anodes. Joule 3, 2761-2776 (2019); Jia, M. et al. Fluorinated Bifunctional Solid Polymer Electrolyte Synthesized under Visible Light for Stable Lithium Deposition and Dendrite-Free All-Solid-State Batteries. Adv. Funct. Mater. 31, 2101736 (2021)) since Li+ ions and their attached (surrounding) FSI-anions are close to these F atoms on —CHF₂ groups, leading to anion shielding effect.

FIG. 88 provides MD simulation results of 1 M LiFSI/FDMB. (a) Probabilities of different Li+ solvates (with different anion and solvent numbers in the first solvation sheath). Blue: solvent surrounded Li+ (SSL, i.e. Li+(FSI)0); green: Li+-anion single pair (LASP, i.e. Li+(FSI)1); yellow: Li+-anion cluster (LAC, i.e. Li+(FSI)≥2). (b) RDF between Li+ and O atoms on FSI−. (c) RDF between Li+ and O/F atoms on FDMB solvent.

Note: These results were from newly conducted MD simulations where “scaled charges” were used for solvent molecules to maximize the electrostatic effect contributed by the solvents. Therefore the results here are different from those in Ref.1.

FIG. 89 provides MD simulation results of 1.2 M LiFSI/DEE. (a) Probabilities of different Li+ solvates (with different anion and solvent numbers in the first solvation sheath). Blue: SSL; green: LASP; yellow: LAC. (b) RDF between Li+ and O atoms on FSI−. (c) RDF between Li+ and O atoms on DEE solvent.

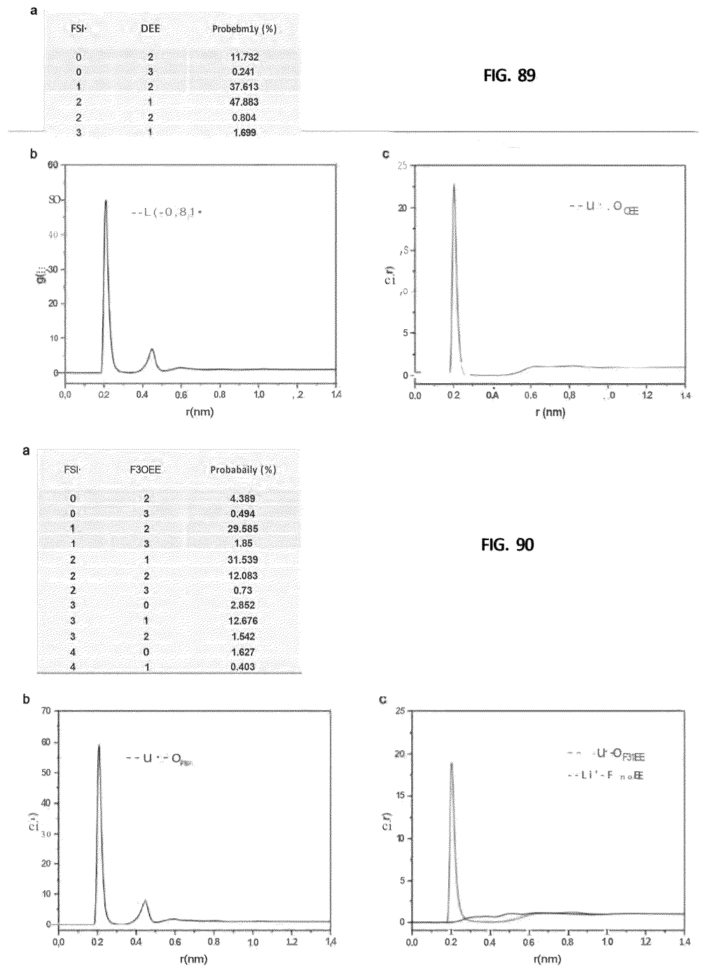

FIG. 90 provides MD simulation results of 1.2 M LiFSI/F3DEE. (a) Probabilities of different Li+ solvates (with different anion and solvent numbers in the first solvation sheath). Blue: SSL; green: LASP; yellow: LAC. (b) RDF between Li+ and O atoms on FSI−. (c) RDF between Li+ and O/F atoms on F3DEE solvent.

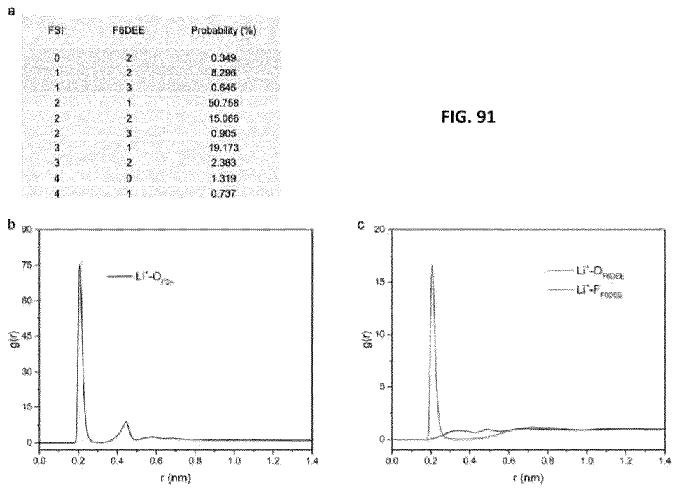

FIG. 91 provides MD simulation results of 1.2 M LiFSI/F6DEE. (a) Probabilities of different Li+ solvates (with different anion and solvent numbers in the first solvation sheath). Blue: SSL; green: LASP; yellow: LAC. (b) RDF between Li+ and O atoms on FSI−. (c) RDF between Li+ and O/F atoms on F6DEE solvent.

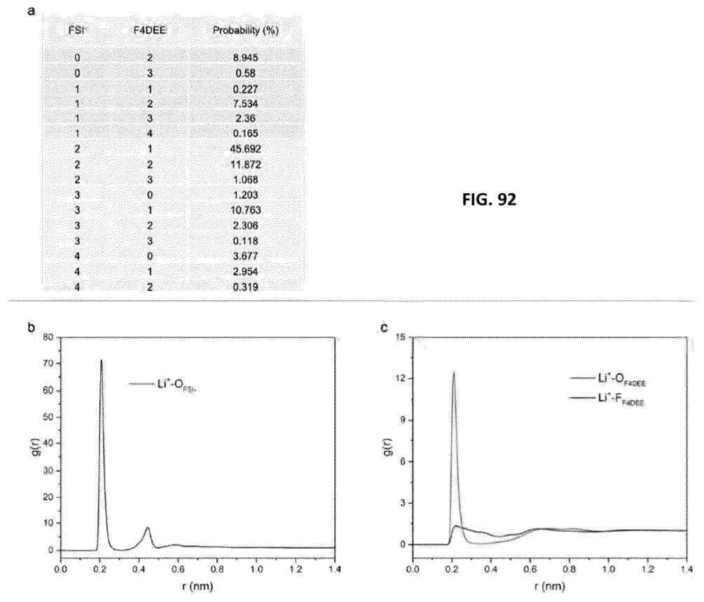

FIG. 92 provides MD simulation results of 1.2 M LiFSI/F4DEE. (a) Probabilities of different Li+ solvates (with different anion and solvent numbers in the first solvation sheath). Blue: SSL; green: LASP; yellow: LAC. (b) RDF between Li+ and O atoms on FSI−. (c) RDF between Li+ and O/F atoms on F4DEE solvent.

Note: Compared with the Li-FF6DEE RDF (Supplementary FIG. 15c), the Li-FF4DEE showed higher peak around 0.2 nm, indicating stronger Li—F interaction between Li⁺ and —CHF₂ than —CF₃. This is consistent with our design logic.

FIG. 93 provides MD simulation results of 1.2 M LiFSI/F5DEE. (a) Probabilities of different Li+ solvates (with different anion and solvent numbers in the first solvation sheath). Blue: SSL; green: LASP; yellow: LAC. (b) RDF between Li+ and O atoms on FSI−. (c) RDF between Li+ and O/F atoms on F5DEE solvent. (d) Separated RDF between Li+ and F atoms on —CHF2 than —CF₃.

Note: As shown in Supplementary FIG. 17d, the Li—F—CHF₂ RDF peak is much higher than Li—F—CF₃ one at around 0.2 nm, indicating more F atoms on —CHF₂ participating into the Li+ solvation than the ones on —CF₃. Such stronger interaction between Li+ and —CHF₂ matches well with the DFT results (lower distance between Li+ and —CHF₂ than —CF₃). This confirms the benefit of —CHF₂ incorporation, and is consistent with our design.

FIG. 94 provides Fitting results of internal reference DOSY NMR (taking the pair of F3DEE and 1.2 M LiFSI/F3DEE as an example here). All the results were summarized in Supplementary Table 2. The method was previously developed by Amine et al. to determine the Li⁺ coordination number in electrolyte solution. y=−2.7705E-06x-5.1136E-02 R²=9.9884E-01

FIG. 95 provides 7Li NMR (194 MHz) results of 1 M LiFSI/FDMB (extracted from Ref.1) and 1.2 M LiFSI in fluorinated-DEEs. The chemical shift positions were plotted in (b), following the design flow (the x-axis order is different from that in main FIG. 3).

Note: The more negative the 7Li shift is (upfield shift), the more shielded the Li+ ion is. Usually this indicates more anions surrounding Li+ ions since negatively charged anions provide more shielding effect for Li+. In 7Li-NMR spectra, more upfield shift was observed for 1 M LiFSI/FDMB and 1.2 M LiFSI/F6DEE, indicating weakly-solvated anion-shielded Li+ (i.e. close Li+-FSI− clustering); on the contrary, downfield chemical shifts in DEE, F3DEE and F4DEE electrolytes confirmed their strong solvation ability (i.e. separating Li+-FSI− ion pairs)25.

FIG. 96 provides Solvation energy (ΔGsolvation) measurements of fluorinated-DEE electrolytes following the design flow (the x-axis order is different from that in main FIG. 3).

Note: The solvation Gibbs free energy (ΔGsolvation) was converted from the measured H-cell open circuit voltage (EH-cell) using equation: ΔG=−nFE. The detailed method and rationales were described in Kim and Cui et al. unpublished. Regardless of the measurement technique, the solvation Gibbs free energy is an overall evaluation of the binding strength (how much the Gibbs energy decreases) between Li+ and surrounding species (both solvent and anion). Since the anion is fixed as FSI− in the measured electrolytes, stronger binding solvents (e.g. DME and DEE herein) will participate more into the Li+ solvation sheath and lead to more negative solvation energies, indicating more Li+-anion dissociation (or less ion pairing). This argument is consistent with both theoretical and other experimental results as well as the discussions in the manuscript.

FIG. 97 provides FTIR results of 1.2 M LiFSI in fluorinated-DEEs. (a) The whole spectra. (b) Zoomed-in region of FSI− anion peaks. (c) Zoomed-in region of ether group peaks.

Note: The zoomed-in region of C—O—C stretching showed higher “solvated” ether peaks in F3DEE (3s), F4DEE (4s), and F5DEE (5s) electrolytes compared to F6DEE one (6s), indicating more solvating solvents in the former three electrolytes. In F6DEE, even only “free” solvent (6f) can be observed.

FIG. 98 provides Long cycling of conventional (thin spring) Li∥Cu half cells at 0.5 mA cm−2 and 1 mAh cm−2, using fluorinated-DEE electrolytes: 1 M LiFSI/FDMB (a, extracted from ref.1), 1.2 M LiFSI/DEE (b), 1.2 M LiFSI/F3DEE (c), 1.2 M LiFSI/F6DEE (d), 1.2 M LiFSI/F4DEE (e), and 1.2 M LiFSI/F5DEE (f). Two parallel cells were shown in each electrolyte.

FIG. 99 provides (a,b) Initial cycling of Li∥Cu half cells at 0.5 mA cm−2 and 1 mAh cm−2 (a) and 0.5 mA cm−2 and 5 mAh cm−2 (b). (c) Long cycling of high-pressure (tough spring shown in (e)) Li∥Cu half cells at 0.5 mA cm−2 and 1 mAh cm−2. (d) CV curves of Li∥Cu cells using different electrolytes in this work. (e) Images of soft and strong springs. Parallel cell results were given in each case. (f) Pressure inside coin cells with different springs measured by pressure-indicating films (Fujifilm Prescale® LLLW, 28-85 μsi).

Note: Under different conditions, the F4DEE and F5DEE electrolytes enabled high CE in Li∥Cu half cells. Particularly, the CE of Li∥Cu half cells using F5DEE under high pressure reached 99.8-99.9% during long cycling. Figure (d) showed FSI− anion decomposition peaks for all electrolytes when scanning CV for Li∥Cu half cells, indicating anion-derived SEI; however, 1.2 M LiFSI/DEE showed earlier and easier FSI− decomposition (gray curve, >1.3 V) compared to fluorinated-DEE electrolytes, which may be responsible for worse Li metal stability in DEE. Figure (f) showed that the strong spring provided higher and more uniform internal pressure for coin cells than the soft one.

FIG. 100 provides Cycling CE of Li∥Cu half cells at high currents and high capacities. (a) Cu foil, 2 mA cm−2 plating, 4 mA cm−2 stripping and 4 mAh cm−2 capacity. (b) Cu foam, 4 mA cm−2 plating, 4 mA cm−2 stripping and 4 mAh cm−2 capacity. (c) Cu foam, 8 mA cm−2 plating, 2 mA cm−2 stripping and 4 mAh cm−2 capacity. (d) Cu foam, 10 mA cm−2 plating, 2 mA cm−2 stripping and 4 mAh cm−2 capacity.

Note: In Li∥Cu half cells, higher currents (>4 mA cm⁻²) can lead to a reasonable decrease in Li metal CE compared to lower currents (Supplementary FIG. 23). However, at 2 mA cm⁻² plating, 4 mA cm⁻² stripping and 4 mAh cm⁻² capacity which are harsher than the cycling condition in anode-free Cu∥LFP cells (main text FIG. 5i-k), the cycling CE using 1.2 M LiFSI/F4DEE or 1.2 M LiFSI/F5DEE was still maintained as high as ˜99%.

FIG. 101 provides Aurbach method using repeated Li∥Cu half cells to obtain average CEs for the fluorinated-DEE based electrolytes: 1 M LiFSI/FDMB (a), 1.2 M LiFSI/DEE (b), 1.2 M LiFSI/F3DEE (c), 1.2 M LiFSI/F6DEE (d), 1.2 M LiFSI/F4DEE (e), and 1.2 M LiFSI/F5DEE (f). Note: For each electrolyte, two parallel cells were tested to show repeatability of Aurbach method. Both cells for each electrolyte showed similar average Li metal CE and similar overpotential.

FIG. 102 provides LSV of Li∥Al coin cells using fluorinated-DEE electrolytes, in which the data of 1 M LiFSI/FDMB and 1 M LiFSI/DME were extracted from ref.1. Note: The leaking current is an indication of Al corrosion at high voltage, which is a critical factor for the stability of cathodes and functionality of high-voltage batteries.

FIG. 103 provides Potatiostatic polarization of Li∥Al coin cells using fluorinated-DEE electrolytes: 1 M LiFSI/FDMB (a, extracted from ref.1), 1.2 M LiFSI/DEE (b), 1.2 M LiFSI/F3DEE (c), 1.2 M LiFSI/F6DEE (d), 1.2 M LiFSI/F4DEE (e), and 1.2 M LiFSI/F5DEE (f).

Note: All developed electrolytes, except 1.2 M LiFSI/DEE and 1.2 M LiFSI/F3DEE, showed decent oxidative stability in these potatiostatic polarization tests by exhibiting either decaying or plateaued current during each voltage holding step.

FIG. 104 provides HOMO and LUMO levels of different fluorinated-DEE molecules.

FIG. 105 provides Cycling performance of thin Li∥4.9 mAh cm−2 NMC811 coin cells using fluorinated-DEE electrolytes. (a) CEs of 50 μm Li∥4.9 mAh cm−2 NMC811 cells at 0.2 C charge 0.3 C discharge, which were extracted from the testing results in main text FIG. 5c. (b,c) Capacity retention (b) and CEs (c) of 50 μm Li∥4.9 mAh cm−2 NMC811 cells at 0.1 C charge 0.3 C discharge. (d,e) Capacity retention (d) and CEs (e) of 20 μm Li∥4.9 mAh cm−2 NMC811 cells at 0.2 C charge 0.3 C discharge. Replicated cell data were shown here.

Note: The 50 μm Li∥4.9 mAh cm⁻² NMC811 cells at 0.1 C charge 0.3 C discharge using 1.2 M LiFSI/F5DEE showed CE fluctuation at the early stage, which was caused by the instrument shutdown and temperature fluctuation.

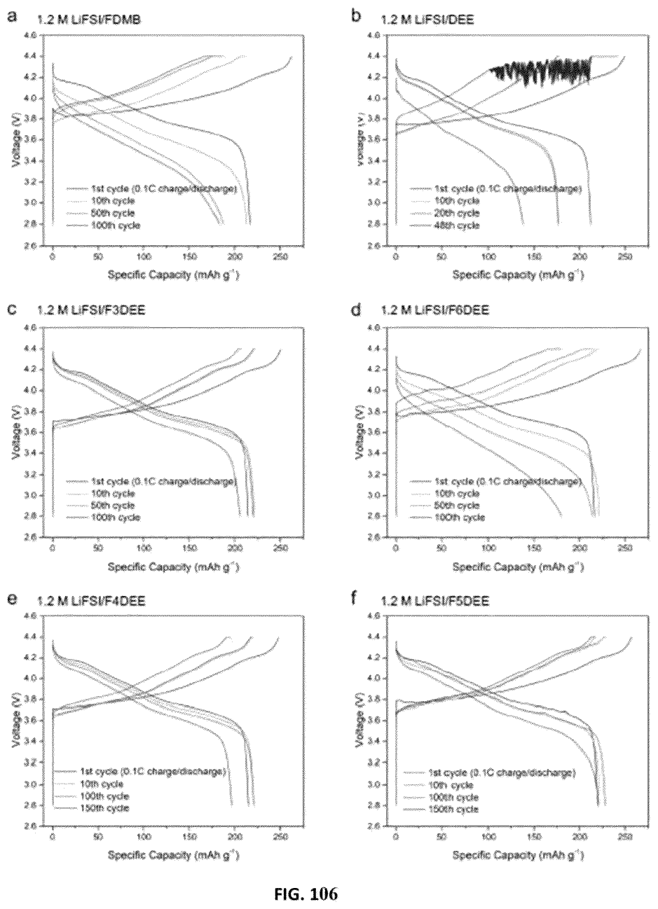

FIG. 106 provides Charge/discharge curves of 50 μm Li∥˜4.9 mAh cm−2 NMC811 coin cells using fluorinated-DEE electrolytes at 0.2 C charge 0.3 C discharge.

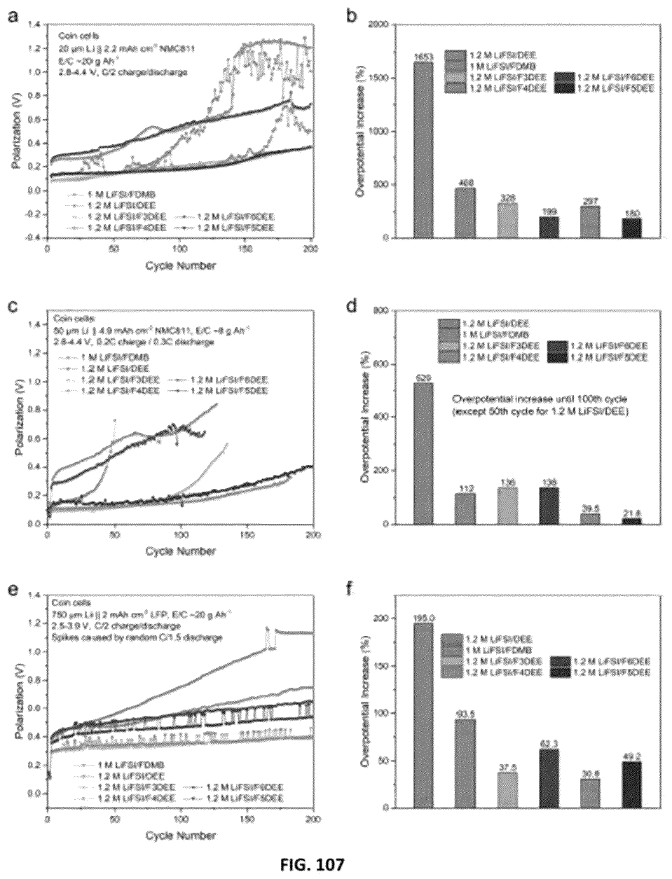

FIG. 107 provides Voltage polarization of Li∥NMC811 or microparticle-LFP coin cells. (a,b) 20 μm Li∥˜2.2 mAh cm−2 NMC811. (c,d) 50 μm Li∥˜4.9 mAh cm−2 NMC811. (e,f) 750 μm Li∥˜2 mAh cm−2 microparticle-LFP. (b,d,f) showed the overpotential increase percentage.

FIG. 108 provides EIS plots (a) and fitting results (b,c) of Cu∥NMC532 pouch cells after 40 cycles at 0.2 C charge 0.3 C discharge. The fitting is based on simplified equivalent circuit28. The Rinterface was the sum of SEI, CEI, and charge transfer resistance, serving as the overall estimation of interfacial impedance.

Note: Similar to the overpotential trend in Li∥NMC811 cells (Supplementary FIGS. 30 and 31), the EIS of anode-free Cu∥NMC532 cells showed impedance trend of FDMB˜F6DEE>>F5DEE˜F3DEE>F4DEE.

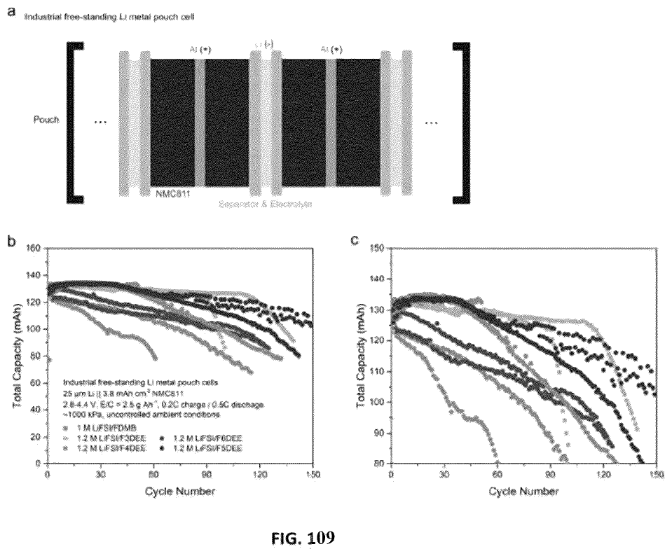

FIG. 109 provides Battery structure (a) and cycling performance (b,c) of 25 μm Li∥3.8 mAh cm−2 NMC811 industrial free-standing pouch cells using fluorinated-DEE electrolytes. (c) is the zoomed-in scale of (b).

Note: The free-standing Li metal foil used in these industrial pouch cells was 50 μm thick but it faced two NMC811 electrodes with its both sides; therefore, we counted it as 25 μm Li 113.8 mAh cm−2 NMC811.

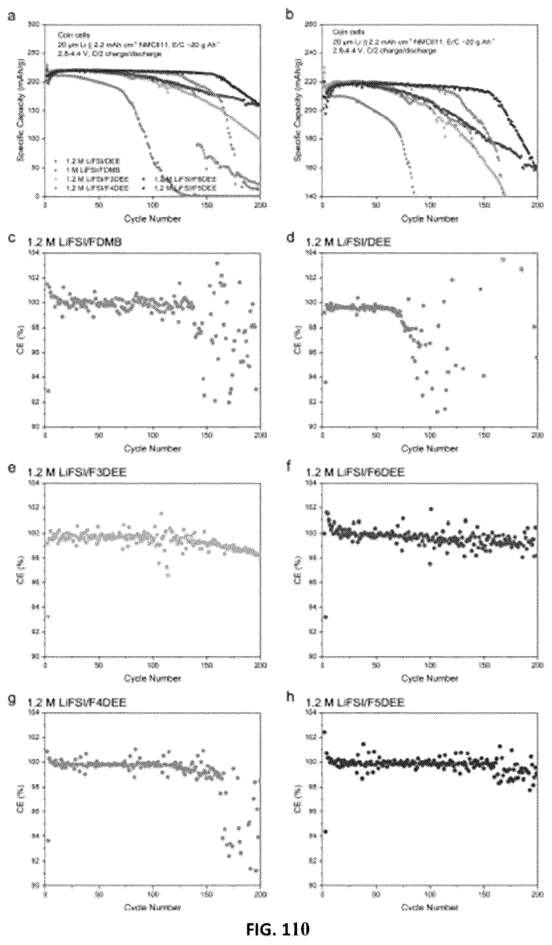

FIG. 110 provides Cycling performance of 20 μm Li∥˜2.2 mAh cm−2 NMC811 coin cells using fluorinated-DEE electrolytes at 0.5 C charge 0.5 C discharge: capacity retention (a,b) and CE (c-h) with cycle number. (b) is the zoomed-in scale of (a).

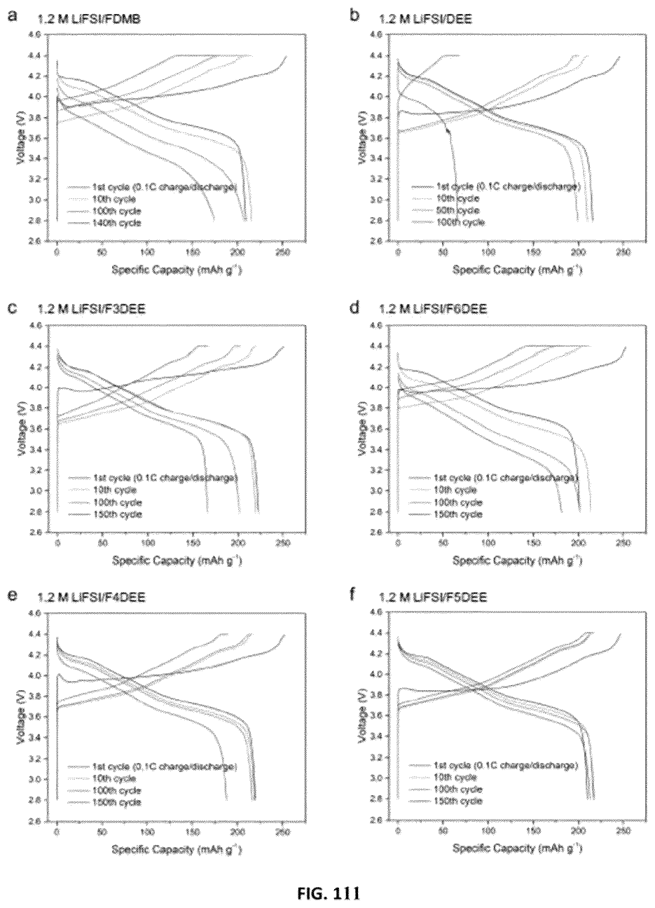

FIG. 111 provides Charge/discharge curves of 20 μm Li∥˜2.2 mAh cm−2 NMC811 coin cells using fluorinated-DEE electrolytes at 0.5 C charge 0.5 C discharge.

FIG. 112 provides Charge/discharge curves of 750 μm Li∥˜2 mAh cm−2 microparticle-LFP coin cells using fluorinated-DEE electrolytes at 0.5 C charge 0.5 C discharge.

FIG. 113 provides Rate capability tests fluorinated-DEE electrolytes using 20 μm Li∥˜2 mAh cm−2 microparticle-LFP coin cells. Symmetric charge and discharge were applied.

FIG. 114 provides Cycling performance of Cu∥˜2.1 mAh cm−2 microparticle-LFP anode-free pouch cells using fluorinated-DEE electrolytes. (a) CE of cells cycled at 0.2 C charge 2 C discharge. (b) CE of cells cycled at 0.5 C charge 2 C discharge. (c,d) Capacity retention and CE of cells cycled at 0.2 C charge 0.3 C discharge. (e,f) Capacity retention and CE of cells cycled at 0.5 C charge 0.5 C discharge.

FIG. 115 provides Optical images of the Cu∥microparticle-LFP pouch cells using 1.2 M LiFSI/F4DEE (left of a and b) and 1.2 M LiFSI/F5DEE (right of a and c) after 140 cycles.

Note: No obvious gassing issue was observed for both electrolytes under different yet fast cycling conditions (0.5 C charge 2 C discharge for F4DEE and 1 C charge 2 C discharge for F5DEE), even though no degassing procedure was implemented after initial cycles.

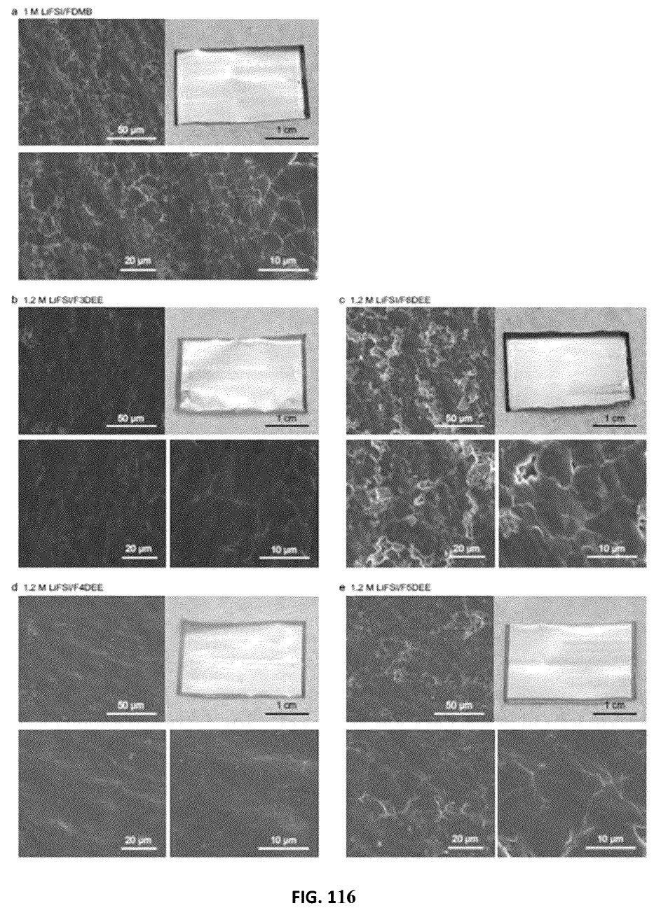

FIG. 116 provides SEM and optical images of the Cu side in Cu∥microparticle-LFP pouch cells cycled at 0.2 C charge 0.3 C discharge for 80 cycles (kept at charged state at last).

FIG. 117 provides SEM and optical images of the Cu side in Cu∥microparticle-LFP pouch cells under fast cycling (kept at charged state at last). (a) 1.2 M LiFSI/F4DEE at 0.5 C charge 2 C discharge for 150 cycles. (b) 1.2 M LiFSI/F5DEE at 0.5 C charge 2 C discharge for 150 cycles. (c) 1.2 M LiFSI/F4DEE at 1 C charge 2 C discharge for 90 cycles. (d) 1.2 M LiFSI/F5DEE at 1 C charge 2 C discharge for 90 cycles.

FIG. 118 provides SEM images of the Cu side in Cu∥NMC532 pouch cells cycled at 0.2 C charge 0.3 C discharge (kept at charged state at last).

FIG. 119 provides XPS O1s depth profiles of cycled Li metal electrodes using fluorinated-DEE electrolytes.

Note: The O1s signals revealed that Li₂O and —SOx species dominated in fluorinated-DEE electrolytes. This feature is consistent with cryo-EDS results and has been reported to be both highly interfacial conductive29,30 and Li metal compatible8,31.

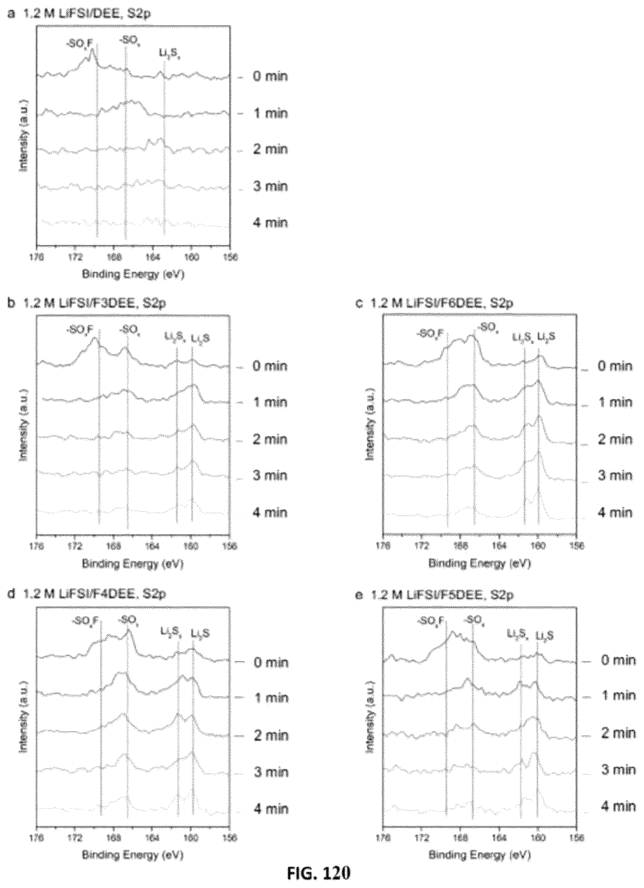

FIG. 120 provides XPS S2p depth profiles of cycled Li metal electrodes using fluorinated-DEE electrolytes.

Note: The S2p signals showed uniformly distributed Li2S and Li2Sx species with depth profiling in fluorinated-DEE electrolytes, indicating anion-derived robust SEIs24,31; by contrast, only trivial S2p signals existed in the DEE electrolyte.

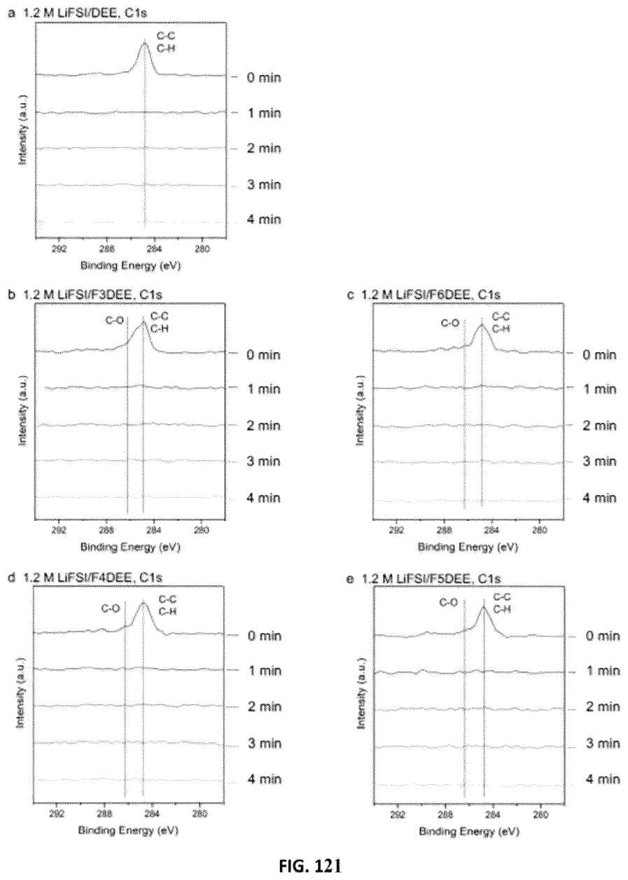

FIG. 121 provides XPS Cis depth profiles of cycled Li metal electrodes using fluorinated-DEE electrolytes.

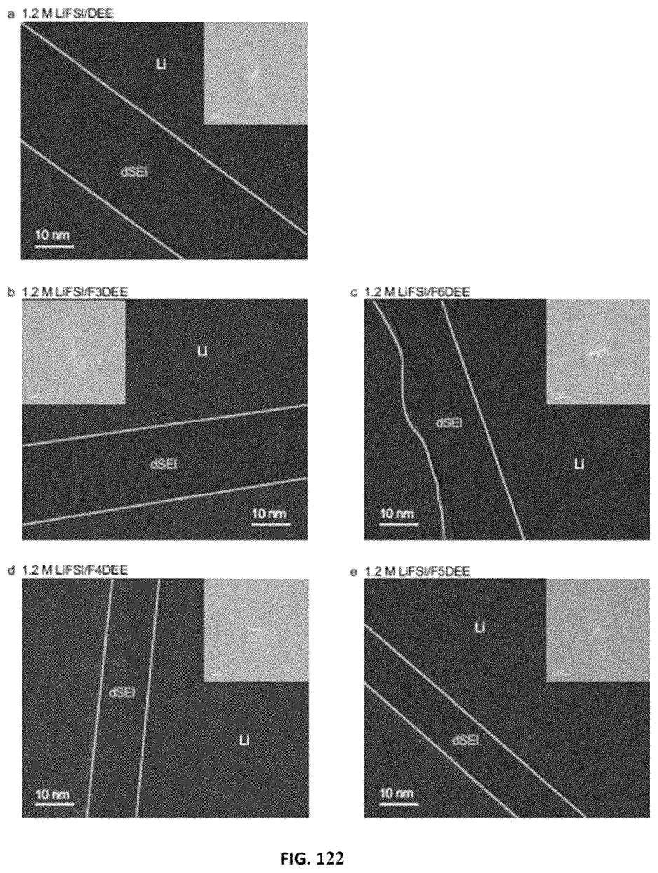

FIG. 122 provides Cryo-TEM images of Li metal deposits using fluorinated-DEE electrolytes.

Note: The direct SEIs (dSEIs) on Li surface in F4DEE and F5DEE electrolytes were thinner than others while that in F6DEE showed wavy structure (non-uniformity). The one in DEE electrolyte was the thickest dSEI. All these facts were consistent with our battery results especially Li metal CE.

FIG. 123 provides Different elemental ratios obtained from cryo-EDS of Li metal deposits using fluorinated-DEE electrolytes.

FIG. 124 provides Cryo-EDS plots of Li metal deposits using fluorinated-DEE electrolytes.

FIG. 125 provides Atomic ratio by XPS with different depths of NMC811 cathodes after 30 cycles.

Note: The atomic ratio of CEI in 1.2 M LiFSI/DEE showed huge fluctuation with depth profiling, and particularly, high Ni content was observed on the initial surface (without sputtering) and after 4-min sputtering, showing the poor passivation of NMC811 in DEE. By contrast, all the fluorinated-DEE and FDMB electrolytes showed high F and C species and negligible Ni content in the CEI, indicating excellent cathode surface protection.

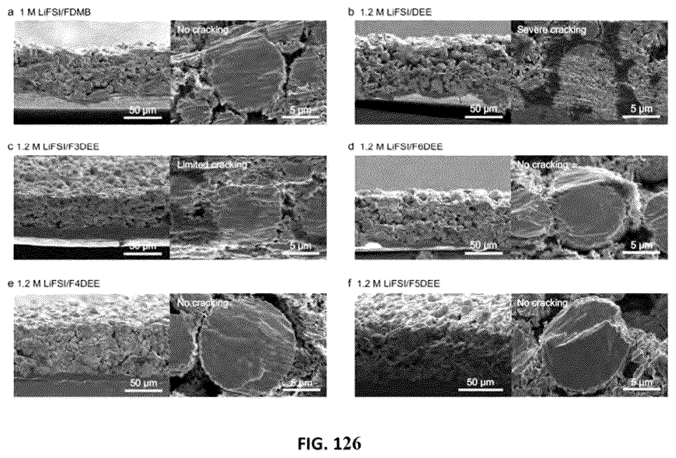

FIG. 126 provides Cross-sectional SEM images of NMC811 cathodes after 30 cycles.

Note: The polycrystalline-NMC811 particles in the 1.2 M LiFSI/DEE electrolyte showed a universal cracking feature and some particles were completely pulverized. By contrast, the cycled NMC811 particles still maintained complete shape or only showed limited cracking in FDMB or fluorinated-DEE electrolytes.

FIG. 127 provides Synthetic scheme of fluorinated-DEEs studied in this work.

FIG. 128 provides 1H-NMR of 2-(2,2-difluoroethoxy)ethanol (400 MHz, CDCl3, δ/ppm): 6.00˜5.70 (tt, 2H), 3.71˜3.60 (m, 6H), 3.05 (s, 1H).

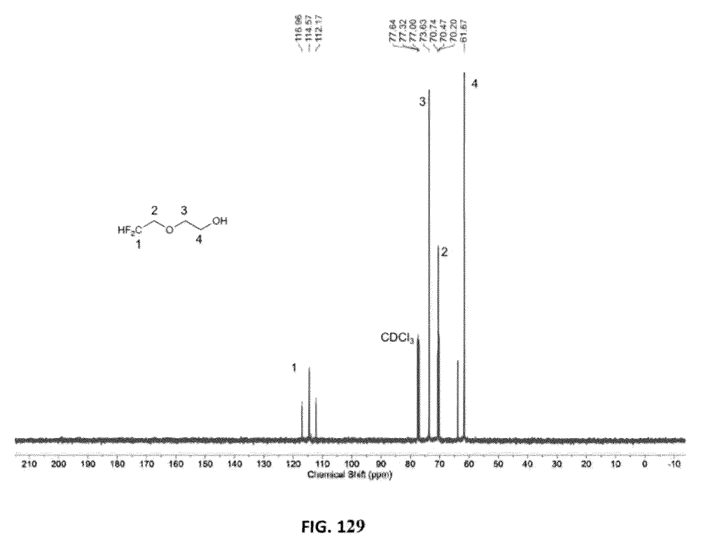

FIG. 129 provides 13C-NMR of 2˜(2,2-difluoroethoxy)ethanol (100 MHz, CDCl3, δ/ppm): 116.96˜112.17, 73.63, 70.74˜70.20, 61.67.

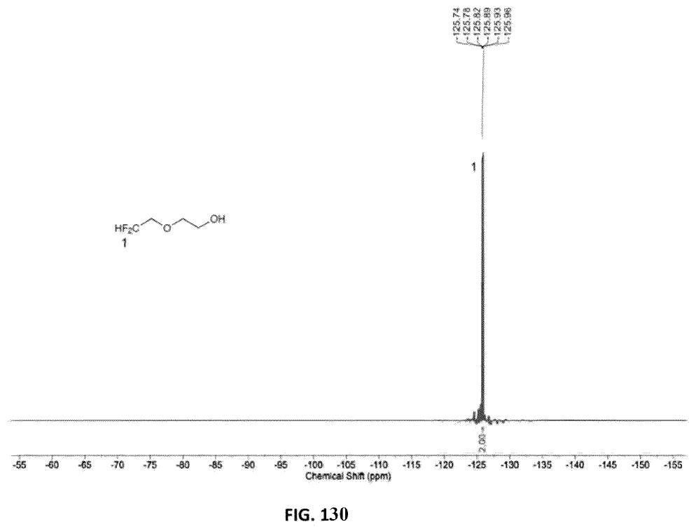

FIG. 130 provides 19F-NMR of 2˜(2,2-difluoroethoxy)ethanol (376 MHz, CDCl3, δ/ppm): −125.74 ˜˜125.96 (dt, 4F).

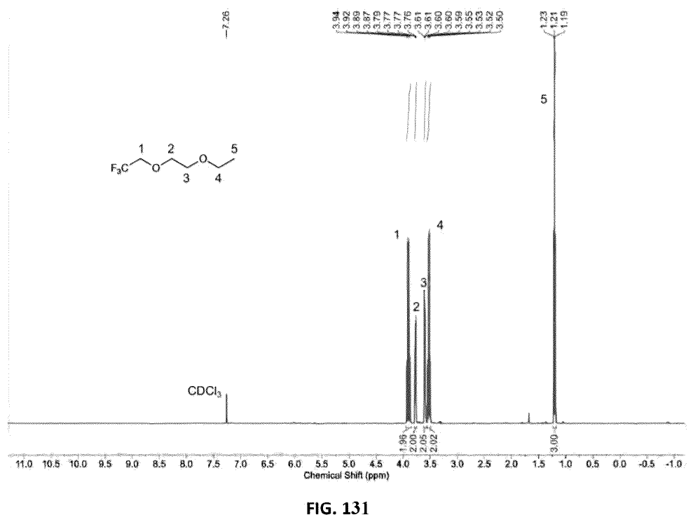

FIG. 131 provides 1H-NMR of F3DEE (400 MHz, CDCl3, δ/ppm): 3.94˜3.87 (q, 2H), 3.77˜3.59 (m, 4H), 3.55˜3.50 (q, 2H), 1.23˜1.19 (3H).

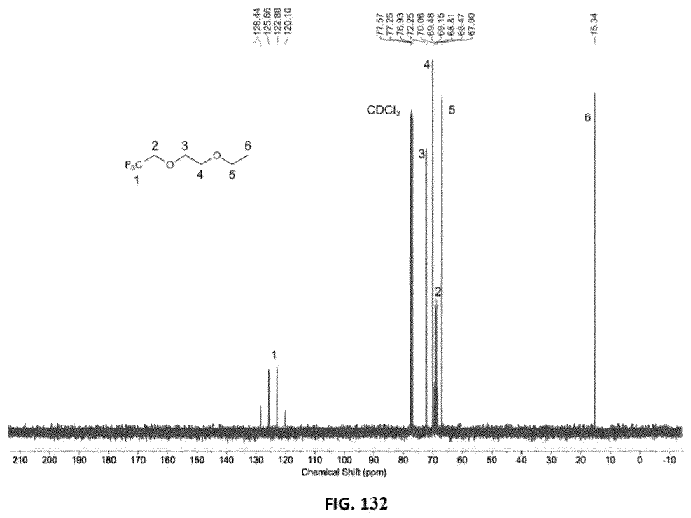

FIG. 132 provides 13C-NMR of F3DEE (100 MHz, CDCl3, δ/ppm): 128.44 ˜120.10, 72.25, 70.06, 69.48˜68.47, 67.00, 15.34.

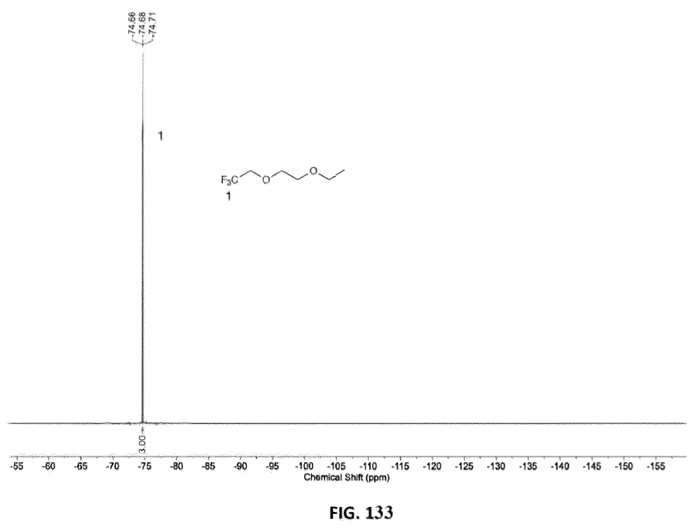

FIG. 133 provides 19F-NMR of F3DEE (376 MHz, CDCl3, δ/ppm): −74.66 ˜-74.71 (t, 3F).

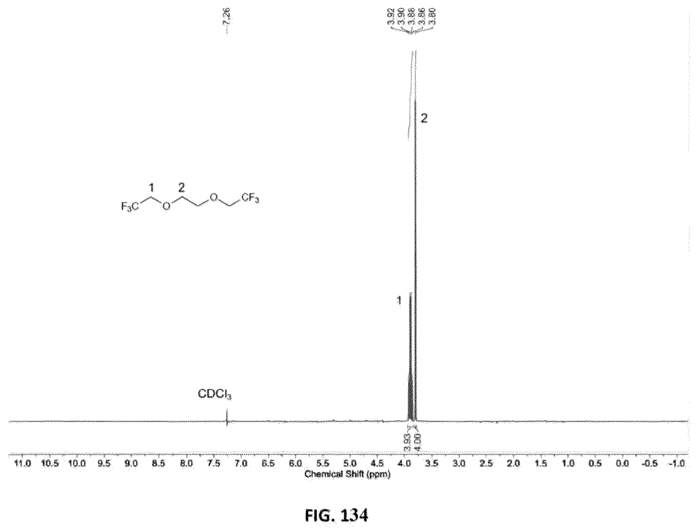

FIG. 134 provides 1H-NMR of F6DEE (400 MHz, CDCl3, δ/ppm): 3.92˜3.86 (q, 4H), 3.80 (s, 4H).

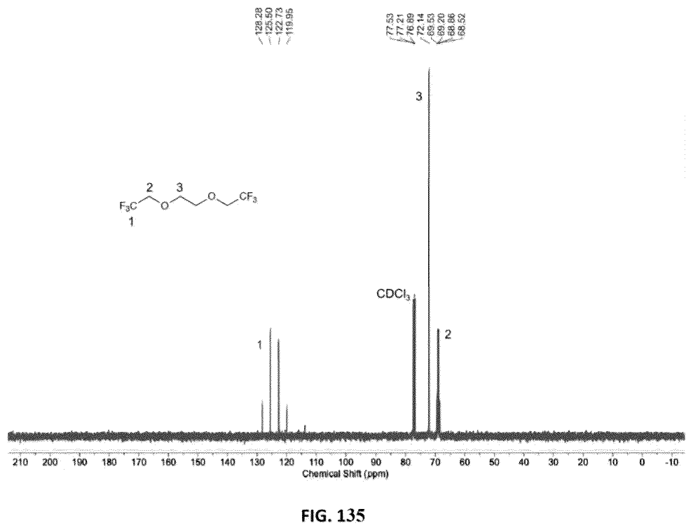

FIG. 135 provides 13C-NMR of F6DEE (100 MHz, CDCl3, δ/ppm): 128.28 ˜119.95, 72.14, 69.53˜68.52.

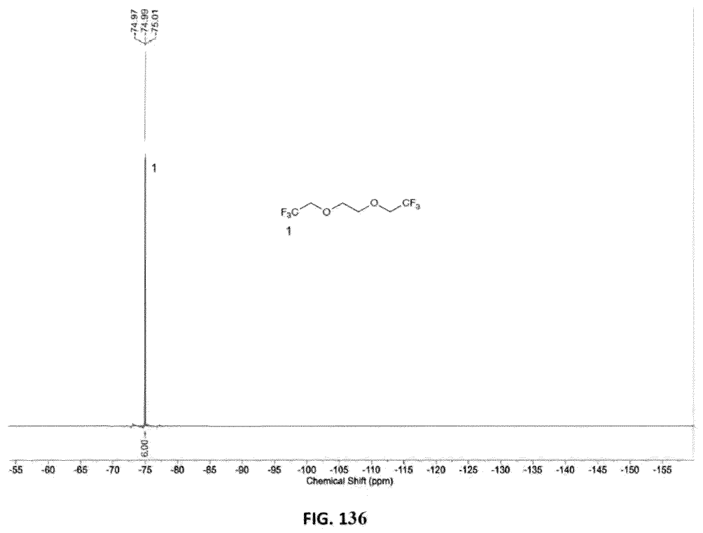

FIG. 136 provides 19F-NMR of F6DEE (376 MHz, CDCl3, δ/ppm): −74.97 ˜−75.01 (t, 6F).

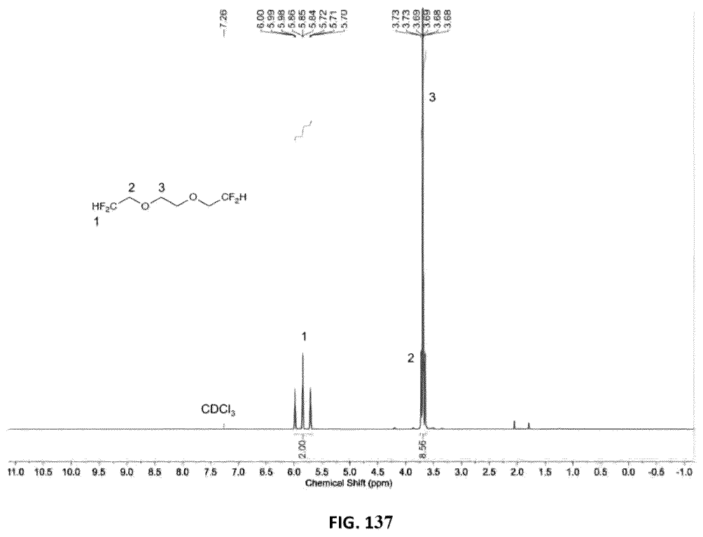

FIG. 137 provides 1H-NMR of F4DEE (400 MHz, CDCl3, δ/ppm): 6.00˜5.70 (tt, 2H), 3.73˜3.68 (td, 4H), 3.69 (s, 4H).

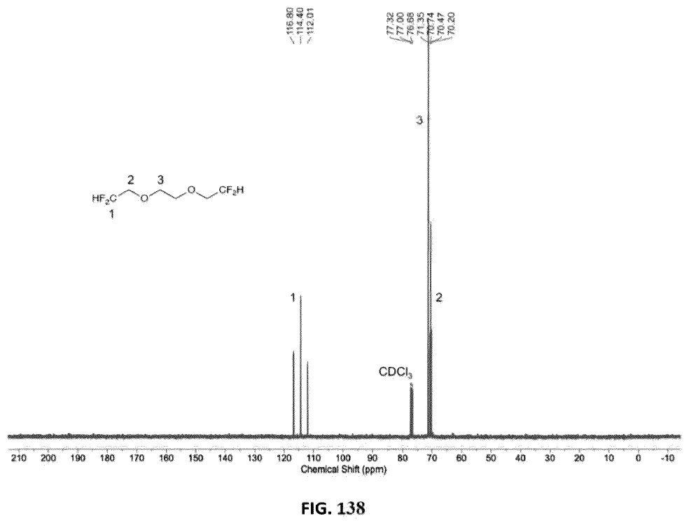

FIG. 138 provides 13C-NMR of F4DEE (100 MHz, CDCl3, δ/ppm): 116.80 ˜112.01, 71.35, 70.74˜70.20.

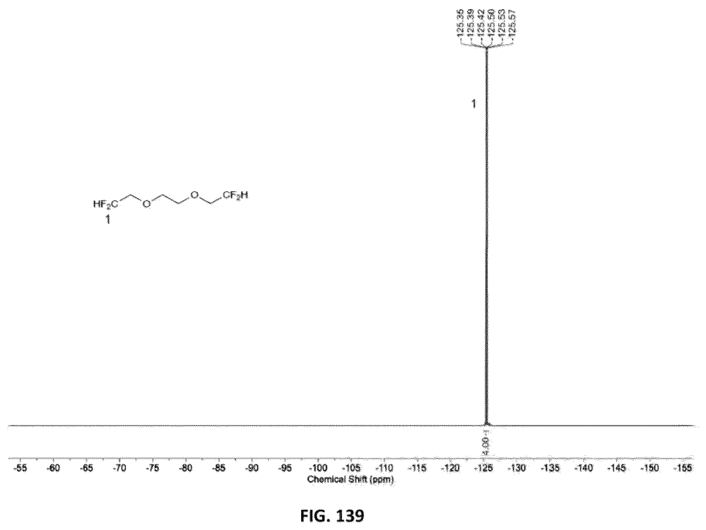

FIG. 139 provides 19F-NMR of F4DEE (376 MHz, CDCl3, δ/ppm): −125.35 ˜−125.57 (dt, 4F).

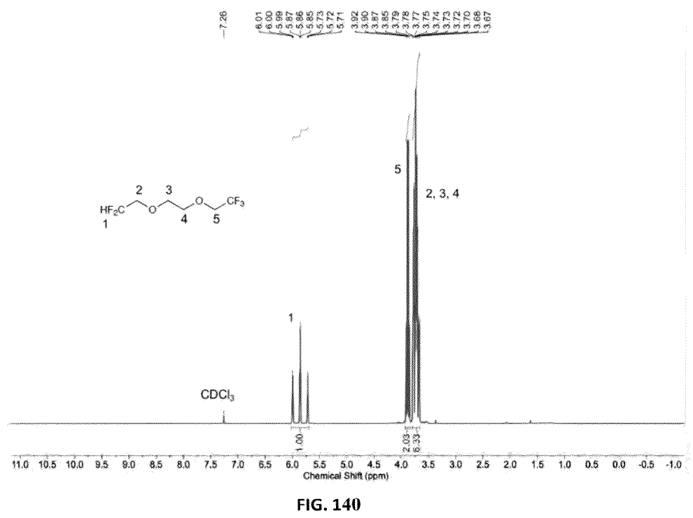

FIG. 140 provides 1H-NMR of F5DEE (400 MHz, CDCl3, δ/ppm): 6.01 ˜5.71 (tt, 1H), 3.92˜3.85 (td, 2H), 3.79˜3.67 (m, 6H).

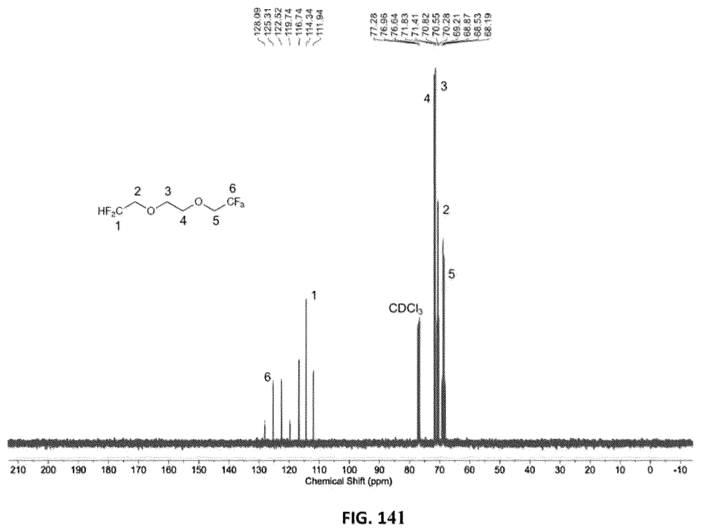

FIG. 141 provides 13C-NMR of F5DEE (100 MHz, CDCl3, δ/ppm): 128.09 ˜119.74, 116.74˜111.94, 71.83, 71.41, 70.82˜70.28, 69.21˜68.19.

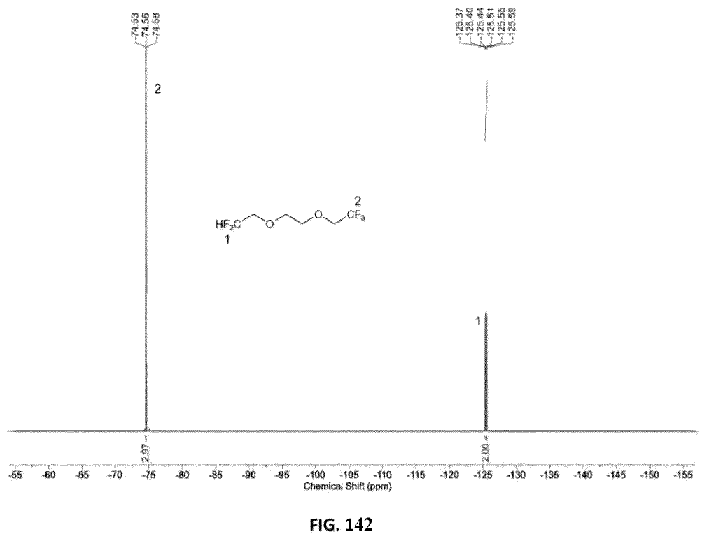

FIG. 142 provides 19F-NMR of F5DEE (376 MHz, CDCl3, δ/ppm): −74.53˜−74.58 (t, 3F), −125.37 ˜−125.59 (dt, 2F).

### II. B. Tuning Fluorination of Linear Carbonate for Lithium-Ion Batteries

### II. B. 1. Abstract

Liquid electrolyte engineering plays a critical role in modern lithium-ion batteries. However, the existing electrolytes fall short when used with some trending battery chemistries such as high-voltage and high-energy-density electrodes. Fluorination of electrolyte solvents has been identified as an effective approach for improved cyclability, but few works systematically studied the effects of fluorination extent of carbonate solvents on battery performance. Here we design and synthesize a family of fluorinated ethyl methyl carbonates. Different numbers of F atoms are finely tuned to yield monofluoroethyl methyl carbonate (F1EMC), difluoroethyl methyl carbonate (F2EMC) and trifluoroethyl methyl carbonate (F3EMC). The cycling behavior of several types of lithium-ion pouch cells, including graphite (Gr)/single-crystalline LiNi_{0.8}Mn_{0.1}Co_{0.1}O₂ (SC-NMC811), Gr-SiOₓ/LiNi_{0.6}Mn_{0.2}Co_{0.2}O₂ (NMC622), high-voltage Gr/LiNi_{0.5}Mn_{1.5}O₄ (LNMO), Gr/layered Li-rich Mn-based oxide (LLMO) and fast-charging Gr/NMC622, were systematically investigated to understand the impact of fluorination degree. Compared to the commercially available F3EMC, we found that the partially-fluorinated F1EMC and F2EMC in some cases showed improved cycling stability, which we attribute to their locally-polar —CH₂F and —CHF₂ groups and thus fast ion conduction than —CF₃. This work suggests that highly or fully fluorinated solvents are not necessarily desirable; instead, fluorination degree needs to be rationally and finely tuned for optimized lithium-ion cell performance.

### II. B. 2. Introduction

Lithium (Li)-ion batteries are the nexus of modern electric power sources (J. B. Goodenough, Y. Kim, Challenges for Rechargeable Li Batteries, Chem. Mater. 22 (2010) 587-603. https://doi.org/10.1021/cm901452z; J.-M. Tarascon, M. Armand, Issues and challenges facing rechargeable lithium batteries, Nature. 414 (2001) 359-367. https://doi.org/10.1038/35104644). They have been widely used in electric vehicles, consumer electronic devices and energy storage grids. Although modern industrial technologies have enabled mass production of high-quality Li-ion batteries, much room still exists for further improving their cycle life, safety and energy density.

Liquid electrolyte engineering (T. R. Jow, K. Xu, O. Borodin, M. Ue, Electrolytes for Lithium and Lithium-Ion Batteries, Springer New York, New York, NY, 2014. https://doi.org/10.1007/978-1-4939-0302-3; K. Xu, Electrolytes and Interphases in Li-Ion Batteries and Beyond, Chem. Rev. 114 (2014) 11503˜11618. https://doi.org/10.1021/cr500003w; J. E. Harlow, X. Ma, J. Li, E. Logan, Y. Liu, N. Zhang, L. Ma, S. L. Glazier, M. M. E. Cormier, M. Genovese, S. Buteau, A. Cameron, J. E. Stark, J. R. Dahn, A Wide Range of Testing Results on an Excellent Lithium-Ion Cell Chemistry to be used as Benchmarks for New Battery Technologies, J. Electrochem. Soc. 166 (2019) A3031-A3044. https://doi.org/10.1149/2.0981913jes) is a pragmatic approach to improve the performances of Li-ion batteries as it is readily incorporated into existing battery manufacturing processes. As the demand for high energy density batteries becomes more pressing, the design and understanding of new electrolytes have become imperative for trending battery chemistries such as Si—C composite anodes, 5 V cathodes, Li-rich cathodes and Co-free cathodes.

Electrolytes are usually composed of Li salts, solvents and additives. While the majority of electrolyte systems uses LiPF₆ as the Li salt due to its overall balanced performance and low cost, the solvents and additives have a wide range of selections to improve cell performances and to meet specific requirements. Additives (S. S. Zhang, A review on electrolyte additives for lithium-ion batteries, J. Power Sources. 162 (2006) 1379-1394. https://doi.org/10.1016/j.jpowsour.2006.07.074) are more intensively investigated since they do not drastically impact the general electrolyte properties. For example, Dahn et al. vastly improved the cycle life of graphite (Gr)/single-crystalline LiNi_{0.5}Mn_{0.3}Co_{0.2}O₂ (SC-NMC532) pouch cells by using additives with a combination of 1% lithium difluorophosphate (LiDFP or LFO, we denoted it as LiDFP hereafter) and 2% fluoroethylene carbonate (FEC) or 1% ethylene sulfate (DTD) and 2% vinylene carbonate (VC) (J. Li, H. Li, W. Stone, S. Glazier, J. R. Dahn, Development of Electrolytes for Single Crystal NMC532/Artificial Graphite Cells with Long Lifetime, J. Electrochem. Soc. 165 (2018) 626-635. https://doi.org/10. I 14912.0971803ies; L. Ma, L. Ellis, S. L. Glazier, X. Ma, J. R. Dahn, Combinations of LiPO2F2 and Other Electrolyte Additives in Li[Ni0.5Mn0.3Co0.2]O2/Graphite Pouch Cells, J. Electrochem. Soc. 165 (2018) A1718-A1724. https://doi.org/10.1149/2.0661809jes; W. Song, J. Harlow, E. Logan, H. Hebecker, M. Coon, L. Molino, M. Johnson, J. Dahn, M. Metzger, A Systematic Study of Electrolyte Additives in Single Crystal and Bimodal LiNi0.8Mn0.1Co0.1O2/Graphite Pouch Cells, J. Electrochem. Soc. 168 (2021) 090503. https://doi.org/10.1149/1945-7111/ac1e55). Lucht et al. studied lithium bis(trimethylsilyl) phosphate as an electrolyte additive for low-temperature Gr/LiNi_{0.8}Mn_{0.1}Co_{0.1}O₂ (NMC811) cells. Although a lot of reports (R. Petibon, J. Harlow, D. B. Le, J. R. Dahn, The use of ethyl acetate and methyl propanoate in combination with vinylene carbonate as ethylene carbonate-free solvent blends for electrolytes in Li-ion batteries, Electrochim. Acta. 154 (2015) 227˜234. https://doi.org/10.1016/j.electacta.2014.12.084; J. Li, H. Li, X. Ma, W. Stone, S. Glazier, E. Logan, E. M. Tonita, K. L. Gering, J. R. Dahn, Methyl Acetate as a Co-Solvent in NMC532/Graphite Cells, J. Electrochem. Soc. 165 (2018) A1027-A1037. https://doi.org/10.1149/2.0861805jes; N.D. Rodrigo, S. Tan, Z. Shadike, E. Hu, X.-Q. Yang, B. L. Lucht, Improved Low Temperature Performance of Graphite/Li Cells Using Isoxazole as a Novel Cosolvent in Electrolytes, J. Electrochem. Soc. 168 (2021) 070527. https://doi.org/0.1149/1945-7111/ac11a6; Q. Zheng, Y. Yamada, R. Shang, S. Ko, Y. Lee, K. Kim, E. Nakamura, A. Yamada, A cyclic phosphate-based battery electrolyte for high voltage and safe operation, Nat. Energy. 5 (2020) 291-298. https://doi.org/10.1038/s41560-020-0567-z) showed the feasibility of various chemicals as the electrolyte solvents, carbonates are still among the most widely used solvents for Li-ion battery electrolytes due to their compatibility with Gr anode to allow reversible Li intercalation/deintercalation. Rationally fine tuning of carbonate molecules, therefore, can be an effective way to improve the performance while minimizing detrimental side effects for Li-ion cells.

Recent works (H. Wang, Z. Yu, X. Kong, S. C. Kim, D. T. Boyle, J. Qin, Z. Bao, Y. Cui, Liquid electrolyte: The nexus of practical lithium metal batteries, Joule. 6 (2022) 588-616. https://doi.org/10.1016/j.joule.2021.12.018; N. Aspern, G.-V. Röschenthaler, M. Winter, I. Cekic-Laskovic, Fluorine and Lithium: Ideal Partners for High-Performance Rechargeable Battery Electrolytes, Angew. Chemie Int. Ed. 58 (2019) 15978-16000. https://doi.org/10.1002/anie.201901381; B. Flamme, G. Rodriguez Garcia, M. Weil, M. Haddad, P. Phansavath, V. Ratovelomanana-Vidal, A. Chagnes, Guidelines to design organic electrolytes for lithium-ion batteries: Environmental impact, physicochemical and electrochemical properties, Green Chem. 19 (2017) 1828-1849. https://doi.org/10.1039/c7gc00252a; Y. Zhang, V. Viswanathan, Design Rules for Selecting Fluorinated Linear Organic Solvents for Li Metal Batteries, J. Phys. Chem. Lett. 12 (2021) 5821-5828. https://doi.org/10.1021/acs.ipclett.1c01522; C.-C. Su, M. He, J. Shi, R. Amine, Z. Yu, L. Cheng, J. Guo, K. Amine, Principle in developing novel fluorinated sulfone electrolyte for high voltage lithium-ion batteries, Energy Environ. Sci. 14 (2021) 3029-3034. https://doi.org/10.1039/D0EE03890C; Z. Yu, P. E. Rudnicki, Z. Zhang, Z. Huang, H. Celik, S. T. Oyakhire, Y. Chen, X. Kong, S. C. Kim, X. Xiao, H. Wang, Y. Zheng, G. A. Kamat, M. S. Kim, S. F. Bent, J. Qin, Y. Cui, Z. Bao, Rational solvent molecule tuning for high-performance lithium metal battery electrolytes, Nat. Energy. 7 (2022) 94-106. https://doi.org/10.1038/s41560-021-00962-y) started to investigate a less-explored area, the effect of solvent molecule fluorination degree on electrolyte performances. While fluorinated cyclic carbonates were recently studied theoretically and experimentally (M. Bolloli, F. Alloin, J. Kalhoff, D. Bresser, S. Passerini, P. Judeinstein, J. C. Lepretre, J. Y. Sanchez, Effect of carbonates fluorination on the properties of LiTFSI-based electrolytes for Li-ion batteries, Electrochim. Acta. 161 (2015) 159-170. https://doi.org/10.1016/j.electacta.2015.02.042; M. He, C.-C. Su, C. Peebles, Z. Zhang, The Impact of Different Substituents in Fluorinated Cyclic Carbonates in the Performance of High Voltage Lithium-Ion Battery Electrolyte, J. Electrochem. Soc. 168 (2021) 010505. https://doi.org/10.1 149/1945-7111/abd44b; Y. Zhang, V. Viswanathan, Not All Fluorination Is the Same: Unique Effects of Fluorine Functionalization of Ethylene Carbonate for Tuning Solid-Electrolyte Interphase in Li Metal Batteries, Langmuir. 36 (2020) 11450-11466. https://doi.org/10.1021/acs.langmuir.0c01652), systematic work on linear carbonates is lacking (Y. Sasaki, Organic Electrolytes of Secondary Lithium Batteries, Electrochemistry. 76 (2008) 2-15. https://doi.org/10.5796/electrochemistry.76.2). As one of the most widely-used linear carbonate solvent, ethyl methyl carbonate (EMC) is a perfect candidate for fine tuning its fluorination degree and studying the structure-property relationships. It is noted that trifluoroethyl methyl carbonate (F3EMC) has been widely used as a solvent or additive in modern Li-ion and Li metal batteries (X. Fan, L. Chen, O. Borodin, X. Ji, J. Chen, S. Hou, T. Deng, J. Zheng, C. Yang, S.-C. Liou, K. Amine, K. Xu, C. Wang, Non-flammable electrolyte enables Li-metal batteries with aggressive cathode chemistries, Nat. Nanotechnol. 13 (2018) 715-722. https://doi.org/10.1038/s41565-018-0183-2; Z. Zhang, L. Hu, H. Wu, W. Weng, M. Koh, P. C. Redfern, L. A. Curtiss, K. Amine, Fluorinated electrolytes for 5 V lithium-ion battery chemistry, Energy Environ. Sci. 6 (2013) 1806. https://doi.org/10.1039/c3ee24414h; D. Hubble, D. E. Brown, Y. Zhao, C. Fang, J. Lau, B. D. McCloskey, G. Liu, Liquid electrolyte development for low-temperature lithium-ion batteries, Energy Environ. Sci. (2022) https://doi.org/10.1039/D1EE01789F); however, few reports to date systematically synthesized and studied the electrolytes using monofluoroethyl methyl carbonate (F1EMC) (M. Takehara, N. Tsukimori, N. Nanbu, M. Ue, Y. Sasaki, Physical and Electrolytic Properties of Fluoroethyl Methyl Carbonate, Electrochemistry. 71 (2003) 1201-1204. https://doi.org/10.5796/electrochemistry.71.1201) and difluoroethyl methyl carbonate (F2EMC).

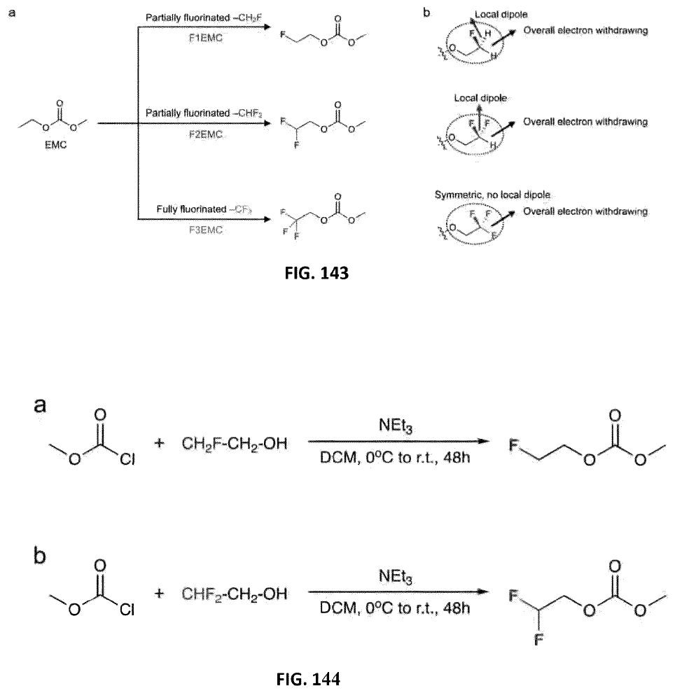

FIG. 143 illustrates Molecular structures of fluorinated-EMCs (a) and schemes to show local and overall dipoles (b).

Herein, we design and synthesize F1EMC and F2EMC as the electrolyte solvents to explore the impact of fluorination degree of linear carbonate on the electrolyte properties and cell performance (FIG. 143a). By finely tuning the number of F atoms, the locally polar —CH₂F and —CHF₂ groups on F1EMC and F2EMC (FIG. 143b), respectively, were found to enable favored Li⁺ ion solvation and faster ion conduction compared to the fully fluorinated —CF₃ group on the commercial and commonly-used F3EMC. We choose different types of state-of-the-art Li-ion pouch cells to demonstrate the effects of different fluorinated-EMCs on cycling behaviors. These cells cover a broad range of interests such as 4.4 V Gr/SC-NMC811, Gr-SiOₓ/LiNi_{0.6}Mn_{0.2}Co_{0.2}O₂ (NMC622), high-voltage Gr/LiNi_{0.5}Mn_{1.5}O₄ (LNMO), Gr/layered Li-rich Mn-based oxide (LLMO) and fast-charging Gr/NMC622. We found that in most cases, F1EMC and F2EMC outperformed F3EMC or control electrolytes, 1 M LiPF₆ in ethylene carbonate/ethyl methyl carbonate (EC/EMC, 3/7 by volume) (LP57). This work suggests that the commonly used, fully fluorinated —CF₃ group may not always be the best choice; instead, partially fluorinated, locally polar —CH₂F and —CHF₂ groups may provide improved battery performance (FIG. 143b). Most importantly, fine tuning of molecular design is an effective method to realize optimal battery performance.

### II. B. 3. Experimental

General materials and pouch cells. 2-Fluoroethanol was purchased from Matrix Scientific. 2,2-Difluoroethanol was purchased from SynQuest. Methyl chloroformate, triethyl amine and other general reagents and solvents were purchased from Sigma-Aldrich and Fisher Scientific. LiPF₆ and lithium difluoro(oxalato)borate (LiDFOB) were purchased from MSE Supplies. Battery-grade EMC was purchased from Sigma-Aldrich. Lithium bis(fluorosulfonyl)imide (LiFSI), LiDFP and F3EMC were purchased from Guangdong Canrd New Energy Technology. The commercial carbonate electrolyte LP57 and FEC were purchased from Gotion. All chemicals were used without further purification. The commercial battery separator Celgard 3501 (25 μm thick, surfactant coated for wettability, polypropylene/polyethylene/polypropylene) was purchased from Celgard and used in all coin cells. Thick Li foils (˜750 μm thick) were purchased from Alfa Aesar. Al current collector (25 μm thick) was purchased from MTI. Industrial dry Gr/SC-NMC811, Gr-SiOₓ/NMC622, Gr/LNMO, Gr/LLMO and Gr/NMC622 pouch cells were purchased from Li-Fun Technology (see Table 1 for detailed pouch cell information provided by the vendor).

TABLE 1
Pouch cell specifications.

| | | Gr/SC-NMC811 | Gr-SiOₓ/NMC622 | | | |
|---|---|---|---|---|---|---|
| | | Artificial graphite | 80% AG + 20% | Gr/LNMO | Gr/LLMO | Gr/NMC622 |
| Anode | Material | (AG) | SiOₓ (~550 mAh g⁻¹) | AG | AG | AG |
| | Active material ratio | 94.8% | 94.8% | 95.7% | 94.8% | 97% |
| | Loading (mg cm⁻²) | 10 | 6.3 | 7.2 | 11 | 8.7 |
| | Press density (g/cc) | 1.5 | 1.5 | 1.5 | 1.5 | 1.6 |
| | | | | | Layered Li-rich | |
| | | Single-crystalline | Poly-crystalline | | Mn-based oxide | Poly-crystalline |
| Cathode | Material | LiNi_{0.8}Mn_{0.1}Co_{0.1}O₂ | LiNi_{0.6}Mn_{0.2}Co_{0.2}O₂ | LiNi_{0.5}Mn_{1.5}O₄ | (~245 mAh g⁻¹) | LiNi_{0.6}Mn_{0.2}Co_{0.2}O₂ |
| | Active material ratio | 95.5% | 96.4% | 94% | 96% | 97.5% |
| | Loading (mg cm⁻²) | 13.5 | 18.7 | 15.8 | 12.5 | 15.3 |
| | Press density (g/cc) | 3.3 | 3.3 | 2.7 | 2.5 | 3.3 |

| | | | | | |
|---|---|---|---|---|---|
| Total capacity at 0.1C (mAh) | 200 | 200 | 220 | 140 | 350 |
| Electrolyte amount (mL) | 0.5 | 0.5 | 0.5 | 0.5 | 0.7 |

Syntheses. F1EMC (FIGS. 144a and 156-158): To a 1000 mL round bottom flask were added 50 g 2-fluoroethanol, 95 g triethyl amine (NEt₃) and 400 mL anhydrous dichloromethane (DCM), and the solution was cooled to 0° C. by ice bath to stir for 10 min. Then 80 g methyl chloroformate was mixed with 50 mL anhydrous DCM and the mixture was added dropwise into the flask. After completing the addition, the ice bath was removed to allow the suspension to warm up to room temperature. The reaction was stirred at room temperature for 48 h. After the completion of reaction, 200 mL deionized water was slowly added into the suspension to dissolve all solids. The DCM layer was separated and washed with brine, dried by anhydrous MgSO₄, and the solvents were removed under vacuum. The crude product underwent vacuum distillation (˜65° C. under ˜1 kPa) three times to yield ˜75 g colorless liquid as the product. Yield: 83%. ¹H-NMR (400 MHz, CDCl3, δ/ppm): 4.67-4.53 (m, 2H), 4.41˜4.31 (m, 2H), 3.79 (s, 3H). ¹³C-NMR (100 MHz, CDCl3, δ/ppm): 155.51, 81.80-80.10, 66.72-66.52, 54.92. ¹⁹F-NMR (376 MHz, CDCl3, δ/ppm): −225.08-−225.48 (m, 1F).

F2EMC (FIGS. 144b and 159-161): To a 1000 mL round bottom flask were added 82 g 2,2-difluoroethanol, 110 g NEt₃ and 400 mL anhydrous DCM, and the solution was cooled to 0° C. by ice bath to stir for 10 min. Then 100 g methyl chloroformate was mixed with 100 mL anhydrous DCM and the mixture was added dropwise into the flask. After completing the addition, the ice bath was removed to allow the suspension to warm up to room temperature. The reaction was stirred at room temperature for 48 h. After the completion of reaction, 200 mL deionized water was slowly added into the suspension to dissolve all solids. The DCM layer was separated and washed with brine, dried by anhydrous MgSO₄, and the solvents were removed under vacuum. The crude product underwent vacuum distillation (˜65° C. under ˜1 kPa) three times to yield ˜71 g colorless liquid as the product. Yield: 51%. ¹H-NMR (400 MHz, CDCl3, δ/ppm): 6.08-5.79 (tt, 1H), 4.33-4.25 (td, 2H), 3.80 (s, 3H). ¹³C-NMR (100 MHz, CDCl3, δ/ppm): 155.00, 114.77-109.97, 65.69-65.09, 55.30. ¹⁹F-NMR (376 MHz, CDCl3, δ/ppm): −126.46-−126.68 (dt, 2F).

FIG. 144 illustrates: Synthetic procedures of F1EMC (a) and F2EMC (b).

Electrolytes. After the syntheses, F1EMC and F2EMC were mixed with 10 w.t. % activated molecular sieves and stored in argon-filled glovebox (Vigor, oxygen <0.5 ppm, water <0.1 ppm) at room temperature. The water contents of F1EMC and F2EMC measured by Karl-Fisher titration were ˜70 ppm and ˜50 ppm, respectively. The developed electrolytes used in this work were listed as follows: 1 M LiPF₆ in FEC/EMC (3/7 by volume), 1 M LiPF₆ in FEC/F1EMC (3/7 by volume), 1 M LiPF₆ in FEC/F2EMC (3/7 by volume), 1 M LiPF₆ in FEC/F3EMC (3/7 by volume), 1 M LiPF₆ in FEC/F2EMC (3/7 by volume)+1% LiDFP (by weight), 1 M LiPF₆ in FEC/F2EMC (3/7 by volume)+1% LiDFOB (by weight), 1 M LiFSI in FEC/F1EMC (3/7 by volume)+2% LiDFOB (by weight), and 1 M LiFSI in FEC/F2EMC (3/7 by volume)+2% LiDFOB (by weight). The control electrolytes used in this work were listed as follows: LP57 and LP57+5% FEC (by weight). All the electrolytes were prepared and stored in argon-filled glovebox at room temperature. The electrolytes were used as soon as they were prepared (usually within 48 h). Since we used 1 M main salt and FEC/fluorinated-EMC=3/7 volume ratio in this work, the molarity, solvent ratio and “%” symbol of the electrolytes will be removed in the figures for abbreviation. The solubility limits were roughly evaluated by dissolving 4 mmol LiPF₆ (˜610 mg) in 1 mL fluorinated EMCs: LiPF₆ was fully or almost fully dissolved in EMC, F1EMC and F2EMC, while precipitate was observed in F3EMC.

Electrochemical characterizations. All electrochemical tests were carried out in a Swagelok cell or 2032-type coin cell configuration. All cells were fabricated in an argon-filled glovebox. The electrochemical impedance spectroscopy (EIS) and cyclic voltammetry (CV) were carried out on a Biologic VMP3 system. The EIS measurements were taken over a frequency range of 1 MHz to 100 mHz. The anodic CV tests were done at a rate of 1 mV s-1 over a voltage range of 3.0 to 5.5 V in Li/Al cells.

Pouch cell cycling. Industrial dry pouch cells were quickly cut under ambient conditions, immediately transferred into the argon-filled glovebox, and used without further drying. The electrolyte was injected into the dry pouch cell in the argon-filled glovebox, and the electrolyte-to-capacity (EC) ratio was controlled to be 2˜3 mL Ah-1. After resting for several hours in the glovebox to allow good wetting of the electrolyte, the pouch cell was quickly transferred out and sealed by pouch cell vacuum sealer. The cycling tests were carried out on Arbin or LAND. The cells were held at 1.5 V for 3 h before activation cycles. Unless specially listed, a constant-current-constant-voltage (CC-CV) protocol was used for cycling: Cells were charged to top voltage and then held at that high voltage until the current dropped below 0.1 C. All cells were clamped in C-shape clamps with a slightly tight yet uncontrolled pressure and cycled under ambient conditions without temperature control. Gr/SC-NMC811: After the first two activation cycles at 0.1 C charge/discharge, the cells were cycled at 1 C charge/discharge between 3.0 and 4.4 V. Gr-SiOₓ/NMC622: After the first two activation cycles at 0.1 C charge/discharge, the cells were cycled at 1 C charge/discharge between 3.0 and 4.2 V. Gr/LNMO: After the first two activation cycles at 0.1 C charge/discharge, the cells were completely degassed and then cycled at 1 C or 0.3 C charge/discharge between 3.5 and 4.9 V or between 3.5 and 4.7 V. Only when using 0.3 C charge/discharge between 3.5 and 4.9 V, no constant-voltage was applied at 4.9 V higher cutoff. Gr/LLMO: After the first three activation cycles at 0.1 C charge/discharge, the cells were cycled at 0.5 C charge/discharge between 3.0 and 4.8 V. Gr/NMC622: After the first three activation cycles at 0.1 C charge/discharge and the second three activation cycles at 0.5 C charge/discharge, the cells were completely degassed and then cycled at 6 C charge 0.5 C discharge between 3.0 and 4.1 V. In this fast-charging protocol, cells were charged to 4.1 V and then held at 4.1 V until the current dropped below 1 C.

Density functional theory (DFT) calculations. The molecular geometries for the ground states were optimized by DFT at the B3LYP/6-311G+(d, p) level, and then the energy and electrostatic potentials (ESPs) of molecules were evaluated at the B3LYP/6-311G+(d, p) level as well. All DFT calculations were carried out with Gaussian 16 on Sherlock server at Stanford University.

General material characterizations. ¹H—, ¹³C—and ¹⁹F-NMR spectra were recorded on a Varian Mercury 400 MHz NMR spectrometer and ⁷Li-NMR spectra were recorded on a UI 500 MHz NMR spectrometer at room temperature. Thermo Fisher Scientific Apreo S LoVac was used for taking scanning electron microscope (SEM) images and performing energy dispersive X-ray spectra (EDS). For X-Ray photoelectron spectroscopy (XPS) measurements, the electrodes were washed with dimethyl carbonate (DMC) for 30 s to remove the remaining electrolytes, and then the samples were transferred and sealed into the XPS holder in the argon-filled glovebox. The XPS profiles were collected with a PHI VersaProbe 3 scanning XPS microprobe. The depth sputtering condition is 2 kV, 1.5 μA, 2*2 mm.

### II. B. 4. Results and Discussion

Molecular design. Fully or highly fluorinated solvents usually have low salt dissolution and sluggish ionic transport, while partially fluorinated —CHF₂ group was recently proved to be beneficial for high ionic conductivity of ether-based electrolytes. We herein extend and implement this design logic in linear carbonate solvents. As shown in FIG. 143b, molecular geometries show that the partially fluorinated —CH₂F and —CHF₂ groups are locally polar while the fully fluorinated —CF₃ group is symmetric without local dipole, although all these fluorinated ethoxy groups show an overall electron withdrawing property for the molecules. This argument is supported by DFT calculations and ⁷Li-NMR as below.

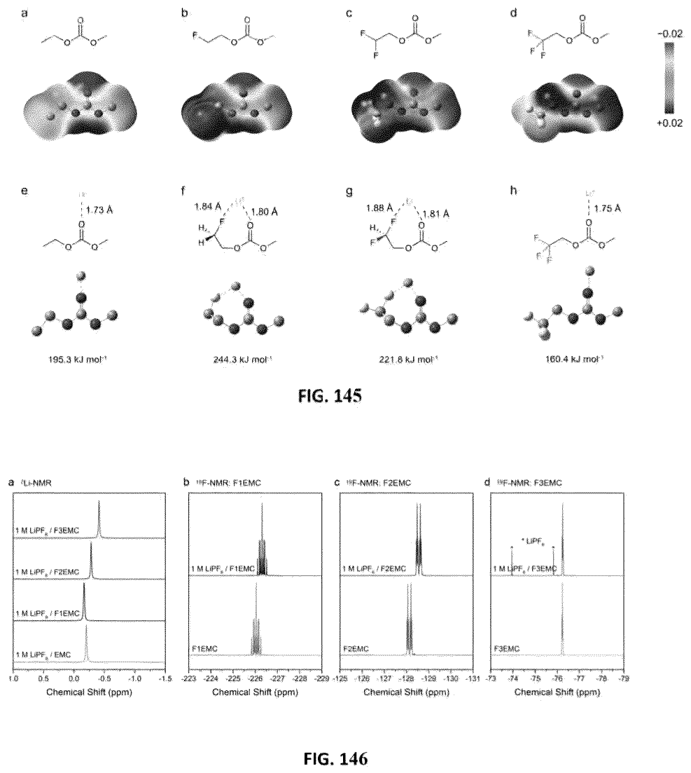

FIGS. 145a-d shows the electrostatic potential (ESP) distribution of fluorinated-EMC molecules. For EMC, the negative charges were only concentrated on O atoms especially C═O group; by contrast, negative charges were also observed on F atoms of fluorinated-EMCs. Careful analysis revealed fine difference between different fluorinated-EMCs. At the same iso-potential scale, the charges on the F atoms of F1EMC and F2EMC were more negative (darker red) than that of F3EMC, indicating stronger Li⁺ coordination ability of F on F1EMC and F2EMC. These ESP results are consistent with the DFT-optimized coordination structures of Li⁺-fluorinated-EMCs (FIGS. 145e-h). While in Li⁺-EMC and Li⁺-F3EMC cases only the C═O group coordinates with Li⁺, the F atom of F1EMC and F2EMC participates in the Li⁺ coordination to form the chelating structure with C═O, indicating a stronger solvation capability of F1EMC and F2EMC. Similar Li—F coordination phenomenon has been observed before (Z. Yu, H. Wang, X. Kong, W. Huang, Y. Tsao, D. G. Mackanic, K. Wang, X. Wang, W. Huang, S. Choudhury, Y. Zheng, C. V Amanchukwu, S. T. Hung, Y. Ma, E. G. Lomeli, J. Qin, Y. Cui, Z. Bao, Molecular design for electrolyte solvents enabling energy-dense and long-cycling lithium metal batteries, Nat. Energy. 5 (2020) 526-533. https://doi.org/10.1038/s41560-020-0634-5; Z. Yu, D. G. Mackanic, W. Michaels, M. Lee, A. Pei, D. Feng, Q. Zhang, Y. Tsao, C. V. Amanchukwu, X. Yan, H. Wang, S. Chen, K. Liu, J. Kang, J. Qin, Y. Cui, Z. Bao, A Dynamic, Electrolyte-Blocking, and Single-Ion-Conductive Network for Stable Lithium-Metal Anodes, Joule. 3 (2019) 2761-2776. https://doi.org/10.1016/j.joule.2019.07.025; H. Wang, Z. Yu, X. Kong, W. Huang, Z. Zhang, D. G. Mackanic, X. Huang, J. Qin, Z. Bao, Y. Cui, Dual-Solvent Li-Ion Solvation Enables High-Performance Li-Metal Batteries, Adv. Mater. 33 (2021) 2008619. https://doi.org/10.1002/adma.202008619; P. Ma, P. Mirmira, C. V Amanchukwu, Effect of Building Block Connectivity and Ion Solvation on Electrochemical Stability and Ionic Conductivity in Novel Fluoroether Electrolytes, ACS Cent. Sci. 7 (2021) 1232-1244. https://doi.org/10.1021/acscentsci.1c00503; G. Zhang, X. Deng, J. Li, J. Wang, G. Shi, Y. Yang, J. Chang, K. Yu, S.-S. Chi, H. Wang, P. Wang, Z. Liu, Y. Gao, Z. Zheng, Y. Deng, C. Wang, A bifunctional fluorinated ether co-solvent for dendrite-free and long-term lithium metal batteries, Nano Energy. 95 (2022) 107014. https://doi.org/10.1016/j.nanoen.2022.107014).

FIG. 145 provides ESP distribution of fluorinated-EMCs (a-d) and coordination structures and binding energies of Li⁺-fluorinated-EMCs (e-h) calculated by DFT.

The findings above are cross-validated by ⁷Li—and ¹⁹F-NMR (FIG. 146). Here FEC was removed and 1 M LiPF₆ was solely dissolved in each fluorinated-EMC to clearly show the influences on solvation. The ⁷Li-NMR showed upfield shift from 1 M LiPF₆ in F1EMC or EMC to F2EMC to F3EMC, indicating weaker solvation capability with the increasing fluorination degree (FIG. 146a). The ⁷Li in F1EMC showed tiny downfield shift compared to that in EMC, which can be attributed to the deshielding effect of F atom on F1EMC. The Li—F interaction between Li⁺ and F atoms on F1EMC and F2EMC was verified by ¹⁹F-NMR in which upfield ¹⁹F peak shift was observed when LiPF₆ was dissolved in F1EMC or F2EMC (FIGS. 146b and c). By contrast, ¹⁹F signal stayed at almost the same position for F3EMC before and after dissolving LiPF₆, confirming that fully fluorinated —CF₃ group did not strongly interact with Li⁺ ions. The NMR results well supported our molecular design.

The above studies are focused on the interactions between fluorinated-EMCs and Li⁺ ions; however, it is noteworthy that the electrolytes we developed herein contain high-content FEC co-solvent which also participates in the solvation, thus affecting electrical double layer, solid-electrolyte interphase (SEI), and battery performance. Detailed studies (especially multiple characterization tools for cross-validation) need to be conducted for these electrolytes and their future derivatives.

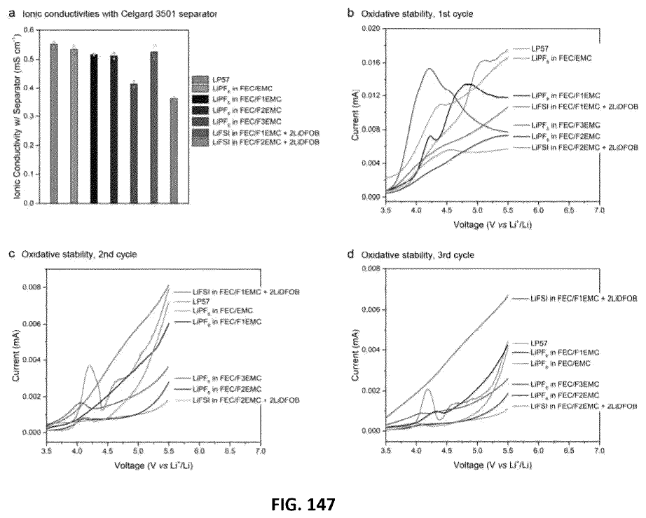

Stronger solvation capability of F1EMC and F2EMC leads to favorable cation-anion separation and higher ionic conductivity. FIG. 147a shows the ionic conductivities measured with Celgard 3501 separator. Celgard 3501 was used here mainly due to its better wettability for high FEC content electrolytes and thus fair comparison. All the developed electrolytes showed similar conductivities compared to the commercial LP57 except for 1 M LiPF₆ in FEC/F3EMC (3/7) and 1 M LiFSI in FEC/F2EMC (3/7)+2% LiDFOB whose ionic conductivities were slightly lower. We also examined the oxidative stability (tolerance towards Al corrosion) of the electrolytes using Li/Al half cells. The leaking currents of developed electrolytes during the first-cycle CV were lower than or at least similar to that of LP57 (FIG. 147b), and during the second and third cycles (FIGS. 147c and d) they followed the same trend of 1 M LiFSI in FEC/F1EMC+2% LiDFOB>LP57>1 M LiPF₆ in FEC/EMC>1 M LiPF₆ in FEC/F1EMC>1 M LiPF₆ in FEC/F3EMC>1 M LiPF₆ in FEC/F2EMC≈1 M LiFSI in FEC/F2EMC+2% LiDFOB. The lower leaking current corresponds to higher oxidative stability, and therefore, fluorinated electrolytes (F2EMC and F3EMC based) were more stable towards high voltage compared to less-fluorinated ones (F1EMC based, LP57 or FEC/EMC). It is worth noting that 1 M LiPF₆ in FEC/EMC showed trivial improvement in high-voltage stability, confirming our design that the fluorination of linear carbonate matters even though cyclic fluorinated carbonate FEC was used in all developed electrolytes. This will be further discussed in the later sections. Overall, the ionic conductivities and high-voltage stabilities of these developed electrolytes showed their promise for Li-ion batteries, as we tested and elaborated in the following sections.

FIG. 146 illustrates: ⁷Li- (a) and ¹⁹F-NMR (b-d) of fluorinated-EMCs and 1 M LiPF₆ in fluorinated-EMCs.

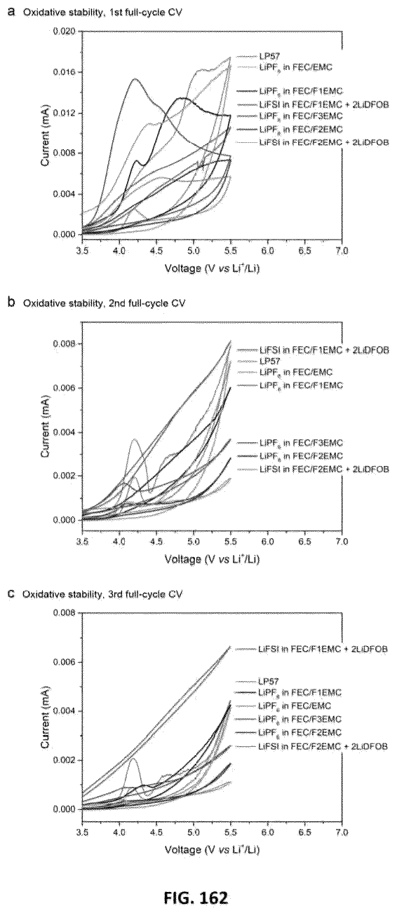

FIG. 147 illustrates: (a) Ionic conductivity of the electrolytes measured in coin cells with Celgard 3501 as the separator. Note: each bar stands for the mean of two replicated measurements and every single measurement is shown with hollow dots. (b-d) Oxidative stability test using CV: the 1st (b), 2nd (c) and 3rd (d) cycle. Note: only half cycles sweeping from low to high voltage are shown here for clarity and the full CV cycles can be found in FIG. 162.

4.4 V Gr/SC-NMC811. Single-crystalline NMC materials have drawn significant research attention recently. Dahn et al. did a series of systematic works (Y. Liu, J. Harlow, J. Dahn, Microstructural Observations of “Single Crystal” Positive Electrode Materials Before and After Long Term Cycling by Cross-section Scanning Electron Microscopy, J. Electrochem. Soc. 167 (2020) 020512. https://doi.org/10.1149/1945-7111/ab6288) on the effects of electrolyte additives on the cycling of single-crystalline NMCs. However, long cycling of Gr/SC-NMC811 pouch cells to an upper charging voltage of 4.4 V is still challenging yet highly desirable. We tested the performance of our developed electrolytes at a voltage range of 3-4.4 V and a cycling rate of 1 C under ambient conditions.

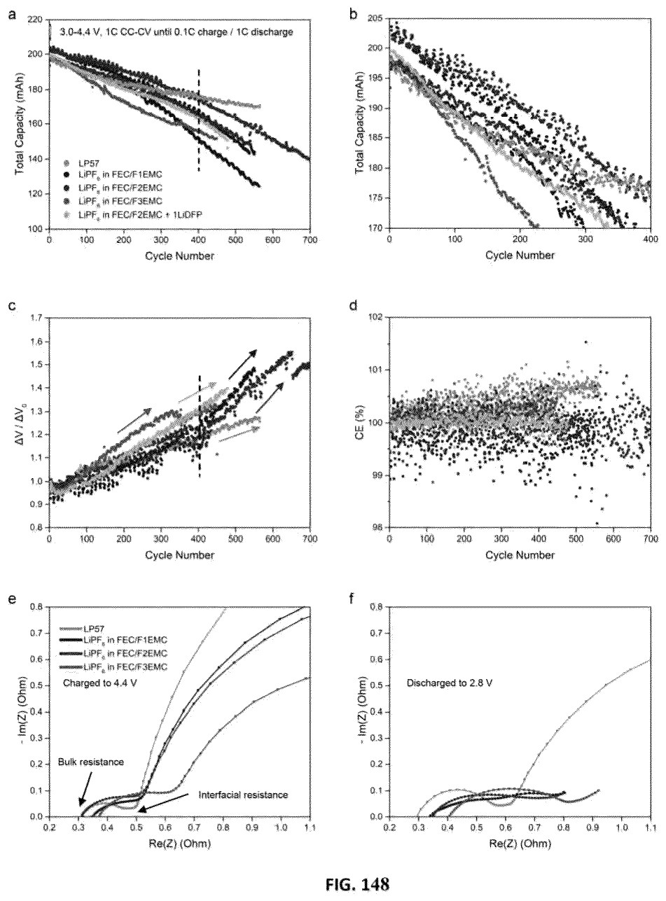

FIG. 148a shows the long-term cycle life of Gr/SC-NMC811 pouch cells using different electrolytes. The conventional and commercial carbonate electrolyte LP57 was still the best performing one. After ˜400 cycles, its capacity retention is the highest compared to all other developed electrolytes. However, the zoomed-in plot (FIG. 148b) shows that the delivered capacity decreased faster during the initial 400 cycles when using LP57 while its decrease speed was mitigated after 400 cycles. Similar decay mode was also observed for the F3EMC electrolyte although its capacity was lower than LP57 due to lower ionic conductivity. By contrast, all the synthesized electrolytes (the ones using F1EMC and F2EMC) exhibited steady decay before 400 cycles with capacities slightly higher than that of LP57 (FIG. 148b). However, their decrease speed accelerated after 400 cycles (FIG. 148a), which may be attributed to the fact that our in-house synthesized chemicals (water contents measured to be ˜50 ppm, Experimental-Electrolytes) were not as high quality as commercial battery-grade products such as LP57 and F3EMC (water content <20 ppm). It is noteworthy that such a water content is low for lab-made solvents and reasonably dry for initial battery testing. These two different decay modes corroborated well with the evolution of cell polarization (ΔV/ΔV₀) with cycling (FIG. 148c). For F1EMC and F2EMC cells, the cell polarization increased steadily before 400 cycles but sharply after that. For LP57 and F3EMC, steady increase in polarization was maintained over the cycle life, although both polarization value and increasing rate were higher for F3EMC.

When we only compare fluorinated-EMC electrolytes, the cycling performance follows the trend of F2EMC≈F2EMC+1% LiDFP>F1EMC>F3EMC (FIGS. 148a-d). The lower capacity of F3EMC, as mentioned above, can be ascribed to its lower ionic conductivity, while the slightly worse cycle life of F1EMC is due to its poor oxidative stability. It is noteworthy that using 1% LiDFP additive in FEC/F2EMC electrolyte did not improve the cycling stability of the Gr/SC-NMC811 pouch cell (FIGS. 148a and b); however, the increase of polarization and Coulombic efficiency (CE) became steadier compared to the no additive one (FIGS. 148c and d). The electrochemical impedance (EIS) results of pouch cells after long-term cycling are consistent with the argument and our design. Although debate is still ongoing for the detailed deconvolution and assignment of the EIS of Li-ion full cells (N. Meddings, M. Heinrich, F. Overney, J.-S. Lee, V. Ruiz, E. Napolitano, S. Seitz, G. Hinds, R. Raccichini, M. GaberMek, J. Park, Application of electrochemical impedance spectroscopy to commercial Li-ion cells: A review, J. Power Sources. 480 (2020) 228742. https://doi.org/10.1016/j.jpowsour.2020.228742), we can generally assign the intercept with −Im(Z)=0 as bulk resistance and the arcs at middle frequency region as interfacial resistance including electrode/electrolyte interfaces and charge transfer reactions, as shown by the arrows in FIG. 148e. FIGS. 148e and f show the highest bulk resistance and interfacial impedance for F3EMC while the lowest for LP57. F2EMC showed lower or similar bulk and interfacial resistance compared to F1EMC even though F1EMC originally possessed higher ionic conductivity (FIG. 147a). This is an indication that F1EMC electrolyte suffered from undesirable decomposition/oxidation during long cycling so that its transport properties evolved towards worse direction.

FIG. 148 illustrates: (a-d) Cycling behavior of Gr/SC-NMC811 pouch cells using different electrolytes: discharge capacity retention (a,b), normalized cell polarization during charge/discharge (c) and cycling CEs (d). Note: the same legend applies for (a-d); (b) is the zoomed-in plot of (a); ΔV₀ in (c) is the polarization of the second cycle at 1 C charge/discharge for each electrolyte. (e,f) EIS of pouch cells using different electrolytes at fully-charged (e) and fully-discharged (f) state after ˜560 cycles.

SiOₓ-based composite anode: Gr-SiOₓ/NMC622. SiOₓ is recognized as a next-generation anode material to increase the energy density of Li-ion cells (Z. Liu, Q. Yu, Y. Zhao, R. He, M. Xu, S. Feng, S. Li, L. Zhou, L. Mai, Silicon oxides: a promising family of anode materials for lithium-ion batteries, Chem. Soc. Rev. 48 (2019) 285-309. https://doi.org/10.1039/C8CS00441B). Here we choose Gr-SiOₓ composite anode with high SiOₓ content (20%) and high specific capacity (>550 mAh g⁻¹) to examine the performance of these new electrolytes. The suggested cycling rate by the vendor was <0.5 C but we chose 1 C charge/discharge here to magnify the difference.

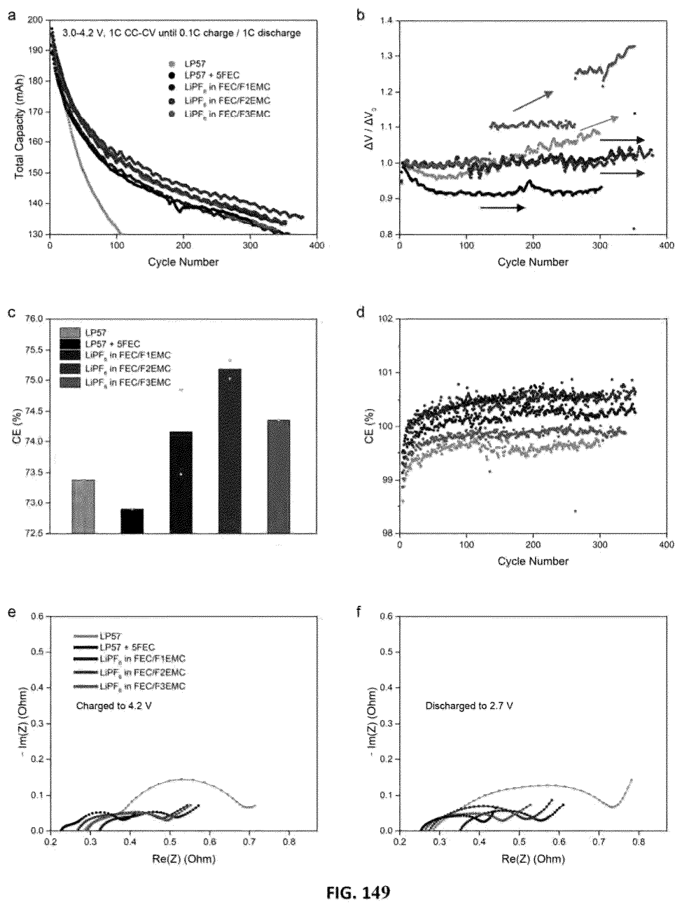

FIG. 149 illustrates: (a-d) Cycling behavior of Gr-SiOₓ/NMC622 pouch cells using different electrolytes: discharge capacity retention (a), normalized cell polarization during charge/discharge (b), first-cycle CE (c) and cycling CEs (d). Note: the same legend applies for (a,b,d); ΔV₀ in (b) is the polarization of the second cycle at 1 C charge/discharge for each electrolyte; each bar in (c) stands for the mean of two replicated measurements and every single measurement is shown with hollow dots. (e,f) EIS of pouch cells using different electrolytes at fully-charged (e) and fully-discharged (f) state after ˜350 cycles (˜300 cycles for LP57 and LP57+5% FEC).

The capacity retention of Gr-SiOₓ/NMC622 pouch cells with cycling was generally worse than that of Gr/SC-NMC811 (FIGS. 148a and 149a) since SiOₓ-based anodes are known to suffer from cycling instability due to volume expansion and particle cracking. As shown in FIG. 149a, we found the cycling performance of Gr-SiOₓ/NMC622 pouch cells follows the trend of F2EMC>F1EMC≈F3EMC>LP57+5% FEC>>LP57. This trend indicates that different fluorinated-EMCs do have unignorable impact on the cycling of Gr-SiOₓ anode regardless of the well-known improvement effect from FEC. The polarization evolution shown in FIG. 149b matched well with the performance trend. The poorly performing LP57 and F3EMC electrolytes showed large overpotential increase and values, while the F1EMC and F2EMC electrolytes exhibited low and stable polarization over the whole cycle life. The polarization of LP57+5% FEC decreased and stabilized with cycling compared to the initial value, which may be attributed to the high ionic conductivity of LP57 base; however, its cycling stability was slightly worse than the developed electrolytes. The underlying reason can be the loss of active Li-ion due to the poor performance of LP57 base for SiOₓ rather than polarization accumulation. This is supported by the fact that the first-cycle CE of LP57+5% FEC is the lowest among all (FIG. 149c). The first-cycle CE (FIG. 149c) and cycling CEs (FIG. 149d) of Gr-SiOₓ/NMC622 pouch cells were consistent with the aforementioned trend as well. Particularly, the F2EMC-based electrolyte showed the highest first-cycle CE as well as high cycling CEs, which agreed well with its best performance among all. It is worth noting that the cycling CEs of good electrolytes (F1EMC and F2EMC) were slightly higher than 100%. This may be a sign that the excess Li ions stored in the anode during the first charging (since the first-cycle CEs were far below 80%) were still active and gradually released back to the cathode during later discharging cycles. The EIS results of Gr-SiOₓ/NMC622 pouch cells at charged (FIG. 149e) or discharged (FIG. 149f) state corroborated with the above arguments. Low bulk and interfacial resistances were observed for highly performing electrolytes especially F2EMC, while the worst LP57 showed the largest interfacial impedance, indicating aggravated interfaces.

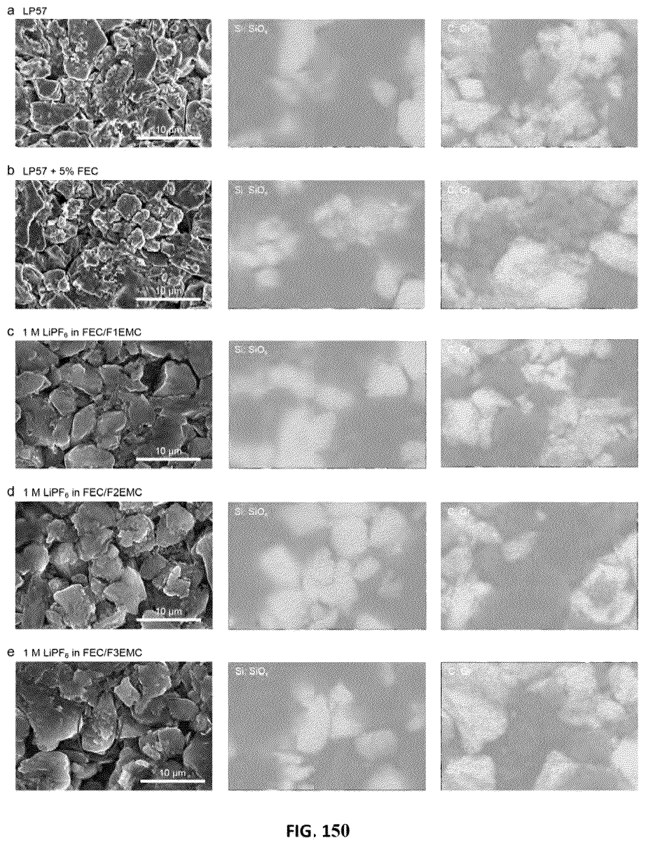

We further took SEM images and corresponding EDS of cycled Gr-SiOₓ anodes in different electrolytes (FIG. 150). The green colored parts are SiOₓ while the light-blue ones are Gr. The cells were fully discharged to 2.7 V to show the cracking of SiOₓ particles at delithiated state since it indicated the accumulation of stress over long-term cycling. The SiOₓ particles in either LP57 (FIG. 150a) or LP57+5% FEC (FIG. 150b) showed severe cracking after just 300 cycles; while F1EMC (FIG. 150c) and F2EMC (FIG. 150d) based electrolytes showed integrated and complete particles after ˜350 cycles. The particles in F3EMC (FIG. 150e) electrolyte also exhibited slight cracking although it was not as severe as that in commercial electrolytes. This can be ascribed to the accumulated particle stress which originated from poor ionic conduction and aggravated polarization in F3EMC based electrolyte.

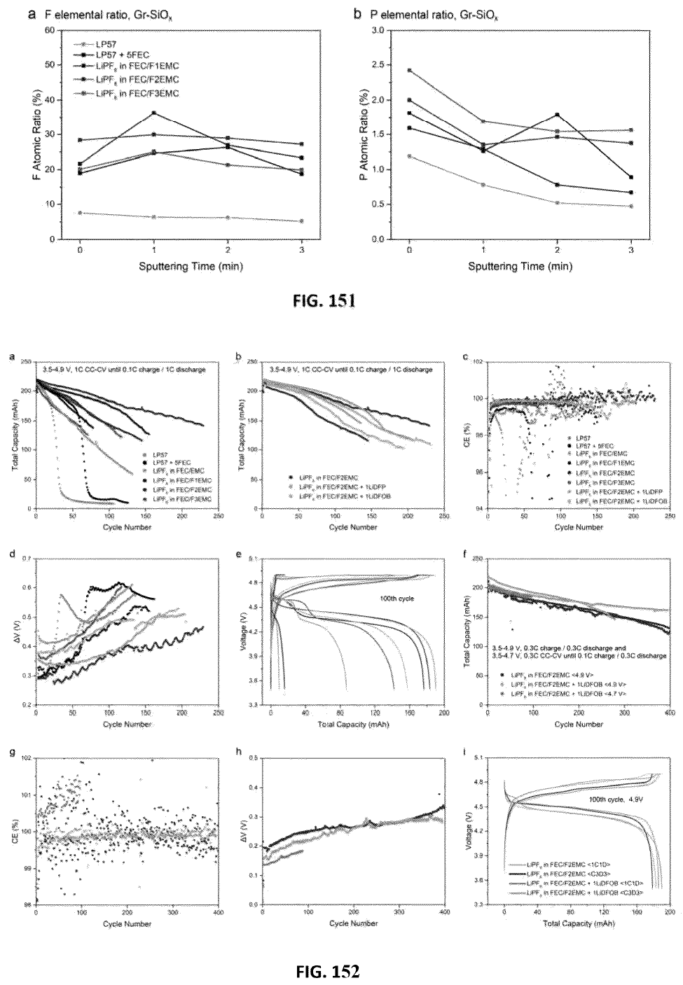

Elemental composition results of these cycled anodes were shown in FIG. 151. As expected, the worst performing FEC-free LP57 electrolyte showed the lowest F content, while LP57+5% FEC and developed electrolytes all contained >20% F species regardless of surface sputtering (FIG. 151a). The best performing F2EMC based electrolyte showed the highest F content and uniformity through depth profiling. The P contents in different electrolytes indicated the effect of solvent on the anion-derived, desired SEI species (FIG. 151b). The developed electrolytes generally showed high P contents although they decayed with sputtering.

FIG. 150 illustrates SEM and EDS images of Gr-SiOₓ anodes after ˜350 cycles using different electrolytes (˜300 cycles for LP57 and LP57+5% FEC) at fully-discharged state. Note: red circles in (a,b,e) indicate the cracking of SiOₓ particles; green shadow in the middle column represents Si element (SiOₓ) and light-blue shadow in the right column represents C element (mainly Gr and a small proportion of conductive carbon additive).

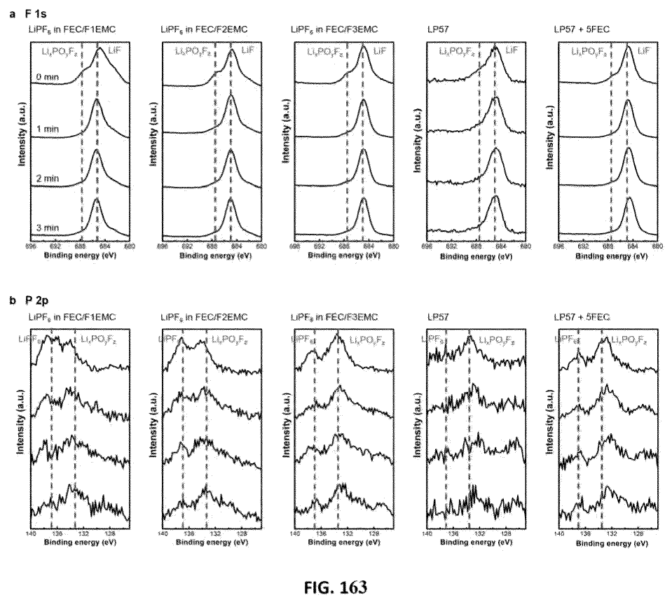

FIG. 151 provides: F (a) and P (b) elemental composition results of Gr-SiOₓ anodes after ˜350 cycles using different electrolytes (300 cycles for LP57 and LP57+5% FEC) by XPS. Note: XPS depth profiling spectra can be found in FIG. 163.

High voltage cathodes: Gr/LNMO and Gr/LLMO. One of the most noteworthy benefits of solvent fluorination is to enhance the oxidative stability. Therefore, we further used high-voltage cathodes, LNMO and Li-rich Mn-based LLMO, to demonstrate the feasibility of our electrolytes. The Gr/LNMO and Gr/LLMO cells were charged up to 4.9/4.7 V and 4.8 V, respectively, to maximize the high-voltage effects.

F3EMC has been commercialized and widely used in LNMO-based cells due to its oxidative stability (X. Yu, W. A. Yu, A. Manthiram, Advances and Prospects of High-Voltage Spinel Cathodes for Lithium-Based Batteries, Small Methods. 5 (2021) 2001196. https://doi.org/10.1002/smtd.202001196); however, no report was found on tuning its fluorination degree to answer a key question: whether F1EMC and F2EMC can outperform F3EMC in Gr/LNMO cells?FIG. 152a shows that the capacity retention of Gr/LNMO pouch cells followed the order of FEC/F2EMC>FEC/F1EMC>FEC/F3EMC>FEC/EMC>>LP57+5% FEC>>LP57, despite variations between replicated cells. Better performance of fluorinated EMCs than EMC confirmed the effectiveness of fluorination. After adding 1% LiDFOB and 1% LiDFP respectively into F2EMC-based electrolyte as the additive, the parallel cells showed more consistent cycling performance yet the improvement in cycle life was trivial at 1 C rate (FIG. 152b). Better cycle life corresponded to more stable and higher cycling CEs (FIG. 152c). The cell polarizations shown in FIG. 152d also reflected the cycling trend. The overpotential ramped up at the moment when the capacity sharply decayed for control electrolytes LP57 and LP57+5% FEC; however, all the other electrolytes showed relatively smooth progression of polarization over cycling. Faster increase was observed for the overpotentials of FEC/EMC, FEC/F1EMC and FEC/F3EMC cells, but may be attributed to two different mechanisms: for FEC/EMC and FEC/F1EMC, its oxidative instability and electrolyte decomposition led to fast overpotential accumulation, and particularly, the FEC/EMC cell showed much higher polarization than FEC/F1EMC, indicating more severe decomposition of non-fluorinated EMC; for FEC/F3EMC, its low ionic conductivity (FIG. 147a) and high interfacial resistance dominated, which was further verified by its extremely high charging plateau and long constant-voltage period at the 100th cycle (FIG. 152e). These points are the pivot of our design in this work. For better performing F2EMC-based electrolytes, smooth and slow increases in polarization were observed. When cycled under milder conditions (0.3 C charge/discharge without constant-voltage holding, or 4.7 V upper cutoff), the Gr/LNMO cells exhibited much more stable and consistent capacity retention and CEs (FIGS. 152f and g). The 1% LiDFOB additive functioned well under these conditions by showing higher delivered capacity and less CE fluctuation. The cycling CEs of 4.7 V cell maintained higher than 100% (FIG. 152g) probably due to the fact that less electrolyte decomposition happened and pre-stored Li-ion at the first-cycle charging slowly released during later discharging cycles. As shown in FIG. 152h, the cell polarization followed the trend of F2EMC+1% LiDFOB (4.7 V)<F2EMC+1% LiDFOB (4.9 V)<F2EMC (4.9 V), indicating that low charging cutoff and LiDFOB additive are beneficial to the cycling performance of LNMO. In addition, slower cycling rate was beneficial to the cycling. Smaller gap between charge and discharge plateau was observed for 0.3 C compared to 1 C (FIG. 152i).

FIG. 152 illustrates: (a-e) Cycling behavior of Gr/LNMO pouch cells using different electrolytes at 1 C charge/discharge: discharge capacity retention (a,b), cycling CEs (c), absolute values of cell polarization during charge/discharge (d), and charge/discharge curves of the 100th cycle (e). Note: the same legend applies for (c-e). (f-i) Cycling behavior at 0.3 C charge/discharge: discharge capacity retention (f), cycling CEs (g), absolute values of polarization during charge/discharge (h), and charge/discharge curves of the 100th cycle (i). Note: the same legend applies for (f-h); <4.9 V> and <4.7 V> represent different charging cutoffs; <1C1D> is 1 C charge/discharge while <C3D3> is 0.3 C.

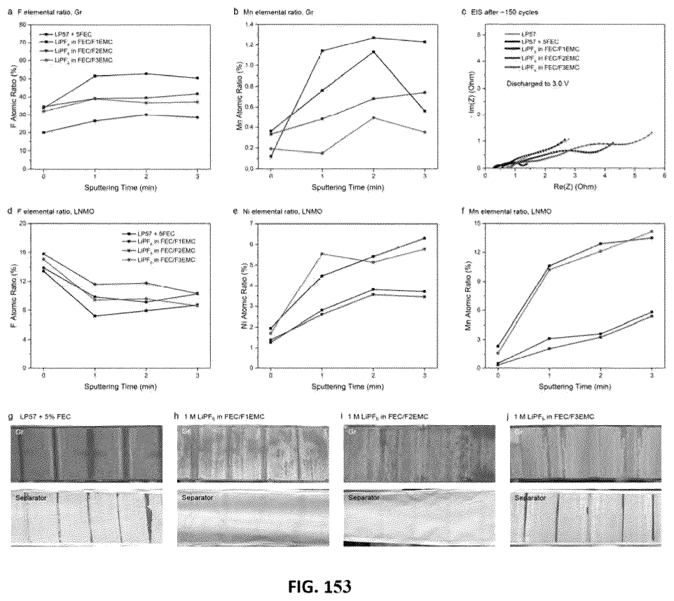

XPS analyses were carried out to quantify surface species on Gr anodes and LNMO cathodes after cycling (FIGS. 153a-e). For Gr anodes (FIGS. 153a and b), the fluorinated-EMC electrolytes showed higher F contents than LP57+5% FEC, indicating better SEI protection. The Mn content detected on Gr surface indicated the extent of transition metal dissolution (F. Zou, H. C. Nallan, A. Dolocan, Q. Xie, J. Li, B. M. Coffey, J. G. Ekerdt, A. Manthiram, Long-life LiNi0.5Mn1.5O4/graphite lithium-ion cells with an artificial graphite-electrolyte interface, Energy Storage Mater. 43 (2021) 499-508. https://doi.org/10.1016/j.ensm.2021.09.033) and it followed the order of F3EMC <F2EMC <LP57+5% FEC <F1EMC (FIG. 153b). The EIS after cycling, however, showed slightly different trend of overall cell resistance: F3EMC>>F1EMC>>LP57 >LP57+5% FEC>>F2EMC (FIG. 153c). These two facts confirm the aforementioned hypothesis that F1EMC mainly suffered from oxidative instability and cathode dissolution, while sluggish bulk and interfacial ion transport dominated in F3EMC cells rather than Mn dissolution. For LNMO cathode surface (FIGS. 153d-f), higher F content and lower Ni/Mn contents were observed in F2EMC-based electrolyte, indicating effective cathode protection layer. The Ni and Mn contents in F3EMC electrolyte were as high as those in the control LP57+5% FEC (FIGS. 153e and f). The low ionic conductivity and high interfacial resistance of F3EMC electrolyte may devastate the LNMO surface. When we opened the pouch cells after cycling, gray-colored species and brownish color were observed on Gr anodes and separators, respectively, indicating transition metal dissolution and shuttling (Id.) (FIGS. 153g-j). This phenomenon was particularly more severe in F1EMC and F3EMC than in F2EMC, which corroborated with the cycle life trend.

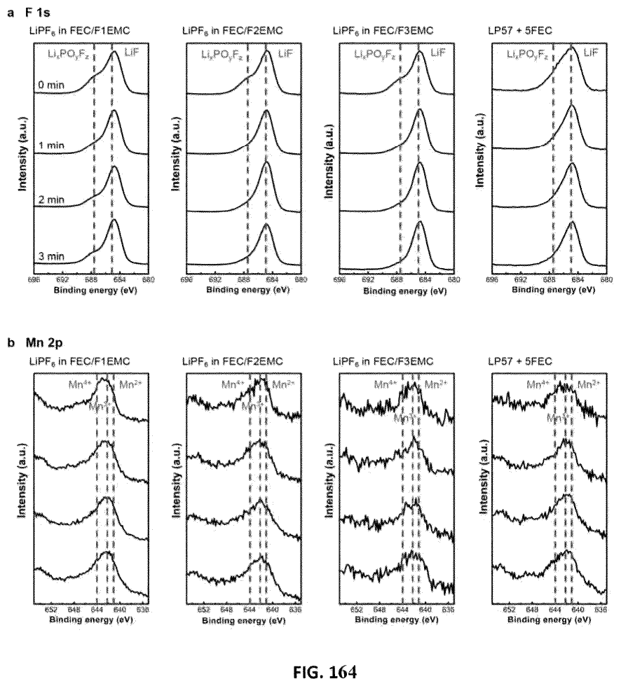

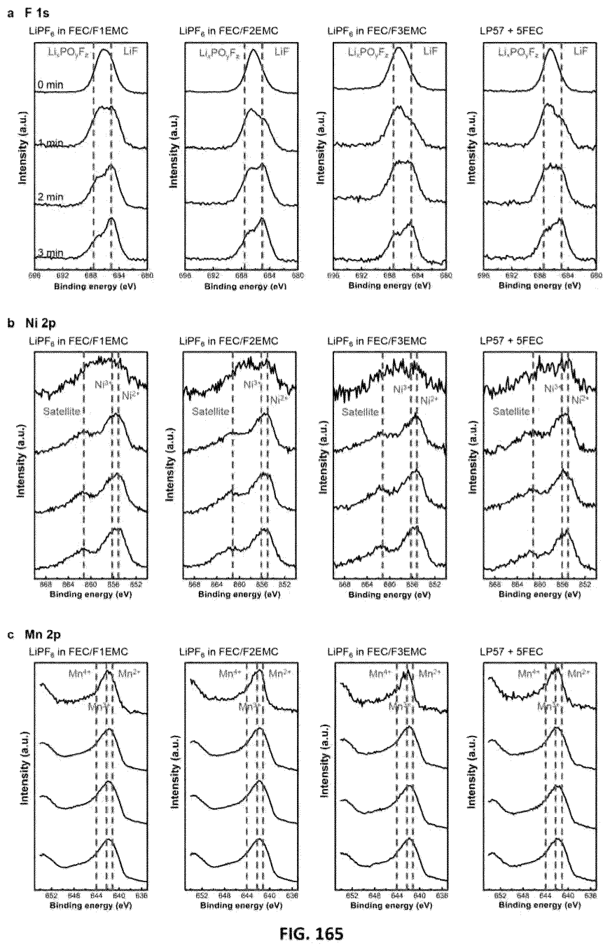

FIG. 153 provides: F (a) and Mn (b) elemental composition results of Gr anodes by XPS. Note: the same legend applies for (a,b). (c) EIS of Gr/LNMO pouch cells at fully-discharged state. F (d), Ni (e) and Mn (f) elemental composition results of LNMO cathodes by XPS. Note: the same legend applies for (d-f). (g-j) Optical images of Gr anodes and separators. Note: all results were obtained after ˜150 cycles at 1 C charge/discharge. Note: XPS depth profiling spectra can be found in FIGS. 164 and 165.

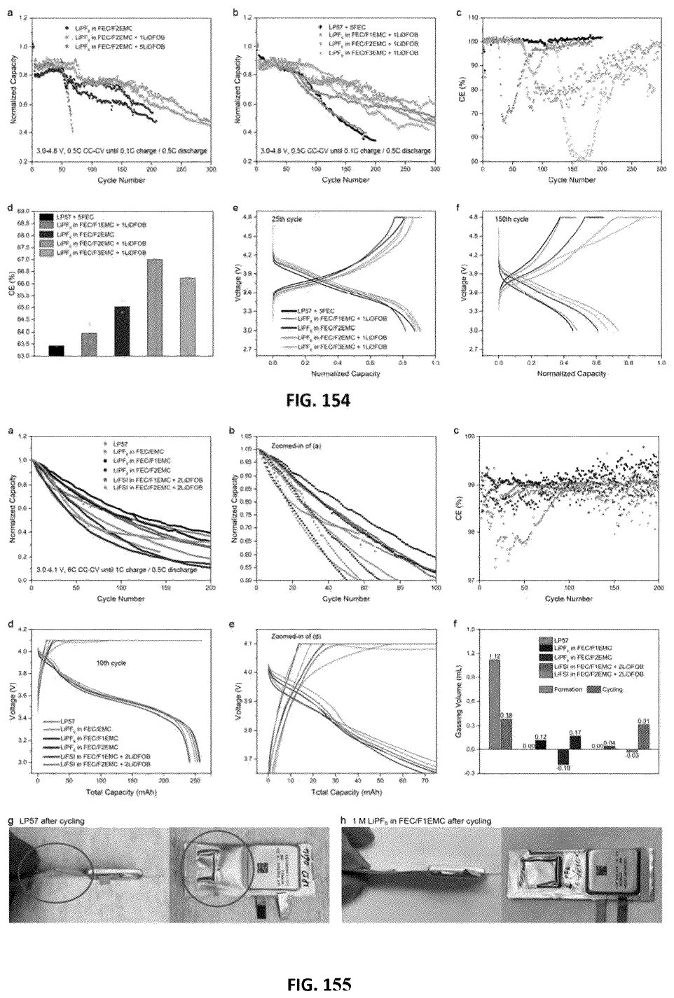

Layered Li-rich Mn-based oxides (LLMOs) are also promising cathode materials for high-energy Li-ion batteries (P. Rozier, J. M. Tarascon, Review-Li-Rich Layered Oxide Cathodes for Next-Generation Li-Ion Batteries: Chances and Challenges, J. Electrochem. Soc. 162 (2015) A2490-A2499. https://doi.org/10.1149/2.0111514ies; P. K. Nayak, E. M. Erickson, F. Schipper, T. R. Penki, N. Munichandraiah, P. Adelhelm, H. Sclar, F. Amalraj, B. Markovsky, D. Aurbach, Review on Challenges and Recent Advances in the Electrochemical Performance of High Capacity Li—and Mn-Rich Cathode Materials for Li-Ion Batteries, Adv. Energy Mater. 8 (2018) 1702397. https://doi.org/10.1002/aenm.201702397; W. He, W. Guo, H. Wu, L. Lin, Q. Liu, X. Han, Q. Xie, P. Liu, H. Zheng, L. Wang, X. Yu, D. Peng, Challenges and Recent Advances in High Capacity Li-Rich Cathode Materials for High Energy Density Lithium-Ion Batteries, Adv. Mater. 33 (2021) 2005937. https://doi.org/10.1002/adma.202005937). Since LiDFOB was reported to improve the stability of LLMO cathodes through its induced solvent polymerization (Y. Zhu, Y. Li, M. Bettge, D. P. Abraham, Positive Electrode Passivation by LiDFOB Electrolyte Additive in High-Capacity Lithium-Ion Cells, J. Electrochem. Soc. 159 (2012) A2109-A2117. https://doi.org/10.1149/2.083212jes), we first added different amounts of LiDFOB into 1 M LiPF₆ in FEC/F2EMC base to examine the effects on the performance of Gr/LLMO pouch cells. As shown in FIG. 154a, 1% LiDFOB additive slightly improved the cycling stability compared to the bare 1 M LiPF₆ in FEC/F2EMC. Surprisingly, 5% additive significantly degraded the performance, probably due to its “mixed-blessing” protection mechanism (Id.)—the decomposition of LiDFOB released large quantity of C02 gas while yielding protective FEC polymerization products. Therefore, 1% LiDFOB was selected as the additive for all fluorinated-EMC electrolytes. FIG. 154b shows that the cycling stability generally followed the order of F2EMC+1% LiDFOB ˜F3EMC+1% LiDFOB>F1EMC+1% LiDFOB>LP57+5% FEC. Unexpectedly, the CEs of fluorinated-EMC cells dropped to <80% during later cycles and then slightly recovered, while the worst LP57+5% FEC cell maintained CE ˜99% over the whole cycle life (FIG. 154c). This situation may be again ascribed to better quality of commercial battery-grade LP57 (water content usually <20 ppm) than lab-made electrolytes whose synthesized solvents contain a water content of ˜50 ppm (Experimental-Electrolytes). Despite the later-cycle CE fluctuation, the first-cycle CEs corroborated with cycling stability by following the order of F2EMC+1% LiDFOB>F3EMC+1% LiDFOB>F2EMC>F1EMC+1% LiDFOB>LP57+5% FEC (FIG. 154d). The charge/discharge curves at cycle 25 (FIG. 154e) and 150 (FIG. 154f) show polarization of Gr/LLMO cells which has been regarded as a key issue of Li-rich cathodes. The less stable electrolytes such as LP57+5% FEC and F1EMC+1% LiDFOB exhibited larger overpotential, while the most stable electrolyte, F2EMC+1% LiDFOB, maintained the highest discharge voltage plateau.

FIG. 154 illustrates: (a-d) Cycling behavior of Gr/LLMO pouch cells using different electrolytes: discharge capacity retention of F2EMC-based electrolytes with different amounts of LiDFOB additive (a), discharge capacity retention of different fluorinated-EMC-based electrolytes (b), cycling CEs (c) and first-cycle CE (d). Note: the same legend applies for (b,c); each bar in (d) stands for the mean of two replicated measurements and every single measurement is shown with hollow dots. (e,f) Charge/discharge curves of pouch cells using different electrolytes at the 25th cycle (e) and 150th cycle (f). Note: the same legend applies for (e,f).

Fast charging: Gr/NMC622. Fast-charging capability is highly desired in the market (Y. Liu, Y. Zhu, Y. Cui, Challenges and opportunities towards fast-charging battery materials, Nat. Energy. 4 (2019) 540-550. https://doi.org/10.1038/s41560-019-0405-3; A. Tomaszewska, Z. Chu, X. Feng, S. O'Kane, X. Liu, J. Chen, C. Ji, E. Endler, R. Li, L. Liu, Y. Li, S. Zheng, S. Vetterlein, M. Gao, J. Du, M. Parkes, M. Ouyang, M. Marinescu, G. Offer, B. Wu, Lithium-ion battery fast charging: A review, ETransportation. 1 (2019) 100011. https://doi.org/10.1016/j.etran.2019.100011). We tested the pouch cells specially made for 4 C fast charging; however, we here used 6 C charge 0.5 C discharge protocol (see Experimental-Pouch cell cycling) to maximize the performance difference. It is worth noting that these cells still suffer significant capacity decay at 6 C charge but the cycle life difference can be magnified.

The capacity retention after 200 cycles followed the order of 1 M LiPF₆ in FEC/F1EMC≈LP57 >1 M LiFSI in FEC/F1EMC+2% LiDFOB>>1 M LiFSI in FEC/F2EMC+2% LiDFOB>1 M LiPF₆ in FEC/EMC>1 M LiPF₆ in FEC/F2EMC (FIG. 155a). It is worth noting that we only charged the cells to 4.1 V and this condition will magnify the fast-charging capability of the electrolyte while eliminating the influence of its oxidative stability. Therefore, this trend is generally consistent with that of ionic conductivities (FIG. 147a), and the highly conductive electrolytes 1 M LiPF₆ in FEC/F1EMC, LP57 and 1 M LiFSI in FEC/F1EMC+2% LiDFOB demonstrated the best 6 C charging performances. Interestingly, we found the performance of FEC/EMC electrolyte is not superior to the fluorinated linear carbonate ones. Although the ionic conductivity of FEC/EMC is comparable to LP57 and other developed electrolytes, the synergistic effect of FEC and fluorinated EMC (particularly F1EMC) may be more beneficial for anode passivation under such fast-charging conditions. This assumption may also be responsible for the fact that F1EMC-based electrolytes delivered higher capacities before 100 cycles compared to the LP57 control which showed drastic decay during initial cycles (FIG. 155b). Detailed mechanisms will be further investigated in the future. The cycling CEs of F1EMC-based electrolytes were significantly higher than LP57 or F2EMC-based ones (FIG. 155c), matching with the capacity retention trend. FIGS. 155d and e show the charge/discharge curves of the 10th cycle. Better performing electrolytes exhibited longer constant-current period and higher initial discharge voltage, both of which corroborated with high ionic conductivity and low overpotential.

FIG. 155 illustrates: (a-e) Fast-charging cycling behavior of Gr/NMC622 pouch cells using different electrolytes: normalized discharge capacity retention (a,b), cycling CEs (c) and charge/discharge curves of the 10th cycle (d,e). Note: (b) is the zoomed-in plot of (a); only one pouch result for each electrolyte is selected in (c) for clarity; (e) is the zoomed-in plot of (d). (f-h) Gassing issue of the pouch cells using different electrolytes: gassing volume after formation and cycling (f) and optical images of pouch cells after cycling using LP57 (g) and 1 M LiPF₆ in FEC/F1EMC (h). Note: for each electrolyte, the left column is the gassing volume after formation cycles while the right column is that after cycling (degassing procedure was implemented after cell formation).

The gassing issue during fast charging is a major concern for Li-ion batteries (Id.). This mainly originates from side reactions between poor electrolytes and Li metal dendrites generated during fast charging. FIGS. 155f-h show the gassing behavior of the pouch cells during testing. While the control electrolyte LP57 released a large quantity of gases during either formation cycles (1.12 mL) or long-term fast cycling (0.38 mL), all the developed fluorinated electrolytes generated limited gases (<0.2 mL except for 1 M LiFSI in FEC/F2EMC+2% LiDFOB). The gas bag of LP57 cell severely expanded after cycling even degassing procedure was implemented after the formation cycles (FIG. 155g); by contrast, little gassing was observed for the best performing 1 M LiPF₆ in FEC/F1EMC (FIG. 155h). It is also noteworthy that electrolytes with LiDFOB additive generated more gases due to its decomposition at high voltage (FIG. 155f). Overall, our developed electrolytes showed effectiveness in suppressing the gassing issue during 6 C charging.

### II. B. 5. Conclusions

In summary, we rationally designed and finely tuned the fluorination degree of ethyl methyl carbonate (EMC) to obtain monofluoroethyl methyl carbonate (F1EMC), difluoroethyl methyl carbonate (F2EMC) and trifluoroethyl methyl carbonate (F3EMC) as a family of fluorinated-EMCs. A variety of industrial Li-ion pouch cells, including 4.4 V Gr/NMC811, Gr-SiOₓ/NMC622, high-voltage Gr/LNMO and Gr/LLMO, and fast-charging Gr/NMC622, were systematically investigated to elaborate the impacts of fluorination degree on battery performance (Supplementary Table 1). We found that, the partially-fluorinated F1EMC and F2EMC are better solvent choices for the Li-ion batteries we studied compared to commercially available, widely-studied F3EMC. This stems from the locally polar —CH₂F and —CHF₂ groups and their better ion conduction capability. This work shows a promising direction for future development of Li-ion battery electrolytes, i.e., fine tuning of fluorination degree for optimizing electrolyte solvent performance.

### II. B. 6. Supplementary Information

Supplemental aspects for Tuning fluorination of linear carbonate for lithium-ion batteries are provided in the figures as follows:

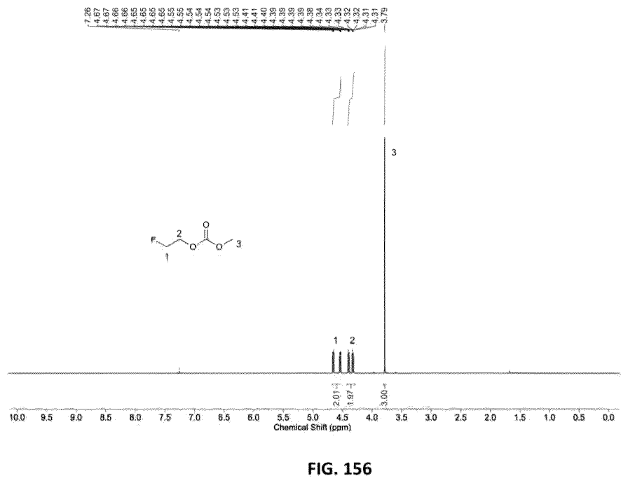

FIG. 156 provides ¹H-NMR of F1EMC (400 MHz, CDCl3, δ/ppm): 4.67-4.53 (m, 2H), 4.41-4.31 (m, 2H), 3.79 (s, 3H).

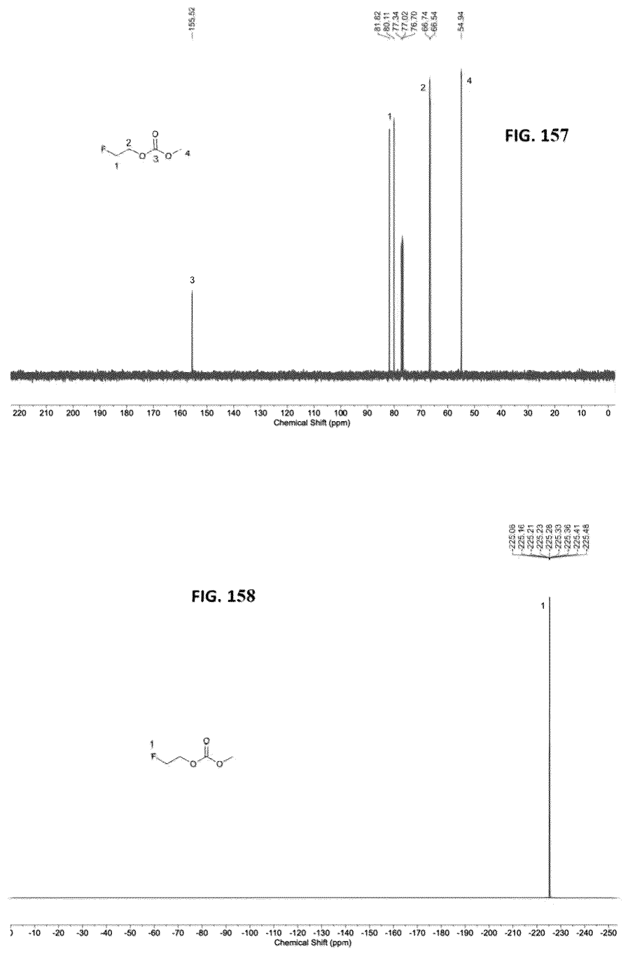

FIG. 157 provides ¹³C-NMR of F1EMC (100 MHz, CDCl3, 8/ppm): 155.51, 81.80-80.10, 66.72-66.52, 54.92.

FIG. 158 provides ¹⁹F-NMR of F1EMC (376 MHz, CDCl3, δ/ppm): −225.08-−225.48 (m, 1F).

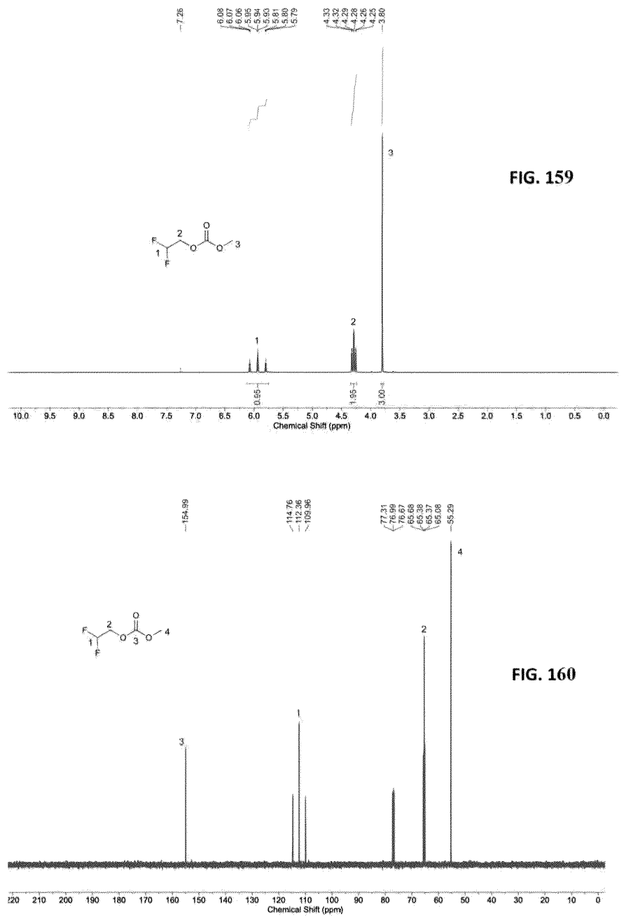

FIG. 159 provides ¹H-NMR of F2EMC (400 MHz, CDCl3, δ/ppm): 6.08-5.79 (tt, 1H), 4.33-4.25 (td, 2H), 3.80 (s, 3H).

FIG. 160 provides ¹³C-NMR of F2EMC (100 MHz, CDCl3, δ/ppm): 155.00, 114.77-109.97, 65.69-65.09, 55.30.

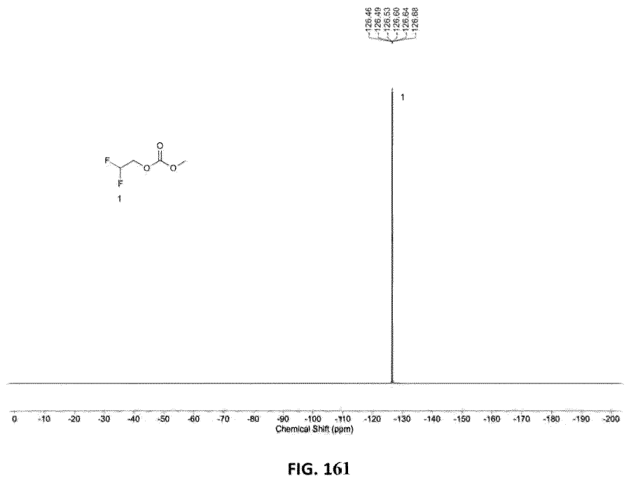

FIG. 161 provides a ¹⁹F-NMR of F2EMC (376 MHz, CDCl₃, δ/ppm): −126.46-−126.68 (dt, 2F).

FIG. 162 provides Oxidative stability test using CV: the 1st (a), 2nd (b) and 3rd (c) complete cycle.

FIG. 163 provides F is (a) and P 2p (b) XPS depth profiling spectra of Gr-SiOₓ anodes after-350 cycles in Gr-SiOₓ/NMC622 pouch cells using different electrolytes (300 cycles for LP57 and LP57+5% FEC).

FIG. 164 provides F is (a) and Mn 2p (b) XPS depth profiling spectra of Gr anodes after-150 cycles in Gr/LNMO pouch cells using different electrolytes.

FIG. 165 provides F is (a), Ni 2p (b), and Mn 2p (c) XPS depth profiling spectra of LNMO cathodes after-150 cycles in Gr/LNMO pouch cells using different electrolytes.

Supplementary TABLE 1
Summary of cycling performance in this work.

| Cell Type^{a} | Electrolyte^{b} | Voltage (V) | Cycling Rate | Cycling Performance^{c} |
|---|---|---|---|---|
| Gr/SC-NMC811 | LP57 | 3-4.4 | 1 C (CC-CV until 0.1 C) / 1D | 85% at cycle 565 |
| | LiPF₆ in FEC/F1EMC | | | 80% at cycle 450 |
| | LiPF₆ in FEC/F2EMC | | | 80% at cycle 550 |
| | LiPF₆ in FEC/F3EMC | | | 80% at cycle 330 |
| | LiPF₆ in FEC/F2EMC + 1% LiDFP | | | 80% at cycle 430 |
| Gr-SiOₓ/NMC622 | LP57 | 3-4.2 | 1 C (CC-CV until 0.1 C) / 1D | 70% at cycle 75 |
| | LP57 + 5% FEC | | | 70% at cycle 185 |
| | LiPF₆ in FEC/F1EMC | | | 70% at cycle 250 |
| | LiPF₆ in FEC/F2EMC | | | 70% at cycle 300 |
| | LiPF₆ in FEC/F3EMC | | | 70% at cycle 250 |
| Gr/LNMO | LP57 | 3.5-4.9 | 1 C (CC-CV until 0.1 C) / 1D | 75% at cycle 24 |
| | LP57 + 5% FEC | | | 75% at cycle 60 |
| | LiPF₆ in FEC/EMC | | | 75% at cycle 46 |
| | LiPF₆ in FEC/F1EMC | | | 75% at cycle 132 |
| | LiPF₆ in FEC/F2EMC | | | 75% at cycle 205 |
| | LiPF₆ in FEC/F3EMC | | | 75% at cycle 90 |
| | LiPF₆ in FEC/F2EMC + 1% LiDFP | | | 75% at cycle 145 |
| | LiPF₆ in FEC/F2EMC + 1% LiDFOB | | | 75% at cycle 160 |
| | LiPF₆ in FEC/F2EMC | 3.5-4.9 | 0.3 C (no CV) / 0.3D | 75% at cycle 300 |
| | LiPF₆ in FEC/F2EMC + 1% LiDFOB | 3.5-4.7 | | 92% at cycle 100 |
| | | 3.5-4.9 | | 80% at cycle 400 |
| Gr/LLMO | LP57 + 5% FEC | 3-4.8 | 0.5 C (CC-CV until 0.1 C) / 0.5D | 70% at cycle 95 |
| | LiPF₆ in FEC/F2EMC | | | 70% at cycle 150 |
| | LiPF₆ in FEC/F1EMC + 1% LiDFOB | | | 70% at cycle 95 |
| | LiPF₆ in FEC/F2EMC + 1% LiDFOB | | | 70% at cycle 200 |
| | LiPF₆ in FEC/F3EMC + 1% LiDFOB | | | 70% at cycle 145 |
| | LiPF₆ in FEC/F2EMC + 5% LiDFOB | | | 70% at cycle 60 |
| Fast-charging | LP57 | 3-4.1 | 6 C (CC-CV until 1 C) / 0.5D | 53% at cycle 100 |
| Gr/NMC622 | LiPF₆ in FEC/EMC | | | 38% at cycle 100 |
| | LiPF₆ in FEC/F1EMC | | | 59% at cycle 100 |
| | LiPF₆ in FEC/F2EMC | | | 35% at cycle 100 |
| | LiFSI in FEC/F1EMC + 2% LiDFOB | | | 53% at cycle 100 |
| | LiFSI in FEC/F2EMC + 2% LiDFOB | | | 42% at cycle 100 |

^{a}Please see Table 1 in the manuscript for detailed pouch cell specifications and electrolyte filling amount.
^{b}The concentrations of main lithium salts were 1M in all cases.
^{b}All the cells were cycled at room temperature without temperature control.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably coupleable,” to each other to achieve the desired functionality. Specific examples of operably coupleable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

Although the present embodiments have been particularly described with reference to preferred examples thereof, it should be readily apparent to those of ordinary skill in the art that changes and modifications in the form and details may be made without departing from the spirit and scope of the present disclosure. It is intended that the appended claims encompass such changes and modifications.

In additional embodiments a solvent for an electrolyte of a battery is a mixture of one or more of the above-embodied fluoro-compounds and at least one of ethylene carbonate (EC), propylene carbonate (PC), dimethyl carbonate (DMC), diethyl carbonate (DEC), ethyl methyl carbonate (EMC), vinyl carbonate (VC), fluoroethylene carbonate (FEC), difluoroethylene carbonate (DFEC), 3,3,3-trifluoropropylene carbonate (TFPC), trifluoroethyl methyl carbonate (FEMC), bis(2,2,2-trifluoroethyl) carbonate (TFEC), 1,2-dimethyoxylethane (DME), 1,3-dioxolane (DOL), 1,4-dioxane (DOX), tetrahydrofuran (THF), 1,3,2-dioxathiolane-2,2-dioxide (DTD), 1,3-propanesultone (PS), acetonitrile (AN), ethyl acetate (EA), methyl acetate (MA), methyl propanoate (MP), succinonitrile (SN), trimethyl phosphate (TMP), triethyl phosphate (TEP); tris(trimethylsilyl)phosphate (TTSP), tris(2,2,2-trifluoroethyl) phosphate (TFEPa), tris(2,2,2-trifluoroethyl) phosphite (TFEPi), prop-i-ene-1,3-sultone (PES), ethylene sulfite (ES), 1,4-butane sultone (BS), dimethyl sulfoxide (DMSO), methylene methanedisulfonate (MMDS), N,N-Dimethylformamide (DMF), and gamma-butyrolactone (BL). In some embodiments, the mixture comprises two, three or four compounds from those listed above.

In some embodiments, the one or more of the above-embodied fluoro-compounds comprise at least 5 wt. %, 10 wt. %, 15 wt. %, 20 wt. %, 25 wt. %, 30 wt. %, 35 wt. %, 40 wt. %, 45 wt. %, 50 wt. %, 55 wt. %, 60 wt. %, 65 wt. %, 70 wt. %, 75 wt. %, 80 wt. %, 85 wt. %, 90 wt. %, 95 wt. %, 98 wt. %, 99 wt. %, 99 wt. %, 99.5 wt. %, or 100 wt. % of the solvent.

In additional embodiments, an electrolyte of a battery includes the solvent of any of the foregoing embodiments, and a salt. In some embodiments, the salt is a lithium salt, potassium salt, sodium salt, or a mixture thereof. For example, in some embodiments, the salt includes one or more of lithium bis(fluorosulfonyl)imide (LiFSI); lithium bis(trifluoromethanesulfonyl)imide (LiTFSI); lithium hexafluorophosphate (LiPF₆); lithium hexafluoroarsenate (LiAsF6); lithium tetrafluoroborate (LiBF4); lithium bis(oxalato)borate (LiBOB); lithium difluoro(oxalato)borate (LiDFOB); lithium difluorophosphate (LiDFP); lithium nitrate (LiNO3); lithium perchlorate (LiClO4); lithium triflate (LiTf); lithium trifluoroacetate (LiTFA); lithium 4,5-dicyano-2˜(trifluoromethyl)imidazole (LiTDI); sodium bis(fluorosulfonyl)imide (NaFSI); sodium bis(trifluoromethanesulfonyl)imide (NaTFSI); potassium bis(fluorosulfonyl)imide (KFSI); and potassium bis(trifluoromethanesulfonyl)imide (KTFSI).

In additional embodiments, an electrolyte of a battery includes the solvent of any of the foregoing embodiments, and a salt of any of the foregoing embodiments (e.g., a lithium salt). In some embodiments, the electrolyte includes a mixture of two or more solvents of the foregoing embodiments, and the salt (e.g., lithium salt). In some embodiments, an amount of the solvent (or the mixture of solvents) in the electrolyte is at least about 60% by weight of a total weight of the electrolyte, such as at least about 65% by weight, at least about 70% by weight, at least about 75% by weight, or at least about 80% by weight. In some embodiments, the electrolyte consists essentially of the solvent (or the mixture of solvents) and the salt (e.g., lithium salt). In some embodiments, the electrolyte includes (i) a mixture of one or more solvents of the foregoing embodiments and one or more additional solvents, such as selected from ethers and carbonates, and (ii) the salt (e.g., lithium salt). Examples of the lithium salt include lithium bis(fluorosulfonyl)imide, lithium bis(trifluoromethanesulfonyl)imide, lithium hexafluorophosphate, lithium hexafluoroarsenate, lithium tetrafluoroborate, lithium perchlorate, and lithium triflate.

In additional embodiments, a battery includes (1) an anode structure including an anode current collector, (2) a cathode structure including a cathode current collector and a cathode material disposed on the cathode current collector, and (3) the electrolyte of any of the foregoing embodiments disposed between the anode structure and the cathode structure. In some embodiments, the anode structure further includes an anode material disposed on the anode current collector. In some embodiments, the anode material comprises lithium metal, graphite, silicon, or a graphite/silicon (silicon can be Si, SiOₓ, SiC, or Si₃N₄) composite anode. In some embodiments, the graphite/silicon (silicon can be Si, SiOₓ, SiC, or Si₃N₄) composite anode includes a weight ratio of graphite/silicon of about 5:95 10:90, 20:80, 30:70, 40:60, 50:50, 60:40, 70:30, 20:80, 90:10, or 95:5. In some embodiments, the cathode material comprises a sulfur-based cathode or an air cathode (e.g., a Li—S, Li-SPAN, or a Li-air battery). In some embodiments, the cathode material comprises a lithium nickel manganese cobalt oxide (e.g., NMC111, NMC532, NMC622, NMC811, NMC900505, NMC95025025, etc.), a lithium nickel cobalt aluminum oxide (NCA), a lithium nickel manganese aluminum oxide (NMA), a lithium nickel manganese cobalt aluminum oxide (NMCA), a lithium nickel oxide (LNO), a lithium nickel manganese oxide (NM), a lithium cobalt ocide (LCO), a lithium manganese oxide (LMO), a lithium and manganese rich cathode (LMR or LLMO), a lithium iron phosphate (LFP), a lithium cobalt phosphate (LCP), a lithium manganese phosphate (LMP), a lithium manganese iron phosphate (LMFP), a transition metal sulfide (e.g., FeS, FeS₂, CuS, MoS₂, MoS₃, TiS₂, TiS₄, etc.), or any mixture combination of above cathode materials.

1-16. (canceled)

17: An electrolyte comprising a compound, wherein the compound is
(i) a fluorinated derivative of 1,2-diethoxyethane, wherein one or more of the hydrogen atoms of the 1,2-diethoxyethane are substituted by fluorine; or
(ii) a fluorinated derivative of 1,1-diethoxymethane, wherein one or more of the hydrogen atoms of the 1,1-diethoxymethane are substituted by fluorine; or
(iii) a fluorinated derivative of 1,3-diethoxypropane, wherein one or more of the hydrogen atoms of the 1,3-diethoxypropane are substituted by fluorine; or
(iv) a fluorinated derivative of dimethyl carbonate, ethyl methyl carbonate, or diethyl carbonate, wherein one or more of the hydrogen atoms of the dimethyl carbonate, ethyl methyl carbonate, or diethyl carbonate are substituted by fluorine; or
(v) a 1,2-dialkoxy ethane compound, wherein each alkoxy group contains between two and four carbon atoms; or
(vi) a compound selected from the group consisting of selected from the group consisting of

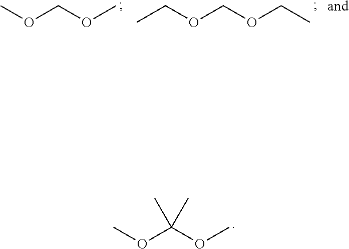

18: The electrolyte of claim 17, wherein the compound is selected from the group consisting of

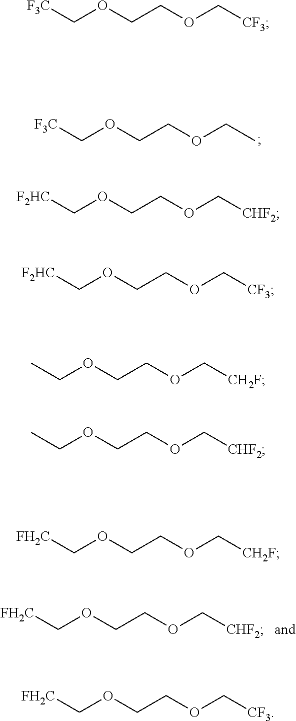

19: The electrolyte of claim 17, wherein the compound is selected from the group consisting of

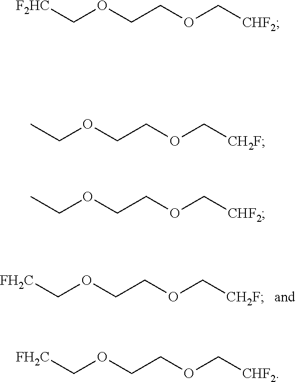

20: The electrolyte of claim 17, wherein the compound is selected from the group consisting of

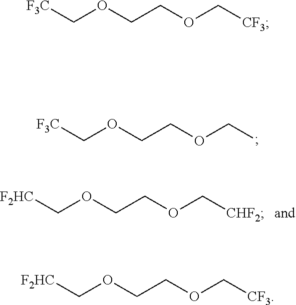

21: The electrolyte of claim 17, wherein the compound is selected from the group consisting of

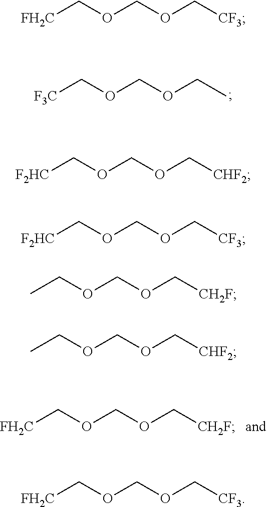

22: The electrolyte of claim 17, wherein the compound is selected from the group consisting of

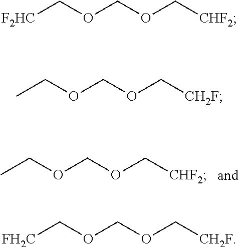

23: The electrolyte of claim 17, wherein the compound is selected from the group consisting of

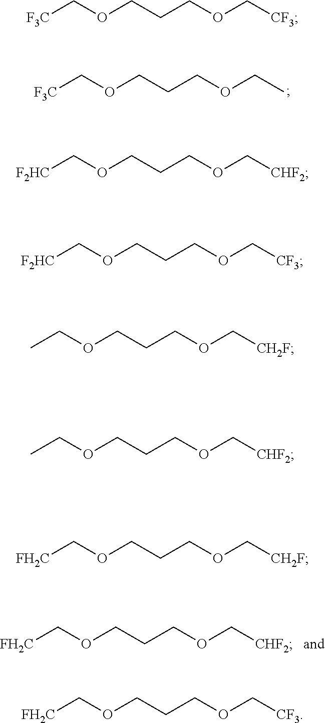

24: The electrolyte of claim 17, wherein the compound is selected from the group consisting of

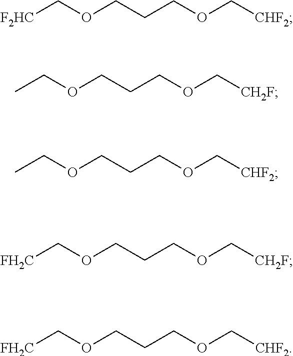

25: The electrolyte of claim 17, wherein the compound is selected from the group consisting of

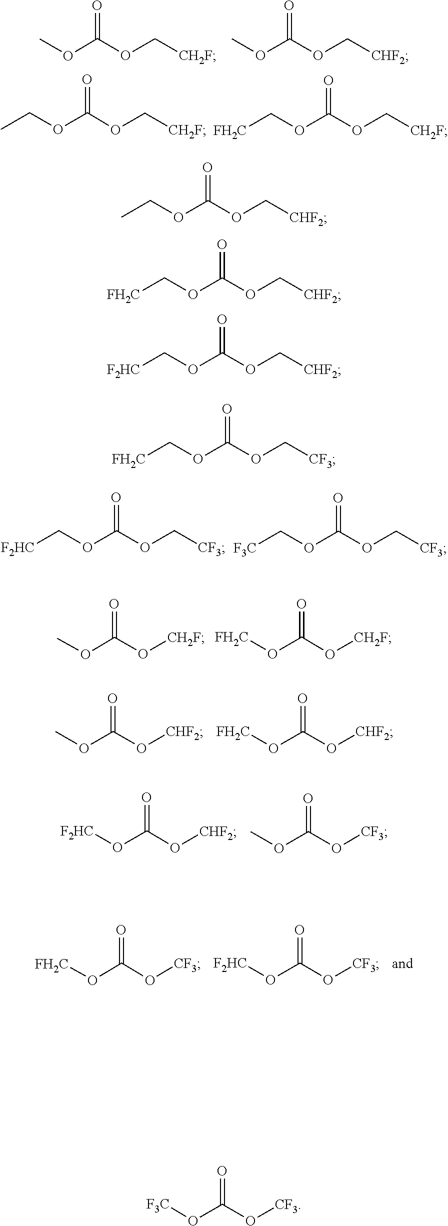

26: The electrolyte of claim 17, wherein the compound is selected from the group consisting of

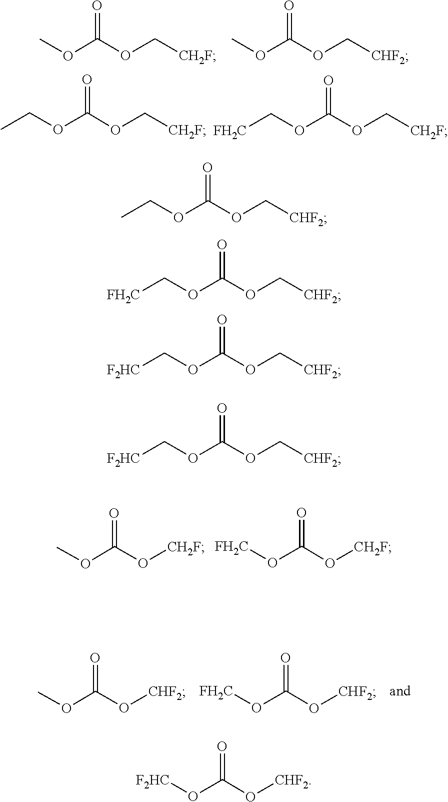

27: The electrolyte of claim 17, wherein the compound is selected from the group consisting of

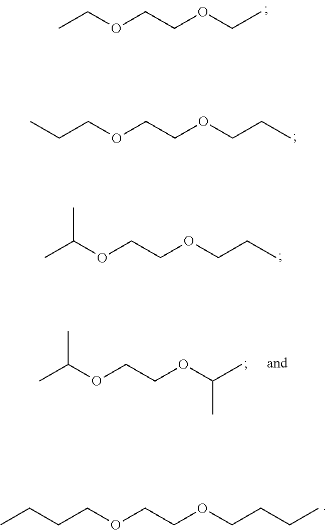

28: The electrolyte of claim 17, wherein the compound is selected from the group consisting of

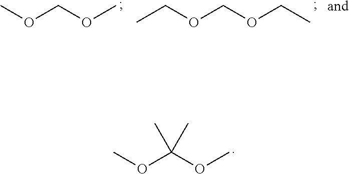

29: The electrolyte of claim 17, wherein the electrolyte comprises one or more additional components selected from the compounds recited in claim 17.

30: The electrolyte of claim 17, wherein the electrolyte comprises a solvent component that is not a compound recited in claim 17.

31: The electrolyte of claim 30, wherein the solvent component is selected from the group consisting of ethylene carbonate (EC), propylene carbonate (PC), dimethyl carbonate (DMC), diethyl carbonate (DEC), ethyl methyl carbonate (EMC), vinyl carbonate (VC), fluoroethylene carbonate (FEC), difluoroethylene carbonate (DFEC), 3,3,3-trifluoropropylene carbonate (TFPC), trifluoroethyl methyl carbonate (FEMC), bis(2,2,2-trifluoroethyl) carbonate (TFEC), 1,2-dimethyoxylethane (DME), 1, 3-di oxolane (DOL), 1,4-di oxane (DOX), tetrahydrofuran (THF), 1,3,2-dioxathiolane-2,2-dioxide (DTD), 1,3-propanesultone (PS), acetonitrile (AN), ethyl acetate (EA), methyl acetate (MA), methyl propanoate (MP), succinonitrile (SN), trimethyl phosphate (TMP), triethyl phosphate (TEP); tris(trimethylsilyl)phosphate (TTSP), tris(2,2,2-trifluoroethyl) phosphate (TFEPa), tris(2,2,2-trifluoroethyl) phosphite (TFEPi), prop-1-ene-1,3-sultone (PES), ethylene sulfite (ES), 1,4-butane sultone (BS), dimethyl sulfoxide (DMSO), methylene methanedisulfonate (MMDS), N,N-Dimethylformamide (DMF), and gamma-butyrolactone (BL).

32: The electrolyte of claim 30, wherein the amount of the solvent component in the electrolyte is between about 5 wt. % and about 99 wt. %.

33: The electrolyte of claim 17, wherein the electrolyte comprises one or more salts.

34: The electrolyte of claim 33, wherein the salt is selected from the group consisting of a lithium salt, a potassium salt, a sodium salt, and mixtures of any of the foregoing.

35: The electrolyte of claim 33, wherein the salt is selected from the group consisting of bis(fluorosulfonyl)imide (LiFSI); lithium bis(trifluoromethanesulfonyl)imide (LiTFSI); lithium hexafluorophosphate (LiPF6); lithium hexafluoroarsenate (LiAsF6); lithium tetrafluoroborate (LiBF4); lithium bis(oxalato)borate (LiBOB); lithium difluoro(oxalato)borate (LiDFOB); lithium difluorophosphate (LiDFP); lithium nitrate (LiNO3); lithium perchlorate (LiClO4); lithium triflate (LiTf); lithium trifluoroacetate (LiTFA); lithium 4,5-dicyano-2-(trifluoromethyl)imidazole (LiTDI); sodium bis(fluorosulfonyl)imide (NaFSI); sodium bis(trifluoromethanesulfonyl)imide (NaTFSI); potassium bis(fluorosulfonyl)imide (KFSI); and potassium bis(trifluoromethanesulfonyl)imide (KTFSI), and mixtures of any of the foregoing.

36: An electrochemical cell comprising:
an anode;
a cathode; and
the electrolyte of claim 17.

37: The electrochemical cell of claim 36, wherein the electrochemical cell is a battery.

38: The electrochemical cell of claim 36, wherein the anode comprises lithium.

39: The electrochemical cell of claim 36, wherein the anode comprises lithium metal.

40: The electrochemical cell of claim 36, wherein the anode comprises a surface protection layer comprising fluorine.

41: The electrochemical cell of claim 36, wherein the anode comprises a material selected from the group consisting of lithium metal, graphite, silicon, silicon oxide (SiOₓ), graphite/silicon composite, graphite/silicon oxide (SiOₓ) composite, graphite/silicon nitride (Si₃N₄) composite, graphite/silicon carbide (SiC) composite, and mixtures of any of the foregoing.

42: The electrochemical cell of claim 36, wherein the cathode comprises the cathode material comprises a sulfur-based cathode or an air cathode (e.g., a Li—S, Li-SPAN, or a Li-air battery), a lithium nickel manganese cobalt oxide (e.g., NMC111, NMC532, NMC622, NMC811, NMC900505, NMC95025025, etc.), a lithium nickel cobalt aluminum oxide (NCA), a lithium nickel manganese aluminum oxide (NMA), a lithium nickel manganese cobalt aluminum oxide (NMCA), a lithium nickel manganese oxide (NM), a lithium nickel oxide (LNO), a lithium nickel manganese oxide (LiNi_{0.5}Mn_{1.5}O₄, LNMO), a lithium cobalt oxide (LCO), a lithium manganese oxide (LMO), a lithium and manganese rich cathode (LMR or LLMO), a lithium iron phosphate (LFP), a lithium cobalt phosphate (LCP), a lithium manganese phosphate (LMP), a lithium manganese iron phosphate (LMFP), a transition metal sulfide (e.g., FeS, FeS₂, CuS, MoS₂, MoS₃, TiS₂, TiS₄, etc.), and mixtures of any of the foregoing.

43: A compound selected from the group consisting of 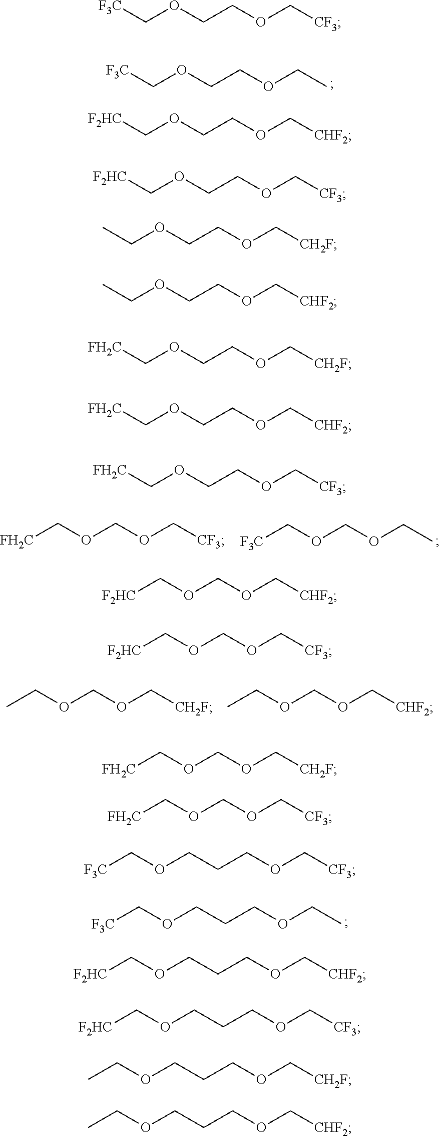 [chemical structure]
